(19) United States
(12) Patent Application Publication
(10) Pub. No.: US 2021/0260182 A1
(43) Pub. Date: Aug. 26, 2021

(54) RECOMBINANT POXVIRUS BASED VACCINE AGAINST SARS-CoV-2 VIRUS

(71) Applicants: Scott J. Goebel, Frederick, MD (US); David Evans, Edmonton (CA); Ryan Noyce, Edmonton (CA); Tonix Pharmaceuticals Holding Corp., Chatham, NJ (US); The Governors of the University of Alberta, Edmonton (CA)

(72) Inventors: Seth Lederman, South Dartmouth, MA (US); Scott J. Goebel, Frederick, MD (US); David Evans, Edmonton (CA); Ryan Noyce, Edmonton (CA)

(21) Appl. No.: 17/187,678

(22) Filed: Feb. 26, 2021

Related U.S. Application Data
(60) Provisional application No. 63/114,514, filed on Nov. 16, 2020.
(60) Provisional application No. 62/981,997, filed on Feb. 26, 2020.

(51) Int. Cl. A61K 39/215; C12N 7/00; C12N 15/85

(52) U.S. Cl. CPC A61K 39/215; C12N 7/00; A61K 2039/70; C12N 2710/24134; C12N 15/85

(57) ABSTRACT

The invention relates in various aspects to a recombinant poxvirus comprising a nucleic acid encoding a SARS-CoV-2 virus protein, methods for producing such viruses and the use of such viruses. The recombinant poxviruses are well suited, among others, as protective virus vaccines against SARS-CoV-2 virus.

### CROSS-REFERENCE TO RELATED APPLICATIONS

This application claims priority and benefit from U.S. Provisional Application No. 62/981,997, filed Feb. 26, 2020 and U.S. Provisional Application No. 63/114,514, filed Nov. 16, 2020, the contents of which are hereby incorporated by reference in its entirety.

### SEQUENCE LISTING

The instant application contains a Sequence Listing which has been submitted electronically in ASCII format and is hereby incorporated by reference in its entirety. Said ASCII copy, created on Feb. 26, 2021, is named 104545-0047-101-SL.txt and is 766,062 bytes in size.

### BACKGROUND OF THE DISCLOSURE

On Dec. 31, 2019 the Wuhan Health Commission reported a cluster of atypical pneumonia cases in the city of Wuhan, China. The first patients began experiencing symptoms of illness in mid-December 2019. Clinical isolates were found to contain a novel coronavirus. As of Jan. 28, 2020, there are in excess of 4,500 laboratory-confirmed cases, with >100 known deaths. The novel coronavirus is currently referred to as SARS-CoV-2 or 2019-nCoV and is related to Severe Acute Respiratory Syndrome coronavirus (SARS-CoV), although with only approximately 80% similarity at the nucleotide level. Ralph et al. J Infect Dev Ctries. 2020 Jan. 31; 14(1):3-17.

Coronaviruses are enveloped single stranded RNA viruses with positive-sense RNA genomes ranging from 25.5 to ˜32 kb in length. The spherical virus particles range from 70-120 nm in diameter with four structural proteins.

Despite the fact that a much effort is currently being invested into methods of providing vaccines and delivery vectors for SARS-CoV-2, there is still a need to provide additional and improved approaches against this coronavirus.

### SUMMARY OF THE DISCLOSURE

An aspect of the present disclosure provides a recombinant poxvirus comprising a nucleic acid encoding a SARS-CoV-2 virus protein, methods for producing such viruses and the use of such viruses, for example, as immunogens, in immunogenic formulations against SARS-CoV-2 virus. Another aspect of the present disclosure provides a recombinant synthetic poxvirus comprising a nucleic acid encoding a SARS-CoV-2 virus protein, methods for producing such viruses and the use of such viruses, for example, as immunogens, in immunogenic formulations against SARS-CoV-2 virus. In some embodiments, the synthetic poxviruses are assembled and replicated from chemically synthesized DNA which are safe, reproducible and free of contaminants. Because chemical genome synthesis is not dependent on a natural template, a plethora of structural and functional modifications of the viral genome are possible. Chemical genome synthesis is particularly useful when a natural template is not available for genetic replication or modification by conventional molecular biology methods.

In one aspect, the disclosure relates to recombinant poxviruses comprising a nucleic acid encoding a SARS-CoV-2 virus protein, wherein the SARS-CoV-2 protein is selected from the group consisting of the spike protein (S), the membrane protein (M) and the nucleocapsid protein (N), or combinations of two or more of said proteins.

In another aspect, the disclosure relates to pharmaceutical compositions comprising the recombinant poxviruses of the disclosure.

In another aspect, the disclosure relates to cells infected with the recombinant poxviruses of the disclosure.

In another aspect, the disclosure relates to methods for selecting a cell that expresses a SARS-CoV-2 virus protein, comprising infecting said cell with the recombinant poxvirus of the disclosure and selecting the infected cell expressing said SARS-CoV-2 virus protein.

In another aspect, the disclosure relates to methods of inducing an immune response against a SARS-CoV-2 virus in a subject in need or at risk therefor, comprising administering to said subject an immunologically effective amount of a recombinant poxvirus of the disclosure.

In another aspect, the disclosure relates to methods of generating the recombinant poxviruses of the disclosure, the methods comprising: (a) infecting a host cell with a poxvirus; (b) transfecting the infected cell of step (a) with a nucleic acid encoding a SARS-CoV-2 virus protein to generate a recombinant poxvirus; and (c) selecting a recombinant poxvirus, wherein the nucleic acid encoding a SARS-CoV-2 virus protein is located, upon transfection, in a region of the poxvirus that is not essential for the replication of the poxvirus.

### BRIEF DESCRIPTION OF THE DRAWINGS

For the purpose of illustrating the disclosure that are shown in the drawings and various embodiment(s) of this disclosure. It should be understood, however, that the disclosure is not limited to the precise arrangements and instrumentalities shown in the drawings.

FIG. 1. Schematic representation of the linear dsDNA synthetic HPXV (GenBank accession Number KY349117) and synthetic VACV (synVACV) (GenBank accession Number MN974381) genomes. The Thymidine Kinase (TK) gene locus is depicted in orange. The TK gene locus in HPXV is located at genome positions: 92077-92610 with gene ID HPXV095 (SEQ ID NO: 1). The TK gene locus in VACV is located at genome positions: 83823-84344 with gene ID synVACV_105 (SEQ ID NO: 2).

FIG. 2. Schematic representation of the TK gene locus (HPXV095) of HPXV of approximately 4 kb, located between the HPXV094 and HPXV096 flanking regions.

FIG. 3. Sequence alignment of the TK gene locus of synthetic HPXV and synthetic VACV ACAM2000, where it is shown that the nucleotide similarity is around 99%. FIG. 3 refers to SEQ ID NOs: 34-36, respectively, in order of appearance.

FIG. 4. Schematic representation of the linear dsDNA HPXV, showing the generation of the PCR fragment encoding the SARS-CoV-2 expression cassette. The expression cassette is introduced in the TK gene locus of the HPXV genome and comprises the SARS-CoV2 Spike S gene that is operatively linked to a vaccinia virus early and late promoter inserted upstream of the SARS-CoV-2 Spike S gene.

FIG. 5. Schematic representation of the HPXV and VACV, ACAM 2000 rescue viruses and the insertion of the synthesized expression cassette encoding the SARS-CoV-2 Spike S protein by recombination with the left and right recombination flanking arms.

FIG. 6. Schematic representation of the method of generating a recombinant HPXV, which comprises (1) infection of BSC-40 cells with the HPXV expressing yfpgpt cassette in the HPXV095 locus; (2) transfection of the infected cells with the synthesized Expression Cassette 24 hours post infection; (3) Harvest the cell lysate, release progeny virus of HPXV and recombinant HPXV expressing SARS-CoV-2 Spike S protein (rHPXV-SARS S) with repeated cycles rounds of freeze/thaw 48 hours post infection/transfection and (4) selection of cells comprising the rHPXV-SARS S.

FIG. 7. Schematic representation of the selection and purification of a recombinant HPXV comprising SARS-CoV-2 S protein, which comprises (1) previous steps of infection/transfection; (2) the harvest and cell lysis of the cells to release the control HPXV and the rHPXV-SARS S progeny; (3) plate titrations of progeny virus on BSC-40 cells; and (4) look for non-fluorescent plaques with a fluorescent microscope. Virus progeny that have replaced the yfpgpt cassette with SARS-CoV-2 S are non-fluorescent.

FIG. 8. Early, late and overlapping early/late Vaccinia Virus promoters. Core, spacer and initiator (init) are shown. Panel A shows the Early promoter nucleotide sequence (SEQ ID NO: 3); specific nucleotides required for optimal expression are indicated using the 4-base code; noncritical nucleotides are indicated by N; a purine must be present within the init region. Panel B shows the Late promoter nucleotide sequence (SEQ ID NO: 4); the T-run and TAAAT init sequence provide high expression. Panel B shows the synthetic Early/Late promoter nucleotide sequence (SEQ ID NO: 5); the elements of the early and late promoter are indicated above and below the sequence, respectively.

FIG. 9. Nucleotide sequence of variations of the overlapping early/late Vaccinia Virus promoters, comprising different spacers 3′ of the late promoter. Panel A shows a 38-nucleotides spacer (SEQ ID NO: 40; full-length sequence of promoter and spacer recited in SEQ ID NO: 37); Panel B shows a 99-nucleotides spacer (SEQ ID NO: 41; full-length sequence of promoter and spacer recited in SEQ ID NO: 38) and Panel C shows a 160-nucleotides spacer (SEQ ID NO: 42; full-length sequence of promoter and spacer recited in SEQ ID NO: 39).

FIG. 10. Schematic representation of the method of generating a recombinant scHPXV or synVACV comprising a nucleic acid encoding a SARS-CoV-2 S protein, which comprises (1) infection of BSC-40 cells with the rescue HPXV or VACV virus and (2) transfection of the infected BSC-40 cells with a PCR-generated fragment in the TK gene locus, wherein the PCR-generated fragment comprises the engineered SARS-CoV-2 S gene expression cassette. The SARS-CoV-2 S gene contains one or more modifications (at least Y459H is present). The resulting modified S protein is adapted to infect mice. The vaccinia Early Transcription Terminator Signal ETTS (T₅NT (SEQ ID NO: 14)) are also removed from the SARS-CoV-2 S gene through coding silent mutagenesis to generate full length transcripts during the early phase of the infection.

FIG. 11. Western blot of SARS-CoV-2 Spike protein expression from BSC-40 cells infected with synVACVΔA2K105^{yfp-gpt} or synVACVΔA2K105^{SARSCoV2-SPIKE-co::nm} (TNX-2200) clones 1.1.1.1.1 or 2.1.1.1.1. “Mock” represents a negative control group with no virus. “Mr” is a set of molecular weight markers in kiloDaltons (kDa). The labels on the right identify various proteins: “S multimer”: the Spike multimer protein; “FL S-G”: the full length glycosylated spike protein; “FL S”: the full length spike protein; “VACV I3”: the single stranded DNA binding 13 protein (an internal control); “SPIKE-co::nm”: a spike protein that is codon optimized and has no marker, indicating there is no YFP-GPT expression.

FIG. 12. Western blot of Spike protein expression from BSC-40 cells infected with synthetic TNX-801, TNX-1800a-1, or TNX-1800b-2. “Mock” represents a negative control group with no virus. “kDa” is kiloDaltons (molecular weight). The labels on the right identify various proteins: “S multimer”: the Spike multimer protein; “FL S-G”: the full length glycosylated spike protein.; “FL S” the full length spike protein; “VACV I3”: the single stranded DNA binding 13 protein (an internal control).

FIG. 13. Schematic of day 7 cutaneous reactions (“takes”) in African Green Monkeys (AGM) vaccinated with a 2.9×10⁶ PFU TNX-801. Panel A shows a female AGM (Animal #: 1F 16986); Panel B shows a female AGM (Animal #: 1F 16994); Panel C shows a male AGM (Animal #: 1M 16975); and Panel D shows a male AGM (Animal #: 1M 16977).

FIG. 14. Schematic of day 7 cutaneous reaction (“takes”) in African Green Monkeys (AGM) vaccinated with 1.06×10⁶ PFU TNX-801. Panel A shows a female AGM (Animal #: 2F 16985); Panel B shows a female AGM (Animal #: 1F 16991); Panel C shows a male AGM (Animal #: 2M 16980); and Panel D shows a male AGM (Animal #: 1M 16983).

FIG. 15. Schematic of day 7 cutaneous reaction (“takes”) in African Green Monkeys (AGM) vaccinated with 2.9×10⁶ PFU TNX-1800b-2. Panel A shows a female AGM (Animal #: 3F 16988); Panel B shows a female AGM (Animal #: 3F 16995); Panel C shows a male AGM (Animal #: 3M 16976); and Panel D shows a male AGM (Animal #: 3M 16982).

FIG. 16. Schematic of day 7 cutaneous reaction (“takes”) in African Green Monkeys (AGM) vaccinated with 1.06×10⁶ PFU TNX-1800b-2. Panel A shows a female AGM (Animal #: 4F 16989); Panel B shows a female AGM (Animal #: 4F 16990); Panel C shows a male AGM (Animal #: 4M 16972); and Panel D shows a male AGM (Animal #: 4M 16973).

FIG. 17. Schematic of day 7 cutaneous reaction (“takes”) in African Green Monkeys (AGM) vaccinated with 0.6×10⁶ PFU TNX-1800a-1. Panel A shows a female AGM (Animal #: 5F 16992); Panel B shows a female AGM (Animal #: 5F 16993); Panel C shows a male AGM (Animal #: 5M 16979); and Panel D shows a male AGM (Animal #: 5M 16981).

FIG. 18. Stained plates showing cytopathic effects in BSC-40, HeLa and HEK 293 cells 48 hours after infection with TNX-801, TNX-1800b-2, TNX-1200, or TNX-2200.

FIGS. 19A, 19B, 19C and 19D. Viral growth curves in BSC-40, HeLa and HEK 293 cells over time. FIG. 19A shows cells infected with TNX-1200; FIG. 19B shows cells infected with TNX-2200; FIG. 19C shows cells infected with TNX-801; and FIG. 19D shows cells infected with TNX-1800b-2.

FIGS. 20A and 20B. Viral growth curves in BSC-40 cells infected with a synthetic horsepox virus (HPXV) over time. FIG. 20A shows viral titer (PFU/mL) measured in cells infected with TNX-801, scHPXVΔ095^{yfp-gpt}, TNX-1800a-1, scHPXVΔ200^{yfp-gpt}, or TNX-1800b-2; FIG. 20B shows fold change from input in infected cells.

FIGS. 21A and 21B. Viral growth curves in BSC-40 cells infected with a synthetic vaccinia virus (VACV) over time. FIG. 21A shows viral titer (PFU/mL) measured in cells infected with TNX-1200, TNX-2200 or synVACVΔA2K105^{yfp-gpt}; FIG. 21B shows fold change from input in infected cells.

FIG. 22. Schematic representation of a linear dsDNA HPXV, showing the generation of a PCR fragment encoding a SARS-CoV-2 expression cassette. The expression cassette is introduced into the TK gene locus of the HPXV genome and comprises a DNA encoding the SARS-CoV2 Spike S gene protein that is operatively linked to a vaccinia virus early and late promoter inserted upstream of the SARS-CoV-2 Spike S DNA. The expression cassette further comprises a 1 kb HPXV left flanking arm (e.g., HPXV092, HPXV093 and HPXV094) and a 1 kb HPXV right flanking arm (e.g., HPXV096).

### DETAILED DESCRIPTION OF THE DISCLOSURE

### General Techniques

Unless otherwise defined herein, scientific and technical terms used in this application shall have the meanings that are commonly understood by those of ordinary skill in the art. Generally, nomenclature used in connection with, and techniques of, pharmacology, cell and tissue culture, molecular biology, cell and cancer biology, neurobiology, neurochemistry, virology, immunology, microbiology, genetics and protein and nucleic acid chemistry, described herein, are those well-known and commonly used in the art. In case of conflict, the present specification, including definitions, will control.

The practice of the present disclosure will employ, unless otherwise indicated, conventional techniques of molecular biology (including recombinant techniques), microbiology, cell biology, biochemistry, virology and immunology, which are within the skill of the art. Such techniques are explained fully in the literature, such as Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Sambrook and Russell, Molecular Cloning: A Laboratory Manual, 3rd. ed., Cold Spring Harbor Laboratory Press, Cold Spring Harbor, N.Y. (2001); Ausubel et al., Current Protocols in Molecular Biology, John Wiley & Sons, N Y (2002); Harlow and Lane Using Antibodies: A Laboratory Manual, Cold Spring Harbor Laboratory Press, Cold Spring Harbor, N.Y. (1998); Coligan et al., Short Protocols in Protein Science, John Wiley & Sons, N Y (2003); Short Protocols in Molecular Biology (Wiley and Sons, 1999).

Enzymatic reactions and purification techniques are performed according to manufacturer's specifications, as commonly accomplished in the art or as described herein. The nomenclatures used in connection with, and the laboratory procedures and techniques of, analytical chemistry, biochemistry, immunology, molecular biology, synthetic organic chemistry, and medicinal and pharmaceutical chemistry described herein are those well-known and commonly used in the art. Standard techniques are used for chemical syntheses, and chemical analyses.

Throughout this specification and embodiments, the word “comprise,” or variations such as “comprises” or “comprising,” will be understood to imply the inclusion of a stated integer or group of integers but not the exclusion of any other integer or group of integers.

The term “including” is used to mean “including but not limited to.” “Including” and “including but not limited to” are used interchangeably.

Any example(s) following the term “e.g.” or “for example” is not meant to be exhaustive or limiting.

Unless otherwise required by context, singular terms shall include pluralities and plural terms shall include the singular.

The articles “a”, “an” and “the” are used herein to refer to one or to more than one (i.e., to at least one) of the grammatical object of the article. By way of example, “an element” means one element or more than one element. Reference to “about” a value or parameter herein includes (and describes) embodiments that are directed to that value or parameter per se. For example, description referring to “about X” includes description of “X.” Numeric ranges are inclusive of the numbers defining the range.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g., 1 to 6.1, and ending with a maximum value of 10 or less, e.g., 5.5 to 10.

Exemplary methods and materials are described herein, although methods and materials similar or equivalent to those described herein can also be used in the practice or testing of the present application. The materials, methods, and examples are illustrative only and not intended to be limiting.

### Definitions

The following terms, unless otherwise indicated, shall be understood to have the following meanings:

The terms “chimeric” or “engineered” or “modified” (e.g., chimeric poxvirus, engineered polypeptide, modified polypeptide, engineered nucleic acid, modified nucleic acid) or grammatical variations thereof are used interchangeably herein to refer to a non-native sequence that has been manipulated to have one or more changes relative a native sequence.

As used herein, the term “essential gene for replication” or “essential region for replication” refers to those gene(s) or region(s) indispensable for the replication of an organism, and therefore are considered a foundation of life. In the context of a virus, a gene or region is considered essential (i.e. has a role in cell culture) if its deletion results in a decrease in virus titer of greater than 10-fold in either a single or multiple step growth curve. Most of the essential genes are thought to encode proteins that maintain a central metabolism, replicate DNA, translate genes into proteins, maintain a basic cellular structure, and mediate transport processes into and out of the cell. Genes involved in virion production, actin tail formation, and extracellular virion release are typically also considered as essential. Two main strategies have been employed to identify essential genes on a genome-wide basis: directed deletion of genes and random mutagenesis using transposons. In the first case, individual genes (or ORFs) are completely deleted from the genome in a systematic way. In random mutagenesis, transposons are randomly inserted in as many positions in a genome as possible, aiming to inactivate the targeted genes. Insertion mutants that are still able to survive or grow are not in essential genes. (Zhang, R., 2009 & Gerdes, S., 2006).

The term “expression cassette” or “transcription unit”, as used herein, defines a nucleic acid sequence region that contains one or more genes to be transcribed. The nucleotide sequences encoding the to be transcribed gene(s), as well as the polynucleotide sequences containing the regulatory elements contained within an expression cassette, are operably linked to each other. The genes are transcribed from a promoter and transcription is terminated by at least one polyadenylation signal. In some embodiments, each of the one or more genes are transcribed from one promoter. In some embodiments, the one or more genes are transcribed from one single promoter. In that case, the different genes are at least transcriptionally linked. More than one protein or product can be transcribed and expressed from each transcription unit (multicistronic transcription unit). Each transcription unit will comprise the regulatory elements necessary for the transcription and translation of any of the selected sequences that are contained within the unit. Each transcription unit may contain the same or different regulatory elements.

“Homologous,” in all its grammatical forms and spelling variations, refers to the relationship between two proteins that possess a “common evolutionary origin,” including proteins from superfamilies in the same species of organism, as well as homologous proteins from different species of organism. Such proteins (and their encoding nucleic acids) have sequence homology, as reflected by their sequence similarity, whether in terms of percent identity or by the presence of specific residues or motifs and conserved positions. “Homologous” may also refer to a nucleic acid which is native to the virus.

In common usage and in the instant application, the term “homologous,” when modified with an adverb such as “highly,” may refer to sequence similarity and may or may not relate to a common evolutionary origin.

“Heterologous,” in all its grammatical forms and spelling variations, may refer to a nucleic acid which is non-native to the virus. It means derived from a different species or a different strain than the nucleic acid of the organism to which the nucleic acid is described as being heterologous relative to. In a non-limiting example, the viral genome of the synVACV comprises heterologous terminal hairpin loops. Those heterologous terminal hairpin loops can be derived from a different viral species or from a different VACV strain.

As used herein, a “host cell” includes an individual cell or cell culture that can be or has been a recipient for the virus of the disclosure. Host cells include progeny of a single host cell, and the progeny may not necessarily be completely identical (in morphology or in genomic DNA complement) to the original parent cell due to natural, accidental, or deliberate mutation. A host cell includes cells transfected and/or transformed in vivo with a poxvirus of this disclosure.

An “immunologically effective amount” refers to the amount to be administered of a composition of matter that comprises at least one antigen, or immunogenic portion thereof, which is able to elicit an immunological response in the host cell or an antibody-mediated immune response to the composition. An immunologically effective amount of a recombinant poxvirus, as disclosed herein, refers to the amount of poxviral particles necessary to deliver a SARS-CoV-2 virus protein and elicit an immune response against said SARS-CoV-2 virus protein. In some embodiments, an immunologically effective amount of the recombinant poxvirus of the present disclosure is an amount within the range of 10²-10⁹ PFU. In some embodiments, an immunologically effective amount of the recombinant poxvirus of the present disclosure is from about 10³-10⁵ PFU. In some embodiments, an immunologically effective amount of the recombinant poxvirus of the present disclosure is about 10⁵ PFU.

The terms “operative linkage” and “operatively linked” (or “operably linked”) or variations thereof, as used herein, are used interchangeably with reference to a juxtaposition of two or more components (such as sequence elements), in which the components are arranged such that both components function normally and allow the possibility that at least one of the components can mediate a function that is exerted upon at least one of the other components. By way of illustration, the nucleic acid encoding a SARS-CoV-2 virus protein may be operatively linked to a promoter. The nucleic acid sequence encoding a SARS-CoV-2 virus protein may be operatively linked in cis with a poxvirus specific promoter nucleic acid sequence, but does not need to be directly adjacent to it. For example, a linker sequence can be located between both sequences.

As used herein, the phrase “multiplicity of infection” or “MOI” is the average number of viruses per infected cell. The MOI is determined by dividing the number of virus added (ml added×plaque forming units (PFU)) by the number of cells added (ml added×cells/ml).

The terms “patient”, “subject”, or “individual” are used interchangeably herein and refer to either a human or a non-human animal. These terms include mammals, such as humans, primates, livestock animals (including bovines, porcines, camels, etc.), companion animals (e.g., canines, felines, etc.) and rodents (e.g., mice and rats).

As known in the art, “polynucleotide,” or “nucleic acid,” as used interchangeably herein, refer to chains of nucleotides of any length, and include DNA and RNA. The nucleotides can be deoxyribonucleotides, ribonucleotides, modified nucleotides or bases, and/or their analogs, or any substrate that can be incorporated into a chain by DNA or RNA polymerase. A polynucleotide may comprise modified nucleotides, such as methylated nucleotides and their analogs. If present, modification to the nucleotide structure may be imparted before or after assembly of the chain. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog; internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.); those containing pendant moieties, such as, for example, proteins (e.g., nucleases, toxins, antibodies, signal peptides, poly-L-lysine, etc.); those with intercalators (e.g., acridine, psoralen, etc.); those containing chelators (e.g., metals, radioactive metals, boron, oxidative metals, etc.); those containing alkylators; those with modified linkages (e.g., alpha anomeric nucleic acids, etc.); as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, alpha- or beta-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S(“thioate”), P(S)S (“dithioate”), (O)NR₂ (“amidate”), P(O)R, P(O)OR′, CO or CH₂ (“formacetal”), in which each R or R′ is independently H or substituted or unsubstituted alkyl (1-20 C) optionally containing an ether and (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

The terms “polypeptide”, “oligopeptide”, “peptide” and “protein” are used interchangeably herein to refer to chains of amino acids of any length. The chain may be linear or branched, it may comprise modified amino acids, and/or may be interrupted by non-amino acids. The terms also encompass an amino acid chain that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that the polypeptides can occur as single chains or associated chains.

“Percent (%) sequence identity” or “sequence % identical to” with respect to a reference polypeptide (or nucleotide) sequence is defined as the percentage of amino acid residues (or nucleic acids) in a candidate sequence that are identical with the amino acid residues (or nucleic acids) in the reference polypeptide (nucleotide) sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

As outlined elsewhere herein, certain positions of the viral genome can be altered. By “position” as used herein is meant a location in the genome sequence. Corresponding positions are generally determined through alignment with other parent sequences.

As used herein, “purify,” and grammatical variations thereof, refers to the removal, whether completely or partially, of at least one impurity from a mixture containing the polypeptide and one or more impurities, which thereby improves the level of purity of the polypeptide in the composition (i.e., by decreasing the amount (ppm) of impurity(ies) in the composition). As used herein “purified” in the context of viruses refers to a virus which is substantially free of cellular material and culture media from the cell or tissue source from which the virus is derived. The language “substantially free of cellular material” includes preparations of virus in which the virus is separated from cellular components of the cells from which it is isolated or recombinantly produced. Thus, a virus that is substantially free of cellular material includes preparations of protein having less than about 30%, 20%, 10%, or 5% (by dry weight) of cellular protein (also referred to herein as a “contaminating protein”). The virus may also be substantially free of culture medium, i.e., culture medium represents less than about 20%, 10%, or 5% of the volume of the virus preparation. A virus can be “purified” using routine methods known to one of skill in the art including, but not limited to, chromatography and centrifugation.

As used herein, the term “recombinant poxvirus” refers to a poxvirus comprising an exogenous or heterologous sequence in its genome generated by artificial manipulation of the viral genome, i.e. generation by recombinant DNA technology. The recombinant poxvirus contains an exogenous polynucleotide sequence encoding a polypeptide of interest. In some embodiments, the recombinant poxvirus comprises a nucleic acid encoding a SARS-CoV-2 virus protein.

As used herein, the term “rescue poxvirus” or “rescue virus” or “rescue system” refers to a virus or system which relies on a helper virus to provide the machinery necessary to produce recombinant viruses, by assembling the fragmented genome, while simultaneously integrating the targeted gene or expression cassette. Rice et al. Viruses. 2011 March; 3(3): 217-232.

As used herein, the term “residue” in the context of a polypeptide refers to an amino-acid unit in the linear polypeptide chain. It is what remains of each amino acid, i.e. —NH—CHR—C—, after water is removed in the formation of the polypeptide from α-amino-acids, i.e. NH2-CHR—COOH.

The term “sequence similarity,” in all its grammatical forms, refers to the degree of identity or correspondence between nucleic acid or amino acid sequences that may or may not share a common evolutionary origin.

As used herein, “synthetic virus” refers to a virus initially derived from synthetic DNA (e.g., chemically synthesized DNA, PCR amplified DNA, engineered DNA, polynucleotides comprising nucleoside analogs, etc., or combinations thereof) and includes its progeny, and the progeny may not necessarily be completely identical (in morphology or in genomic DNA complement) to the original parent synthetic virus due to natural, accidental, or deliberate mutation. In some embodiments, the synthetic virus refers to a virus where substantially all of the viral genome is initially derived from synthetic DNA (e.g., chemically synthesized DNA, PCR amplified DNA, engineered DNA, polynucleotides comprising nucleoside analogs, etc., or combinations thereof). In a preferred embodiment, the synthetic virus is derived from chemically synthesized DNA.

As used herein, “substantially pure” refers to material which is at least 50% pure (i.e., free from contaminants), more preferably, at least 90% pure, more preferably, at least 95% pure, yet more preferably, at least 98% pure, and most preferably, at least 99% pure.

The term “vaccine”, as used herein, refers to a composition comprising at least one immunologically active component that induces an immunological response in an animal and possibly, but not necessarily, one or more additional components that enhance the immunological activity of the active component. A vaccine may additionally comprise further components typical to pharmaceutical compositions. The immunologically active component of a vaccine may comprise complete virus particles in either their original form or as attenuated particles (modified live vaccine), or particles inactivated by appropriate methods (killed or inactivated vaccine). In other embodiments, the immunologically active component of a vaccine may comprise appropriate elements of the organisms (subunit vaccines) that best stimulate the immune system. The immunologically active component may be a protein of the viral envelope. The immunologically active component may be a protein forming part of the nucleocapsid. In some embodiments, the immunologically active component of a vaccine against SARS-CoV-2 is an envelope protein. Non-limiting examples of such proteins are the Spike protein (S), the Membrane protein (M) and the Hemagglutinin-Esterase protein (HE). In some embodiments, the immunologically active component of a vaccine against SARS-CoV-2 is the nucleocapsid protein (N).

The term “viral vector”, as used herein, describes a genetically modified virus which was manipulated by a recombinant DNA technique in a way so that its entry into a host cell is capable of resulting in a specific biological activity, e.g. the expression of a foreign target gene carried by the vector. A viral vector may or may not be replication competent in the target cell, tissue, or organism. A viral vector can incorporate sequences from the genome of any known organism. The sequences can be incorporated in their native form or can be modified in any way to obtain a desired activity. For example, the sequences can comprise insertions, deletions or substitutions. A viral vector can also incorporate an insertion site for an exogenous polynucleotide sequence. In some embodiments, the viral vector is a poxvirus. In some embodiments, the viral vector is a horsepox viral vector. In some embodiments, the viral vector is a synthetic horsepox viral vector.

As used herein, the terms “wild type virus”, “wild type genome”, “wild type protein,” or “wild type nucleic acid” refer to a sequence of amino or nucleic acids that occurs naturally within a certain population (e.g., a particular viral species, etc.).

Each embodiment described herein may be used individually or in combination with any other embodiment described herein.

### Overview

Poxviruses are large (˜200 kbp) DNA viruses that replicate in the cytoplasm of infected cells. The Orthopoxvirus (OPV) genus comprises a number of poxviruses that vary greatly in their ability to infect different hosts. Vaccinia virus (VACV), for example, can infect a broad group of hosts, whereas variola virus (VARV), the causative agent of smallpox, only infects humans. A feature common to many, if not all poxviruses, is their ability to non-genetically “reactivate” within a host. Non-genetic reactivation refers to a process wherein cells infected by one poxvirus can promote the recovery of a second “dead” virus (for example one inactivated by heat) that would be non-infectious on its own.

Purified poxvirus DNA is not infectious because the virus life cycle requires transcription of early genes via the virus-encoded RNA polymerases that are packaged in virions. However, this deficiency can be overcome if virus DNA is transfected into cells previously or subsequently infected with a helper poxvirus, providing the necessary factors needed to transcribe, replicate, and package the transfected genome in trans (Sam C K, Dumbell K R. Expression of poxvirus DNA in coinfected cells and marker rescue of thermosensitive mutants by subgenomic fragments of DNA. Ann Virol (Inst Past). 1981; 132:135-50). Although this produces mixed viral progeny, a desired virus can be obtained by performing a reactivation reaction in a cell line that supports the propagation of both viruses, and then eliminating the helper virus by plating the mixture of viruses on cells that do not support the helper virus' growth (Scheiflinger F, Dorner F, Falkner F G. Construction of chimeric vaccinia viruses by molecular cloning and packaging. Proceedings of the National Academy of Sciences of the United States of America. 1992; 89(21):9977-81).

### Preparation of Poxviruses

Any of the synthetic poxviruses disclosed in US 2018/0251736 and WO 2019/213452, the entire disclosure of each is incorporated by reference herein, may be used in the present disclosure.

In one aspect, the present disclosure provides recombinant poxviruses comprising a nucleic acid encoding a SARS-CoV-2 virus protein, wherein the SARS-CoV-2 protein is selected from the group consisting of the spike protein (S), the membrane protein (M) and the nucleocapsid protein (N), or combinations of two or more of said proteins.

In some embodiments, the poxvirus belongs to the Chordopoxvirinae subfamily. In some embodiments, the poxvirus belongs to a genus of Chordopoxvirinae subfamily selected from Avipoxvirus, Capripoxvirus, Cervidpoxvirus, Crocodylipoxvirus, Leporipoxvirus, Molluscipoxvirus, Orthopoxvirus, Parapoxvirus, Suipoxvirus, or Yatapoxvirus. In some embodiments, the recombinant poxvirus is an Orthopoxvirus. In some embodiments, the Orthopoxvirus is selected from the group consisting of camelpox virus (CMLV), cowpox virus (CPXV), ectromelia virus (ECTV, “mousepox agent”), horsepox virus (HPXV), monkeypox virus (MPXV), rabbitpox virus (RPXV), raccoonpox virus, skunkpox virus, Taterapox virus, Uasin Gishu disease virus, vaccinia virus (VACV), variola virus (VARV) and volepox virus (VPV). In some embodiments, the poxvirus is a Parapoxvirus. In some embodiments, the Parapoxvirus is selected from orf virus (ORFV), pseudocowpox virus (PCPV), bovine popular stomatitis virus (BPSV), squirrel parapoxvirus (SPPV), red deer parapoxvirus, Ausdyk virus, Chamois contagious ecythema virus, reindeer parapoxvirus, or sealpox virus. In some embodiments, the poxvirus is a Molluscipoxvirus. In some embodiments, the Molluscipoxvirus is molluscum contagiousum virus (MCV). In some embodiments, the poxvirus is a Yatapoxvirus. In some embodiments, the Yatapoxvirus is selected from Tanapox virus or Yaba monkey tumor virus (YMTV). In some embodiments, the poxvirus is a Capripoxvirus. In some embodiments, the Capripoxvirus is selected from sheepox, goatpox, or lumpy skin disease virus. In some embodiments, the poxvirus is a Suipoxvirus. In some embodiments, the Suipoxvirus is swinepox virus. In some embodiments, the poxvirus is a Leporipoxvirus. In some embodiments, the Leporipoxvirus is selected from myxoma virus, Shope fibroma virus (SFV), squirrel fibroma virus, or hare fibroma virus. In some embodiments, the poxvirus is an HPXV. In some embodiments, the horsepox virus is strain MNR-76. In other embodiments, the poxvirus is a VACV. In some embodiments, the VACV is selected from the group of strains consisting of: Western Reserve, Western Reserve Clone 3, Tian Tian, Tian Tian clone TP5, Tian Tian clone TP3, NYCBH, NYCBH clone Acambis 2000, Wyeth, Copenhagen, Lister, Lister 107, Lister-LO, Lister GL-ONC1, Lister GL-ONC2, Lister GL-ONC3, Lister GL-ONC4, Lister CTC1, Lister IMG2 (Turbo FP635), IHD-W, LC16m18, Lederle, Tashkent clone TKT3, Tashkent clone TKT4, USSR, Evans, Praha, L-IVP, V-VET1 or LIVP 6.1.1, Ikeda, EM-63, Malbran, Duke, 3737, CV-1, Connaught Laboratories, Serro 2, CM-01, NYCBH Dryvax clone DPP13, NYCBH Dryvax clone DPP15, NYCBH Dryvax clone DPP20, NYCBH Dryvax clone DPP17, NYCBH Dryvax clone DPP21, VACV-IOC, Chorioallantoid Vaccinia virus Ankara (CVA), Modified vaccinia Ankara (MVA), and MVA-BN. New poxviruses (e.g. Orthopoxviruses) are still being constantly discovered. It is understood that a poxvirus of the disclosure may be based on such a newly discovered poxvirus.

Chemical viral genome synthesis opens up the possibility of introducing a large number of useful modifications to the resulting genome or to specific parts of it. The modifications may improve ease of cloning to generate the virus, provide sites for introduction of recombinant gene products, improve ease of identifying reactivated viral clones and/or confer a plethora of other useful features (e.g. introducing a desired antigen, producing an oncolytic virus, etc.). In some embodiments, the modifications may include the attenuation or deletion of one or more virulence factors. In some embodiments, the modifications may include the addition or insertion of one or more virulence regulatory genes or gene-encoding regulatory factors.

Traditionally, the terminal hairpins of poxviruses have been difficult to clone and to sequence. As a result, some of the published genome sequences (e.g., VACV, ACAM 2000 and HPXV MNR-76) are incomplete. The published sequence of the HPXV genome is likewise incomplete, probably missing ˜60 bp from the terminal ends. In an exemplary embodiment, 129 nt ssDNA fragments were chemically synthesized using the published sequence of the VACV terminal hairpins as a guide and ligated onto dsDNA fragments comprising left and right ends of the HPXV genome. In some embodiments, the terminal hairpins of the poxvirus of the disclosure are derived from VACV. In some embodiments, the terminal hairpins are derived from CMLV, CPXV, ECTV, HPXV, MPXV, RPXV, raccoonpox virus, skunkpox virus, Taterapox virus, Uasin Gishu disease virus or VPV. In some embodiments, the terminal hairpins are based on the terminal hairpins of any poxvirus whose genome has been completely sequenced or a natural isolate of which is available for genome sequencing. In some embodiments, the poxviruses are synthetic versions of HPXV comprising the terminal hairpins of VACV (GenBank accession number KY349117; see US 2018/0251736, incorporated by reference herein).

In some embodiments, the modifications introduced in a poxvirus genome may include the deletion of one or more restriction sites. In some embodiments, the modifications may include the introduction of one or more restriction sites. In some embodiments, the restriction sites to be deleted from the genome or added to the genome may be selected from one or more of restriction sites such as but not limited to AanI, AarI, AasI, AatI, AatII, AbaSI, AbsI, Acc65I, AccI, AccII, AccIII, AciI, AcII, AcuI, AfeI, AflII, AflIII, AgeI, AhdI, AleI, AluI, AlwI, AlwNI, ApaI, ApaLI, ApeKI, ApoI, AscI, AseI, AsiSI, AvaI, AvaII, AvrII, BaeGI, BaeI, BamHI BanI, BanII, BbsI, BbvCI, BbvI, BccI, BceAI, BcgI, BciVI, BclI, BcoDI, BfaI, BfuAI, BfuCI, BglI, BglII, BlpI, BmgBI, BmrI, BmtI, BpmI, Bpu10I, BpuEI, BsaAI, BsaBI, BsaHI, BsaI, BsaXI, BsaWI, BsaXI, BseRI, BseYI, BsgI, BsiEI, BsiHKAI, BsiWI, BslI, BsmAI, BsmBI, BsmFI, BsmI, BsoBI, Bsp1286I, BspCNI, BspDI, BspEI, BspHI, BspMI, BspQI, BsrBI, BsrDI, BsrFαI, BsrGI, BsrI, BssHII, BssSαI, BstAPI, BstBI, BstEII, BstNI, BstUI, BstXI, BstYI, BstZ171, Bsu36I, BtgI, BtgZI, BtsαI, BtsCI, BtsIMutI, Cac8I, ClaI, CspCI, CviAII, CviKI-1, CviQI, DdeI, DpnI, DpnII, DraI, DrdI, EaeI, EagI, EarI, EciI, Eco53kI, EcoNI, EcoO1091, EcoP15I, EcoRI, EcoRV, FatI, FauI, Fnu4HI, FokI, FseI, FspEI, FspI, HaeII, HaeIII, HgaI, HhaI, HincII, HindIII, HinfI, HinP1I, HpaI, HpaII, HphI, Hpy166II, Hpy188I, Hpy188III, Hpy99I, HpyAV, HpyCH4III, HpyCH4IV, HpyCH4V, I-CeuI, I-SceI, KasI, KpnI, LpnPI, MboI, MboII, MfeI, MluCI, MluI, MlyI, MmeI, MnlI, MscI, MseI, MslI, MspA1I, MspI, MspJI, MwoI, NaeI, NarI, NciI, NcoI, NdeI, NgoMIV, NheI, NlaIII, NlaIV, NmeAIII, NotI, NruI, NsiI, NspI, PacI, PaeR7I, PciI, PflFI, PflMI, PleI, PluTI, PmeI, PmII, PpuMI, PshAI, PsiI, PspGI, PspOMI, PspXI, PstI, PvuI, PvuII, RsaI, RsnII, SacI, SacII, SalI, SapI, Sau3AI, Sau96I, SbfI, ScrFI, SexAI, SfaNI, SfcI, SfiI, SfoI, SgrAI, SmaI, SmII, SnaBI, SpeI, SphI, SrfI, SspI, StuI, StyD4I, StyI, SwaI, TaqαI, TfiI, TseI, Tsp45I, TspMI, TspRI, Tth111I, XbaI, XcmI, XhoI, XmaI, XmnI, or ZraI. It is understood that any desired restriction site(s) or combination of restriction sites may be inserted into the genome or mutated and/or eliminated from the genome. In some embodiments, one or more AarI sites are deleted from the viral genome. In some embodiments, one or more BsaI sites are deleted from the viral genome. In some embodiments, one or more restriction sites are completely eliminated from the genome (e.g. all the AarI sites in the viral genome may be eliminated). In some embodiments, one or more AvaI restriction sites are introduced into the viral genome. In some embodiments, one or more StuI sites are introduced into the viral genome. In some embodiments, the one or more modifications may include the incorporation of recombineering targets including but not limited to loxP or FRT sites.

In some embodiments, the poxvirus modifications may include the introduction of fluorescence markers such as but not limited to green fluorescent protein (GFP), enhanced GFP, yellow fluorescent protein (YFP), cyan/blue fluorescent protein (BFP), red fluorescent protein (RFP), or variants thereof, etc.; selectable markers such as but not limited to drug resistance markers (e.g. E. coli xanthine-guanine phosphoribosyl transferase gene (gpt), Streptomyces alboniger puromycin acetyltransferase gene (pac), neomycin phosphotransferase I gene (nptI), neomycin phosphotransferase gene II (nptII), hygromycin phosphotransferase (hpt), sh ble gene, etc.; protein or peptide tags such as but not limited to MBP (maltose-binding protein), CBD (cellulose-binding domain), GST (glutathione-S-transferase), poly(His), FLAG, V5, c-Myc, HA (hemagglutinin), NE-tag, CAT (chloramphenicol acetyl transferase), DHFR (dihydrofolate reductase), HSV (Herpes simplex virus), VSV-G (Vesicular stomatitis virus glycoprotein), luciferase, protein A, protein G, streptavidin, T7, thioredoxin, Yeast 2-hybrid tags such as B42, GAL4, LexA, or VP16; localization tags such as an NLS-tag, SNAP-tag, Myr-tag, etc. It is understood that other selectable markers and/or tags known in the art may be used. In some embodiments, the modifications include one or more selectable markers to aid in the selection of reactivated clones (e.g. a fluorescence marker such as YFP, a drug selection marker such as gpt, etc.) to aid in the selection of reactivated viral clones. In some embodiments, the one or more selectable markers are deleted from the reactivated clones after the selection step.

In some embodiments, the poxviruses are synthetic horsepox viruses (scHPXV). In some embodiments, the synthetic horsepox viruses have been produced by recombination of overlapping DNA fragments of the viral genome and reactivation of the functional poxvirus is carried out in cells previously infected with a helper virus. Briefly, overlapping DNA fragments that encompass all or substantially all of the viral genome of the horsepox are chemically synthesized and transfected into helper virus-infected cells. The transfected cells are cultured to produce mixed viral progeny comprising the helper virus and reactivated horsepox virus. Next, the mixed viral progeny is plated on host cells that do not support the growth of the helper virus but allow the synthetic poxvirus to grow, in order to eliminate the helper virus and recover the synthetic poxviruses.

In some embodiments, substantially all of the synthetic poxviral genome is derived from chemically synthesized DNA. In some embodiments, about 40%, about 50%, about 60%, about 70%, about 75%, about 80%, about 85%, about 90%, about 95%, about 96%, about 97%, about 98%, about 99%, over 99%, or 100% of the synthetic poxviral genome is derived from chemically synthesized DNA. In some embodiments, the poxviral genome is derived from a combination of chemically synthesized DNA and naturally occurring DNA.

The number of overlapping DNA fragments used to generate the synthetic poxvirus will depend on the size of the poxviral genome. Practical considerations such as reduction in recombination efficiency as the number of fragments increases on the one hand and difficulties in synthesizing very large DNA fragments as the number of fragments decreases on the other hand will also inform the number of overlapping fragments used. In some embodiments, the synthetic poxviral genome may be synthesized as a single fragment. In some embodiments, the synthetic poxviral genome is assembled from 2-14 overlapping DNA fragments. In some embodiments, the synthetic poxviral genome is assembled from 4-12 overlapping DNA fragments. In some embodiments, the synthetic poxviral genome is assembled from 6-10 overlapping DNA fragments. In some embodiments, the synthetic poxviral genome is assembled from 8-12 overlapping DNA fragments. In some embodiments, the synthetic poxviral genome is assembled from 10 overlapping DNA fragments. In an exemplary embodiment of the disclosure, a synthetic horsepox virus (scHPXV) is reactivated from 10 chemically synthesized overlapping double-stranded DNA fragments. In some embodiments, all of the fragments encompassing the poxviral genome are chemically synthesized. In some embodiments, one or more of the fragments are chemically synthesized and one or more of the fragments are derived from naturally occurring DNA (e.g. by PCR amplification or by well-established recombinant DNA techniques).

In some embodiments, the terminal hairpin loops are synthesized separately and ligated onto the fragments comprising the left and right ends of the poxviral genome. In some embodiments, terminal hairpin loops may be derived from a naturally occurring template. In some embodiments, the terminal hairpins of the synthetic poxvirus are derived from VACV. In some embodiments, the terminal hairpins of the recombinant synthetic poxvirus are derived from CMLV, CPXV, ECTV, HPXV, MPXV, RPXV, raccoonpox virus, skunkpox virus, Taterapox virus, Uasin Gishu disease virus or VPV. In some embodiments, the terminal hairpins of the recombinant scHPXV are derived from VACV. In some embodiments, the terminal hairpins of the recombinant scHPXV are derived from CMLV, CPXV, ECTV, HPXV, MPXV, RPXV, raccoonpox virus, skunkpox virus, Taterapox virus, Uasin Gishu disease virus or VPV. In some embodiments, the terminal hairpins of the poxvirus are based on the terminal hairpins of any poxvirus whose genome has been completely sequenced or a natural isolate of which is available for genome sequencing.

The size of the overlapping fragments used to generate the poxvirus of the disclosure will depend on the size of the poxviral genome. It is understood that there can be wide variations in fragment sizes and various practical considerations such as the ability to chemically synthesize very large DNA fragments, will inform the choice of fragment sizes. In some embodiments, the fragments range in size is from about 2000 bp to about 50000 bp. In some embodiments, the fragments range in size is from about 3000 bp to about 45000 bp. In some embodiments, the fragments range in size is from about 4000 bp to 40000 bp. In some embodiments, the fragments range in size is from about 5000 bp to 35000 bp. In some embodiments, the largest fragments are about 20000 bp, 21000 bp, 22000 bp, 23000 bp, 24 000 bp, 25000 bp, 26000 bp, 27000 bp, 28000 bp, 29000 bp, 30000 bp, 31000 bp, 32000 bp, 33000 bp, 34000 bp, 35000 bp, 36000 bp, 37000 bp, 38000 bp, 39000 bp, 40000 bp, 41000 bp, 42000 bp, 43000 bp, 44000 bp, 45000 bp, 46000 bp, 47000 bp, 48000 bp, 49000 bp, or 50000 bp. In some embodiments, a scHPXV is reactivated from 10 chemically synthesized overlapping double-stranded DNA fragments ranging in size from about 8500 bp to about 32000 bp (Table 2).

The poxviruses of the present disclosure can be propagated in any substrate that allows the virus to grow to titers that permit the uses of the recombinant poxvirus described herein. The poxvirus of the present disclosure may be grown in cells (e.g. avian cells, bat cells, bovine cells, camel cells, canary cells, cat cells, deer cells, equine cells, fowl cells, gerbil cells, goat cells, human cells, monkey cells, pig cells, rabbit cells, raccoon cells, seal cells, sheep cells, skunk cells, vole cells, etc.) that are susceptible to infection by the poxviruses. In some embodiments, the poxvirus is grown in adherent cells. In some embodiments, the poxvirus is grown in suspension cells. In some embodiments, the poxvirus is grown in mammalian cells. Such methods are well-known to those skilled in the art. Representative mammalian cells include, but are not limited to, BHK, MRC, BGMK, BRL3A, BSC-40, CEF, CEK, CHO, COS, CVI, HaCaT, HEL, HeLa cells, HEK293, human bone osteosarcoma cell line 143B, MDCK, NIH/3T3, Vero cells, etc. For virus isolation, the recombinant poxvirus is removed from cell culture and separated from cellular components, typically by well-known clarification procedures, e.g., such as gradient centrifugation and column chromatography, and may be further purified as desired using procedures well known to those skilled in the art, e.g., plaque assays. In some embodiments, the poxvirus is grown in Vero cells. In some embodiments, the poxvirus is grown in ACE2 Knockout Vero cells. In some embodiments, the poxvirus is grown in Vero adherent cells. In other embodiments, the poxvirus is grown in Vero suspension cells. In some embodiments, the poxvirus is grown in BSC-40 cells. In some embodiments, the poxvirus is grown in BHK-21 cells. In some embodiments, the poxvirus is grown in MRC-5 cells. In some embodiments, the poxvirus is grown in MRC-5 cells in the presence of for example, 5% serum, including but not limited to fetal calf serum. In some embodiments, the poxvirus is grown in avian cells. Such methods are well-known to those skilled in the art. Representative avian cells include, but are not limited to, chicken embryo fibroblasts, DF-1 cells (see, e.g., Himly et al., Virology, (1998) 248:295-304), duck embryo-derived cells, EB66® cells (see, e.g., Leon et al. Vaccine, (2016) 34: 5878-5885), AGE1. CR cells, including but not limited to AGE1.CRpIX® cells, DF-1 cells (see, e.g., Lohr et al., Vaccine, (2009) 36:4975-4982), etc. In some embodiments, the poxvirus is grown in chicken embryo fibroblasts. In some embodiments, the poxvirus is grown in duck embryo-derived cells. In some embodiments, the poxvirus is grown in EB66® cells. In some embodiments, the poxvirus is grown in AGE1.CRpIX® cells. In some embodiments, the poxvirus is grown in DF-1 cells.

In some embodiments, the method of producing a synthetic poxvirus comprises a step of chemically synthesizing overlapping DNA fragments that correspond to substantially all of the viral genome of the poxvirus and, optionally, chemically synthesizing the terminal hairpin loops from another virus or from another strain of virus; (ii) transfecting the overlapping DNA fragments into helper virus-infected cells; (iii) culturing said cells to produce a mixture of helper virus and synthetic poxvirus particles in said cells; and (iv) plating the mixture on host cells specific to the poxvirus to recover the synthetic poxvirus.

In some embodiments, the method of producing a synthetic horsepox virus comprises a step of (i) chemically synthesizing overlapping DNA fragments that correspond to substantially all of the viral genome of the horsepox virus and chemically synthesizing the terminal hairpin loops from another poxvirus (such as VACV, strain WB or NYCBH clone ACAM 2000); (ii) transfecting the overlapping DNA fragments into helper virus-infected cells; (iii) culturing said cells to produce a mixture of helper virus and synthetic horsepox virus particles in said cells; and (iv) plating the mixture on host cells specific to the horsepox virus to recover the synthetic horsepox virus.

In some embodiments, the poxvirus is a synthetic horsepox virus. In some embodiments, the synthetic horsepox virus genome is based on the published genome sequence described for horsepox virus (GenBank accession DQ792504) and the terminal hairpins are based on the published genome sequence similar to VACV strain NYCBH clone ACAM2000 (GenBank accession MN974380). In some embodiments, the synthetic horsepox virus comprises the sequence deposited in GenBank as accession number KY349117; see US 2018/0251736, incorporated by reference herein. In some embodiments, the synthetic horsepox virus is characterized by a nucleic acid encoding a SARS-CoV-2 virus S protein comprises the sequence set forth in SEQ ID NO: 43.

In some embodiments, the poxvirus is a synthetic recombinant vaccinia virus (synVACV). In some embodiments, the synthetic vaccinia genome is based on the published genome sequence described for VACV strain NYCBH clone ACAM2000 (GenBank accession AY313847; Osborne J D et al. Vaccine. 2007; 25(52):8807-32). In some embodiments, the synthetic vaccinia genome is based on the published genome sequence similar to VACV strain NYCBH clone ACAM2000 (GenBank accession MN974380; see WO 2019/213452, incorporated by reference herein). In some embodiments, the synthetic vaccinia virus comprises the sequence deposited in GenBank as accession number MN974381 (see WO 2019/213452, incorporated by reference herein). In some embodiments, the synthetic vaccinia virus is characterized by a nucleic acid encoding a SARS-CoV-2 virus S protein comprises the sequence set forth in SEQ ID NO: 44.

### Generation of the Recombinant Poxvirus Comprising a SARS-CoV-2 Protein

Any of the synthetic poxviruses disclosed in US 2018/0251736 and WO 2019/213452, may be used to generate a recombinant poxvirus comprising a SARS-CoV-2 protein, as disclosed herein.

In one aspect, the present disclosure relates to a recombinant poxvirus comprising a nucleic acid encoding a SARS-CoV-2 virus protein, wherein the SARS-CoV-2 protein is selected from the group consisting of the spike protein (S), the membrane protein (M) and the nucleocapsid protein (N), or combinations of two or more of said proteins. In some embodiments, the nucleotide sequence of the SARS-CoV-2 virus is any one of the published genome sequences, including, but not limited, to the genome sequences of the Wuhan strain, the UK strain B.1.1.7 strain, the South African B. 1.351 strain, the Brazilian B.1.1.28 strain, other emerging variants and any of their variants. In some embodiments, the nucleotide sequence of the SARS-CoV-2 virus is selected from the group consisting of GenBank accession numbers NC045512.2, LC521925.1, MN988668.1, MN985325.1, MN975262.1, MN938384.1, LR757998.1, LR757996.1, LR757995.1 and MN908947.3. In some embodiments, the nucleotide sequence of the SARS-CoV-2 virus is characterized by the sequence set forth in GenBank Accession Number MN988668.1; SEQ ID NO: 46. In some embodiments, the nucleotide sequence of the SARS-CoV-2 virus is further selected from the group consisting of GenBank accession numbers QQX99439 (e.g., B.1.1.7 United Kingdom variant), TEGALLY (e.g., B.1.351 South Africa variant), YP_009724390 (e.g., a Wuhan variant), and FARIA (e.g., B.1.1.28 Brazil variant).

The viral envelope of the SARS-CoV-2 virus is covered by characteristic spike-shaped glycoproteins (S) as well as the envelope (E) and membrane (M) proteins. The S protein mediates host cell attachment and entry. The helical nucleocapsid, comprised of the viral genome encapsidated by the nucleocapsid protein (N), resides within the viral envelope. In some embodiments, the poxvirus or synthetic poxvirus comprises a nucleic acid encoding a SARS-CoV-2 envelope protein. Non-limiting examples of such proteins are the Spike protein (S), the Membrane protein (M) and the Hemagglutinin-Esterase protein (HE). In some embodiments, the poxviruses or synthetic poxviruses comprise a nucleic acid encoding the S protein (SEQ ID NO: 9). In some embodiments, the poxviruses or synthetic poxviruses comprise a nucleic acid encoding the S protein (SEQ ID NO: 47). In some embodiments, the poxviruses or synthetic poxviruses comprise a nucleic acid encoding the M protein (SEQ ID NO: 10). In some embodiments, the poxviruses or synthetic poxviruses comprise a nucleic acid encoding the M protein (SEQ ID NO: 48). In some embodiments, the poxviruses or synthetic poxviruses comprise a nucleic acid encoding the N protein (SEQ ID NO: 11). In some embodiments, the poxviruses or synthetic poxviruses comprise a nucleic acid encoding the N protein (SEQ ID NO: 49). In some embodiments, the poxviruses or synthetic poxviruses comprise a nucleic acid encoding the HE protein (protein E or HE of Wuhan-HU-1, Accession LC521925.1; SEQ ID NO: 12). In some embodiments, the poxviruses or synthetic poxviruses comprise a combination of S protein and M protein. In some embodiments, the poxviruses or synthetic poxviruses comprise a combination of S protein and N protein. In some embodiments, the poxviruses or synthetic poxviruses comprises a combination of M protein and N protein.

In some embodiments, the SARS-CoV-2 virus is a Wuhan seafood market pneumonia virus 2019-nCoV isolate. GenBank accession number LC521925.1; SEQ ID NO: 13. In some embodiments, the SARS-CoV-2 virus is a Wuhan seafood market pneumonia virus 2019-nCoV isolate. GenBank accession number MN988668.1; SEQ ID NO: 46.

In some embodiments, the amino acid sequence of the SARS-CoV-2 virus protein is modified with reference to a wild type protein.

In some embodiments, the nucleotide sequence encoding the S protein is modified with reference to a wild type nucleotide sequence. In some embodiments, the amino acid sequence of the S protein is modified with reference to the wild type protein (protein S of Wuhan-HU-1, Accession LC521925.1; SEQ ID NO: 9). In some embodiments, the amino acid sequence of the S protein is modified with reference to the wild type protein (protein S of Wuhan-HU-1, Accession MN988668.1; SEQ ID NO: 47). In some embodiments, the amino acid sequence of the S protein is modified with reference to the wild type protein (protein S of Wuhan-Hu-1, Accession NC_045512.2; SEQ ID NO: 53) In some embodiments, the amino acid sequence of the SARS-CoV-2 virus protein is modified with reference to a wild type protein, so that the modified protein is adapted to infect mice. See Roberts et al. PLoS Pathog 3(1): e5. doi:10.1371; incorporated herein by reference in its entirety. In some embodiments, Tyrosine at position 459 is substituted by Histidine (Y459H) in the S protein with reference to the wild type protein (SEQ ID NO: 47). In some embodiments, the S protein comprises one or more mutations that enable antibody-dependent enhancement. In some embodiments, Aspartic acid at position 614 is substituted by Glycine (D614G) in the S protein with reference to the wild type protein (SEQ ID NO: 47). See Korber et al. bioRxiv 2020.04.29.069054; incorporated herein by reference in its entirety. In some embodiments, the S protein comprises one or more mutations in the fusion core of the HR1 region. In some embodiments, Serine at position 943 is substituted by Proline (S943P) in the S protein with reference to the wild type protein (SEQ ID NO: 47). In some embodiments, the S protein comprises one or more mutations that stabilize the S protein in an antigenically optimal prefusion conformation, which results in increased expression, conformational homogeneity and elicitation of potent antibody responses. In some embodiments, the mutations that stabilize the S protein in the prefusion conformation are located at the beginning of the central helix. See Pallesen et al. Proc Natl Acad Sci USA. 2017; 114(35); incorporated herein by reference in its entirety. In some embodiments, Lysine at position 986 is substituted by Proline (K986P) in the S protein with reference to the wild type protein (SEQ ID NO: 47). In some embodiments, Valine at position 987 is substituted by Proline (V987P) in the S protein with reference to the wild type protein (SEQ ID NO: 47). In some embodiments, the S protein comprises any one of substitutions Y459H, D614G, S943P, K986P and V987P, or a combination thereof, with reference to the wild type protein (SEQ ID NO: 47).

In some embodiments, the amino acid sequence of the M protein is modified with reference to the wild type protein (protein M of Wuhan-HU-1, Accession LC521925.1; SEQ ID NO: 10). In some embodiments, the amino acid sequence of the M protein is modified with reference to the wild type protein (protein M of Wuhan-HU-1, Accession MN988668.1; SEQ ID NO: 48). In some embodiments, Glutamic acid at position 11 is substituted by a Lysine in the M protein with reference to the wild type protein. In some embodiments, Glutamic acid at position 11 is substituted by a Lysine in the M protein with reference to the wild type protein (SEQ ID NO: 10). In some embodiments, Glutamic acid at position 11 is substituted by a Lysine in the M protein with reference to the wild type protein (SEQ ID NO: 48).

In some embodiments, the amino acid sequence of the N protein is modified with reference to the wild type protein (protein N of Wuhan-HU-1, Accession LC521925.1; SEQ ID NO: 11). In some embodiments, the amino acid sequence of the N protein is modified with reference to the wild type protein (protein N of Wuhan-HU-1, Accession MN988668.1; SEQ ID NO: 49).

In some embodiments, the nucleic acid sequence encoding the SARS-CoV-2 virus protein is modified with reference to the wild type protein. In some embodiments, the nucleic acid sequence encoding the SARS-CoV-2 virus protein is modified with reference to the wild type protein (SEQ ID NO: 9). In some embodiments, the nucleic acid sequence encoding the SARS-CoV-2 virus protein is modified with reference to the wild type protein (SEQ ID NO: 47). In some embodiments, the nucleic acid sequence encoding the SARS-CoV-2 virus protein is modified with reference to the wild type protein for efficient expression of transgenes in poxviruses. In some embodiments, the heterologous gene coding sequences containing the vaccinia Early Transcription Terminator Signal (ETTS) (TTTTTNT; also called T₅NT (SEQ ID NO: 14)) are removed. See Earl et al. Journal of Virology, 1990; 2448-2451; incorporated herein by reference in its entirety. In some embodiments, the poxvirus genome retains two overlapping endogenous ETTS. In some embodiments, the heterologous gene coding sequences containing the vaccinia Early Transcription Terminator Signal (ETTS) (TTTTTNT; also called T₅NT (SEQ ID NO: 14)) are removed with reference to the nucleic sequence encoding the S protein of the SARS-CoV-2 virus (protein S of Wuhan-HU-1, Accession MN988668.1; SEQ ID NO: 47).

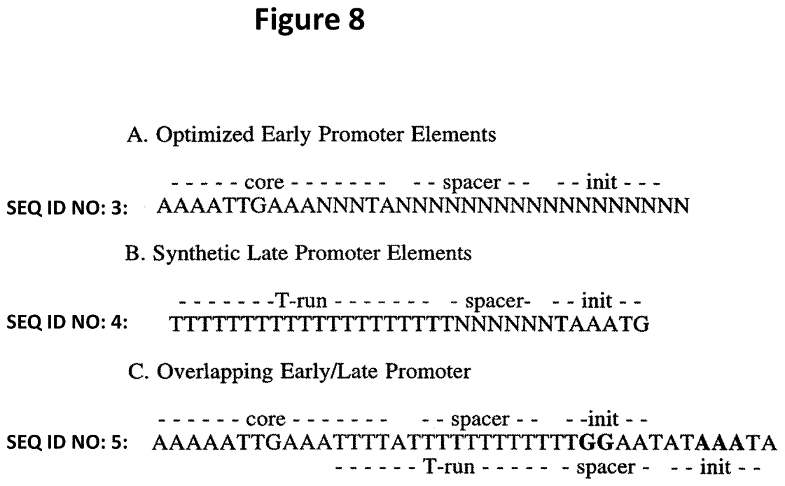

In some embodiments, the nucleic acid encoding a SARS-CoV-2 virus protein is operatively linked to a promoter. In some embodiments, the promoter is a poxvirus-specific promoter. In some embodiments, the promoter is located between the left flanking arm and the ATG of the transgene expression cassette. In some embodiments, the poxvirus promoter is a vaccinia virus early promoter. In some embodiments, the poxvirus promoter is an optimized vaccinia virus early promoter (AAAATTGAAANNNTANNNNNNNNNNNNNNNNNN; SEQ ID NO: 3). In some embodiments, the poxvirus promoter is a synthetic vaccinia virus late promoter (TTTTTTTTTTTTTTTTTTTNNNNNNTAAATG; SEQ ID NO: 4). In some embodiments, the poxvirus promoter is an overlapping synthetic early/late promoter (AAAAATTGAAATTTTATTTTTTTTTTTTGGAATATAAATA; SEQ ID NO: 5). See FIG. 8. See Chakrabarti et al. BioTechniques 23:1094-1097; incorporated herein by reference in its entirety.

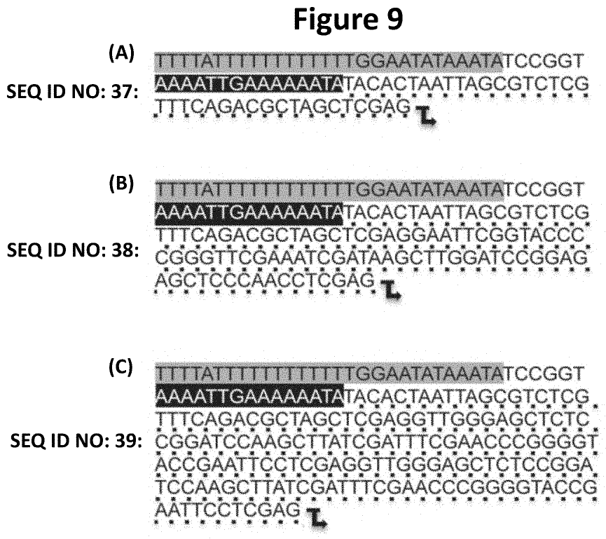

In some embodiments, the vaccinia virus late promoter nucleotide sequence comprises the sequence set forth in SEQ ID NO: 6 (TTTTATTTTTTTTTTTTGGAATATAAATA). In some embodiments, the vaccinia virus late promoter is the sequence set forth in SEQ ID NO: 6. In some embodiments, the vaccinia virus late promoter nucleotide sequence comprises the sequence set forth in SEQ ID NO: 7 (AAAATTGAAAAAATA). In some embodiments, the poxvirus promoter is an overlapping synthetic early/late promoter comprising the sequence set forth in SEQ ID NO: 8 (TTTTATTTTTTTTTTTTGGAATATAAATATCCGGT AAAATTGAAAAAATA). In some embodiments, the poxvirus promoter is an overlapping synthetic early/late promoter comprising a nucleic acid spacer sequence of 38-160 nucleotides 3′ of the early promoter and between the RNA start site and the ATG. In some embodiments, the spacer is 160 nucleotides long, resulting in enhanced levels of expression. See FIG. 9. See Di Pilato et al. Journal of General Virology (2015), 96, 2360-2371; incorporated herein by reference in its entirety. In some embodiments, the vaccinia virus late promoter and the spacer comprises the sequence set forth in SEQ ID NO: 39. In some embodiments, the vaccinia virus late promoter and the spacer is the sequence set forth in SEQ ID NO: 39.

In some embodiments, the protein of the SARS-CoV-2 is inserted into a non-essential gene for replication. In some embodiments, the SARS-CoV-2 protein is inserted into the Thymidine Kinase (TK) locus (Gene ID HPXV095; positions 992077-92610; SEQ ID NO: 1) of the horsepox virus or the synthetic horsepox virus. In some embodiments, the SARS-CoV-2 protein is inserted into the Thymidine Kinase (TK) locus (Gene ID synVACV_105; positions 83823-84344; SEQ ID NO: 2) of the vaccinia virus or the synthetic vaccinia virus. The TK locus provides a stable insertion site for foreign genes of interest. The TK locus also provides a selection marker to identify those clones where the nucleic acid encoding a SARS-CoV-2 protein has been inserted. The clones where the nucleic acid encoding a SARS-CoV-2 protein is inserted are not capable of growing in the presence of 5-bromo-2-deoxyuridine (BrdU), which is an analogue of the pyrimidine deoxynucleoside thymidine, due to not having the TK gene.

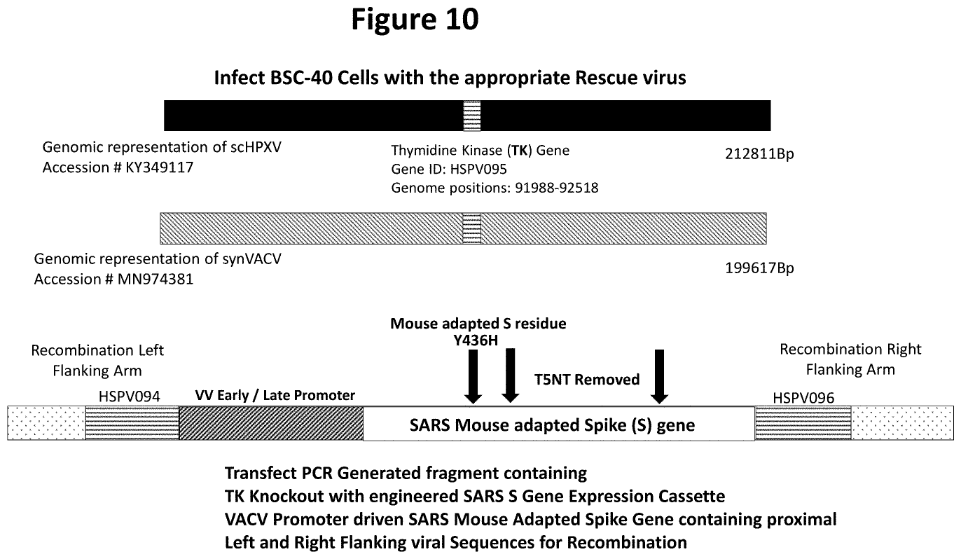

An exemplary method to generate a recombinant poxvirus of the disclosure comprising the S protein of SARS-CoV-2 virus comprises:
a) Infect cells (e.g., Vero cells or BSC-40 cells) with the poxvirus (such as horsepox virus).
b) Obtain an expression cassette comprising: a nucleotide fragment comprising the nucleotide sequence encoding the S protein, wherein the resulting S protein comprises any one of the amino acid substitutions (i) Y459H, so that it is adapted for infection in mice; (ii) D614G; (iii) S943P; (iv) K986P or (v) V987P, or a combination thereof; and wherein the nucleotide sequence encoding the S protein comprises the deletion of two T5NT (SEQ ID NO: 14) sequences.
c) Obtain a nucleotide fragment comprising the vaccinia virus early/late promoter and position it upstream of the modified S protein. This expression cassette comprising the vaccinia virus early/late promoter and the engineered S gene is called “engineered SARS-CoV-2 S gene expression cassette”.
d) Transfect the infected cells (e.g., Vero cells or BSC-40 cells) with a PCR generated nucleotide fragment comprising the “engineered SARS-CoV-2 S gene expression cassette”. The helper virus catalyzes the recombination between fragments sharing flanking homologous sequences (the sequence between the left and right arm). Therefore, the expression cassette will be inserted into the TK gene via recombination between the left (HPXV094) and right (HPXV096) homologous sequences (arms). The left and right arms are approximately 400 bp sequences flanking the TK locus and are specific of the poxvirus to be generated. See FIG. 10.

### Methods of the Disclosure

Any of the synthetic poxviruses disclosed in US 2018/0251736 and WO 2019/213452, may be used in any of the methods disclosed herein.

Any of the recombinant poxviruses comprising a nucleic acid encoding a SARS-CoV-2 virus protein described in the present disclosure may be used in any of the methods disclosed herein.

In one aspect, the disclosure relates to a method for selecting a cell that expresses a SARS-CoV-2 virus protein, comprising infecting said cell with the recombinant poxvirus of the disclosure and selecting the infected cell expressing said SARS-CoV-2 virus protein.

In another aspect, the disclosure relates to a method of inducing an immune response against a SARS-CoV-2 virus in a subject, comprising administering to said subject an immunologically effective amount of the recombinant poxvirus of the disclosure.

In another aspect, the disclosure relates to a method of generating a recombinant poxvirus of the disclosure, the method comprising:

(a) Infecting a host cell with a poxvirus;
(b) Transfecting the infected cell of step (a) with a nucleic acid encoding a SARS-CoV-2 virus protein to generate a recombinant poxvirus; and
(c) Selecting a recombinant poxvirus, wherein the nucleic acid encoding a SARS-CoV-2 virus protein is located, upon transfection, in a region of the poxvirus that is not essential for the replication of the poxvirus.

In some embodiments, the recombinant poxvirus of the disclosure is used as a vaccine to express a SARS-CoV-2 virus protein. Methods to assess the safety, immunogenicity and protective capacity of the recombinant poxvirus are known in the art. See Kremer M et al. 2012. p 59-92. In Isaacs S N (ed), Vaccinia virus and poxvirology, vol 890. Humana Press, Totowa, N.J. In some embodiments, the immunization is via a subcutaneous route. In some embodiments, the immunization is via an intramuscular route. In some embodiments, the immunization is via an intranasal route. In some embodiments, the immunization is via scarification. In some embodiments, a range between about 10⁴ and about 10⁸ PFU of the recombinant poxvirus is used. In some embodiments, about 10⁴, about 10⁵, about 10⁶, about about 10⁷ or about 10⁸ PFU of recombinant poxvirus is used for the immunization. In some embodiments, about 10⁵ PFU of the recombinant poxvirus is used for the immunization. A physician will be able to determine the adequate PFU dosage for each subject. In some embodiments, one dose is administered to the subject. In some embodiments, more than one dose is administered to the subject.

In some embodiments, the recombinant poxvirus is useful towards the method of inducing an immune response against a SARS-CoV-2 virus in a subject, comprising administering to said subject an immunologically effective amount of a recombinant poxvirus or a pharmaceutical composition. In some embodiments, the recombinant poxvirus is useful towards the method of inducing an immune response against the SARS-CoV-2 virus in a subject, wherein the immunologically effective amount of the recombinant poxvirus is administered by scarification. In some embodiments, the recombinant poxvirus is useful towards the method of inducing an immune response against a SARS-CoV-2 virus in a subject, wherein the immune response comprises antibodies that are capable of neutralizing the SARS-CoV-2 virus. In some embodiments, the recombinant poxvirus is useful towards the method of inducing an immune response against a SARS-CoV-2 virus in a subject, wherein the immunologically effective amount of a recombinant poxvirus is capable of protecting the subject from SARS-CoV-2 virus. In some embodiments, the recombinant poxvirus is useful towards the method of inducing an immune response against a SARS-CoV-2 virus in a subject, wherein the immunologically effective amount of a recombinant poxvirus reduces or prevents the progression of the virus after SARS-CoV-2 infection in the subject. In some embodiments, the recombinant poxvirus is useful towards the method of inducing an immune response against a SARS-CoV-2 virus in a subject, wherein the immune response is a T-cell immune response.

In some embodiments, the recombinant poxvirus is useful towards the method of inducing an immune response against a SARS-CoV-2 virus and a poxvirus comprising administering to said subject an immunologically effective amount of a recombinant poxvirus or pharmaceutical composition. In some embodiments, the recombinant poxvirus is useful towards the method of inducing an immune response against the SARS-CoV-2 virus and the poxvirus, wherein said immunologically effective amount of the recombinant poxvirus is administered by scarification. In some embodiments, the recombinant poxvirus is useful towards the method of inducing an immune response against the SARS-CoV-2 virus and the poxvirus, wherein said immune response comprises antibodies that are capable of neutralizing the SARS-CoV-2 virus and the poxvirus. In some embodiments, the recombinant poxvirus is useful towards the method of inducing an immune response against the SARS-CoV-2 virus and the poxvirus, wherein the immunologically effective amount of a recombinant poxvirus is capable of protecting the subject from the SARS-CoV-2 virus and the variola virus. In some embodiments, the recombinant poxvirus is useful towards the method of inducing an immune response against the SARS-CoV-2 virus and the poxvirus, wherein the immunologically effective amount of a recombinant poxvirus reduces or prevents the progression of the SARS-CoV-2 virus infection and/or poxvirus infection in the subject. In some embodiments, the recombinant poxvirus is useful towards the method of inducing an immune response against the SARS-CoV-2 virus and the poxvirus, wherein the immune response is a T-cell immune response. In some embodiments, the recombinant poxvirus is useful towards the method of inducing an immune response against the SARS-CoV-2 virus and the poxvirus, wherein the poxvirus is vaccinia virus, variola, horsepox virus or monkeypox virus.

In some embodiments, the recombinant poxvirus is useful towards the method of inducing T cell immunity against a SARS-CoV-2 virus comprising administering to said subject an immunologically effective amount of a recombinant poxvirus or pharmaceutical composition. In some embodiments, the recombinant poxvirus is useful towards the method of inducing T cell immunity against the SARS-CoV-2 virus, wherein said immunologically effective amount of the recombinant poxvirus is administered by scarification. In some embodiments, the recombinant poxvirus is useful towards the method of inducing T cell immunity against the SARS-CoV-2 virus, wherein the immunologically effective amount of a recombinant poxvirus is capable of protecting the subject from SARS-CoV-2 virus. In some embodiments, the recombinant poxvirus is useful towards the method of inducing T cell immunity against the SARS-CoV-2 virus, wherein the immunologically effective amount of a recombinant poxvirus reduces or prevents the progression of the virus after SARS-CoV-2 infection in the subject.

In some embodiments, the recombinant poxvirus is useful towards the method of inducing T cell immunity against a SARS-CoV-2 virus and a poxvirus comprising administering to a subject an immunologically effective amount of the recombinant poxvirus reduces or pharmaceutical composition. In some embodiments, the recombinant poxvirus is useful towards the method of inducing T cell immunity against the SARS-CoV-2 virus and the poxvirus, wherein said immunologically effective amount of the recombinant poxvirus is administered by scarification. In some embodiments, the recombinant poxvirus is useful towards the method of inducing T cell immunity against the SARS-CoV-2 virus and the poxvirus, wherein the immunologically effective amount of a recombinant poxvirus is capable of protecting the subject from the SARS-CoV-2 virus and the poxvirus. In some embodiments, the recombinant poxvirus is useful towards the method of inducing T cell immunity against the SARS-CoV-2 virus and the poxvirus, wherein the immunologically effective amount of a recombinant poxvirus reduces or prevents the progression of the virus after SARS-CoV-2 infection and/or variola virus infection in the subject. In some embodiments, the recombinant poxvirus is useful towards the method of inducing T cell immunity against the SARS-CoV-2 virus and the poxvirus, wherein the poxvirus is vaccinia virus, variola, horsepox virus or monkeypox virus.

In some embodiments, the recombinant poxvirus is useful towards the method of reducing or preventing the progression of a SARS-CoV-2 virus infection in a subject in need or at risk thereof comprising administering to said subject an immunologically effective amount of the recombinant poxvirus or pharmaceutical composition.

In some embodiments, the recombinant poxvirus is useful towards the method of reducing or preventing the progression of a SARS-CoV-2 virus and a poxvirus infection in a subject in risk thereof comprising administering to said subject an immunologically effective amount of the recombinant poxvirus or pharmaceutical composition. In some embodiments, the recombinant poxvirus is useful towards the method of reducing or preventing the progression of the SARS-CoV-2 virus and the poxvirus infection, wherein the poxvirus is vaccinia virus, variola, horsepox virus or monkeypox virus.

In some embodiments, the recombinant poxvirus is useful for a vaccine against a SARS-CoV-2 virus comprising a recombinant virus or a pharmaceutical composition.

In some embodiments, the recombinant poxvirus is useful for a bivalent vaccine against a SARS-CoV-2 virus and a poxvirus comprising a recombinant virus or a pharmaceutical composition. In some embodiments, the recombinant poxvirus is useful for a bivalent vaccine against a SARS-CoV-2 virus, wherein the poxvirus is a vaccinia virus, variola, horsepox virus or monkeypox.

TABLE 1
Compilation of some of the sequences of the present disclosure.

| | | | |
|---|---|---|---|
| Synthetic horsepox virus | 1 | ATTTACGGATTCACCAATAAAAATAAACTAGAGAAACTTAGTACTAATAAGGAAC | 55 |
| comprising a nucleic acid | 56 | TAGAATCGTATAGTTCTAGCCCTCTTCAAGAACCCATTAGGTTAAATGATTTTCT | 110 |
| encoding a SARS-CoV-2 | 111 | GGGACTATTGGAATGTATTAAAAAGAATATTCCTCTAACAGATATTCCGACAAAG | 165 |
| virus S protein. | 166 | GATTGATTACTATAAATGGAGAATGTTCCTAATGTATACTTTAATCCTGTGTTTA | 220 |
| SEQ ID NO: 43 | 221 | TAGAGCCCACGTTTAAACATTCTTTATTAAGTGTTTATAAACACAGATTAATAGT | 275 |
| | 276 | TTTATTTGAAGTATTCATTGTATTCATTCTAATATATGTATTTTTTAGATCTGAA | 330 |
| | 331 | TTAAATATGTTCTTCATGCCTAAACGAAAAATACCCGATCCTATTGATAGATTAC | 385 |
| | 386 | GACGTGCTAATCTAGCGTGTGAAGACGATAAGTTAATGATCTATGGATTACCATG | 440 |
| | 441 | GATGACAACTCAAACATCTGCGTTATCAATAAATAGTAAACCGATAGTGTATAAA | 495 |
| | 496 | GATTGTGCAAAGCTTTTGCGATCAATAAATGGATCACAACCAGTATCTCTTAACG | 550 |
| | 551 | ATGTTCTTCGCAGATGATGATTCATTTTTTAAGTATTTGGCTAGTCAAGATGATG | 605 |
| | 606 | AATCTTCATTATCTGATATATTGCAAATCACTCAATATCTAGACTTTCTGTTATT | 660 |
| | 661 | ATTATTGATCCAATCAAAAAATAAATTAGAAGCCGTGGGTCATTGTTATGAATCT | 715 |
| | 716 | CTTTCAGAGGAATACAGACAATTGACAAAATTCACAGACTTTCAAGATTTTAAAA | 770 |
| | 771 | AACTGTTTAACAAGGTCCCTATTGTTACAGATGGAAGGGTCAAACTTAATAAAGG | 825 |
| | 826 | ATATTTGTTCGACTTTGTGATTAGTTTGATGCGATTCAAAAAAGAATCCTCTCTA | 880 |
| | 881 | GCTACCACCGCAATAGATCCTATTAGATACATAGATCCTCGTCGTGATATCGCAT | 935 |
| | 936 | TTTCTAACGTGATGGATATATTAAAGTTGAATAAAGTGAACAATAATTAATTCTT | 990 |
| | 991 | TATTGTCATCTTTTATTTTTTTTTTTTGGAATATAAATATCCGGTAAAATTGAAA | 1045 |
| | 1046 | AAATATACACTAATTAGCGTCTCGTTTCAGACGCTAGCTCGAGGTTGGGAGCTCT | 1100 |
| | 1101 | CCGGATCCAAGCTTATCGATTTCGAACCCGGGGTACCGAATTCCTCGAGGTTGGG | 1155 |
| | 1156 | AGCTCTCCGGATCCAAGCTTATCGATTTCGAACCCGGGGTACCGAATTCCTCGAG | 1210 |
| | 1211 | ATGTTTATTTTCTTATTATTTCTTACTCTCACTAGTGGTAGTGACCTTGACCGGT | 1265 |
| | 1266 | GCACCACTTTTGATGATGTTCAAGCTCCTAATTACACTCAACATACTTCATCTAT | 1320 |
| | 1321 | GAGGGGGGTTTACTATCCTGATGAAATTTTTAGATCAGACACTCTTTATTTAACT | 1375 |
| | 1376 | CAGGATTTATTTCTTCCATTTTATTCTAATGTTACAGGGTTTCATACTATTAATC | 1430 |
| | 1431 | ATACGTTTGGCAACCCTGTCATACCTTTTAAGGATGGTATTTATTTTGCTGCCAC | 1485 |
| | 1486 | AGAGAAATCAAATGTTGTCCGTGGTTGGGTTTTTGGTTCTACCATGAACAACAAG | 1540 |
| | 1541 | TCACAGTCGGTGATTATTATTAACAATTCTACTAATGTTGTTATACGAGCATGTA | 1595 |
| | 1596 | ACTTTGAATTGTGTGACAACCCTTTCTTTGCTGTTTCTAAACCCATGGGTACACA | 1650 |
| | 1651 | GACACATACTATGATATTCGATAATGCATTTAATTGCACTTTCGAGTACATATCT | 1705 |
| | 1706 | GATGCCTTTTCGCTTGATGTTTCAGAAAAGTCAGGTAATTTTAAACACTTACGAG | 1760 |
| | 1761 | AGTTTGTGTTTAAAAATAAAGATGGGTTTCTCTATGTTTATAAGGGCTATCAACC | 1815 |
| | 1816 | TATAGATGTAGTTCGTGATCTACCTTCTGGTTTTAACACTTTGAAACCTATTTTT | 1870 |
| | 1871 | AAGTTGCCTCTTGGTATTAACATTACAAATTTTAGAGCCATTCTTACAGCCTTTT | 1925 |
| | 1926 | CACCTGCTCAAGACATTTGGGGCACGTCAGCTGCAGCCTATTTTGTTGGCTATTT | 1980 |
| | 1981 | AAAGCCAACTACATTTATGCTCAAGTATGATGAAAATGGTACAATCACAGATGCT | 2035 |
| | 2036 | GTTGATTGTTCTCAAAATCCACTTGCTGAACTCAAATGCTCTGTTAAGAGCTTTG | 2090 |
| | 2091 | AGATTGACAAAGGAATTTACCAGACCTCTAATTTCAGGGTTGTTCCCTCAGGAGA | 2145 |
| | 2146 | TGTTGTGAGATTCCCTAATATTACAAACTTGTGTCCTTTTGGAGAGGTTTTTAAT | 2200 |
| | 2201 | GCTACTAAATTCCCTTCTGTCTATGCATGGGAGAGAAAAAAAATTTCTAATTGTG | 2255 |
| | 2256 | TTGCTGATTACTCTGTGCTCTACAACTCAACATTCTTTTCAACCTTTAAGTGCTA | 2310 |
| | 2311 | TGGCGTTTCTGCCACTAAGTTGAATGATCTTTGCTTCTCCAATGTCTATGCAGAT | 2365 |
| | 2366 | TCTTTTGTAGTCAAGGGAGATGATGTAAGACAAATAGCGCCAGGACAAACTGGTG | 2420 |
| | 2421 | TTATTGCTGATTATAATTATAAATTGCCAGATGATTTCATGGGTTGTGTCCTTGC | 2475 |
| | 2476 | TTGGAATACTAGGAACATTGATGCTACTTCAACTGGTAATCATAATTATAAATAT | 2530 |
| | 2531 | AGGTATCTTAGACATGGCAAGCTTAGGCCCTTTGAGAGAGACATATCTAATGTGC | 2585 |
| | 2586 | CTTTCTCCCCTGATGGCAAACCTTGCACCCCACCTGCTCTTAATTGTTATTGGCC | 2640 |
| | 2641 | ATTAAATGATTATGGTTTTTACACCACTACTGGCATTGGCTACCAACCTTACAGA | 2695 |
| | 2696 | GTTGTAGTACTTTCTTTTGAACTTTTAAATGCACCGGCCACGGTTTGTGGACCAA | 2750 |
| | 2751 | AATTATCCACTGACCTTATTAAGAACCAGTGTGTCAATTTTAATTTTAATGGACT | 2805 |
| | 2806 | CACTGGTACTGGTGTGTTAACTCCTTCTTCAAAGAGATTTCAACCATTTCAACAA | 2860 |
| | 2861 | TTTGGCCGTGATGTTTCTGATTTCACTGATTCCGTTCGAGATCCTAAAACATCTG | 2915 |
| | 2916 | AAATATTAGACATTTCACCTTGCTCTTTTGGGGGTGTAAGTGTAATTACACCTGG | 2970 |
| | 2971 | AACAAATGCTTCATCTGAAGTTGCTGTTCTATATCAAGATGTTAACTGCACTGAT | 3025 |
| | 3026 | GTTTCTACAGCAATTCATGCAGATCAACTCACACCAGCTTGGCGCATATATTCTA | 3080 |
| | 3081 | CTGGAAACAATGTATTCCAGACTCAAGCAGGCTGTCTTATAGGAGCTGAGCATGT | 3135 |
| | 3136 | CGACACTTCTTATGAGTGCGACATTCCTATTGGAGCTGGCATTTGTGCTAGTTAC | 3190 |
| | 3191 | CATACAGTTTCTTTATTACGTAGTACTAGCCAAAAATCTATTGTGGCTTATACTA | 3245 |
| | 3246 | TGTCTTTAGGTGCTGATAGTTCAATTGCTTACTCTAATAACACCATTGCTATACC | 3300 |
| | 3301 | TACTAACTTTTCAATTAGCATTACTACAGAAGTAATGCCTGTTTCTATGGCTAAA | 3355 |
| | 3356 | ACCTCCGTAGATTGTAATATGTACATCTGCGGAGATTCTACTGAATGTGCTAATT | 3410 |
| | 3411 | TGCTTCTCCAATATGGTAGCTTTTGCACACAACTAAATCGTGCACTCTCAGGTAT | 3465 |
| | 3466 | TGCTGCTGAACAGGATCGCAACACACGTGAAGTGTTCGCTCAAGTCAAACAAATG | 3520 |
| | 3521 | TACAAAACCCCAACTTTGAAATATTTTGGTGGTTTTAATTTTTCACAAATATTAC | 3575 |
| | 3576 | CTGACCCTCTAAAGCCAACTAAGAGGTCTTTTATTGAGGACTTGCTCTTTAATAA | 3630 |
| | 3631 | GGTGACACTCGCTGATGCTGGCTTCATGAAGCAATATGGCGAATGCCTAGGTGAT | 3685 |
| | 3686 | ATTAATGCTAGAGATCTCATTTGTGCGCAGAAGTTCAATGGACTTACAGTGTTGC | 3740 |
| | 3741 | CACCTCTGCTCACTGATGATATGATTGCTGCCTACACTGCTGCTCTAGTTAGTGG | 3795 |
| | 3796 | TACTGCCACTGCTGGATGGACATTTGGTGCTGGCGCTGCTCTTCAAATACCTTTT | 3850 |
| | 3851 | GCTATGCAAATGGCATATAGGTTCAATGGCATTGGAGTTACCCAAAATGTTCTCT | 3905 |
| | 3906 | ATGAGAACCAAAAACAAATCGCCAACCAATTTAACAAGGCGATTAGTCAAATTCA | 3960 |
| | 3961 | AGAATCACTTACAACAACATCAACTGCATTGGGCAAGCTGCAAGACGTTGTTAAC | 4015 |
| | 4016 | CAGAATGCTCAAGCATTAAACACACTTGTTAAACAACTTAGCTCTAATTTTGGTG | 4070 |
| | 4071 | CAATTTCAAGTGTGCTAAATGATATCCTTTCGCGACTTGATAAAGTCGAGGCGGA | 4125 |
| | 4126 | GGTACAAATTGACAGGTTAATTACAGGCAGACTTCAAAGCCTTCAAACCTATGTA | 4180 |
| | 4181 | ACACAACAACTAATCAGGGCTGCTGAAATCAGGGCTTCTGCTAATCTTGCTGCTA | 4235 |
| | 4236 | CTAAAATGTCTGAGTGTGTTCTTGGACAATCAAAAAGAGTTGACTTTTGTGGAAA | 4290 |
| | 4291 | GGGCTACCACCTTATGTCCTTCCCACAAGCAGCCCCGCATGGTGTTGTCTTCCTA | 4345 |
| | 4346 | CATGTCACGTATGTGCCATCCCAGGAGAGGAACTTCACCACAGCGCCAGCAATTT | 4400 |
| | 4401 | GTCATGAAGGCAAAGCATACTTCCCTCGTGAAGGTGTTTTCGTGTTTAATGGCAC | 4455 |
| | 4456 | TTCTTGGTTTATTACACAGAGGAACTTCTTTTCTCCACAAATAATTACTACAGAC | 4510 |
| | 4511 | AATACATTTGTCTCAGGAAATTGTGATGTCGTTATTGGCATCATTAACAACACAG | 4565 |
| | 4566 | TTTATGATCCTCTGCAACCTGAGCTCGACTCATTCAAAGAAGAGCTGGACAAGTA | 4620 |
| | 4621 | CTTCAAAAATCATACATCACCAGATGTTGATCTTGGCGACATTTCAGGCATTAAC | 4675 |
| | 4676 | GCTTCTGTCGTCAACATTCAAAAAGAAATTGACCGCCTCAATGAGGTCGCTAAAA | 4730 |
| | 4731 | ATTTAAATGAATCACTCATTGACCTTCAAGAATTGGGAAAATATGAGCAATATAT | 4785 |
| | 4786 | TAAATGGCCTTGGTATGTTTGGCTCGGCTTCATTGCTGGACTAATTGCCATCGTC | 4840 |
| | 4841 | ATGGTTACAATCTTGCTTTGTTGCATGACTAGTTGTTGCAGTTGCCTCAAGGGTG | 4895 |
| | 4896 | CATGCTCTTGTGGTTCTTGCTGCAAGTTTGATGAGGATGACTCTGAGCCAGTTCT | 4950 |
| | 4951 | CAAGGGTGTCAAATTACATTACACATAATATTATATTTTTTATCTAAAAAACTAA | 5005 |
| | 5006 | AAATAAACATTGATTAAATTTTAATATAATACTTAAAAATGGATGTTGTGTCGTT | 5060 |
| | 5061 | AGATAAACCGTTTATGTATTTTGAGGAAATTGATAATGAGTTAGATTACGAACCA | 5115 |
| | 5116 | GAAAGTGCAAATGAGGTCGCAAAAAAACTACCGTATCAAGGACAGTTAAAACTAT | 5170 |
| | 5171 | TACTAGGAGAATTATTTTTTCTTAGTAAGTTACAGCGACACGGTATATTAGATGG | 5225 |
| | 5226 | TGCCACCGTAGTGTATATAGGATCGGCTCCTGGTACACATATACGTTATTTGAGA | 5280 |
| | 5281 | GATCATTTCTATAATTTAGGAATGATTATCAAATGGATGCTAATTGACGGACGCC | 5335 |
| | 5336 | ATCATGATCCTATTCTAAATGGATTGCGTGATGTGACTCTAGTGACTCGGTTCGT | 5390 |
| | 5391 | TGATGAGGAATATCTACGATCCATCAAAAAACAACTGCATCCTTCTAAGATTATT | 5445 |
| | 5446 | TTAATTTCTGATGTAAGATCCAAACGAGGAGGAAATGAACCTAGTACGGCGGATT | 5500 |
| | 5501 | TACTAAGTAATTACGCTCTACAAAATGTCATGATTAGTATTTTAAACCCCGTGGC | 5555 |
| | 5556 | ATCTAGTCTTAAATGGAGATGCCCGTTTCCAGATCAATGGATCAAGGACTTTTAT | 5610 |
| | 5611 | ATCCCACACGGTAATAAAATGTTACAACCTTTTGCTCCTTCATATTCAGCTGAAA | 5665 |
| | 5666 | TGAGATTATTAAGTATTTATACCGGTGAGAACATGAGACTGACTCGAGTTACCAA | 5720 |
| | 5721 | ATTAGACGCTGTAAATTATGAAAAAAAGATGTACTACCTTAATAAGATCGTCCGT | 5775 |
| | 5776 | AACAAAGTAGTTGTTAACTTTGATTATCCTAATCAGGAATATGACTATTTTCACA | 5830 |
| | 5831 | TGTACTTTATGCTGAGGACCGTATACTGCAATAAAACATTTCCTACTACTAAAGC | 5885 |
| | 5886 | AAAGGTACTATTTCTACAACAATCTATATTTCGTTTCTTAAATATTCCAACAACA | 5940 |
| | 5941 | TCAACTGAAAAAGTTAGTCATGAACCAATACAACGTAA | 5978 |
| Synthetic vaccinia virus | 1 | ATTTACGGATTCACCAATAAAAATAAACTAGAGAAACTTAGTACTAATAAGGAAC | 55 |
| comprising a nucleic acid | 56 | TAGAATCGTATAGTTCTAGCCCTCTTCAAGAACCCATTAGGTTAAATGATTTTCT | 110 |
| encoding a SARS-CoV-2 | 111 | GGGACTATTGGAATGTGTTAAAAAGAATATTCCTCTAACAGATATTCCGACAAAG | 165 |
| virus S protein. | 166 | GATTGATTACTATAAATGGAGAATGTTCCTAATGTATACTTTAATCCTGTGTTTA | 220 |
| SEQ ID NO: 44 | 221 | TAGAGCCCACGTTTAAACATTCTTTATTAAGTGTTTATAAACACAGATTAATAGT | 275 |
| | 276 | TTTATTTGAAGTATTCGTTGTATTCATTCTAATATATGTATTTTTTAGATCTGAA | 330 |
| | 331 | TTAAATATGTTCTTCATGCCTAAACGAAAAATACCCGATCCTATTGATAGATTAC | 385 |
| | 386 | GACGTGCTAATCTAGCGTGTGAAGACGATAAATTAATGATCTATGGATTACCATG | 440 |
| | 441 | GATGACAACTCAAACATCTGCGTTATCAATAAATAGTAAACCGATAGTGTATAAA | 495 |
| | 496 | GATTGTGCAAAGCTTTTGCGATCAATAAATGGATCACAACCAGTATCTCTTAACG | 550 |
| | 551 | ATGTTCTTCGCAGATGATGATTCATTTTTTAAGTATTTGGCTAGTCAAGATGATG | 605 |
| | 606 | AATCTTCATTATCTGATATATTGCAAATCACTCAATATCTAGACTTTCTGTTATT | 660 |
| | 661 | ATTATTGATCCAATCAAAAAATAAATTAGAAGCCGTGGGTCATTGTTATGAATCT | 715 |
| | 716 | CTTTCAGAGGAATACAGACAATTGACAAAATTCACAGACTCTCAAGATTTTAAAA | 770 |
| | 771 | AACTGTTTAACAAGGTCCCTATTGTTACAGATGGAAGGGTCAAACTTAATAAAGG | 825 |
| | 826 | ATATTTGTTCGACTTTGTGATTAGTTTGATGCGATTCAAAAAAGAATCAGCTCTA | 880 |
| | 881 | GCTACCACCGCAATAGATCCTGTTAGATACATAGATCCTCGTCGCGATATCGCAT | 935 |
| | 936 | TTTCTAACGTGATGGATATATTAAAGTCGAATAAAGTGAACAATAATTAATTCTT | 990 |
| | 991 | TATTGTCATCTTTTATTTTTTTTTTTTGGAATATAAATATCCGGTAAAATTGAAA | 1045 |
| | 1046 | AAATATACACTAATTAGCGTCTCGTTTCAGACGCTAGCTCGAGGTTGGGAGCTCT | 1100 |
| | 1101 | CCGGATCCAAGCTTATCGATTTCGAACCCGGGGTACCGAATTCCTCGAGGTTGGG | 1155 |
| | 1156 | AGCTCTCCGGATCCAAGCTTATCGATTTCGAACCCGGGGTACCGAATTCCTCGAG | 1210 |
| | 1211 | ATGTTTATTTTCTTATTATTTCTTACTCTCACTAGTGGTAGTGACCTTGACCGGT | 1265 |
| | 1266 | GCACCACTTTTGATGATGTTCAAGCTCCTAATTACACTCAACATACTTCATCTAT | 1320 |
| | 1321 | GAGGGGGGTTTACTATCCTGATGAAATTTTTAGATCAGACACTCTTTATTTAACT | 1375 |
| | 1376 | CAGGATTTATTTCTTCCATTTTATTCTAATGTTACAGGGTTTCATACTATTAATC | 1430 |
| | 1431 | ATACGTTTGGCAACCCTGTCATACCTTTTAAGGATGGTATTTATTTTGCTGCCAC | 1485 |
| | 1486 | AGAGAAATCAAATGTTGTCCGTGGTTGGGTTTTTGGTTCTACCATGAACAACAAG | 1540 |
| | 1541 | TCACAGTCGGTGATTATTATTAACAATTCTACTAATGTTGTTATACGAGCATGTA | 1595 |
| | 1596 | ACTTTGAATTGTGTGACAACCCTTTCTTTGCTGTTTCTAAACCCATGGGTACACA | 1650 |
| | 1651 | GACACATACTATGATATTCGATAATGCATTTAATTGCACTTTCGAGTACATATCT | 1705 |
| | 1706 | GATGCCTTTTCGCTTGATGTTTCAGAAAAGTCAGGTAATTTTAAACACTTACGAG | 1760 |
| | 1761 | AGTTTGTGTTTAAAAATAAAGATGGGTTTCTCTATGTTTATAAGGGCTATCAACC | 1815 |
| | 1816 | TATAGATGTAGTTCGTGATCTACCTTCTGGTTTTAACACTTTGAAACCTATTTTT | 1870 |
| | 1871 | AAGTTGCCTCTTGGTATTAACATTACAAATTTTAGAGCCATTCTTACAGCCTTTT | 1925 |
| | 1926 | CACCTGCTCAAGACATTTGGGGCACGTCAGCTGCAGCCTATTTTGTTGGCTATTT | 1980 |
| | 1981 | AAAGCCAACTACATTTATGCTCAAGTATGATGAAAATGGTACAATCACAGATGCT | 2035 |
| | 2036 | GTTGATTGTTCTCAAAATCCACTTGCTGAACTCAAATGCTCTGTTAAGAGCTTTG | 2090 |
| | 2091 | AGATTGACAAAGGAATTTACCAGACCTCTAATTTCAGGGTTGTTCCCTCAGGAGA | 2145 |
| | 2146 | TGTTGTGAGATTCCCTAATATTACAAACTTGTGTCCTTTTGGAGAGGTTTTTAAT | 2200 |
| | 2201 | GCTACTAAATTCCCTTCTGTCTATGCATGGGAGAGAAAAAAAATTTCTAATTGTG | 2255 |
| | 2256 | TTGCTGATTACTCTGTGCTCTACAACTCAACATTCTTTTCAACCTTTAAGTGCTA | 2310 |
| | 2311 | TGGCGTTTCTGCCACTAAGTTGAATGATCTTTGCTTCTCCAATGTCTATGCAGAT | 2365 |
| | 2366 | TCTTTTGTAGTCAAGGGAGATGATGTAAGACAAATAGCGCCAGGACAAACTGGTG | 2420 |
| | 2421 | TTATTGCTGATTATAATTATAAATTGCCAGATGATTTCATGGGTTGTGTCCTTGC | 2475 |
| | 2476 | TTGGAATACTAGGAACATTGATGCTACTTCAACTGGTAATCATAATTATAAATAT | 2530 |
| | 2531 | AGGTATCTTAGACATGGCAAGCTTAGGCCCTTTGAGAGAGACATATCTAATGTGC | 2585 |
| | 2586 | CTTTCTCCCCTGATGGCAAACCTTGCACCCCACCTGCTCTTAATTGTTATTGGCC | 2640 |
| | 2641 | ATTAAATGATTATGGTTTTTACACCACTACTGGCATTGGCTACCAACCTTACAGA | 2695 |
| | 2696 | GTTGTAGTACTTTCTTTTGAACTTTTAAATGCACCGGCCACGGTTTGTGGACCAA | 2750 |
| | 2751 | AATTATCCACTGACCTTATTAAGAACCAGTGTGTCAATTTTAATTTTAATGGACT | 2805 |
| | 2806 | CACTGGTACTGGTGTGTTAACTCCTTCTTCAAAGAGATTTCAACCATTTCAACAA | 2860 |
| | 2861 | TTTGGCCGTGATGTTTCTGATTTCACTGATTCCGTTCGAGATCCTAAAACATCTG | 2915 |
| | 2916 | AAATATTAGACATTTCACCTTGCTCTTTTGGGGGTGTAAGTGTAATTACACCTGG | 2970 |
| | 2971 | AACAAATGCTTCATCTGAAGTTGCTGTTCTATATCAAGATGTTAACTGCACTGAT | 3025 |
| | 3026 | GTTTCTACAGCAATTCATGCAGATCAACTCACACCAGCTTGGCGCATATATTCTA | 3080 |
| | 3081 | CTGGAAACAATGTATTCCAGACTCAAGCAGGCTGTCTTATAGGAGCTGAGCATGT | 3135 |
| | 3136 | CGACACTTCTTATGAGTGCGACATTCCTATTGGAGCTGGCATTTGTGCTAGTTAC | 3190 |
| | 3191 | CATACAGTTTCTTTATTACGTAGTACTAGCCAAAAATCTATTGTGGCTTATACTA | 3245 |
| | 3246 | TGTCTTTAGGTGCTGATAGTTCAATTGCTTACTCTAATAACACCATTGCTATACC | 3300 |
| | 3301 | TACTAACTTTTCAATTAGCATTACTACAGAAGTAATGCCTGTTTCTATGGCTAAA | 3355 |
| | 3356 | ACCTCCGTAGATTGTAATATGTACATCTGCGGAGATTCTACTGAATGTGCTAATT | 3410 |
| | 3411 | TGCTTCTCCAATATGGTAGCTTTTGCACACAACTAAATCGTGCACTCTCAGGTAT | 3465 |
| | 3466 | TGCTGCTGAACAGGATCGCAACACACGTGAAGTGTTCGCTCAAGTCAAACAAATG | 3520 |
| | 3521 | TACAAAACCCCAACTTTGAAATATTTTGGTGGTTTTAATTTTTCACAAATATTAC | 3575 |
| | 3576 | CTGACCCTCTAAAGCCAACTAAGAGGTCTTTTATTGAGGACTTGCTCTTTAATAA | 3630 |
| | 3631 | GGTGACACTCGCTGATGCTGGCTTCATGAAGCAATATGGCGAATGCCTAGGTGAT | 3685 |
| | 3686 | ATTAATGCTAGAGATCTCATTTGTGCGCAGAAGTTCAATGGACTTACAGTGTTGC | 3740 |
| | 3741 | CACCTCTGCTCACTGATGATATGATTGCTGCCTACACTGCTGCTCTAGTTAGTGG | 3795 |
| | 3796 | TACTGCCACTGCTGGATGGACATTTGGTGCTGGCGCTGCTCTTCAAATACCTTTT | 3850 |
| | 3851 | GCTATGCAAATGGCATATAGGTTCAATGGCATTGGAGTTACCCAAAATGTTCTCT | 3905 |
| | 3906 | ATGAGAACCAAAAACAAATCGCCAACCAATTTAACAAGGCGATTAGTCAAATTCA | 3960 |
| | 3961 | AGAATCACTTACAACAACATCAACTGCATTGGGCAAGCTGCAAGACGTTGTTAAC | 4015 |
| | 4016 | CAGAATGCTCAAGCATTAAACACACTTGTTAAACAACTTAGCTCTAATTTTGGTG | 4070 |
| | 4071 | CAATTTCAAGTGTGCTAAATGATATCCTTTCGCGACTTGATAAAGTCGAGGCGGA | 4125 |
| | 4126 | GGTACAAATTGACAGGTTAATTACAGGCAGACTTCAAAGCCTTCAAACCTATGTA | 4180 |
| | 4181 | ACACAACAACTAATCAGGGCTGCTGAAATCAGGGCTTCTGCTAATCTTGCTGCTA | 4235 |
| | 4236 | CTAAAATGTCTGAGTGTGTTCTTGGACAATCAAAAAGAGTTGACTTTTGTGGAAA | 4290 |
| | 4291 | GGGCTACCACCTTATGTCCTTCCCACAAGCAGCCCCGCATGGTGTTGTCTTCCTA | 4345 |
| | 4346 | CATGTCACGTATGTGCCATCCCAGGAGAGGAACTTCACCACAGCGCCAGCAATTT | 4400 |
| | 4401 | GTCATGAAGGCAAAGCATACTTCCCTCGTGAAGGTGTTTTCGTGTTTAATGGCAC | 4455 |
| | 4456 | TTCTTGGTTTATTACACAGAGGAACTTCTTTTCTCCACAAATAATTACTACAGAC | 4510 |
| | 4511 | AATACATTTGTCTCAGGAAATTGTGATGTCGTTATTGGCATCATTAACAACACAG | 4565 |
| | 4566 | TTTATGATCCTCTGCAACCTGAGCTCGACTCATTCAAAGAAGAGCTGGACAAGTA | 4620 |
| | 4621 | CTTCAAAAATCATACATCACCAGATGTTGATCTTGGCGACATTTCAGGCATTAAC | 4675 |
| | 4676 | GCTTCTGTCGTCAACATTCAAAAAGAAATTGACCGCCTCAATGAGGTCGCTAAAA | 4730 |
| | 4731 | ATTTAAATGAATCACTCATTGACCTTCAAGAATTGGGAAAATATGAGCAATATAT | 4785 |
| | 4786 | TAAATGGCCTTGGTATGTTTGGCTCGGCTTCATTGCTGGACTAATTGCCATCGTC | 4840 |
| | 4841 | ATGGTTACAATCTTGCTTTGTTGCATGACTAGTTGTTGCAGTTGCCTCAAGGGTG | 4895 |
| | 4896 | CATGCTCTTGTGGTTCTTGCTGCAAGTTTGATGAGGATGACTCTGAGCCAGTTCT | 4950 |
| | 4951 | CAAGGGTGTCAAATTACATTACACATAATATTATATTTTTTATCTAAAAAACTAA | 5005 |
| | 5006 | AAATAAACATTGATTAAATTTTAATATAATACTTAAAAATGGATGTTGTGTCGTT | 5060 |
| | 5061 | AGATAAACCGTTTATGTATTTTGAGGAAATTGATAATGAGTTAGATTACGAACCA | 5115 |
| | 5116 | GAAAGTGCAAATGAGGTCGCAAAAAAACTGCCGTATCAAGGACAGTTAAAACTAT | 5170 |
| | 5171 | TACTAGGAGAATTATTTTTTCTTAGTAAGTTACAGCGACACGGTATATTAGATGG | 5225 |
| | 5226 | TGCCACCGTAGTGTATATAGGATCTGCTCCCGGTACACATATACGTTATTTGAGA | 5280 |
| | 5281 | GATCATTTCTATAATTTAGGAGTGATCATCAAATGGATGCTAATTGACGGCCGCC | 5335 |
| | 5336 | ATCATGATCCTATTTTAAATGGATTGCGTGATGTGACTCTAGTGACTCGGTTCGT | 5390 |
| | 5391 | TGATGAGGAATATCTACGATCCATCAAAAAACAACTGCATCCTTCTAAGATTATT | 5445 |
| | 5446 | TTAATTTCTGATGTGAGATCCAAACGAGGAGGAAATGAACCTAGTACGGCGGATT | 5500 |
| | 5501 | TACTAAGTAATTACGCTCTACAAAATGTCATGATTAGTATTTTAAACCCCGTGGC | 5555 |
| | 5556 | ATCTAGTCTTAAATGGAGATGCCCGTTTCCAGATCAATGGATCAAGGACTTTTAT | 5610 |
| | 5611 | ATCCCACACGGTAATAAAATGTTACAACCTTTTGCTCCTTCATATTCAGCTGAAA | 5665 |
| | 5666 | TGAGATTATTAAGTATTTATACCGGTGAGAACATGAGACTGACTCGAGTTACCAA | 5720 |
| | 5721 | ATTAGACGCTGTAAATTATGAAAAAAAGATGTACTACCTTAATAAGATCGTCCGT | 5775 |
| | 5776 | AACAAAGTAGTTGTTAACTTTGATTATCCTAATCAGGAATATGACTATTTTCACA | 5830 |
| | 5831 | TGTACTTTATGCTGAGGACCGTGTACTGCAATAAAACATTTCCTACTACTAAAGC | 5885 |
| | 5886 | AAAGGTACTATTTCTACAACAATCTATATTTCGTTTCTTAAATATTCCAACAACA | 5940 |
| | 5941 | TCAACTGAAAAAGTTAGTCATGAACCAATACAACGTAA | 5978 |

| Nucleic acid encoding S | 21562 | atgtttgtt tttcttgttt tattgccact agtctctagt |
|---|---|---|
| protein (21562-25383) | 21601 | cagtgtgtta atcttacaac cagaactcaa ttaccccctg catacactaa ttctttcaca |
| Gene Bank accession | 21661 | cgtggtgttt attaccctga caaagttttc agatcctcag ttttacattc aactcaggac |
| number MN988668 or | 21721 | ttgttcttac ctttcttttc caatgttact tggttccatg ctatacatgt ctctgggacc |
| (21579-25400) Gene Bank | 21781 | aatggtacta agaggtttga taaccctgtc ctaccattta atgatggtgt ttattttgct |
| accession number | 21841 | tccactgaga agtctaacat aataagaggc tggatttttg gtactacttt agattcgaag |
| NC_045512. SEQ ID NO: 45 | 21901 | acccagtccc tacttattgt taataacgct actaatgttg ttattaaagt ctgtgaattt |
| | 21961 | caattttgta atgatccatt tttgggtgtt tattaccaca aaaacaacaa aagttggatg |
| | 22021 | gaaagtgagt tcagagttta ttctagtgcg aataattgca cttttgaata tgtctctcag |
| | 22081 | ccttttctta tggaccttga aggaaaacag ggtaatttca aaaatcttag ggaatttgtg |
| | 22141 | tttaagaata ttgatggtta ttttaaaata tattctaagc acacgcctat taatttagtg |
| | 22201 | cgtgatctcc ctcagggttt ttcggcttta gaaccattgg tagatttgcc aataggtatt |
| | 22261 | aacatcacta ggtttcaaac tttacttgct ttacatagaa gttatttgac tcctggtgat |
| | 22321 | tcttcttcag gttggacagc tggtgctgca gcttattatg tgggttatct tcaacctagg |
| | 22381 | acttttctat taaaatataa tgaaaatgga accattacag atgctgtaga ctgtgcactt |
| | 22441 | gaccctctct cagaaacaaa gtgtacgttg aaatccttca ctgtagaaaa aggaatctat |
| | 22501 | caaacttcta actttagagt ccaaccaaca gaatctattg ttagatttcc taatattaca |
| | 22561 | aacttgtgcc cttttggtga agtttttaac gccaccagat ttgcatctgt ttatgcttgg |
| | 22621 | aacaggaaga gaatcagcaa ctgtgttgct gattattctg tcctatataa ttccgcatca |
| | 22681 | ttttccactt ttaagtgtta tggagtgtct cctactaaat taaatgatct ctgctttact |
| | 22741 | aatgtctatg cagattcatt tgtaattaga ggtgatgaag tcagacaaat cgctccaggg |
| | 22801 | caaactggaa agattgctga ttataattat aaattaccag atgattttac aggctgcgtt |
| | 22861 | atagcttgga attctaacaa tcttgattct aaggttggtg gtaattataa ttacctgtat |
| | 22921 | agattgttta ggaagtctaa tctcaaacct tttgagagag atatttcaac tgaaatctat |
| | 22981 | caggccggta gcacaccttg taatggtgtt gaaggtttta attgttactt tcctttacaa |
| | 23041 | tcatatggtt tccaacccac taatggtgtt ggttaccaac catacagagt agtagtactt |
| | 23101 | tcttttgaac ttctacatgc accagcaact gtttgtggac ctaaaaagtc tactaatttg |
| | 23161 | gttaaaaaca aatgtgtcaa tttcaacttc aatggtttaa caggcacagg tgttcttact |
| | 23221 | gagtctaaca aaaagtttct gcctttccaa caatttggca gagacattgc tgacactact |
| | 23281 | gatgctgtcc gtgatccaca gacacttgag attcttgaca ttacaccatg ttcttttggt |
| | 23341 | ggtgtcagtg ttataacacc aggaacaaat acttctaacc aggttgctgt tctttatcag |
| | 23401 | gatgttaact gcacagaagt ccctgttgct attcatgcag atcaacttac tcctacttgg |
| | 23461 | cgtgtttatt ctacaggttc taatgttttt caaacacgtg caggctgttt aataggggct |
| | 23521 | gaacatgtca acaactcata tgagtgtgac atacccattg gtgcaggtat atgcgctagt |
| | 23581 | tatcagactc agactaattc tcctcggcgg gcacgtagtg tagctagtca atccatcatt |
| | 23641 | gcctacacta tgtcacttgg tgcagaaaat tcagttgctt actctaataa ctctattgcc |
| | 23701 | atacccacaa attttactat tagtgttacc acagaaattc taccagtgtc tatgaccaag |
| | 23761 | acatcagtag attgtacaat gtacatttgt ggtgattcaa ctgaatgcag caatcttttg |
| | 23821 | ttgcaatatg gcagtttttg tacacaatta aaccgtgctt taactggaat agctgttgaa |
| | 23881 | caagacaaaa acacccaaga agtttttgca caagtcaaac aaatttacaa aacaccacca |
| | 23941 | attaaagatt ttggtggttt taatttttca caaatattac cagatccatc aaaaccaagc |
| | 24001 | aagaggtcat ttattgaaga tctacttttc aacaaagtga cacttgcaga tgctggcttc |
| | 24061 | atcaaacaat atggtgattg ccttggtgat attgctgcta gagacctcat ttgtgcacaa |
| | 24121 | aagtttaacg gccttactgt tttgccacct ttgctcacag atgaaatgat tgctcaatac |
| | 24181 | acttctgcac tgttagcggg tacaatcact tctggttgga cctttggtgc aggtgctgca |
| | 24241 | ttacaaatac catttgctat gcaaatggct tataggttta atggtattgg agttacacag |
| | 24301 | aatgttctct atgagaacca aaaattgatt gccaaccaat ttaatagtgc tattggcaaa |
| | 24361 | attcaagact cactttcttc cacagcaagt gcacttggaa aacttcaaga tgtggtcaac |
| | 24421 | caaaatgcac aagctttaaa cacgcttgtt aaacaactta gctccaattt tggtgcaatt |
| | 24481 | tcaagtgttt taaatgatat cctttcacgt cttgacaaag ttgaggctga agtgcaaatt |
| | 24541 | gataggttga tcacaggcag acttcaaagt ttgcagacat atgtgactca acaattaatt |
| | 24601 | agagctgcag aaatcagagc ttctgctaat cttgctgcta ctaaaatgtc agagtgtgta |
| | 24661 | cttggacaat caaaaagagt tgatttttgt ggaaagggct atcatcttat gtccttccct |
| | 24721 | cagtcagcac ctcatggtgt agtcttcttg catgtgactt atgtccctgc acaagaaaag |
| | 24781 | aacttcacaa ctgctcctgc catttgtcat gatggaaaag cacactttcc tcgtgaaggt |
| | 24841 | gtctttgttt caaatggcac acactggttt gtaacacaaa ggaattttta tgaaccacaa |
| | 24901 | atcattacta cagacaacac atttgtgtct ggtaactgtg atgttgtaat aggaattgtc |
| | 24961 | aacaacacag tttatgatcc tttgcaacct gaattagact cattcaagga ggagttagat |
| | 25021 | aaatatttta agaatcatac atcaccagat gttgatttag gtgacatctc tggcattaat |
| | 25081 | gcttcagttg taaacattca aaaagaaatt gaccgcctca atgaggttgc caagaattta |
| | 25141 | aatgaatctc tcatcgatct ccaagaactt ggaaagtatg agcagtatat aaaatggcca |
| | 25201 | tggtacattt ggctaggttt tatagctggc ttgattgcca tagtaatggt gacaattatg |
| | 25261 | ctttgctgta tgaccagttg ctgtagttgt ctcaagggct gttgttcttg tggatcctgc |
| | 25321 | tgcaaatttg atgaagacga ctctgagcca gtgctcaaag gagtcaaatt acattacaca |
| | 25381 | taa |
| Nucleotide Sequence of | 1 | ttaaaggttt ataccttccc aggtaacaaa ccaaccaact ttcgatctct tgtagatctg |
| SARS-CoV2 isolate 2019- | 61 | ttctctaaac gaactttaaa atctgtgtgg ctgtcactcg gctgcatgct tagtgcactc |
| nCoV WHU01, complete | 121 | acgcagtata attaataact aattactgtc gttgacagga cacgagtaac tcgtctatct |
| genome. GenBank Accession | 181 | tctgcaggct gcttacggtt tcgtccgtgt tgcagccgat catcagcaca tctaggtttc |
| Number MN988668.1. | 241 | gtccgggtgt gaccgaaagg taagatggag agccttgtcc ctggtttcaa cgagaaaaca |
| SEQ ID NO: 46 | 301 | cacgtccaac tcagtttgcc tgttttacag gttcgcgacg tgctcgtacg tggctttgga |
| | 361 | gactccgtgg aggaggtctt atcagaggca cgtcaacatc ttaaagatgg cacttgtggc |
| | 421 | ttagtagaag ttgaaaaagg cgttttgcct caacttgaac agccctatgt gttcatcaaa |
| | 481 | cgttcggatg ctcgaactgc acctcatggt catgttatgg ttgagctggt agcagaactc |
| | 541 | gaaggcattc agtacggtcg tagtggtgag acacttggtg tccttgtccc tcatgtgggc |
| | 601 | gaaataccag tggcttaccg caaggttctt cttcgtaaga acggtaataa aggagctggt |
| | 661 | ggccatagtt acggcgccga tctaaagtca tttgacttag gcgacgagct tggcactgat |
| | 721 | ccttatgaag attttcaaga aaactggaac actaaacata gcagtggtgt tacccgtgaa |
| | 781 | ctcatgcgtg agcttaacgg aggggcatac actcgctatg tcgataacaa cttctgtggc |
| | 841 | cctgatggct accctcttga gtgcattaaa gaccttctag cacgtgctgg taaagcttca |
| | 901 | tgcactttgt ccgaacaact ggactttatt gacactaaga ggggtgtata ctgctgccgt |
| | 961 | gaacatgagc atgaaattgc ttggtacacg gaacgttctg aaaagagcta tgaattgcag |
| | 1021 | acaccttttg aaattaaatt ggcaaagaaa tttgacacct tcaatgggga atgtccaaat |
| | 1081 | tttgtatttc ccttaaattc cataatcaag actattcaac caagggttga aaagaaaaag |
| | 1141 | cttgatggct ttatgggtag aattcgatct gtctatccag ttgcgtcacc aaatgaatgc |
| | 1201 | aaccaaatgt gcctttcaac tctcatgaag tgtgatcatt gtggtgaaac ttcatggcag |
| | 1261 | acgggcgatt ttgttaaagc cacttgcgaa ttttgtggca ctgagaattt gactaaagaa |
| | 1321 | ggtgccacta cttgtggtta cttaccccaa aatgctgttg ttaaaattta ttgtccagca |
| | 1381 | tgtcacaatt cagaagtagg acctgagcat agtcttgccg aataccataa tgaatctggc |
| | 1441 | ttgaaaacca ttcttcgtaa gggtggtcgc actattgcct ttggaggctg tgtgttctct |
| | 1501 | tatgttggtt gccataacaa gtgtgcctat tgggttccac gtgctagcgc taacataggt |
| | 1561 | tgtaaccata caggtgttgt tggagaaggt tccgaaggtc ttaatgacaa ccttcttgaa |
| | 1621 | atactccaaa aagagaaagt caacatcaat attgttggtg actttaaact taatgaagag |
| | 1681 | atcgccatta ttttggcatc tttttctgct tccacaagtg cttttgtgga aactgtgaaa |
| | 1741 | ggtttggatt ataaagcatt caaacaaatt gttgaatcct gtggtaattt taaagttaca |
| | 1801 | aaaggaaaag ctaaaaaagg tgcctggaat attggtgaac agaaatcaat actgagtcct |
| | 1861 | ctttatgcat ttgcatcaga ggctgctcgt gttgtacgat caattttctc ccgcactctt |
| | 1921 | gaaactgctc aaaattctgt gcgtgtttta cagaaggccg ctataacaat actagatgga |
| | 1981 | atttcacagt attcactgag actcattgat gctatgatgt tcacatctga tttggctact |
| | 2041 | aacaatctag ttgtaatggc ctacattaca ggtggtgttg ttcagttgac ttcgcagtgg |
| | 2101 | ctaactaaca tctttggcac tgtttatgaa aaactcaaac ccgtccttga ttggcttgaa |
| | 2161 | gagaagttta aggaaggtgt agagtttctt agagacggtt gggaaattgt taaatttatc |
| | 2221 | tcaacctgtg cttgtgaaat tgtcggtgga caaattgtca cctgtgcaaa ggaaattaag |
| | 2281 | gagagtgttc agacattctt taagcttgta aataaatttt tggctttgtg tgctgactct |
| | 2341 | atcattattg gtggagctaa acttaaagcc ttgaatttag gtgaaacatt tgtcacgcac |
| | 2401 | tcaaagggat tgtacagaaa gtgtgttaaa tccagagaag aaactggcct actcatgcct |
| | 2461 | ctaaaagccc caaaagaaat tatcttctta gagggagaaa cacttcccac agaagtgtta |
| | 2521 | acagaggaag ttgtcttgaa aactggtgat ttacaaccat tagaacaacc tactagtgaa |
| | 2581 | gctgttgaag ctccattggt tggtacacca gtttgtatta acgggcttat gttgctcgaa |
| | 2641 | atcaaagaca cagaaaagta ctgtgccctt gcacctaata tgatggtaac aaacaatacc |
| | 2701 | ttcacactca aaggcggtgc accaacaaag gttacttttg gtgatgacac tgtgatagaa |
| | 2761 | gtgcaaggtt acaagagtgt gaatatcact tttgaacttg atgaaaggat tgataaagta |
| | 2821 | cttaatgaga agtgctctgc ctatacagtt gaactcggta cagaagtaaa tgagttcgcc |
| | 2881 | tgtgttgtgg cagatgctgt cataaaaact ttgcaaccag tatctgaatt acttacacca |
| | 2941 | ctgggcattg atttagatga gtggagtatg gctacatact acttatttga tgagtctggt |
| | 3001 | gagtttaaat tggcttcaca tatgtattgt tctttctacc ctccagatga ggatgaagaa |
| | 3061 | gaaggtgatt gtgaagaaga agagtttgag ccatcaactc aatatgagta tggtactgaa |
| | 3121 | gatgattacc aaggtaaacc tttggaattt ggtgccactt ctgctgctct tcaacctgaa |
| | 3181 | gaagagcaag aagaagattg gttagatgat gatagtcaac aaactgttgg tcaacaagac |
| | 3241 | ggcagtgagg acaatcagac aactactatt caaacaattg ttgaggttca acctcaatta |
| | 3301 | gagatggaac ttacaccagt tgttcagact attgaagtga atagttttag tggttattta |
| | 3361 | aaacttactg acaatgtata cattaaaaat gcagacattg tggaagaagc taaaaaggta |
| | 3421 | aaaccaacag tggttgttaa tgcagccaat gtttacctta aacatggagg aggtgttgca |
| | 3481 | ggagccttaa ataaggctac taacaatgcc atgcaagttg aatctgatga ttacatagct |
| | 3541 | actaatggac cacttaaagt gggtggtagt tgtgttttaa gcggacacaa tcttgctaaa |
| | 3601 | cactgtcttc atgttgtcgg cccaaatgtt aacaaaggtg aagacattca acttcttaag |
| | 3661 | agtgcttatg aaaattttaa tcagcacgaa gttctacttg caccattatt atcagctggt |
| | 3721 | atttttggtg ctgaccctat acattcttta agagtttgtg tagatactgt tcgcacaaat |
| | 3781 | gtctacttag ctgtctttga taaaaatctc tatgacaaac ttgtttcaag ctttttggaa |
| | 3841 | atgaagagtg aaaagcaagt tgaacaaaag atcgctgaga ttcctaaaga ggaagttaag |
| | 3901 | ccatttataa ctgaaagtaa accttcagtt gaacagagaa aacaagatga taagaaaatc |
| | 3961 | aaagcttgtg ttgaagaagt tacaacaact ctggaagaaa ctaagttcct cacagaaaac |
| | 4021 | ttgttacttt atattgacat taatggcaat cttcatccag attctgccac tcttgttagt |
| | 4081 | gacattgaca tcactttctt aaagaaagat gctccatata tagtgggtga tgttgttcaa |
| | 4141 | gagggtgttt taactgctgt ggttatacct actaaaaagg ctggtggcac tactgaaatg |
| | 4201 | ctagcgaaag ctttgagaaa agtgccaaca gacaattata taaccactta cccgggtcag |
| | 4261 | ggtttaaatg gttacactgt agaggaggca aagacagtgc ttaaaaagtg taaaagtgcc |
| | 4321 | ttttacattc taccatctat tatctctaat gagaagcaag aaattcttgg aactgtttct |
| | 4381 | tggaatttgc gagaaatgct tgcacatgca gaagaaacac gcaaattaat gcctgtctgt |
| | 4441 | gtggaaacta aagccatagt ttcaactata cagcgtaaat ataagggtat taaaatacaa |
| | 4501 | gagggtgtgg ttgattatgg tgctagattt tacttttaca ccagtaaaac aactgtagcg |
| | 4561 | tcacttatca acacacttaa cgatctaaat gaaactcttg ttacaatgcc acttggctat |
| | 4621 | gtaacacatg gcttaaattt ggaagaagct gctcggtata tgagatctct caaagtgcca |
| | 4681 | gctacagttt ctgtttcttc acctgatgct gttacagcgt ataatggtta tcttacttct |
| | 4741 | tcttctaaaa cacctgaaga acattttatt gaaaccatct cacttgctgg ttcctataaa |
| | 4801 | gattggtcct attctggaca atctacacaa ctaggtatag aatttcttaa gagaggtgat |
| | 4861 | aaaagtgtat attacactag taatcctacc acattccacc tagatggtga agttatcacc |
| | 4921 | tttgacaatc ttaagacact tctttctttg agagaagtga ggactattaa ggtgtttaca |
| | 4981 | acagtagaca acattaacct ccacacgcaa gttgtggaca tgtcaatgac atatggacaa |
| | 5041 | cagtttggtc caacttattt ggatggagct gatgttacta aaataaaacc tcataattca |
| | 5101 | catgaaggta aaacatttta tgttttacct aatgatgaca ctctacgtgt tgaggctttt |
| | 5161 | gagtactacc acacaactga tcctagtttt ctgggtaggt acatgtcagc attaaatcac |
| | 5221 | actaaaaagt ggaaataccc acaagttaat ggtttaactt ctattaaatg ggcagataac |
| | 5281 | aactgttatc ttgccactgc attgttaaca ctccaacaaa tagagttgaa gtttaatcca |
| | 5341 | cctgctctac aagatgctta ttacagagca agggctggtg aagctgctaa cttttgtgca |
| | 5401 | cttatcttag cctactgtaa taagacagta ggtgagttag gtgatgttag agaaacaatg |
| | 5461 | agttacttgt ttcaacatgc caatttagat tcttgcaaaa gagtcttgaa cgtggtgtgt |
| | 5521 | aaaacttgtg gacaacagca gacaaccctt aagggtgtag aagctgttat gtacatgggc |
| | 5581 | acactttctt atgaacaatt taagaaaggt gttcagatac cttgtacgtg tggtaaacaa |
| | 5641 | gctacaaaat atctagtaca acaggagtca ccttttgtta tgatgtcagc accacctgct |
| | 5701 | cagtatgaac ttaagcatgg tacatttact tgtgctagtg agtacactgg taattaccag |
| | 5761 | tgtggtcact ataaacatat aacttctaaa gaaactttgt attgcataga cggtgcttta |
| | 5821 | cttacaaagt cctcagaata caaaggtcct attacggatg ttttctacaa agaaaacagt |
| | 5881 | tacacaacaa ccataaaacc agttacttat aaattggatg gtgttgtttg tacagaaatt |
| | 5941 | gaccctaagt tggacaatta ttataagaaa gacaattctt atttcacaga gcaaccaatt |
| | 6001 | gatcttgtac caaaccaacc atatccaaac gcaagcttcg ataattttaa gtttgtatgt |
| | 6061 | gataatatca aatttgctga tgatttaaac cagttaactg gttataagaa acctgcttca |
| | 6121 | agagagctta aagttacatt tttccctgac ttaaatggtg atgtggtggc tattgattat |
| | 6181 | aaacactaca caccctcttt taagaaagga gctaaattgt tacataaacc tattgtttgg |
| | 6241 | catgttaaca atgcaactaa taaagccacg tataaaccaa atacctggtg tatacgttgt |
| | 6301 | ctttggagca caaaaccagt tgaaacatca aattcgtttg atgtactgaa gtcagaggac |
| | 6361 | gcgcagggaa tggataatct tgcctgcgaa gatctaaaac cagtctctga agaagtagtg |
| | 6421 | gaaaatccta ccatacagaa agacgttctt gagtgtaatg tgaaaactac cgaagttgta |
| | 6481 | ggagacatta tacttaaacc agcaaataat agtttaaaaa ttacagaaga ggttggccac |
| | 6541 | acagatctaa tggctgctta tgtagacaat tctagtctta ctattaagaa acctaatgaa |
| | 6601 | ttatctagag tattaggttt gaaaaccctt gctactcatg gtttagctgc tgttaatagt |
| | 6661 | gtcccttggg atactatagc taattatgct aagccttttc ttaacaaagt tgttagtaca |
| | 6721 | actactaaca tagttacacg gtgtttaaac cgtgtttgta ctaattatat gccttatttc |
| | 6781 | tttactttat tgctacaatt gtgtactttt actagaagta caaattctag aattaaagca |
| | 6841 | tctatgccga ctactatagc aaagaatact gttaagagtg tcggtaaatt ttgtctagag |
| | 6901 | gcttcattta attatttgaa gtcacctaat ttttctaaac tgataaatat tataatttgg |
| | 6961 | tttttactat taagtgtttg cctaggttct ttaatctact caaccgctgc tttaggtgtt |
| | 7021 | ttaatgtcta atttaggcat gccttcttac tgtactggtt acagagaagg ctatttgaac |
| | 7081 | tctactaatg tcactattgc aacctactgt actggttcta taccttgtag tgtttgtctt |
| | 7141 | agtggtttag attctttaga cacctatcct tctttagaaa ctatacaaat taccatttca |
| | 7201 | tcttttaaat gggatttaac tgcttttggc ttagttgcag agtggttttt ggcatatatt |
| | 7261 | cttttcacta ggtttttcta tgtacttgga ttggctgcaa tcatgcaatt gtttttcagc |
| | 7321 | tattttgcag tacattttat tagtaattct tggcttatgt ggttaataat taatcttgta |
| | 7381 | caaatggccc cgatttcagc tatggttaga atgtacatct tctttgcatc attttattat |
| | 7441 | gtatggaaaa gttatgtgca tgttgtagac ggttgtaatt catcaacttg tatgatgtgt |
| | 7501 | tacaaacgta atagagcaac aagagtcgaa tgtacaacta ttgttaatgg tgttagaagg |
| | 7561 | tccttttatg tctatgctaa tggaggtaaa ggcttttgca aactacacaa ttggaattgt |
| | 7621 | gttaattgtg atacattctg tgctggtagt acatttatta gtgatgaagt tgcgagagac |
| | 7681 | ttgtcactac agtttaaaag accaataaat cctactgacc agtcttctta catcgttgat |
| | 7741 | agtgttacag tgaagaatgg ttccatccat ctttactttg ataaagctgg tcaaaagact |
| | 7801 | tatgaaagac attctctctc tcattttgtt aacttagaca acctgagagc taataacact |
| | 7861 | aaaggttcat tgcctattaa tgttatagtt tttgatggta aatcaaaatg tgaagaatca |
| | 7921 | tctgcaaaat cagcgtctgt ttactacagt cagcttatgt gtcaacctat actgttacta |
| | 7981 | gatcaggcat tagtgtctga tgttggtgat agtgcggaag ttgcagttaa aatgtttgat |
| | 8041 | gcttacgtta atacgttttc atcaactttt aacgtaccaa tggaaaaact caaaacacta |
| | 8101 | gttgcaactg cagaagctga acttgcaaag aatgtgtcct tagacaatgt cttatctact |
| | 8161 | tttatttcag cagctcggca agggtttgtt gattcagatg tagaaactaa agatgttgtt |
| | 8221 | gaatgtctta aattgtcaca tcaatctgac atagaagtta ctggcgatag ttgtaataac |
| | 8281 | tatatgctca cctataacaa agttgaaaac atgacacccc gtgaccttgg tgcttgtatt |
| | 8341 | gactgtagtg cgcgtcatat taatgcgcag gtagcaaaaa gtcacaacat tgctttgata |
| | 8401 | tggaacgtta aagatttcat gtcattgtct gaacaactac gaaaacaaat acgtagtgct |
| | 8461 | gctaaaaaga ataacttacc ttttaagttg acatgtgcaa ctactagaca agttgttaat |
| | 8521 | gttgtaacaa caaagatagc acttaagggt ggtaaaattg ttaataattg gttgaagcag |
| | 8581 | ttaattaaag ttacacttgt gttccttttt gttgctgcta ttttctattt aataacacct |
| | 8641 | gttcatgtca tgtctaaaca tactgacttt tcaagtgaaa tcataggata caaggctatt |
| | 8701 | gatggtggtg tcactcgtga catagcatct acagatactt gttttgctaa caaacatgct |
| | 8761 | gattttgaca catggtttag ccagcgtggt ggtagttata ctaatgacaa agcttgccca |
| | 8821 | ttgattgctg cagtcataac aagagaagtg ggttttgtcg tgcctggttt gcctggcacg |
| | 8881 | atattacgca caactaatgg tgactttttg catttcttac ctagagtttt tagtgcagtt |
| | 8941 | ggtaacatct gttacacacc atcaaaactt atagagtaca ctgactttgc aacatcagct |
| | 9001 | tgtgttttgg ctgctgaatg tacaattttt aaagatgctt ctggtaagcc agtaccatat |
| | 9061 | tgttatgata ccaatgtact agaaggttct gttgcttatg aaagtttacg ccctgacaca |
| | 9121 | cgttatgtgc tcatggatgg ctctattatt caatttccta acacctacct tgaaggttct |
| | 9181 | gttagagtgg taacaacttt tgattctgag tactgtaggc acggcacttg tgaaagatca |
| | 9241 | gaagctggtg tttgtgtatc tactagtggt agatgggtac ttaacaatga ttattacaga |
| | 9301 | tctttaccag gagttttctg tggtgtagat gctgtaaatt tacttactaa tatgtttaca |
| | 9361 | ccactaattc aacctattgg tgctttggac atatcagcat ctatagtagc tggtggtatt |
| | 9421 | gtagctatcg tagtaacatg ccttgcctac tattttatga ggtttagaag agcttttggt |
| | 9481 | gaatacagtc atgtagttgc ctttaatact ttactattcc ttatgtcatt cactgtactc |
| | 9541 | tgtttaacac cagtttactc attcttacct ggtgtttatt ctgttattta cttgtacttg |
| | 9601 | acattttatc ttactaatga tgtttctttt ttagcacata ttcagtggat ggttatgttc |
| | 9661 | acacctttag tacctttctg gataacaatt gcttatatca tttgtatttc cacaaagcat |
| | 9721 | ttctattggt tctttagtaa ttacctaaag agacgtgtag tctttaatgg tgtttccttt |
| | 9781 | agtacttttg aagaagctgc gctgtgcacc tttttgttaa ataaagaaat gtatctaaag |
| | 9841 | ttgcgtagtg atgtgctatt acctcttacg caatataata gatacttagc tctttataat |
| | 9901 | aagtacaagt attttagtgg agcaatggat acaactagct acagagaagc tgcttgttgt |
| | 9961 | catctcgcaa aggctctcaa tgacttcagt aactcaggtt ctgatgttct ttaccaacca |
| | 10021 | ccacaaacct ctatcacctc agctgttttg cagagtggtt ttagaaaaat ggcattccca |
| | 10081 | tctggtaaag ttgagggttg tatggtacaa gtaacttgtg gtacaactac acttaacggt |
| | 10141 | ctttggcttg atgacgtagt ttactgtcca agacatgtga tctgcacctc tgaagacatg |
| | 10201 | cttaacccta attatgaaga tttactcatt cgtaagtcta atcataattt cttggtacag |
| | 10261 | gctggtaatg ttcaactcag ggttattgga cattctatgc aaaattgtgt acttaagctt |
| | 10321 | aaggttgata cagccaatcc taagacacct aagtataagt ttgttcgcat tcaaccagga |
| | 10381 | cagacttttt cagtgttagc ttgttacaat ggttcaccat ctggtgttta ccaatgtgct |
| | 10441 | atgaggccca atttcactat taagggttca ttccttaatg gttcatgtgg tagtgttggt |
| | 10501 | tttaacatag attatgactg tgtctctttt tgttacatgc accatatgga attaccaact |
| | 10561 | ggagttcatg ctggcacaga cttagaaggt aacttttatg gaccttttgt tgacaggcaa |
| | 10621 | acagcacaag cagctggtac ggacacaact attacagtta atgttttagc ttggttgtac |
| | 10681 | gctgctgtta taaatggaga caggtggttt ctcaatcgat ttaccacaac tcttaatgac |
| | 10741 | tttaaccttg tggctatgaa gtacaattat gaacctctaa cacaagacca tgttgacata |
| | 10801 | ctaggacctc tttctgctca aactggaatt gccgttttag atatgtgtgc ttcattaaaa |
| | 10861 | gaattactgc aaaatggtat gaatggacgt accatattgg gtagtgcttt attagaagat |
| | 10921 | gaatttacac cttttgatgt tgttagacaa tgctcaggtg ttactttcca aagtgcagtg |
| | 10981 | aaaagaacaa tcaagggtac acaccactgg ttgttactca caattttgac ttcactttta |
| | 11041 | gttttagtcc agagtactca atggtctttg ttcttttttt tgtatgaaaa tgccttttta |
| | 11101 | ccttttgcta tgggtattat tgctatgtct gcttttgcaa tgatgtttgt caaacataag |
| | 11161 | catgcatttc tctgtttgtt tttgttacct tctcttgcca ctgtagctta ttttaatatg |
| | 11221 | gtctatatgc ctgctagttg ggtgatgcgt attatgacat ggttggatat ggttgatact |
| | 11281 | agtttgtctg gttttaagct aaaagactgt gttatgtatg catcagctgt agtgttacta |
| | 11341 | atccttatga cagcaagaac tgtgtatgat gatggtgcta ggagagtgtg gacacttatg |
| | 11401 | aatgtcttga cactcgttta taaagtttat tatggtaatg ctttagatca agccatttcc |
| | 11461 | atgtgggctc ttataatctc tgttacttct aactactcag gtgtagttac aactgtcatg |
| | 11521 | tttttggcca gaggtattgt ttttatgtgt gttgagtatt gccctatttt cttcataact |
| | 11581 | ggtaatacac ttcagtgtat aatgctagtt tattgtttct taggctattt ttgtacttgt |
| | 11641 | tactttggcc tcttttgttt actcaaccgc tactttagac tgactcttgg tgtttatgat |
| | 11701 | tacttagttt ctacacagga gtttagatat atgaattcac agggactact cccacccaag |
| | 11761 | aatagcatag atgccttcaa actcaacatt aaattgttgg gtgttggtgg caaaccttgt |
| | 11821 | atcaaagtag ccactgtaca gtctaaaatg tcagatgtaa agtgcacatc agtagtctta |
| | 11881 | ctctcagttt tgcaacaact cagagtagaa tcatcatcta aattgtgggc tcaatgtgtc |
| | 11941 | cagttacaca atgacattct cttagctaaa gatactactg aagcctttga aaaaatggtt |
| | 12001 | tcactacttt ctgttttgct ttccatgcag ggtgctgtag acataaacaa gctttgtgaa |
| | 12061 | gaaatgctgg acaacagggc aaccttacaa gctatagcct cagagtttag ttcccttcca |
| | 12121 | tcatatgcag cttttgctac tgctcaagaa gcttatgagc aggctgttgc taatggtgat |
| | 12181 | tctgaagttg ttcttaaaaa gttgaagaag tctttgaatg tggctaaatc tgaatttgac |
| | 12241 | cgtgatgcag ccatgcaacg taagttggaa aagatggctg atcaagctat gacccaaatg |
| | 12301 | tataaacagg ctagatctga ggacaagagg gcaaaagtta ctagtgctat gcagacaatg |
| | 12361 | cttttcacta tgcttagaaa gttggataat gatgcactca acaacattat caacaatgca |
| | 12421 | agagatggtt gtgttccctt gaacataata cctcttacaa cagcagccaa actaatggtt |
| | 12481 | gtcataccag actataacac atataaaaat acgtgtgatg gtacaacatt tacttatgca |
| | 12541 | tcagcattgt gggaaatcca acaggttgta gatgcagata gtaaaattgt tcaacttagt |
| | 12601 | gaaattagta tggacaattc acctaattta gcatggcctc ttattgtaac agctttaagg |
| | 12661 | gccaattctg ctgtcaaatt acagaataat gagcttagtc ctgttgcact acgacagatg |
| | 12721 | tcttgtgctg ccggtactac acaaactgct tgcactgatg acaatgcgtt agcttactac |
| | 12781 | aacacaacaa agggaggtag gtttgtactt gcactgttat ccgatttaca ggatttgaaa |
| | 12841 | tgggctagat tccctaagag tgatggaact ggtactatct atacagaact ggaaccacct |
| | 12901 | tgtaggtttg ttacagacac acctaaaggt cctaaagtga agtatttata ctttattaaa |
| | 12961 | ggattaaaca acctaaatag aggtatggta cttggtagtt tagctgccac agtacgtcta |
| | 13021 | caagctggta atgcaacaga agtgcctgcc aattcaactg tattatcttt ctgtgctttt |
| | 13081 | gctgtagatg ctgctaaagc ttacaaagat tatctagcta gtgggggaca accaatcact |
| | 13141 | aattgtgtta agatgttgtg tacacacact ggtactggtc aggcaataac agttacaccg |
| | 13201 | gaagccaata tggatcaaga atcctttggt ggtgcatcgt gttgtctgta ctgccgttgc |
| | 13261 | cacatagatc atccaaatcc taaaggattt tgtgacttaa aaggtaagta tgtacaaata |
| | 13321 | cctacaactt gtgctaatga ccctgtgggt tttacactta aaaacacagt ctgtaccgtc |
| | 13381 | tgcggtatgt ggaaaggtta tggctgtagt tgtgatcaac tccgcgaacc catgcttcag |
| | 13441 | tcagctgatg cacaatcgtt tttaaacggg tttgcggtgt aagtgcagcc cgtcttacac |
| | 13501 | cgtgcggcac aggcactagt actgatgtcg tatacagggc ttttgacatc tacaatgata |
| | 13561 | aagtagctgg ttttgctaaa ttcctaaaaa ctaattgttg tcgcttccaa gaaaaggacg |
| | 13621 | aagatgacaa tttaattgat tcttactttg tagttaagag acacactttc tctaactacc |
| | 13681 | aacatgaaga aacaatttat aatttactta aggattgtcc agctgttgct aaacatgact |
| | 13741 | tctttaagtt tagaatagac ggtgacatgg taccacatat atcacgtcaa cgtcttacta |
| | 13801 | aatacacaat ggcagacctc gtctatgctt taaggcattt tgatgaaggt aattgtgaca |
| | 13861 | cattaaaaga aatacttgtc acatacaatt gttgtgatga tgattatttc aataaaaagg |
| | 13921 | actggtatga ttttgtagaa aacccagata tattacgcgt atacgccaac ttaggtgaac |
| | 13981 | gtgtacgcca agctttgtta aaaacagtac aattctgtga tgccatgcga aatgctggta |
| | 14041 | ttgttggtgt actgacatta gataatcaag atctcaatgg taactggtat gatttcggtg |
| | 14101 | atttcataca aaccacgcca ggtagtggag ttcctgttgt agattcttat tattcattgt |
| | 14161 | taatgcctat attaaccttg accagggctt taactgcaga gtcacatgtt gacactgact |
| | 14221 | taacaaagcc ttacattaag tgggatttgt taaaatatga cttcacggaa gagaggttaa |
| | 14281 | aactctttga ccgttatttt aaatattggg atcagacata ccacccaaat tgtgttaact |
| | 14341 | gtttggatga cagatgcatt ctgcattgtg caaactttaa tgttttattc tctacagtgt |
| | 14401 | tcccacctac aagttttgga ccactagtga gaaaaatatt tgttgatggt gttccatttg |
| | 14461 | tagtttcaac tggataccac ttcagagagc taggtgttgt acataatcag gatgtaaact |
| | 14521 | tacatagctc tagacttagt tttaaggaat tacttgtgta tgctgctgac cctgctatgc |
| | 14581 | acgctgcttc tggtaatcta ttactagata aacgcactac gtgcttttca gtagctgcac |
| | 14641 | ttactaacaa tgttgctttt caaactgtca aacccggtaa ttttaacaaa gacttctatg |
| | 14701 | actttgctgt gtctaagggt ttctttaagg aaggaagttc tgttgaatta aaacacttct |
| | 14761 | tctttgctca ggatggtaat gctgctatca gcgattatga ctactatcgt tataatctac |
| | 14821 | caacaatgtg tgatatcaga caactactat ttgtagttga agttgttgat aagtactttg |
| | 14881 | attgttacga tggtggctgt attaatgcta accaagtcat cgtcaacaac ctagacaaat |
| | 14941 | cagctggttt tccatttaat aaatggggta aggctagact ttattatgat tcaatgagtt |
| | 15001 | atgaggatca agatgcactt ttcgcatata caaaacgtaa tgtcatccct actataactc |
| | 15061 | aaatgaatct taagtatgcc attagtgcaa agaatagagc tcgcaccgta gctggtgtct |
| | 15121 | ctatctgtag tactatgacc aatagacagt ttcatcaaaa attattgaaa tcaatagccg |
| | 15181 | ccactagagg agctactgta gtaattggaa caagcaaatt ctatggtggt tggcacaaca |
| | 15241 | tgttaaaaac tgtttatagt gatgtagaaa accctcacct tatgggttgg gattatccta |
| | 15301 | aatgtgatag agccatgcct aacatgctta gaattatggc ctcacttgtt cttgctcgca |
| | 15361 | aacatacaac gtgttgtagc ttgtcacacc gtttctatag attagctaat gagtgtgctc |
| | 15421 | aagtattgag tgaaatggtc atgtgtggcg gttcactata tgttaaacca ggtggaacct |
| | 15481 | catcaggaga tgccacaact gcttatgcta atagtgtttt taacatttgt caagctgtca |
| | 15541 | cggccaatgt taatgcactt ttatctactg atggtaacaa aattgccgat aagtatgtcc |
| | 15601 | gcaatttaca acacagactt tatgagtgtc tctatagaaa tagagatgtt gacacagact |
| | 15661 | ttgtgaatga gttttacgca tatttgcgta aacatttctc aatgatgata ctctctgacg |
| | 15721 | atgctgttgt gtgtttcaat agcacttatg catctcaagg tctagtggct agcataaaga |
| | 15781 | actttaagtc agttctttat tatcaaaaca atgtttttat gtctgaagca aaatgttgga |
| | 15841 | ctgagactga ccttactaaa ggacctcatg aattttgctc tcaacataca atgctagtta |
| | 15901 | aacagggtga tgattatgtg taccttcctt acccagatcc atcaagaatc ctaggggccg |
| | 15961 | gctgttttgt agatgatatc gtaaaaacag atggtacact tatgattgaa cggttcgtgt |
| | 16021 | ctttagctat agatgcttac ccacttacta aacatcctaa tcaggagtat gctgatgtct |
| | 16081 | ttcatttgta cttacaatac ataagaaagc tacatgatga gttaacagga cacatgttag |
| | 16141 | acatgtattc tgttatgctt actaatgata acacttcaag gtattgggaa cctgagtttt |
| | 16201 | atgaggctat gtacacaccg catacagtct tacaggctgt tggggcttgt gttctttgca |
| | 16261 | attcacagac ttcattaaga tgtggtgctt gcatacgtag accattctta tgttgtaaat |
| | 16321 | gctgttacga ccatgtcata tcaacatcac ataaattagt cttgtctgtt aatccgtatg |
| | 16381 | tttgcaatgc tccaggttgt gatgtcacag atgtgactca actttactta ggaggtatga |
| | 16441 | gctattattg taaatcacat aaaccaccca ttagttttcc attgtgtgct aatggacaag |
| | 16501 | tttttggttt atataaaaat acatgtgttg gtagcgataa tgttactgac tttaatgcaa |
| | 16561 | ttgcaacatg tgactggaca aatgctggtg attacatttt agctaacacc tgtactgaaa |
| | 16621 | gactcaagct ttttgcagca gaaacgctca aagctactga ggagacattt aaactgtctt |
| | 16681 | atggtattgc tactgtacgt gaagtgctgt ctgacagaga attacatctt tcatgggaag |
| | 16741 | ttggtaaacc tagaccacca cttaaccgaa attatgtctt tactggttat cgtgtaacta |
| | 16801 | aaaacagtaa agtacaaata ggagagtaca cctttgaaaa aggtgactat ggtgatgctg |
| | 16861 | ttgtttaccg aggtacaaca acttacaaat taaatgttgg tgattatttt gtgctgacat |
| | 16921 | cacatacagt aatgccatta agtgcaccta cactagtgcc acaagagcac tatgttagaa |
| | 16981 | ttactggctt atacccaaca ctcaatatct cagatgagtt ttctagcaat gttgcaaatt |
| | 17041 | atcaaaaggt tggtatgcaa aagtattcta cactccaggg accacctggt actggtaaga |
| | 17101 | gtcattttgc tattggccta gctctctact acccttctgc tcgcatagtg tatacagctt |
| | 17161 | gctctcatgc cgctgttgat gcactatgtg agaaggcatt aaaatatttg cctatagata |
| | 17221 | aatgtagtag aattatacct gcacgtgctc gtgtagagtg ttttgataaa ttcaaagtga |
| | 17281 | attcaacatt agaacagtat gtcttttgta ctgtaaatgc attgcctgag acgacagcag |
| | 17341 | atatagttgt ctttgatgaa atttcaatgg ccacaaatta tgatttgagt gttgtcaatg |
| | 17401 | ccagattacg tgctaagcac tatgtgtaca ttggcgaccc tgctcaatta cctgcaccac |
| | 17461 | gcacattgct aactaagggc acactagaac cagaatattt caattcagtg tgtagactta |
| | 17521 | tgaaaactat aggtccagac atgttcctcg gaacttgtcg gcgttgtcct gctgaaattg |
| | 17581 | ttgacactgt gagtgctttg gtttatgata ataagcttaa agcacataaa gacaaatcag |
| | 17641 | ctcaatgctt taaaatgttt tataagggtg ttatcacgca tgatgtttca tctgcaatta |
| | 17701 | acaggccaca aataggcgtg gtaagagaat tccttacacg taaccctgct tggagaaaag |
| | 17761 | ctgtctttat ttcaccttat aattcacaga atgctgtagc ctcaaagatt ttgggactac |
| | 17821 | caactcaaac tgttgattca tcacagggct cagaatatga ctatgtcata ttcactcaaa |
| | 17881 | ccactgaaac agctcactct tgtaatgtaa acagatttaa tgttgctatt accagagcaa |
| | 17941 | aagtaggcat actttgcata atgtctgata gagaccttta tgacaagttg caatttacaa |
| | 18001 | gtcttgaaat tccacgtagg aatgtggcaa ctttacaagc tgaaaatgta acaggactct |
| | 18061 | ttaaagattg tagtaaggta atcactgggt tacatcctac acaggcacct acacacctca |
| | 18121 | gtgttgacac taaattcaaa actgaaggtt tatgtgttga catacctggc atacctaagg |
| | 18181 | acatgaccta tagaagactc atctctatga tgggttttaa aatgaattat caagttaatg |
| | 18241 | gttaccctaa catgtttatc acccgcgaag aagctataag acatgtacgt gcatggattg |
| | 18301 | gcttcgatgt cgaggggtgt catgctacta gagaagctgt tggtaccaat ttacctttac |
| | 18361 | agctaggttt ttctacaggt gttaacctag ttgctgtacc tacaggttat gttgatacac |
| | 18421 | ctaataatac agatttttcc agagttagtg ctaaaccacc gcctggagat caatttaaac |
| | 18481 | acctcatacc acttatgtac aaaggacttc cttggaatgt agtgcgtata aagattgtac |
| | 18541 | aaatgttaag tgacacactt aaaaatctct ctgacagagt cgtatttgtc ttatgggcac |
| | 18601 | atggctttga gttgacatct atgaagtatt ttgtgaaaat aggacctgag cgcacctgtt |
| | 18661 | gtctatgtga tagacgtgcc acatgctttt ccactgcttc agacacttat gcctgttggc |
| | 18721 | atcattctat tggatttgat tacgtctata atccgtttat gattgatgtt caacaatggg |
| | 18781 | gttttacagg taacctacaa agcaaccatg atctgtattg tcaagtccat ggtaatgcac |
| | 18841 | atgtagctag ttgtgatgca atcatgacta ggtgtctagc tgtccacgag tgctttgtta |
| | 18901 | agcgtgttga ctggactatt gaatatccta taattggtga tgaactgaag attaatgcgg |
| | 18961 | cttgtagaaa ggttcaacac atggttgtta aagctgcatt attagcagac aaattcccag |
| | 19021 | ttcttcacga cattggtaac cctaaagcta ttaagtgtgt acctcaagct gatgtagaat |
| | 19081 | ggaagttcta tgatgcacag ccttgtagtg acaaagctta taaaatagaa gaattattct |
| | 19141 | attcttatgc cacacattct gacaaattca cagatggtgt atgcctattt tggaattgca |
| | 19201 | atgtcgatag atatcctgct aattccattg tttgtagatt tgacactaga gtgctatcta |
| | 19261 | accttaactt gcctggttgt gatggtggca gtttgtatgt aaataaacat gcattccaca |
| | 19321 | caccagcttt tgataaaagt gcttttgtta atttaaaaca attaccattt ttctattact |
| | 19381 | ctgacagtcc atgtgagtct catggaaaac aagtagtgtc agatatagat tatgtaccac |
| | 19441 | taaagtctgc tacgtgtata acacgttgca atttaggtgg tgctgtctgt agacatcatg |
| | 19501 | ctaatgagta cagattgtat ctcgatgctt ataacatgat gatctcagct ggctttagct |
| | 19561 | tgtgggttta caaacaattt gatacttata acctctggaa cacttttaca agacttcaga |
| | 19621 | gtttagaaaa tgtggctttt aatgttgtaa ataagggaca ctttgatgga caacagggtg |
| | 19681 | aagtaccagt ttctatcatt aataacactg tttacacaaa agttgatggt gttgatgtag |
| | 19741 | aattgtttga aaataaaaca acattacctg ttaatgtagc atttgagctt tgggctaagc |
| | 19801 | gcaacattaa accagtacca gaggtgaaaa tactcaataa tttgggtgtg gacattgctg |
| | 19861 | ctaatactgt gatctgggac tacaaaagag atgctccagc acatatatct actattggtg |
| | 19921 | tttgttctat gactgacata gccaagaaac caactgaaac gatttgtgca ccactcactg |
| | 19981 | tcttttttga tggtagagtt gatggtcaag tagacttatt tagaaatgcc cgtaatggtg |
| | 20041 | ttcttattac agaaggtagt gttaaaggtt tacaaccatc tgtaggtccc aaacaagcta |
| | 20101 | gtcttaatgg agtcacatta attggagaag ccgtaaaaac acagttcaat tattataaga |
| | 20161 | aagttgatgg tgttgtccaa caattacctg aaacttactt tactcagagt agaaatttac |
| | 20221 | aagaatttaa acccaggagt caaatggaaa ttgatttctt agaattagct atggatgaat |
| | 20281 | tcattgaacg gtataaatta gaaggctatg ccttcgaaca tatcgtttat ggagatttta |
| | 20341 | gtcatagtca gttaggtggt ttacatctac tgattggact agctaaacgt tttaaggaat |
| | 20401 | caccttttga attagaagat tttattccta tggacagtac agttaaaaac tatttcataa |
| | 20461 | cagatgcgca aacaggttca tctaagtgtg tgtgttctgt tattgattta ttacttgatg |
| | 20521 | attttgttga aataataaaa tcccaagatt tatctgtagt ttctaaggtt gtcaaagtga |
| | 20581 | ctattgacta tacagaaatt tcatttatgc tttggtgtaa agatggccat gtagaaacat |
| | 20641 | tttacccaaa attacaatct agtcaagcgt ggcaaccggg tgttgctatg cctaatcttt |
| | 20701 | acaaaatgca aagaatgcta ttagaaaagt gtgaccttca aaattatggt gatagtgcaa |
| | 20761 | cattacctaa aggcataatg atgaatgtcg caaaatatac tcaactgtgt caatatttaa |
| | 20821 | acacattaac attagctgta ccctataata tgagagttat acattttggt gctggttctg |
| | 20881 | ataaaggagt tgcaccaggt acagctgttt taagacagtg gttgcctacg ggtacgctgc |
| | 20941 | ttgtcgattc agatcttaat gactttgtct ctgatgcaga ttcaactttg attggtgatt |
| | 21001 | gtgcaactgt acatacagct aataaatggg atctcattat tagtgatatg tacgacccta |
| | 21061 | agactaaaaa tgttacaaaa gaaaatgact ctaaagaggg ttttttcact tacatttgtg |
| | 21121 | ggtttataca acaaaagcta gctcttggag gttccgtggc tataaagata acagaacatt |
| | 21181 | cttggaatgc tgatctttat aagctcatgg gacacttcgc atggtggaca gcctttgtta |
| | 21241 | ctaatgtgaa tgcgtcatca tctgaagcat ttttaattgg atgtaattat cttggcaaac |
| | 21301 | cacgcgaaca aatagatggt tatgtcatgc atgcaaatta catattttgg aggaatacaa |
| | 21361 | atccaattca gttgtcttcc tattctttat ttgacatgag taaatttccc cttaaattaa |
| | 21421 | ggggtactgc tgttatgtct ttaaaagaag gtcaaatcaa tgatatgatt ttatctcttc |
| | 21481 | ttagtaaagg tagacttata attagagaaa acaacagagt tgttatttct agtgatgttc |
| | 21541 | ttgttaacaa ctaaacgaac aatgtttgtt tttcttgttt tattgccact agtctctagt |
| | 21601 | cagtgtgtta atcttacaac cagaactcaa ttaccccctg catacactaa ttctttcaca |
| | 21661 | cgtggtgttt attaccctga caaagttttc agatcctcag ttttacattc aactcaggac |
| | 21721 | ttgttcttac ctttcttttc caatgttact tggttccatg ctatacatgt ctctgggacc |
| | 21781 | aatggtacta agaggtttga taaccctgtc ctaccattta atgatggtgt ttattttgct |
| | 21841 | tccactgaga agtctaacat aataagaggc tggatttttg gtactacttt agattcgaag |
| | 21901 | acccagtccc tacttattgt taataacgct actaatgttg ttattaaagt ctgtgaattt |
| | 21961 | caattttgta atgatccatt tttgggtgtt tattaccaca aaaacaacaa aagttggatg |
| | 22021 | gaaagtgagt tcagagttta ttctagtgcg aataattgca cttttgaata tgtctctcag |
| | 22081 | ccttttctta tggaccttga aggaaaacag ggtaatttca aaaatcttag ggaatttgtg |
| | 22141 | tttaagaata ttgatggtta ttttaaaata tattctaagc acacgcctat taatttagtg |
| | 22201 | cgtgatctcc ctcagggttt ttcggcttta gaaccattgg tagatttgcc aataggtatt |
| | 22261 | aacatcacta ggtttcaaac tttacttgct ttacatagaa gttatttgac tcctggtgat |
| | 22321 | tcttcttcag gttggacagc tggtgctgca gcttattatg tgggttatct tcaacctagg |
| | 22381 | acttttctat taaaatataa tgaaaatgga accattacag atgctgtaga ctgtgcactt |
| | 22441 | gaccctctct cagaaacaaa gtgtacgttg aaatccttca ctgtagaaaa aggaatctat |
| | 22501 | caaacttcta actttagagt ccaaccaaca gaatctattg ttagatttcc taatattaca |
| | 22561 | aacttgtgcc cttttggtga agtttttaac gccaccagat ttgcatctgt ttatgcttgg |
| | 22621 | aacaggaaga gaatcagcaa ctgtgttgct gattattctg tcctatataa ttccgcatca |
| | 22681 | ttttccactt ttaagtgtta tggagtgtct cctactaaat taaatgatct ctgctttact |
| | 22741 | aatgtctatg cagattcatt tgtaattaga ggtgatgaag tcagacaaat cgctccaggg |
| | 22801 | caaactggaa agattgctga ttataattat aaattaccag atgattttac aggctgcgtt |
| | 22861 | atagcttgga attctaacaa tcttgattct aaggttggtg gtaattataa ttacctgtat |
| | 22921 | agattgttta ggaagtctaa tctcaaacct tttgagagag atatttcaac tgaaatctat |
| | 22981 | caggccggta gcacaccttg taatggtgtt gaaggtttta attgttactt tcctttacaa |
| | 23041 | tcatatggtt tccaacccac taatggtgtt ggttaccaac catacagagt agtagtactt |
| | 23101 | tcttttgaac ttctacatgc accagcaact gtttgtggac ctaaaaagtc tactaatttg |
| | 23161 | gttaaaaaca aatgtgtcaa tttcaacttc aatggtttaa caggcacagg tgttcttact |
| | 23221 | gagtctaaca aaaagtttct gcctttccaa caatttggca gagacattgc tgacactact |
| | 23281 | gatgctgtcc gtgatccaca gacacttgag attcttgaca ttacaccatg ttcttttggt |
| | 23341 | ggtgtcagtg ttataacacc aggaacaaat acttctaacc aggttgctgt tctttatcag |
| | 23401 | gatgttaact gcacagaagt ccctgttgct attcatgcag atcaacttac tcctacttgg |
| | 23461 | cgtgtttatt ctacaggttc taatgttttt caaacacgtg caggctgttt aataggggct |
| | 23521 | gaacatgtca acaactcata tgagtgtgac atacccattg gtgcaggtat atgcgctagt |
| | 23581 | tatcagactc agactaattc tcctcggcgg gcacgtagtg tagctagtca atccatcatt |
| | 23641 | gcctacacta tgtcacttgg tgcagaaaat tcagttgctt actctaataa ctctattgcc |
| | 23701 | atacccacaa attttactat tagtgttacc acagaaattc taccagtgtc tatgaccaag |
| | 23761 | acatcagtag attgtacaat gtacatttgt ggtgattcaa ctgaatgcag caatcttttg |
| | 23821 | ttgcaatatg gcagtttttg tacacaatta aaccgtgctt taactggaat agctgttgaa |
| | 23881 | caagacaaaa acacccaaga agtttttgca caagtcaaac aaatttacaa aacaccacca |
| | 23941 | attaaagatt ttggtggttt taatttttca caaatattac cagatccatc aaaaccaagc |
| | 24001 | aagaggtcat ttattgaaga tctacttttc aacaaagtga cacttgcaga tgctggcttc |
| | 24061 | atcaaacaat atggtgattg ccttggtgat attgctgcta gagacctcat ttgtgcacaa |
| | 24121 | aagtttaacg gccttactgt tttgccacct ttgctcacag atgaaatgat tgctcaatac |
| | 24181 | acttctgcac tgttagcggg tacaatcact tctggttgga cctttggtgc aggtgctgca |
| | 24241 | ttacaaatac catttgctat gcaaatggct tataggttta atggtattgg agttacacag |
| | 24301 | aatgttctct atgagaacca aaaattgatt gccaaccaat ttaatagtgc tattggcaaa |
| | 24361 | attcaagact cactttcttc cacagcaagt gcacttggaa aacttcaaga tgtggtcaac |
| | 24421 | caaaatgcac aagctttaaa cacgcttgtt aaacaactta gctccaattt tggtgcaatt |
| | 24481 | tcaagtgttt taaatgatat cctttcacgt cttgacaaag ttgaggctga agtgcaaatt |
| | 24541 | gataggttga tcacaggcag acttcaaagt ttgcagacat atgtgactca acaattaatt |
| | 24601 | agagctgcag aaatcagagc ttctgctaat cttgctgcta ctaaaatgtc agagtgtgta |
| | 24661 | cttggacaat caaaaagagt tgatttttgt ggaaagggct atcatcttat gtccttccct |
| | 24721 | cagtcagcac ctcatggtgt agtcttcttg catgtgactt atgtccctgc acaagaaaag |
| | 24781 | aacttcacaa ctgctcctgc catttgtcat gatggaaaag cacactttcc tcgtgaaggt |
| | 24841 | gtctttgttt caaatggcac acactggttt gtaacacaaa ggaattttta tgaaccacaa |
| | 24901 | atcattacta cagacaacac atttgtgtct ggtaactgtg atgttgtaat aggaattgtc |
| | 24961 | aacaacacag tttatgatcc tttgcaacct gaattagact cattcaagga ggagttagat |
| | 25021 | aaatatttta agaatcatac atcaccagat gttgatttag gtgacatctc tggcattaat |
| | 25081 | gcttcagttg taaacattca aaaagaaatt gaccgcctca atgaggttgc caagaattta |
| | 25141 | aatgaatctc tcatcgatct ccaagaactt ggaaagtatg agcagtatat aaaatggcca |
| | 25201 | tggtacattt ggctaggttt tatagctggc ttgattgcca tagtaatggt gacaattatg |
| | 25261 | ctttgctgta tgaccagttg ctgtagttgt ctcaagggct gttgttcttg tggatcctgc |
| | 25321 | tgcaaatttg atgaagacga ctctgagcca gtgctcaaag gagtcaaatt acattacaca |
| | 25381 | taaacgaact tatggatttg tttatgagaa tcttcacaat tggaactgta actttgaagc |
| | 25441 | aaggtgaaat caaggatgct actccttcag attttgttcg cgctactgca acgataccga |
| | 25501 | tacaagcctc actccctttc ggatggctta ttgttggcgt tgcacttctt gctgtttttc |
| | 25561 | agagcgcttc caaaatcata accctcaaaa agagatggca actagcactc tccaagggtg |
| | 25621 | ttcactttgt ttgcaacttg ctgttgttgt ttgtaacagt ttactcacac cttttgctcg |
| | 25681 | ttgctgctgg ccttgaagcc ccttttctct atctttatgc tttagtctac ttcttgcaga |
| | 25741 | gtataaactt tgtaagaata ataatgaggc tttggctttg ctggaaatgc cgttccaaaa |
| | 25801 | acccattact ttatgatgcc aactattttc tttgctggca tactaattgt tacgactatt |
| | 25861 | gtatacctta caatagtgta acttcttcaa ttgtcattac ttcaggtgat ggcacaacaa |
| | 25921 | gtcctatttc tgaacatgac taccagattg gtggttatac tgaaaaatgg gaatctggag |
| | 25981 | taaaagactg tgttgtatta cacagttact tcacttcaga ctattaccag ctgtactcaa |
| | 26041 | ctcaattgag tacagacact ggtgttgaac atgttacctt cttcatctac aataaaattg |
| | 26101 | ttgatgagcc tgaagaacat gtccaaattc acacaatcga cggttcatcc ggagttgtta |
| | 26161 | atccagtaat ggaaccaatt tatgatgaac cgacgacgac tactagcgtg cctttgtaag |
| | 26221 | cacaagctga tgagtacgaa cttatgtact cattcgtttc ggaagagaca ggtacgttaa |
| | 26281 | tagttaatag cgtacttctt tttcttgctt tcgtggtatt cttgctagtt acactagcca |
| | 26341 | tccttactgc gcttcgattg tgtgcgtact gctgcaatat tgttaacgtg agtcttgtaa |
| | 26401 | aaccttcttt ttacgtttac tctcgtgtta aaaatctgaa ttcttctaga gttcctgatc |
| | 26461 | ttctggtcta aacgaactaa atattatatt agtttttctg tttggaactt taattttagc |
| | 26521 | catggcagat tccaacggta ctattaccgt tgaagagctt aaaaagctcc ttgaacaatg |
| | 26581 | gaacctagta ataggtttcc tattccttac atggatttgt cttctacaat ttgcctatgc |
| | 26641 | caacaggaat aggtttttgt atataattaa gttaattttc ctctggctgt tatggccagt |
| | 26701 | aactttagct tgttttgtgc ttgctgctgt ttacagaata aattggatca ccggtggaat |
| | 26761 | tgctatcgca atggcttgtc ttgtaggctt gatgtggctc agctacttca ttgcttcttt |
| | 26821 | cagactgttt gcgcgtacgc gttccatgtg gtcattcaat ccagaaacta acattcttct |
| | 26881 | caacgtgcca ctccatggca ctattctgac cagaccgctt ctagaaagtg aactcgtaat |
| | 26941 | cggagctgtg atccttcgtg gacatcttcg tattgctgga caccatctag gacgctgtga |
| | 27001 | catcaaggac ctgcctaaag aaatcactgt tgctacatca cgaacgcttt cttattacaa |
| | 27061 | attgggagct tcgcagcgtg tagcaggtga ctcaggtttt gctgcataca gtcgctacag |
| | 27121 | gattggcaac tataaattaa acacagacca ttccagtagc agtgacaata ttgctttgct |
| | 27181 | tgtacagtaa gtgacaacag atgtttcatc tcgttgactt tcaggttact atagcagaga |
| | 27241 | tattactaat tattatgagg acttttaaag tttccatttg gaatcttgat tacatcataa |
| | 27301 | acctcataat taaaaattta tctaagtcac taactgagaa taaatattct caattagatg |
| | 27361 | aagagcaacc aatggagatt gattaaacga acatgaaaat tattcttttc ttggcactga |
| | 27421 | taacactcgc tacttgtgag ctttatcact accaagagtg tgttagaggt acaacagtac |
| | 27481 | ttttaaaaga accttgctct tctggaacat acgagggcaa ttcaccattt catcctctag |
| | 27541 | ctgataacaa atttgcactg acttgcttta gcactcaatt tgcttttgct tgtcctgacg |
| | 27601 | gcgtaaaaca cgtctatcag ttacgtgcca gatcagtttc acctaaactg ttcatcagac |
| | 27661 | aagaggaagt tcaagaactt tactctccaa tttttcttat tgttgcggca atagtgttta |
| | 27721 | taacactttg cttcacactc aaaagaaaga cagaatgatt gaactttcat taattgactt |
| | 27781 | ctatttgtgc tttttagcct ttctgctatt ccttgtttta attatgctta ttatcttttg |
| | 27841 | gttctcactt gaactgcaag atcataatga aacttgtcac gcctaaacga acatgaaatt |
| | 27901 | tcttgttttc ttaggaatca tcacaactgt agctgcattt caccaagaat gtagtttaca |
| | 27961 | gtcatgtact caacatcaac catatgtagt tgatgacccg tgtcctattc acttctattc |
| | 28021 | taaatggtat attagagtag gagctagaaa atcagcacct ttaattgaat tgtgcgtgga |
| | 28081 | tgaggctggt tctaaatcac ccattcagta catcgatatc ggtaattata cagtttcctg |
| | 28141 | tttacctttt acaattaatt gccaggaacc taaattgggt agtcttgtag tgcgttgttc |
| | 28201 | gttctatgaa gactttttag agtatcatga cgttcgtgtt gttttagatt tcatctaaac |
| | 28261 | gaacaaacta aaatgtctga taatggaccc caaaatcagc gaaatgcacc ccgcattacg |
| | 28321 | tttggtggac cctcagattc aactggcagt aaccagaatg gagaacgcag tggggcgcga |
| | 28381 | tcaaaacaac gtcggcccca aggtttaccc aataatactg cgtcttggtt caccgctctc |
| | 28441 | actcaacatg gcaaggaaga ccttaaattc cctcgaggac aaggcgttcc aattaacacc |
| | 28501 | aatagcagtc cagatgacca aattggctac taccgaagag ctaccagacg aattcgtggt |
| | 28561 | ggtgacggta aaatgaaaga tctcagtcca agatggtatt tctactacct aggaactggg |
| | 28621 | ccagaagctg gacttcccta tggtgctaac aaagacggca tcatatgggt tgcaactgag |
| | 28681 | ggagccttga atacaccaaa agatcacatt ggcacccgca atcctgctaa caatgctgca |
| | 28741 | atcgtgctac aacttcctca aggaacaaca ttgccaaaag gcttctacgc agaagggagc |
| | 28801 | agaggcggca gtcaagcctc ttctcgttcc tcatcacgta gtcgcaacag ttcaagaaat |
| | 28861 | tcaactccag gcagcagtag gggaacttct cctgctagaa tggctggcaa tggcggtgat |
| | 28921 | gctgctcttg ctttgctgct gcttgacaga ttgaaccagc ttgagagcaa aatgtctggt |
| | 28981 | aaaggccaac aacaacaagg ccaaactgtc actaagaaat ctgctgctga ggcttctaag |
| | 29041 | aagcctcggc aaaaacgtac tgccactaaa gcatacaatg taacacaagc tttcggcaga |
| | 29101 | cgtggtccag aacaaaccca aggaaatttt ggggaccagg aactaatcag acaaggaact |
| | 29161 | gattacaaac attggccgca aattgcacaa tttgccccca gcgcttcagc gttcttcgga |
| | 29221 | atgtcgcgca ttggcatgga agtcacacct tcgggaacgt ggttgaccta cacaggtgcc |
| | 29281 | atcaaattgg atgacaaaga tccaaatttc aaagatcaag tcattttgct gaataagcat |
| | 29341 | attgacgcat acaaaacatt cccaccaaca gagcctaaaa aggacaaaaa gaagaaggct |
| | 29401 | gatgaaactc aagccttacc gcagagacag aagaaacagc aaactgtgac tcttcttcct |
| | 29461 | gctgcagatt tggatgattt ctccaaacaa ttgcaacaat ccatgagcag tgctgactca |
| | 29521 | actcaggcct aaactcatgc agaccacaca aggcagatgg gctatataaa cgttttcgct |
| | 29581 | tttccgttta cgatatatag tctactcttg tgcagaatga attctcgtaa ctacatagca |
| | 29641 | caagtagatg tagttaactt taatctcaca tagcaatctt taatcagtgt gtaacattag |
| | 29701 | ggaggacttg aaagagccac cacattttca ccgaggccac gcggagtacg atcgagtgta |
| | 29761 | cagtgaacaa tgctagggag agctgcctat atggaagagc cctaatgtgt aaaattaatt |
| | 29821 | ttagtagtgc tatccccatg tgattttaat agcttcttag gagaatgaca aaaaaaaaaa |
| | 29881 | a |
| Amino acid sequence of | 1 | MFVFLVLLPL VSSQCVNLTT RTQLPPAYTN SFTRGVYYPD KVFRSSVLHS TQDLFLPFFS NVTWFHAIHV SGTNGTKRFD |
| the S protein from | 81 | NPVLPFNDGV YFASTEKSNI IRGWIFGTTL DSKTQSLLIV NNATNVVIKV CEFQFCNDPF LGVYYHKNNK SWMESEFRVY |
| MN988668 Accession | 161 | SSANNCTFEY VSQPFLMDLE GKQGNFKNLR EFVFKNIDGY FKIYSKHTPI NLVRDLPQGF SALEPLVDLP IGINITRFQT |
| number. SEQ ID NO: 47 | 241 | LLALHRSYLT PGDSSSGWTA GAAAYYVGYL QPRTFLLKYN ENGTITDAVD CALDPLSETK CTLKSFTVEK GIYQTSNFRV |
| | 321 | QPTESIVRFP NITNLCPFGE VFNATRFASV YAWNRKRISN CVADYSVLYN SASFSTFKCY GVSPTKLNDL CFTNVYADSF |
| | 401 | VIRGDEVRQI APGQTGKIAD YNYKLPDDFT GCVIAWNSNN LDSKVGGNYN YLYRLFRKSN LKPFERDIST EIYQAGSTPC |
| | 481 | NGVEGFNCYF PLQSYGFQPT NGVGYQPYRV VVLSFELLHA PATVCGPKKS TNLVKNKCVN FNFNGLTGTG VLTESNKKFL |
| | 561 | PFQQFGRDIA DTTDAVRDPQ TLEILDITPC SFGGVSVITP GTNTSNQVAV LYQDVNCTEV PVAIHADQLT PTWRVYSTGS |
| | 641 | NVFQTRAGCL IGAEHVNNSY ECDIPIGAGI CASYQTQTNS PRRARSVASQ SIIAYTMSLG AENSVAYSNN SIAIPTNFTI |
| | 721 | SVTTEILPVS MTKTSVDCTM YICGDSTECS NLLLQYGSFC TQLNRALTGI AVEQDKNTQE VFAQVKQIYK TPPIKDFGGF |
| | 801 | NFSQILPDPS KPSKRSFIED LLFNKVTLAD AGFIKQYGDC LGDIAARDLI CAQKFNGLTV LPPLLTDEMI AQYTSALLAG |
| | 881 | TITSGWTFGA GAALQIPFAM QMAYRFNGIG VTQNVLYENQ KLIANQFNSA IGKIQDSLSS TASALGKLQD VVNQNAQALN |
| | 961 | TLVKQLSSNF GAISSVLNDI LSRLDKVEAE VQIDRLITGR LQSLQTYVTQ QLIRAAEIRA SANLAATKMS ECVLGQSKRV |
| | 1041 | DFCGKGYHLM SFPQSAPHGV VFLHVTYVPA QEKNFTTAPA ICHDGKAHFP REGVFVSNGT HWFVTQRNFY EPQIITTDNT |
| | 1121 | FVSGNCDVVI GIVNNTVYDP LQPELDSFKE ELDKYFKNHT SPDVDLGDIS GINASVVNIQ KEIDRLNEVA KNLNESLIDL |
| | 1201 | QELGKYEQYI KWPWYIWLGF IAGLIAIVMV TIMLCCMTSC CSCLKGCCSC GSCCKFDEDD SEPVLKGVKL HYT |
| Amino acid sequence of | MADSNGTITVEELKKLLEQWNLVIGFLFLTWICLLQFAYANRNR | |
| the M protein from | FLYIIKLIFLWLLWPVTLACFVLAAVYRINWITGGIAIAMACLVGLMWLSYFIASFRL | |
| MN988668 Accession | FARTRSMWSFNPETNILLNVPLHGTILTRPLLESELVIGAVILRGHLRIAGHHLGRCD | |
| number. SEQ ID NO: 48 | IKDLPKEITVATSRTLSYYKLGASQRVAGDSGFAAYSRYRIGNYKLNTDHSSSSDNIA | |
| | LLVQ | |
| Amino acid sequence of | MSDNGPQNQRNAPRITFGGPSDSTGSNQNGERSGARSKQRRPQG | |
| the N protein from | LPNNTASWFTALTQHGKEDLKFPRGQGVPINTNSSPDDQIGYYRRATRRIRGGDGKMK | |
| MN988668 Accession | DLSPRWYFYYLGTGPEAGLPYGANKDGIIWVATEGALNTPKDHIGTRNPANNAAIVLQ | |
| number. SEQ ID NO: 49 | LPQGTTLPKGFYAEGSRGGSQASSRSSSRSRNSSRNSTPGSSRGTSPARMAGNGGDAA | |
| | LALLLLDRLNQLESKMSGKGQQQQGQTVTKKSAAEASKKPRQKRTATKAYNVTQAFGR | |
| | RGPEQTQGNFGDQELIRQGTDYKHWPQIAQFAPSASAFFGMSRIGMEVTPSGTWLTYT | |
| | GAIKLDDKDPNFKDQVILLNKHIDAYKTFPPTEPKKDKKKKADETQALPQRQKKQQTV | |
| | TLLPAADLDDFSKQLQQSMSSADSTQA | |
| Nucleotide sequence | aGCGGCCGCaaaattgaaattttattttttttttttggaatataaataATGTTCGTGTT | |
| of SARS-CoV-2-Spike-co | CCTAGTCCTACTACCGCTAGT | |
| (codon-optimized | CTCTTCCCAGTGTGTAAACCTAACAACGAGAACACAACTACCACCGGCGTACACCAATT | |
| for VACV expression). | CTTTCACAAGAGGAGTATATT | |
| SEQ ID NO: 50 | ACCCGGACAAGGTGTTCAGATCCTCCGTACTACATTCTACCCAGGACCTATTCCTACCG | |
| | TTCTTCTCTAACGTAACATGG | |
| | TTCCACGCGATCCATGTCTCTGGAACAAACGGAACGAAGAGATTCGATAACCCGGTCTT | |
| | GCCGTTCAACGATGGTGTATA | |
| | CTTTGCGTCCACCGAGAAGTCCAACATCATCAGAGGATGGATCTTCGGAACCACCTTGG | |
| | ATTCTAAGACCCAGTCCTTGC | |
| | TAATCGTCAACAACGCGACCAACGTCGTCATCAAAGTCTGCGAATTCCAGTTCTGTAAC | |
| | GACCCGTTTTTGGGAGTCTAC | |
| | TACCACAAGAACAACAAGTCCTGGATGGAATCCGAGTTCAGAGTCTACTCTTCCGCGAA | |
| | CAACTGCACCTTCGAATATGT | |
| | ATCTCAGCCGTTCCTAATGGACCTAGAGGGAAAGCAGGGAAACTTCAAGAACCTAAGAG | |
| | AGTTCGTATTCAAGAACATCG | |
| | ACGGATACTTCAAGATCTACTCCAAGCACACCCCGATCAACCTAGTTAGAGATCTACCG | |
| | CAAGGATTCTCTGCGCTAGAA | |
| | CCGTTAGTAGATTTGCCGATCGGAATCAACATCACCAGATTCCAGACACTACTAGCGCT | |
| | ACACAGATCTTACCTAACGCC | |
| | GGGAGATTCTTCTTCTGGATGGACTGCTGGTGCTGCGGCTTATTATGTAGGATACCTAC | |
| | AGCCGAGAACCTTCCTATTGA | |
| | AGTACAACGAAAACGGAACCATCACCGATGCCGTAGATTGTGCTCTAGATCCGCTATCC | |
| | GAAACGAAGTGCACCCTAAAG | |
| | TCTTTCACCGTCGAGAAGGGAATCTACCAGACCTCCAACTTTAGAGTACAGCCGACCGA | |
| | ATCCATCGTCAGATTTCCGAA | |
| | CATCACGAACCTATGTCCGTTCGGAGAAGTGTTCAACGCGACAAGATTTGCGTCTGTCT | |
| | ATGCGTGGAACAGAAAAAGAA | |
| | TCAGTAACTGCGTCGCGGACTACTCCGTCCTATACAACTCTGCCTCTTTCTCCACGTTC | |
| | AAATGCTACGGTGTATCCCCG | |
| | ACAAAGCTAAACGATCTATGCTTCACCAACGTCTACGCGGACTCCTTCGTAATCAGAGG | |
| | AGATGAAGTTAGACAGATTGC | |
| | GCCGGGACAAACTGGAAAGATCGCGGATTATAACTACAAGCTACCGGACGACTTCACCG | |
| | GATGTGTAATTGCGTGGAATT | |
| | CGAACAACCTAGACTCCAAAGTCGGAGGAAACTACAACTACTTGTACAGACTATTCAGA | |
| | AAGTCCAACCTAAAGCCGTTC | |
| | GAGAGAGACATCTCCACCGAAATCTATCAGGCTGGATCTACACCGTGTAATGGTGTCGA | |
| | AGGATTCAACTGCTACTTCCC | |
| | GCTACAGTCTTACGGATTTCAACCGACAAACGGTGTAGGATATCAGCCGTACAGAGTCG | |
| | TCGTACTATCCTTCGAACTAC | |
| | TACATGCTCCGGCGACAGTATGTGGACCGAAAAAGTCTACCAACCTAGTCAAGAACAAA | |
| | TGCGTCAACTTTAACTTCAAC | |
| | GGACTAACCGGAACCGGTGTCCTAACCGAATCTAACAAGAAGTTTCTACCGTTCCAGCA | |
| | GTTCGGAAGAGATATCGCGGA | |
| | TACAACAGACGCTGTCAGAGATCCGCAAACCTTGGAGATCCTAGATATCACCCCGTGTT | |
| | CTTTCGGTGGTGTCTCTGTAA | |
| | TTACTCCGGGAACGAACACCTCCAATCAAGTAGCGGTACTATACCAGGACGTGAACTGT | |
| | ACAGAAGTACCGGTAGCTATT | |
| | CACGCGGATCAACTAACACCAACTTGGAGAGTGTACTCCACCGGATCTAACGTATTCCA | |
| | AACAAGAGCGGGATGTCTAAT | |
| | CGGAGCGGAACACGTAAACAACTCCTACGAATGTGATATCCCGATTGGAGCGGGAATCT | |
| | GTGCGTCTTACCAAACACAAA | |
| | CAAACTCCCCGAGAAGAGCGAGATCTGTAGCCTCTCAATCTATTATCGCCTACACCATG | |
| | TCCTTGGGAGCCGAAAATTCT | |
| | GTCGCGTACTCCAACAATTCTATCGCGATCCCGACAAACTTCACCATCTCTGTAACAAC | |
| | CGAGATCCTACCGGTGTCTAT | |
| | GACCAAGACATCTGTCGATTGCACCATGTACATCTGCGGAGATTCCACCGAGTGCTCCA | |
| | ACCTACTACTACAGTACGGAT | |
| | CTTTCTGTACCCAGCTAAACAGAGCGTTGACTGGAATCGCTGTAGAGCAGGATAAGAAC | |
| | ACCCAAGAGGTATTCGCGCAA | |
| | GTCAAGCAGATCTATAAGACTCCGCCGATCAAGGACTTCGGAGGTTTTAACTTCTCTCA | |
| | GATCTTGCCGGATCCGTCCAA | |
| | ACCGTCTAAGAGATCTTTCATCGAGGACCTACTATTCAACAAAGTCACCCTAGCTGACG | |
| | CGGGATTCATCAAACAATACG | |
| | GAGATTGCTTGGGAGACATTGCGGCGAGAGATCTAATTTGCGCGCAGAAGTTTAACGGA | |
| | TTGACAGTACTACCGCCGCTA | |
| | CTAACCGATGAGATGATTGCGCAGTACACGTCTGCTCTATTGGCGGGAACAATTACAAG | |
| | TGGATGGACATTTGGAGCCGG | |
| | TGCCGCTCTACAAATTCCGTTTGCTATGCAAATGGCGTACAGATTCAACGGAATCGGAG | |
| | TAACCCAGAACGTCTTGTACG | |
| | AGAACCAGAAGCTAATCGCGAACCAGTTCAATTCCGCGATCGGAAAGATCCAGGACAGT | |
| | CTATCTTCTACTGCTTCGGCG | |
| | TTGGGAAAGCTACAGGATGTAGTAAATCAAAACGCGCAGGCGCTAAACACCTTGGTCAA | |
| | GCAACTATCCTCTAACTTCGG | |
| | AGCGATCTCGTCCGTCCTAAACGACATCTTATCCAGACTAGATAAGGTCGAAGCGGAGG | |
| | TCCAGATCGATAGACTAATCA | |
| | CTGGAAGATTGCAGTCCCTACAGACCTACGTAACACAGCAACTAATTAGAGCGGCGGAG | |
| | ATTAGAGCCTCTGCTAATCTA | |
| | GCTGCGACCAAGATGTCCGAATGTGTCTTGGGACAATCCAAGAGAGTCGACTTTTGCGG | |
| | AAAGGGATACCACCTAATGTC | |
| | TTTTCCACAATCTGCGCCGCATGGTGTCGTATTCCTACATGTAACATATGTGCCGGCGC | |
| | AAGAAAAGAACTTTACAACAG | |
| | CTCCAGCGATCTGCCATGATGGAAAAGCTCATTTTCCGAGAGAGGGAGTCTTTGTCTCT | |
| | AACGGAACTCATTGGTTCGTC | |
| | ACCCAGAGAAACTTTTACGAGCCGCAGATCATCACCACCGACAACACATTTGTTTCGGG | |
| | AAACTGCGACGTGGTCATCGG | |
| | AATCGTAAACAATACCGTCTACGATCCGTTGCAGCCGGAACTAGACTCCTTCAAAGAAG | |
| | AGTTGGACAAGTACTTTAAGA | |
| | ACCACACCTCTCCGGATGTCGACTTGGGAGATATTTCTGGAATCAACGCGTCCGTCGTC | |
| | AACATCCAGAAAGAAATCGAT | |
| | AGATTGAACGAGGTCGCGAAGAACTTGAACGAGTCCCTAATCGACCTACAAGAGCTAGG | |
| | AAAATACGAGCAGTACATCAA | |
| | GTGGCCGTGGTACATTTGGCTAGGATTCATTGCTGGACTAATTGCGATCGTCATGGTCA | |
| | CCATCATGCTATGCTGTATGA | |
| | CCTCCTGTTGCTCCTGTCTAAAGGGATGTTGTTCCTGCGGATCCTGTTGCAAGTTCGAT | |
| | GAAGATGATAGTGAACCGGTC | |
| | CTAAAGGGTGTCAAGCTACACTACACATAAAAGCTT | |
| Nucleotide sequence of | tttggctagtcaagatgatgaatcttcattatctgatatattgcaaatcactcaatatc | |
| HPXV095 gene locus target | tagactttctgttattattat | |
| for SARS-CoV-2 Spike | tgatccaatcaaaaaataaattagaagccgtgggtcattgttatgaatctctttcagag | |
| insertion. SEQ ID NO: 51 | gaatacagacaattgacaaaa | |
| | ttcacagactttcaagattttaaaaaactgtttaacaaggtccctattgttacagatgg | |
| | aagggtcaaacttaataaagg | |
| | atatttgttcgactttgtgattagtttgatgcgattcaaaaaagaatcctctctagcta | |
| | ccaccgcaatagatcctatta | |
| | gatacatagatcctcgtcgtgatatcgcattttctaacgtgatggatatattaaagttg | |
| | aataaagtgaacaataattaa | |
| | ttctttattgtcatcGGATCCCACgatGTGctaGACtctctcGTCtacGCGGCCGCaAc | |
| | tgagagaccAAGCTTGTCGAC | |
| | tattatattttttatctaaaaaactaaaaataaacattgattaaattttaatataatac | |
| | ttaaaaatggatgttgtgtcg | |
| | ttagataaaccgtttatgtattttgaggaaattgataatgagttagattacgaaccaga | |
| | aagtgcaaatgaggtcgcaaa | |
| | aaaactaccgtatcaaggacagttaaaactattactaggagaattattttttcttagta | |
| | agttacagcgacacggtatat | |
| | tagatggtgccaccgtagtgtatataggatcggctcctggtacacatatacgttatttg | |
| | agagatcatttctataattta | |
| | ggaatgattatcaaatggatgctaattgacggacgccatcatgatcctattctaaatgg | |
| | attgcgtgatgtgactctagt | |
| Nucleotide sequence of | gagtattctaggtgtttctatagaatgtaagaagtcAtcgacattacttacttttttga | |
| HPXV200 gene locus target | ccgtgcgtaaaatgacCcgag | |
| for SARS-COV-2 Spike | tatttaatagatttccagatatggcttattatcgaggagactgtttaaaagccgtttat | |
| insertion. SEQ ID NO: 52 | gtaacaatgacttataaaaat | |
| | actaaaactggagagactgattacacgtacctctctaatgggggttgcctgcatactat | |
| | cgtaatggggtcgatggttga | |
| | ttattgattagtatattccttattctttttattcacacaaaaagaacatttttataaac | |
| | atgaaaccactgtctaaatgt | |
| | aattatgatcttgatttatagatgaagatcagcctttagaggattttaaccagtatgtt | |
| | taatatgaaaaaaataaacat | |
| | aacatattttgagattaagcgctattgtgcttaattattttgctctataaactgaatat | |
| | atagccacaattattgacggg | |
| | cttgtttatgaccggcaatcGGATCCCACgatGTGctaGACtctctcGTCtacGCGGCC | |
| | GCaActgagagaccAAGCTTG | |
| | TCGACtaaaatagtttaactcttttaaaaccagtttggtactggaatttcagttcatta | |
| | ctcgttgagaaattgatgatt | |
| | tttttaaaatgatattacttttatatgcttgcatcgcagaatgatattcacaagtatta | |
| | ttaaaaatgagtatcggtagt | |
| | tacattaccatatcatccatgctcatatggatctccatccattatataatcaatgatac | |
| | atgtattaaaatactttccga | |
| | ataagtcttttaaatattgtattaattatgaaaaactatgctatgcgagtatgatgcaa | |
| | agatgtttaatgatacgatac | |
| | tagattttatctctagcgagagatgtcgttagaatcatttatcataactacgtttaata | |
| | ataattcatcaacgaatatcg | |
| | ataacatgtgtcatttatactttaaatacgttaaagtctgtccgtcttctctattgttt | |
| | agactgtttgtagaatgctgt | |
| | gatataaacaaactagtagaaggta | |
| Nucleotide sequence of | 1 | attaaaggtt tataccttcc caggtaacaa accaaccaac tttcgatctc ttgtagatct |
| SARS-CoV-2 Wuhan-Hu-1 | 61 | gttctctaaa cgaactttaa aatctgtgtg gctgtcactc ggctgcatgc ttagtgcact |
| (Accession NC_045512.2). | 121 | cacgcagtat aattaataac taattactgt cgttgacagg acacgagtaa ctcgtctatc |
| SEQ ID NO: 53 | 181 | ttctgcaggc tgcttacggt ttcgtccgtg ttgcagccga tcatcagcac atctaggttt |
| | 241 | cgtccgggtg tgaccgaaag gtaagatgga gagccttgtc cctggtttca acgagaaaac |
| | 301 | acacgtccaa ctcagtttgc ctgttttaca ggttcgcgac gtgctcgtac gtggctttgg |
| | 361 | agactccgtg gaggaggtct tatcagaggc acgtcaacat cttaaagatg gcacttgtgg |
| | 421 | cttagtagaa gttgaaaaag gcgttttgcc tcaacttgaa cagccctatg tgttcatcaa |
| | 481 | acgttcggat gctcgaactg cacctcatgg tcatgttatg gttgagctgg tagcagaact |
| | 541 | cgaaggcatt cagtacggtc gtagtggtga gacacttggt gtccttgtcc ctcatgtggg |
| | 601 | cgaaatacca gtggcttacc gcaaggttct tcttcgtaag aacggtaata aaggagctgg |
| | 661 | tggccatagt tacggcgccg atctaaagtc atttgactta ggcgacgagc ttggcactga |
| | 721 | tccttatgaa gattttcaag aaaactggaa cactaaacat agcagtggtg ttacccgtga |
| | 781 | actcatgcgt gagcttaacg gaggggcata cactcgctat gtcgataaca acttctgtgg |
| | 841 | ccctgatggc taccctcttg agtgcattaa agaccttcta gcacgtgctg gtaaagcttc |
| | 901 | atgcactttg tccgaacaac tggactttat tgacactaag aggggtgtat actgctgccg |
| | 961 | tgaacatgag catgaaattg cttggtacac ggaacgttct gaaaagagct atgaattgca |
| | 1021 | gacacctttt gaaattaaat tggcaaagaa atttgacacc ttcaatgggg aatgtccaaa |
| | 1081 | ttttgtattt cccttaaatt ccataatcaa gactattcaa ccaagggttg aaaagaaaaa |
| | 1141 | gcttgatggc tttatgggta gaattcgatc tgtctatcca gttgcgtcac caaatgaatg |
| | 1201 | caaccaaatg tgcctttcaa ctctcatgaa gtgtgatcat tgtggtgaaa cttcatggca |
| | 1261 | gacgggcgat tttgttaaag ccacttgcga attttgtggc actgagaatt tgactaaaga |
| | 1321 | aggtgccact acttgtggtt acttacccca aaatgctgtt gttaaaattt attgtccagc |
| | 1381 | atgtcacaat tcagaagtag gacctgagca tagtcttgcc gaataccata atgaatctgg |
| | 1441 | cttgaaaacc attcttcgta agggtggtcg cactattgcc tttggaggct gtgtgttctc |
| | 1501 | ttatgttggt tgccataaca agtgtgccta ttgggttcca cgtgctagcg ctaacatagg |
| | 1561 | ttgtaaccat acaggtgttg ttggagaagg ttccgaaggt cttaatgaca accttcttga |
| | 1621 | aatactccaa aaagagaaag tcaacatcaa tattgttggt gactttaaac ttaatgaaga |
| | 1681 | gatcgccatt attttggcat ctttttctgc ttccacaagt gcttttgtgg aaactgtgaa |
| | 1741 | aggtttggat tataaagcat tcaaacaaat tgttgaatcc tgtggtaatt ttaaagttac |
| | 1801 | aaaaggaaaa gctaaaaaag gtgcctggaa tattggtgaa cagaaatcaa tactgagtcc |
| | 1861 | tctttatgca tttgcatcag aggctgctcg tgttgtacga tcaattttct cccgcactct |
| | 1921 | tgaaactgct caaaattctg tgcgtgtttt acagaaggcc gctataacaa tactagatgg |
| | 1981 | aatttcacag tattcactga gactcattga tgctatgatg ttcacatctg atttggctac |
| | 2041 | taacaatcta gttgtaatgg cctacattac aggtggtgtt gttcagttga cttcgcagtg |
| | 2101 | gctaactaac atctttggca ctgtttatga aaaactcaaa cccgtccttg attggcttga |
| | 2161 | agagaagttt aaggaaggtg tagagtttct tagagacggt tgggaaattg ttaaatttat |
| | 2221 | ctcaacctgt gcttgtgaaa ttgtcggtgg acaaattgtc acctgtgcaa aggaaattaa |
| | 2281 | ggagagtgtt cagacattct ttaagcttgt aaataaattt ttggctttgt gtgctgactc |
| | 2341 | tatcattatt ggtggagcta aacttaaagc cttgaattta ggtgaaacat ttgtcacgca |
| | 2401 | ctcaaaggga ttgtacagaa agtgtgttaa atccagagaa gaaactggcc tactcatgcc |
| | 2461 | tctaaaagcc ccaaaagaaa ttatcttctt agagggagaa acacttccca cagaagtgtt |
| | 2521 | aacagaggaa gttgtcttga aaactggtga tttacaacca ttagaacaac ctactagtga |
| | 2581 | agctgttgaa gctccattgg ttggtacacc agtttgtatt aacgggctta tgttgctcga |
| | 2641 | aatcaaagac acagaaaagt actgtgccct tgcacctaat atgatggtaa caaacaatac |
| | 2701 | cttcacactc aaaggcggtg caccaacaaa ggttactttt ggtgatgaca ctgtgataga |
| | 2761 | agtgcaaggt tacaagagtg tgaatatcac ttttgaactt gatgaaagga ttgataaagt |
| | 2821 | acttaatgag aagtgctctg cctatacagt tgaactcggt acagaagtaa atgagttcgc |
| | 2881 | ctgtgttgtg gcagatgctg tcataaaaac tttgcaacca gtatctgaat tacttacacc |
| | 2941 | actgggcatt gatttagatg agtggagtat ggctacatac tacttatttg atgagtctgg |
| | 3001 | tgagtttaaa ttggcttcac atatgtattg ttctttctac cctccagatg aggatgaaga |
| | 3061 | agaaggtgat tgtgaagaag aagagtttga gccatcaact caatatgagt atggtactga |
| | 3121 | agatgattac caaggtaaac ctttggaatt tggtgccact tctgctgctc ttcaacctga |
| | 3181 | agaagagcaa gaagaagatt ggttagatga tgatagtcaa caaactgttg gtcaacaaga |
| | 3241 | cggcagtgag gacaatcaga caactactat tcaaacaatt gttgaggttc aacctcaatt |
| | 3301 | agagatggaa cttacaccag ttgttcagac tattgaagtg aatagtttta gtggttattt |
| | 3361 | aaaacttact gacaatgtat acattaaaaa tgcagacatt gtggaagaag ctaaaaaggt |
| | 3421 | aaaaccaaca gtggttgtta atgcagccaa tgtttacctt aaacatggag gaggtgttgc |
| | 3481 | aggagcctta aataaggcta ctaacaatgc catgcaagtt gaatctgatg attacatagc |
| | 3541 | tactaatgga ccacttaaag tgggtggtag ttgtgtttta agcggacaca atcttgctaa |
| | 3601 | acactgtctt catgttgtcg gcccaaatgt taacaaaggt gaagacattc aacttcttaa |
| | 3661 | gagtgcttat gaaaatttta atcagcacga agttctactt gcaccattat tatcagctgg |
| | 3721 | tatttttggt gctgacccta tacattcttt aagagtttgt gtagatactg ttcgcacaaa |
| | 3781 | tgtctactta gctgtctttg ataaaaatct ctatgacaaa cttgtttcaa gctttttgga |
| | 3841 | aatgaagagt gaaaagcaag ttgaacaaaa gatcgctgag attcctaaag aggaagttaa |
| | 3901 | gccatttata actgaaagta aaccttcagt tgaacagaga aaacaagatg ataagaaaat |
| | 3961 | caaagcttgt gttgaagaag ttacaacaac tctggaagaa actaagttcc tcacagaaaa |
| | 4021 | cttgttactt tatattgaca ttaatggcaa tcttcatcca gattctgcca ctcttgttag |
| | 4081 | tgacattgac atcactttct taaagaaaga tgctccatat atagtgggtg atgttgttca |
| | 4141 | agagggtgtt ttaactgctg tggttatacc tactaaaaag gctggtggca ctactgaaat |
| | 4201 | gctagcgaaa gctttgagaa aagtgccaac agacaattat ataaccactt acccgggtca |
| | 4261 | gggtttaaat ggttacactg tagaggaggc aaagacagtg cttaaaaagt gtaaaagtgc |
| | 4321 | cttttacatt ctaccatcta ttatctctaa tgagaagcaa gaaattcttg gaactgtttc |
| | 4381 | ttggaatttg cgagaaatgc ttgcacatgc agaagaaaca cgcaaattaa tgcctgtctg |
| | 4441 | tgtggaaact aaagccatag tttcaactat acagcgtaaa tataagggta ttaaaataca |
| | 4501 | agagggtgtg gttgattatg gtgctagatt ttacttttac accagtaaaa caactgtagc |
| | 4561 | gtcacttatc aacacactta acgatctaaa tgaaactctt gttacaatgc cacttggcta |
| | 4621 | tgtaacacat ggcttaaatt tggaagaagc tgctcggtat atgagatctc tcaaagtgcc |
| | 4681 | agctacagtt tctgtttctt cacctgatgc tgttacagcg tataatggtt atcttacttc |
| | 4741 | ttcttctaaa acacctgaag aacattttat tgaaaccatc tcacttgctg gttcctataa |
| | 4801 | agattggtcc tattctggac aatctacaca actaggtata gaatttctta agagaggtga |
| | 4861 | taaaagtgta tattacacta gtaatcctac cacattccac ctagatggtg aagttatcac |
| | 4921 | ctttgacaat cttaagacac ttctttcttt gagagaagtg aggactatta aggtgtttac |
| | 4981 | aacagtagac aacattaacc tccacacgca agttgtggac atgtcaatga catatggaca |
| | 5041 | acagtttggt ccaacttatt tggatggagc tgatgttact aaaataaaac ctcataattc |
| | 5101 | acatgaaggt aaaacatttt atgttttacc taatgatgac actctacgtg ttgaggcttt |
| | 5161 | tgagtactac cacacaactg atcctagttt tctgggtagg tacatgtcag cattaaatca |
| | 5221 | cactaaaaag tggaaatacc cacaagttaa tggtttaact tctattaaat gggcagataa |
| | 5281 | caactgttat cttgccactg cattgttaac actccaacaa atagagttga agtttaatcc |
| | 5341 | acctgctcta caagatgctt attacagagc aagggctggt gaagctgcta acttttgtgc |
| | 5401 | acttatctta gcctactgta ataagacagt aggtgagtta ggtgatgtta gagaaacaat |
| | 5461 | gagttacttg tttcaacatg ccaatttaga ttcttgcaaa agagtcttga acgtggtgtg |
| | 5521 | taaaacttgt ggacaacagc agacaaccct taagggtgta gaagctgtta tgtacatggg |
| | 5581 | cacactttct tatgaacaat ttaagaaagg tgttcagata ccttgtacgt gtggtaaaca |
| | 5641 | agctacaaaa tatctagtac aacaggagtc accttttgtt atgatgtcag caccacctgc |
| | 5701 | tcagtatgaa cttaagcatg gtacatttac ttgtgctagt gagtacactg gtaattacca |
| | 5761 | gtgtggtcac tataaacata taacttctaa agaaactttg tattgcatag acggtgcttt |
| | 5821 | acttacaaag tcctcagaat acaaaggtcc tattacggat gttttctaca aagaaaacag |
| | 5881 | ttacacaaca accataaaac cagttactta taaattggat ggtgttgttt gtacagaaat |
| | 5941 | tgaccctaag ttggacaatt attataagaa agacaattct tatttcacag agcaaccaat |
| | 6001 | tgatcttgta ccaaaccaac catatccaaa cgcaagcttc gataatttta agtttgtatg |
| | 6061 | tgataatatc aaatttgctg atgatttaaa ccagttaact ggttataaga aacctgcttc |
| | 6121 | aagagagctt aaagttacat ttttccctga cttaaatggt gatgtggtgg ctattgatta |
| | 6181 | taaacactac acaccctctt ttaagaaagg agctaaattg ttacataaac ctattgtttg |
| | 6241 | gcatgttaac aatgcaacta ataaagccac gtataaacca aatacctggt gtatacgttg |
| | 6301 | tctttggagc acaaaaccag ttgaaacatc aaattcgttt gatgtactga agtcagagga |
| | 6361 | cgcgcaggga atggataatc ttgcctgcga agatctaaaa ccagtctctg aagaagtagt |
| | 6421 | ggaaaatcct accatacaga aagacgttct tgagtgtaat gtgaaaacta ccgaagttgt |
| | 6481 | aggagacatt atacttaaac cagcaaataa tagtttaaaa attacagaag aggttggcca |
| | 6541 | cacagatcta atggctgctt atgtagacaa ttctagtctt actattaaga aacctaatga |
| | 6601 | attatctaga gtattaggtt tgaaaaccct tgctactcat ggtttagctg ctgttaatag |
| | 6661 | tgtcccttgg gatactatag ctaattatgc taagcctttt cttaacaaag ttgttagtac |
| | 6721 | aactactaac atagttacac ggtgtttaaa ccgtgtttgt actaattata tgccttattt |
| | 6781 | ctttacttta ttgctacaat tgtgtacttt tactagaagt acaaattcta gaattaaagc |
| | 6841 | atctatgccg actactatag caaagaatac tgttaagagt gtcggtaaat tttgtctaga |
| | 6901 | ggcttcattt aattatttga agtcacctaa tttttctaaa ctgataaata ttataatttg |
| | 6961 | gtttttacta ttaagtgttt gcctaggttc tttaatctac tcaaccgctg ctttaggtgt |
| | 7021 | tttaatgtct aatttaggca tgccttctta ctgtactggt tacagagaag gctatttgaa |
| | 7081 | ctctactaat gtcactattg caacctactg tactggttct ataccttgta gtgtttgtct |
| | 7141 | tagtggttta gattctttag acacctatcc ttctttagaa actatacaaa ttaccatttc |
| | 7201 | atcttttaaa tgggatttaa ctgcttttgg cttagttgca gagtggtttt tggcatatat |
| | 7261 | tcttttcact aggtttttct atgtacttgg attggctgca atcatgcaat tgtttttcag |
| | 7321 | ctattttgca gtacatttta ttagtaattc ttggcttatg tggttaataa ttaatcttgt |
| | 7381 | acaaatggcc ccgatttcag ctatggttag aatgtacatc ttctttgcat cattttatta |
| | 7441 | tgtatggaaa agttatgtgc atgttgtaga cggttgtaat tcatcaactt gtatgatgtg |
| | 7501 | ttacaaacgt aatagagcaa caagagtcga atgtacaact attgttaatg gtgttagaag |
| | 7561 | gtccttttat gtctatgcta atggaggtaa aggcttttgc aaactacaca attggaattg |
| | 7621 | tgttaattgt gatacattct gtgctggtag tacatttatt agtgatgaag ttgcgagaga |
| | 7681 | cttgtcacta cagtttaaaa gaccaataaa tcctactgac cagtcttctt acatcgttga |
| | 7741 | tagtgttaca gtgaagaatg gttccatcca tctttacttt gataaagctg gtcaaaagac |
| | 7801 | ttatgaaaga cattctctct ctcattttgt taacttagac aacctgagag ctaataacac |
| | 7861 | taaaggttca ttgcctatta atgttatagt ttttgatggt aaatcaaaat gtgaagaatc |
| | 7921 | atctgcaaaa tcagcgtctg tttactacag tcagcttatg tgtcaaccta tactgttact |
| | 7981 | agatcaggca ttagtgtctg atgttggtga tagtgcggaa gttgcagtta aaatgtttga |
| | 8041 | tgcttacgtt aatacgtttt catcaacttt taacgtacca atggaaaaac tcaaaacact |
| | 8101 | agttgcaact gcagaagctg aacttgcaaa gaatgtgtcc ttagacaatg tcttatctac |
| | 8161 | ttttatttca gcagctcggc aagggtttgt tgattcagat gtagaaacta aagatgttgt |
| | 8221 | tgaatgtctt aaattgtcac atcaatctga catagaagtt actggcgata gttgtaataa |
| | 8281 | ctatatgctc acctataaca aagttgaaaa catgacaccc cgtgaccttg gtgcttgtat |
| | 8341 | tgactgtagt gcgcgtcata ttaatgcgca ggtagcaaaa agtcacaaca ttgctttgat |
| | 8401 | atggaacgtt aaagatttca tgtcattgtc tgaacaacta cgaaaacaaa tacgtagtgc |
| | 8461 | tgctaaaaag aataacttac cttttaagtt gacatgtgca actactagac aagttgttaa |
| | 8521 | tgttgtaaca acaaagatag cacttaaggg tggtaaaatt gttaataatt ggttgaagca |
| | 8581 | gttaattaaa gttacacttg tgttcctttt tgttgctgct attttctatt taataacacc |
| | 8641 | tgttcatgtc atgtctaaac atactgactt ttcaagtgaa atcataggat acaaggctat |
| | 8701 | tgatggtggt gtcactcgtg acatagcatc tacagatact tgttttgcta acaaacatgc |
| | 8761 | tgattttgac acatggttta gccagcgtgg tggtagttat actaatgaca aagcttgccc |
| | 8821 | attgattgct gcagtcataa caagagaagt gggttttgtc gtgcctggtt tgcctggcac |
| | 8881 | gatattacgc acaactaatg gtgacttttt gcatttctta cctagagttt ttagtgcagt |
| | 8941 | tggtaacatc tgttacacac catcaaaact tatagagtac actgactttg caacatcagc |
| | 9001 | ttgtgttttg gctgctgaat gtacaatttt taaagatgct tctggtaagc cagtaccata |
| | 9061 | ttgttatgat accaatgtac tagaaggttc tgttgcttat gaaagtttac gccctgacac |
| | 9121 | acgttatgtg ctcatggatg gctctattat tcaatttcct aacacctacc ttgaaggttc |
| | 9181 | tgttagagtg gtaacaactt ttgattctga gtactgtagg cacggcactt gtgaaagatc |
| | 9241 | agaagctggt gtttgtgtat ctactagtgg tagatgggta cttaacaatg attattacag |
| | 9301 | atctttacca ggagttttct gtggtgtaga tgctgtaaat ttacttacta atatgtttac |
| | 9361 | accactaatt caacctattg gtgctttgga catatcagca tctatagtag ctggtggtat |
| | 9421 | tgtagctatc gtagtaacat gccttgccta ctattttatg aggtttagaa gagcttttgg |
| | 9481 | tgaatacagt catgtagttg cctttaatac tttactattc cttatgtcat tcactgtact |
| | 9541 | ctgtttaaca ccagtttact cattcttacc tggtgtttat tctgttattt acttgtactt |
| | 9601 | gacattttat cttactaatg atgtttcttt tttagcacat attcagtgga tggttatgtt |
| | 9661 | cacaccttta gtacctttct ggataacaat tgcttatatc atttgtattt ccacaaagca |
| | 9721 | tttctattgg ttctttagta attacctaaa gagacgtgta gtctttaatg gtgtttcctt |
| | 9781 | tagtactttt gaagaagctg cgctgtgcac ctttttgtta aataaagaaa tgtatctaaa |
| | 9841 | gttgcgtagt gatgtgctat tacctcttac gcaatataat agatacttag ctctttataa |
| | 9901 | taagtacaag tattttagtg gagcaatgga tacaactagc tacagagaag ctgcttgttg |
| | 9961 | tcatctcgca aaggctctca atgacttcag taactcaggt tctgatgttc tttaccaacc |
| | 10021 | accacaaacc tctatcacct cagctgtttt gcagagtggt tttagaaaaa tggcattccc |
| | 10081 | atctggtaaa gttgagggtt gtatggtaca agtaacttgt ggtacaacta cacttaacgg |
| | 10141 | tctttggctt gatgacgtag tttactgtcc aagacatgtg atctgcacct ctgaagacat |
| | 10201 | gcttaaccct aattatgaag atttactcat tcgtaagtct aatcataatt tcttggtaca |
| | 10261 | ggctggtaat gttcaactca gggttattgg acattctatg caaaattgtg tacttaagct |
| | 10321 | taaggttgat acagccaatc ctaagacacc taagtataag tttgttcgca ttcaaccagg |
| | 10381 | acagactttt tcagtgttag cttgttacaa tggttcacca tctggtgttt accaatgtgc |
| | 10441 | tatgaggccc aatttcacta ttaagggttc attccttaat ggttcatgtg gtagtgttgg |
| | 10501 | ttttaacata gattatgact gtgtctcttt ttgttacatg caccatatgg aattaccaac |
| | 10561 | tggagttcat gctggcacag acttagaagg taacttttat ggaccttttg ttgacaggca |
| | 10621 | aacagcacaa gcagctggta cggacacaac tattacagtt aatgttttag cttggttgta |
| | 10681 | cgctgctgtt ataaatggag acaggtggtt tctcaatcga tttaccacaa ctcttaatga |
| | 10741 | ctttaacctt gtggctatga agtacaatta tgaacctcta acacaagacc atgttgacat |
| | 10801 | actaggacct ctttctgctc aaactggaat tgccgtttta gatatgtgtg cttcattaaa |
| | 10861 | agaattactg caaaatggta tgaatggacg taccatattg ggtagtgctt tattagaaga |
| | 10921 | tgaatttaca ccttttgatg ttgttagaca atgctcaggt gttactttcc aaagtgcagt |
| | 10981 | gaaaagaaca atcaagggta cacaccactg gttgttactc acaattttga cttcactttt |
| | 11041 | agttttagtc cagagtactc aatggtcttt gttctttttt ttgtatgaaa atgccttttt |
| | 11101 | accttttgct atgggtatta ttgctatgtc tgcttttgca atgatgtttg tcaaacataa |
| | 11161 | gcatgcattt ctctgtttgt ttttgttacc ttctcttgcc actgtagctt attttaatat |
| | 11221 | ggtctatatg cctgctagtt gggtgatgcg tattatgaca tggttggata tggttgatac |
| | 11281 | tagtttgtct ggttttaagc taaaagactg tgttatgtat gcatcagctg tagtgttact |
| | 11341 | aatccttatg acagcaagaa ctgtgtatga tgatggtgct aggagagtgt ggacacttat |
| | 11401 | gaatgtcttg acactcgttt ataaagttta ttatggtaat gctttagatc aagccatttc |
| | 11461 | catgtgggct cttataatct ctgttacttc taactactca ggtgtagtta caactgtcat |
| | 11521 | gtttttggcc agaggtattg tttttatgtg tgttgagtat tgccctattt tcttcataac |
| | 11581 | tggtaataca cttcagtgta taatgctagt ttattgtttc ttaggctatt tttgtacttg |
| | 11641 | ttactttggc ctcttttgtt tactcaaccg ctactttaga ctgactcttg gtgtttatga |
| | 11701 | ttacttagtt tctacacagg agtttagata tatgaattca cagggactac tcccacccaa |
| | 11761 | gaatagcata gatgccttca aactcaacat taaattgttg ggtgttggtg gcaaaccttg |
| | 11821 | tatcaaagta gccactgtac agtctaaaat gtcagatgta aagtgcacat cagtagtctt |
| | 11881 | actctcagtt ttgcaacaac tcagagtaga atcatcatct aaattgtggg ctcaatgtgt |
| | 11941 | ccagttacac aatgacattc tcttagctaa agatactact gaagcctttg aaaaaatggt |
| | 12001 | ttcactactt tctgttttgc tttccatgca gggtgctgta gacataaaca agctttgtga |
| | 12061 | agaaatgctg gacaacaggg caaccttaca agctatagcc tcagagttta gttcccttcc |
| | 12121 | atcatatgca gcttttgcta ctgctcaaga agcttatgag caggctgttg ctaatggtga |
| | 12181 | ttctgaagtt gttcttaaaa agttgaagaa gtctttgaat gtggctaaat ctgaatttga |
| | 12241 | ccgtgatgca gccatgcaac gtaagttgga aaagatggct gatcaagcta tgacccaaat |
| | 12301 | gtataaacag gctagatctg aggacaagag ggcaaaagtt actagtgcta tgcagacaat |
| | 12361 | gcttttcact atgcttagaa agttggataa tgatgcactc aacaacatta tcaacaatgc |
| | 12421 | aagagatggt tgtgttccct tgaacataat acctcttaca acagcagcca aactaatggt |
| | 12481 | tgtcatacca gactataaca catataaaaa tacgtgtgat ggtacaacat ttacttatgc |
| | 12541 | atcagcattg tgggaaatcc aacaggttgt agatgcagat agtaaaattg ttcaacttag |
| | 12601 | tgaaattagt atggacaatt cacctaattt agcatggcct cttattgtaa cagctttaag |
| | 12661 | ggccaattct gctgtcaaat tacagaataa tgagcttagt cctgttgcac tacgacagat |
| | 12721 | gtcttgtgct gccggtacta cacaaactgc ttgcactgat gacaatgcgt tagcttacta |
| | 12781 | caacacaaca aagggaggta ggtttgtact tgcactgtta tccgatttac aggatttgaa |
| | 12841 | atgggctaga ttccctaaga gtgatggaac tggtactatc tatacagaac tggaaccacc |
| | 12901 | ttgtaggttt gttacagaca cacctaaagg tcctaaagtg aagtatttat actttattaa |
| | 12961 | aggattaaac aacctaaata gaggtatggt acttggtagt ttagctgcca cagtacgtct |
| | 13021 | acaagctggt aatgcaacag aagtgcctgc caattcaact gtattatctt tctgtgcttt |
| | 13081 | tgctgtagat gctgctaaag cttacaaaga ttatctagct agtgggggac aaccaatcac |
| | 13141 | taattgtgtt aagatgttgt gtacacacac tggtactggt caggcaataa cagttacacc |
| | 13201 | ggaagccaat atggatcaag aatcctttgg tggtgcatcg tgttgtctgt actgccgttg |
| | 13261 | ccacatagat catccaaatc ctaaaggatt ttgtgactta aaaggtaagt atgtacaaat |
| | 13321 | acctacaact tgtgctaatg accctgtggg ttttacactt aaaaacacag tctgtaccgt |
| | 13381 | ctgcggtatg tggaaaggtt atggctgtag ttgtgatcaa ctccgcgaac ccatgcttca |
| | 13441 | gtcagctgat gcacaatcgt ttttaaacgg gtttgcggtg taagtgcagc ccgtcttaca |
| | 13501 | ccgtgcggca caggcactag tactgatgtc gtatacaggg cttttgacat ctacaatgat |
| | 13561 | aaagtagctg gttttgctaa attcctaaaa actaattgtt gtcgcttcca agaaaaggac |
| | 13621 | gaagatgaca atttaattga ttcttacttt gtagttaaga gacacacttt ctctaactac |
| | 13681 | caacatgaag aaacaattta taatttactt aaggattgtc cagctgttgc taaacatgac |
| | 13741 | ttctttaagt ttagaataga cggtgacatg gtaccacata tatcacgtca acgtcttact |
| | 13801 | aaatacacaa tggcagacct cgtctatgct ttaaggcatt ttgatgaagg taattgtgac |
| | 13861 | acattaaaag aaatacttgt cacatacaat tgttgtgatg atgattattt caataaaaag |
| | 13921 | gactggtatg attttgtaga aaacccagat atattacgcg tatacgccaa cttaggtgaa |
| | 13981 | cgtgtacgcc aagctttgtt aaaaacagta caattctgtg atgccatgcg aaatgctggt |
| | 14041 | attgttggtg tactgacatt agataatcaa gatctcaatg gtaactggta tgatttcggt |
| | 14101 | gatttcatac aaaccacgcc aggtagtgga gttcctgttg tagattctta ttattcattg |
| | 14161 | ttaatgccta tattaacctt gaccagggct ttaactgcag agtcacatgt tgacactgac |
| | 14221 | ttaacaaagc cttacattaa gtgggatttg ttaaaatatg acttcacgga agagaggtta |
| | 14281 | aaactctttg accgttattt taaatattgg gatcagacat accacccaaa ttgtgttaac |
| | 14341 | tgtttggatg acagatgcat tctgcattgt gcaaacttta atgttttatt ctctacagtg |
| | 14401 | ttcccaccta caagttttgg accactagtg agaaaaatat ttgttgatgg tgttccattt |
| | 14461 | gtagtttcaa ctggatacca cttcagagag ctaggtgttg tacataatca ggatgtaaac |
| | 14521 | ttacatagct ctagacttag ttttaaggaa ttacttgtgt atgctgctga ccctgctatg |
| | 14581 | cacgctgctt ctggtaatct attactagat aaacgcacta cgtgcttttc agtagctgca |
| | 14641 | cttactaaca atgttgcttt tcaaactgtc aaacccggta attttaacaa agacttctat |
| | 14701 | gactttgctg tgtctaaggg tttctttaag gaaggaagtt ctgttgaatt aaaacacttc |
| | 14761 | ttctttgctc aggatggtaa tgctgctatc agcgattatg actactatcg ttataatcta |
| | 14821 | ccaacaatgt gtgatatcag acaactacta tttgtagttg aagttgttga taagtacttt |
| | 14881 | gattgttacg atggtggctg tattaatgct aaccaagtca tcgtcaacaa cctagacaaa |
| | 14941 | tcagctggtt ttccatttaa taaatggggt aaggctagac tttattatga ttcaatgagt |
| | 15001 | tatgaggatc aagatgcact tttcgcatat acaaaacgta atgtcatccc tactataact |
| | 15061 | caaatgaatc ttaagtatgc cattagtgca aagaatagag ctcgcaccgt agctggtgtc |
| | 15121 | tctatctgta gtactatgac caatagacag tttcatcaaa aattattgaa atcaatagcc |
| | 15181 | gccactagag gagctactgt agtaattgga acaagcaaat tctatggtgg ttggcacaac |
| | 15241 | atgttaaaaa ctgtttatag tgatgtagaa aaccctcacc ttatgggttg ggattatcct |
| | 15301 | aaatgtgata gagccatgcc taacatgctt agaattatgg cctcacttgt tcttgctcgc |
| | 15361 | aaacatacaa cgtgttgtag cttgtcacac cgtttctata gattagctaa tgagtgtgct |
| | 15421 | caagtattga gtgaaatggt catgtgtggc ggttcactat atgttaaacc aggtggaacc |
| | 15481 | tcatcaggag atgccacaac tgcttatgct aatagtgttt ttaacatttg tcaagctgtc |
| | 15541 | acggccaatg ttaatgcact tttatctact gatggtaaca aaattgccga taagtatgtc |
| | 15601 | cgcaatttac aacacagact ttatgagtgt ctctatagaa atagagatgt tgacacagac |
| | 15661 | tttgtgaatg agttttacgc atatttgcgt aaacatttct caatgatgat actctctgac |
| | 15721 | gatgctgttg tgtgtttcaa tagcacttat gcatctcaag gtctagtggc tagcataaag |
| | 15781 | aactttaagt cagttcttta ttatcaaaac aatgttttta tgtctgaagc aaaatgttgg |
| | 15841 | actgagactg accttactaa aggacctcat gaattttgct ctcaacatac aatgctagtt |
| | 15901 | aaacagggtg atgattatgt gtaccttcct tacccagatc catcaagaat cctaggggcc |
| | 15961 | ggctgttttg tagatgatat cgtaaaaaca gatggtacac ttatgattga acggttcgtg |
| | 16021 | tctttagcta tagatgctta cccacttact aaacatccta atcaggagta tgctgatgtc |
| | 16081 | tttcatttgt acttacaata cataagaaag ctacatgatg agttaacagg acacatgtta |
| | 16141 | gacatgtatt ctgttatgct tactaatgat aacacttcaa ggtattggga acctgagttt |
| | 16201 | tatgaggcta tgtacacacc gcatacagtc ttacaggctg ttggggcttg tgttctttgc |
| | 16261 | aattcacaga cttcattaag atgtggtgct tgcatacgta gaccattctt atgttgtaaa |
| | 16321 | tgctgttacg accatgtcat atcaacatca cataaattag tcttgtctgt taatccgtat |
| | 16381 | gtttgcaatg ctccaggttg tgatgtcaca gatgtgactc aactttactt aggaggtatg |
| | 16441 | agctattatt gtaaatcaca taaaccaccc attagttttc cattgtgtgc taatggacaa |
| | 16501 | gtttttggtt tatataaaaa tacatgtgtt ggtagcgata atgttactga ctttaatgca |
| | 16561 | attgcaacat gtgactggac aaatgctggt gattacattt tagctaacac ctgtactgaa |
| | 16621 | agactcaagc tttttgcagc agaaacgctc aaagctactg aggagacatt taaactgtct |
| | 16681 | tatggtattg ctactgtacg tgaagtgctg tctgacagag aattacatct ttcatgggaa |
| | 16741 | gttggtaaac ctagaccacc acttaaccga aattatgtct ttactggtta tcgtgtaact |
| | 16801 | aaaaacagta aagtacaaat aggagagtac acctttgaaa aaggtgacta tggtgatgct |
| | 16861 | gttgtttacc gaggtacaac aacttacaaa ttaaatgttg gtgattattt tgtgctgaca |
| | 16921 | tcacatacag taatgccatt aagtgcacct acactagtgc cacaagagca ctatgttaga |
| | 16981 | attactggct tatacccaac actcaatatc tcagatgagt tttctagcaa tgttgcaaat |
| | 17041 | tatcaaaagg ttggtatgca aaagtattct acactccagg gaccacctgg tactggtaag |
| | 17101 | agtcattttg ctattggcct agctctctac tacccttctg ctcgcatagt gtatacagct |
| | 17161 | tgctctcatg ccgctgttga tgcactatgt gagaaggcat taaaatattt gcctatagat |
| | 17221 | aaatgtagta gaattatacc tgcacgtgct cgtgtagagt gttttgataa attcaaagtg |
| | 17281 | aattcaacat tagaacagta tgtcttttgt actgtaaatg cattgcctga gacgacagca |
| | 17341 | gatatagttg tctttgatga aatttcaatg gccacaaatt atgatttgag tgttgtcaat |
| | 17401 | gccagattac gtgctaagca ctatgtgtac attggcgacc ctgctcaatt acctgcacca |
| | 17461 | cgcacattgc taactaaggg cacactagaa ccagaatatt tcaattcagt gtgtagactt |
| | 17521 | atgaaaacta taggtccaga catgttcctc ggaacttgtc ggcgttgtcc tgctgaaatt |
| | 17581 | gttgacactg tgagtgcttt ggtttatgat aataagctta aagcacataa agacaaatca |
| | 17641 | gctcaatgct ttaaaatgtt ttataagggt gttatcacgc atgatgtttc atctgcaatt |
| | 17701 | aacaggccac aaataggcgt ggtaagagaa ttccttacac gtaaccctgc ttggagaaaa |
| | 17761 | gctgtcttta tttcacctta taattcacag aatgctgtag cctcaaagat tttgggacta |
| | 17821 | ccaactcaaa ctgttgattc atcacagggc tcagaatatg actatgtcat attcactcaa |
| | 17881 | accactgaaa cagctcactc ttgtaatgta aacagattta atgttgctat taccagagca |
| | 17941 | aaagtaggca tactttgcat aatgtctgat agagaccttt atgacaagtt gcaatttaca |
| | 18001 | agtcttgaaa ttccacgtag gaatgtggca actttacaag ctgaaaatgt aacaggactc |
| | 18061 | tttaaagatt gtagtaaggt aatcactggg ttacatccta cacaggcacc tacacacctc |
| | 18121 | agtgttgaca ctaaattcaa aactgaaggt ttatgtgttg acatacctgg catacctaag |
| | 18181 | gacatgacct atagaagact catctctatg atgggtttta aaatgaatta tcaagttaat |
| | 18241 | ggttacccta acatgtttat cacccgcgaa gaagctataa gacatgtacg tgcatggatt |
| | 18301 | ggcttcgatg tcgaggggtg tcatgctact agagaagctg ttggtaccaa tttaccttta |
| | 18361 | cagctaggtt tttctacagg tgttaaccta gttgctgtac ctacaggtta tgttgataca |
| | 18421 | cctaataata cagatttttc cagagttagt gctaaaccac cgcctggaga tcaatttaaa |
| | 18481 | cacctcatac cacttatgta caaaggactt ccttggaatg tagtgcgtat aaagattgta |
| | 18541 | caaatgttaa gtgacacact taaaaatctc tctgacagag tcgtatttgt cttatgggca |
| | 18601 | catggctttg agttgacatc tatgaagtat tttgtgaaaa taggacctga gcgcacctgt |
| | 18661 | tgtctatgtg atagacgtgc cacatgcttt tccactgctt cagacactta tgcctgttgg |
| | 18721 | catcattcta ttggatttga ttacgtctat aatccgttta tgattgatgt tcaacaatgg |
| | 18781 | ggttttacag gtaacctaca aagcaaccat gatctgtatt gtcaagtcca tggtaatgca |
| | 18841 | catgtagcta gttgtgatgc aatcatgact aggtgtctag ctgtccacga gtgctttgtt |
| | 18901 | aagcgtgttg actggactat tgaatatcct ataattggtg atgaactgaa gattaatgcg |
| | 18961 | gcttgtagaa aggttcaaca catggttgtt aaagctgcat tattagcaga caaattccca |
| | 19021 | gttcttcacg acattggtaa ccctaaagct attaagtgtg tacctcaagc tgatgtagaa |
| | 19081 | tggaagttct atgatgcaca gccttgtagt gacaaagctt ataaaataga agaattattc |
| | 19141 | tattcttatg ccacacattc tgacaaattc acagatggtg tatgcctatt ttggaattgc |
| | 19201 | aatgtcgata gatatcctgc taattccatt gtttgtagat ttgacactag agtgctatct |
| | 19261 | aaccttaact tgcctggttg tgatggtggc agtttgtatg taaataaaca tgcattccac |
| | 19321 | acaccagctt ttgataaaag tgcttttgtt aatttaaaac aattaccatt tttctattac |
| | 19381 | tctgacagtc catgtgagtc tcatggaaaa caagtagtgt cagatataga ttatgtacca |
| | 19441 | ctaaagtctg ctacgtgtat aacacgttgc aatttaggtg gtgctgtctg tagacatcat |
| | 19501 | gctaatgagt acagattgta tctcgatgct tataacatga tgatctcagc tggctttagc |
| | 19561 | ttgtgggttt acaaacaatt tgatacttat aacctctgga acacttttac aagacttcag |
| | 19621 | agtttagaaa atgtggcttt taatgttgta aataagggac actttgatgg acaacagggt |
| | 19681 | gaagtaccag tttctatcat taataacact gtttacacaa aagttgatgg tgttgatgta |
| | 19741 | gaattgtttg aaaataaaac aacattacct gttaatgtag catttgagct ttgggctaag |
| | 19801 | cgcaacatta aaccagtacc agaggtgaaa atactcaata atttgggtgt ggacattgct |
| | 19861 | gctaatactg tgatctggga ctacaaaaga gatgctccag cacatatatc tactattggt |
| | 19921 | gtttgttcta tgactgacat agccaagaaa ccaactgaaa cgatttgtgc accactcact |
| | 19981 | gtcttttttg atggtagagt tgatggtcaa gtagacttat ttagaaatgc ccgtaatggt |
| | 20041 | gttcttatta cagaaggtag tgttaaaggt ttacaaccat ctgtaggtcc caaacaagct |
| | 20101 | agtcttaatg gagtcacatt aattggagaa gccgtaaaaa cacagttcaa ttattataag |
| | 20161 | aaagttgatg gtgttgtcca acaattacct gaaacttact ttactcagag tagaaattta |
| | 20221 | caagaattta aacccaggag tcaaatggaa attgatttct tagaattagc tatggatgaa |
| | 20281 | ttcattgaac ggtataaatt agaaggctat gccttcgaac atatcgttta tggagatttt |
| | 20341 | agtcatagtc agttaggtgg tttacatcta ctgattggac tagctaaacg ttttaaggaa |
| | 20401 | tcaccttttg aattagaaga ttttattcct atggacagta cagttaaaaa ctatttcata |
| | 20461 | acagatgcgc aaacaggttc atctaagtgt gtgtgttctg ttattgattt attacttgat |
| | 20521 | gattttgttg aaataataaa atcccaagat ttatctgtag tttctaaggt tgtcaaagtg |
| | 20581 | actattgact atacagaaat ttcatttatg ctttggtgta aagatggcca tgtagaaaca |
| | 20641 | ttttacccaa aattacaatc tagtcaagcg tggcaaccgg gtgttgctat gcctaatctt |
| | 20701 | tacaaaatgc aaagaatgct attagaaaag tgtgaccttc aaaattatgg tgatagtgca |
| | 20761 | acattaccta aaggcataat gatgaatgtc gcaaaatata ctcaactgtg tcaatattta |
| | 20821 | aacacattaa cattagctgt accctataat atgagagtta tacattttgg tgctggttct |
| | 20881 | gataaaggag ttgcaccagg tacagctgtt ttaagacagt ggttgcctac gggtacgctg |
| | 20941 | cttgtcgatt cagatcttaa tgactttgtc tctgatgcag attcaacttt gattggtgat |
| | 21001 | tgtgcaactg tacatacagc taataaatgg gatctcatta ttagtgatat gtacgaccct |
| | 21061 | aagactaaaa atgttacaaa agaaaatgac tctaaagagg gttttttcac ttacatttgt |
| | 21121 | gggtttatac aacaaaagct agctcttgga ggttccgtgg ctataaagat aacagaacat |
| | 21181 | tcttggaatg ctgatcttta taagctcatg ggacacttcg catggtggac agcctttgtt |
| | 21241 | actaatgtga atgcgtcatc atctgaagca tttttaattg gatgtaatta tcttggcaaa |
| | 21301 | ccacgcgaac aaatagatgg ttatgtcatg catgcaaatt acatattttg gaggaataca |
| | 21361 | aatccaattc agttgtcttc ctattcttta tttgacatga gtaaatttcc ccttaaatta |
| | 21421 | aggggtactg ctgttatgtc tttaaaagaa ggtcaaatca atgatatgat tttatctctt |
| | 21481 | cttagtaaag gtagacttat aattagagaa aacaacagag ttgttatttc tagtgatgtt |
| | 21541 | cttgttaaca actaaacgaa caatgtttgt ttttcttgtt ttattgccac tagtctctag |
| | 21601 | tcagtgtgtt aatcttacaa ccagaactca attaccccct gcatacacta attctttcac |
| | 21661 | acgtggtgtt tattaccctg acaaagtttt cagatcctca gttttacatt caactcagga |
| | 21721 | cttgttctta cctttctttt ccaatgttac ttggttccat gctatacatg tctctgggac |
| | 21781 | caatggtact aagaggtttg ataaccctgt cctaccattt aatgatggtg tttattttgc |
| | 21841 | ttccactgag aagtctaaca taataagagg ctggattttt ggtactactt tagattcgaa |
| | 21901 | gacccagtcc ctacttattg ttaataacgc tactaatgtt gttattaaag tctgtgaatt |
| | 21961 | tcaattttgt aatgatccat ttttgggtgt ttattaccac aaaaacaaca aaagttggat |
| | 22021 | ggaaagtgag ttcagagttt attctagtgc gaataattgc acttttgaat atgtctctca |
| | 22081 | gccttttctt atggaccttg aaggaaaaca gggtaatttc aaaaatctta gggaatttgt |
| | 22141 | gtttaagaat attgatggtt attttaaaat atattctaag cacacgccta ttaatttagt |
| | 22201 | gcgtgatctc cctcagggtt tttcggcttt agaaccattg gtagatttgc caataggtat |
| | 22261 | taacatcact aggtttcaaa ctttacttgc tttacataga agttatttga ctcctggtga |
| | 22321 | ttcttcttca ggttggacag ctggtgctgc agcttattat gtgggttatc ttcaacctag |
| | 22381 | gacttttcta ttaaaatata atgaaaatgg aaccattaca gatgctgtag actgtgcact |
| | 22441 | tgaccctctc tcagaaacaa agtgtacgtt gaaatccttc actgtagaaa aaggaatcta |
| | 22501 | tcaaacttct aactttagag tccaaccaac agaatctatt gttagatttc ctaatattac |
| | 22561 | aaacttgtgc ccttttggtg aagtttttaa cgccaccaga tttgcatctg tttatgcttg |
| | 22621 | gaacaggaag agaatcagca actgtgttgc tgattattct gtcctatata attccgcatc |
| | 22681 | attttccact tttaagtgtt atggagtgtc tcctactaaa ttaaatgatc tctgctttac |
| | 22741 | taatgtctat gcagattcat ttgtaattag aggtgatgaa gtcagacaaa tcgctccagg |
| | 22801 | gcaaactgga aagattgctg attataatta taaattacca gatgatttta caggctgcgt |
| | 22861 | tatagcttgg aattctaaca atcttgattc taaggttggt ggtaattata attacctgta |
| | 22921 | tagattgttt aggaagtcta atctcaaacc ttttgagaga gatatttcaa ctgaaatcta |
| | 22981 | tcaggccggt agcacacctt gtaatggtgt tgaaggtttt aattgttact ttcctttaca |
| | 23041 | atcatatggt ttccaaccca ctaatggtgt tggttaccaa ccatacagag tagtagtact |
| | 23101 | ttcttttgaa cttctacatg caccagcaac tgtttgtgga cctaaaaagt ctactaattt |
| | 23161 | ggttaaaaac aaatgtgtca atttcaactt caatggttta acaggcacag gtgttcttac |
| | 23221 | tgagtctaac aaaaagtttc tgcctttcca acaatttggc agagacattg ctgacactac |
| | 23281 | tgatgctgtc cgtgatccac agacacttga gattcttgac attacaccat gttcttttgg |
| | 23341 | tggtgtcagt gttataacac caggaacaaa tacttctaac caggttgctg ttctttatca |
| | 23401 | ggatgttaac tgcacagaag tccctgttgc tattcatgca gatcaactta ctcctacttg |
| | 23461 | gcgtgtttat tctacaggtt ctaatgtttt tcaaacacgt gcaggctgtt taataggggc |
| | 23521 | tgaacatgtc aacaactcat atgagtgtga catacccatt ggtgcaggta tatgcgctag |
| | 23581 | ttatcagact cagactaatt ctcctcggcg ggcacgtagt gtagctagtc aatccatcat |
| | 23641 | tgcctacact atgtcacttg gtgcagaaaa ttcagttgct tactctaata actctattgc |
| | 23701 | catacccaca aattttacta ttagtgttac cacagaaatt ctaccagtgt ctatgaccaa |
| | 23761 | gacatcagta gattgtacaa tgtacatttg tggtgattca actgaatgca gcaatctttt |
| | 23821 | gttgcaatat ggcagttttt gtacacaatt aaaccgtgct ttaactggaa tagctgttga |
| | 23881 | acaagacaaa aacacccaag aagtttttgc acaagtcaaa caaatttaca aaacaccacc |
| | 23941 | aattaaagat tttggtggtt ttaatttttc acaaatatta ccagatccat caaaaccaag |
| | 24001 | caagaggtca tttattgaag atctactttt caacaaagtg acacttgcag atgctggctt |
| | 24061 | catcaaacaa tatggtgatt gccttggtga tattgctgct agagacctca tttgtgcaca |
| | 24121 | aaagtttaac ggccttactg ttttgccacc tttgctcaca gatgaaatga ttgctcaata |
| | 24181 | cacttctgca ctgttagcgg gtacaatcac ttctggttgg acctttggtg caggtgctgc |
| | 24241 | attacaaata ccatttgcta tgcaaatggc ttataggttt aatggtattg gagttacaca |
| | 24301 | gaatgttctc tatgagaacc aaaaattgat tgccaaccaa tttaatagtg ctattggcaa |
| | 24361 | aattcaagac tcactttctt ccacagcaag tgcacttgga aaacttcaag atgtggtcaa |
| | 24421 | ccaaaatgca caagctttaa acacgcttgt taaacaactt agctccaatt ttggtgcaat |
| | 24481 | ttcaagtgtt ttaaatgata tcctttcacg tcttgacaaa gttgaggctg aagtgcaaat |
| | 24541 | tgataggttg atcacaggca gacttcaaag tttgcagaca tatgtgactc aacaattaat |
| | 24601 | tagagctgca gaaatcagag cttctgctaa tcttgctgct actaaaatgt cagagtgtgt |
| | 24661 | acttggacaa tcaaaaagag ttgatttttg tggaaagggc tatcatctta tgtccttccc |
| | 24721 | tcagtcagca cctcatggtg tagtcttctt gcatgtgact tatgtccctg cacaagaaaa |
| | 24781 | gaacttcaca actgctcctg ccatttgtca tgatggaaaa gcacactttc ctcgtgaagg |
| | 24841 | tgtctttgtt tcaaatggca cacactggtt tgtaacacaa aggaattttt atgaaccaca |
| | 24901 | aatcattact acagacaaca catttgtgtc tggtaactgt gatgttgtaa taggaattgt |
| | 24961 | caacaacaca gtttatgatc ctttgcaacc tgaattagac tcattcaagg aggagttaga |
| | 25021 | taaatatttt aagaatcata catcaccaga tgttgattta ggtgacatct ctggcattaa |
| | 25081 | tgcttcagtt gtaaacattc aaaaagaaat tgaccgcctc aatgaggttg ccaagaattt |
| | 25141 | aaatgaatct ctcatcgatc tccaagaact tggaaagtat gagcagtata taaaatggcc |
| | 25201 | atggtacatt tggctaggtt ttatagctgg cttgattgcc atagtaatgg tgacaattat |
| | 25261 | gctttgctgt atgaccagtt gctgtagttg tctcaagggc tgttgttctt gtggatcctg |
| | 25321 | ctgcaaattt gatgaagacg actctgagcc agtgctcaaa ggagtcaaat tacattacac |
| | 25381 | ataaacgaac ttatggattt gtttatgaga atcttcacaa ttggaactgt aactttgaag |
| | 25441 | caaggtgaaa tcaaggatgc tactccttca gattttgttc gcgctactgc aacgataccg |
| | 25501 | atacaagcct cactcccttt cggatggctt attgttggcg ttgcacttct tgctgttttt |
| | 25561 | cagagcgctt ccaaaatcat aaccctcaaa aagagatggc aactagcact ctccaagggt |
| | 25621 | gttcactttg tttgcaactt gctgttgttg tttgtaacag tttactcaca ccttttgctc |
| | 25681 | gttgctgctg gccttgaagc cccttttctc tatctttatg ctttagtcta cttcttgcag |
| | 25741 | agtataaact ttgtaagaat aataatgagg ctttggcttt gctggaaatg ccgttccaaa |
| | 25801 | aacccattac tttatgatgc caactatttt ctttgctggc atactaattg ttacgactat |
| | 25861 | tgtatacctt acaatagtgt aacttcttca attgtcatta cttcaggtga tggcacaaca |
| | 25921 | agtcctattt ctgaacatga ctaccagatt ggtggttata ctgaaaaatg ggaatctgga |
| | 25981 | gtaaaagact gtgttgtatt acacagttac ttcacttcag actattacca gctgtactca |
| | 26041 | actcaattga gtacagacac tggtgttgaa catgttacct tcttcatcta caataaaatt |
| | 26101 | gttgatgagc ctgaagaaca tgtccaaatt cacacaatcg acggttcatc cggagttgtt |
| | 26161 | aatccagtaa tggaaccaat ttatgatgaa ccgacgacga ctactagcgt gcctttgtaa |
| | 26221 | gcacaagctg atgagtacga acttatgtac tcattcgttt cggaagagac aggtacgtta |
| | 26281 | atagttaata gcgtacttct ttttcttgct ttcgtggtat tcttgctagt tacactagcc |
| | 26341 | atccttactg cgcttcgatt gtgtgcgtac tgctgcaata ttgttaacgt gagtcttgta |
| | 26401 | aaaccttctt tttacgttta ctctcgtgtt aaaaatctga attcttctag agttcctgat |
| | 26461 | cttctggtct aaacgaacta aatattatat tagtttttct gtttggaact ttaattttag |
| | 26521 | ccatggcaga ttccaacggt actattaccg ttgaagagct taaaaagctc cttgaacaat |
| | 26581 | ggaacctagt aataggtttc ctattcctta catggatttg tcttctacaa tttgcctatg |
| | 26641 | ccaacaggaa taggtttttg tatataatta agttaatttt cctctggctg ttatggccag |
| | 26701 | taactttagc ttgttttgtg cttgctgctg tttacagaat aaattggatc accggtggaa |
| | 26761 | ttgctatcgc aatggcttgt cttgtaggct tgatgtggct cagctacttc attgcttctt |
| | 26821 | tcagactgtt tgcgcgtacg cgttccatgt ggtcattcaa tccagaaact aacattcttc |
| | 26881 | tcaacgtgcc actccatggc actattctga ccagaccgct tctagaaagt gaactcgtaa |
| | 26941 | tcggagctgt gatccttcgt ggacatcttc gtattgctgg acaccatcta ggacgctgtg |
| | 27001 | acatcaagga cctgcctaaa gaaatcactg ttgctacatc acgaacgctt tcttattaca |
| | 27061 | aattgggagc ttcgcagcgt gtagcaggtg actcaggttt tgctgcatac agtcgctaca |
| | 27121 | ggattggcaa ctataaatta aacacagacc attccagtag cagtgacaat attgctttgc |
| | 27181 | ttgtacagta agtgacaaca gatgtttcat ctcgttgact ttcaggttac tatagcagag |
| | 27241 | atattactaa ttattatgag gacttttaaa gtttccattt ggaatcttga ttacatcata |
| | 27301 | aacctcataa ttaaaaattt atctaagtca ctaactgaga ataaatattc tcaattagat |
| | 27361 | gaagagcaac caatggagat tgattaaacg aacatgaaaa ttattctttt cttggcactg |
| | 27421 | ataacactcg ctacttgtga gctttatcac taccaagagt gtgttagagg tacaacagta |
| | 27481 | cttttaaaag aaccttgctc ttctggaaca tacgagggca attcaccatt tcatcctcta |
| | 27541 | gctgataaca aatttgcact gacttgcttt agcactcaat ttgcttttgc ttgtcctgac |
| | 27601 | ggcgtaaaac acgtctatca gttacgtgcc agatcagttt cacctaaact gttcatcaga |
| | 27661 | caagaggaag ttcaagaact ttactctcca atttttctta ttgttgcggc aatagtgttt |
| | 27721 | ataacacttt gcttcacact caaaagaaag acagaatgat tgaactttca ttaattgact |
| | 27781 | tctatttgtg ctttttagcc tttctgctat tccttgtttt aattatgctt attatctttt |
| | 27841 | ggttctcact tgaactgcaa gatcataatg aaacttgtca cgcctaaacg aacatgaaat |
| | 27901 | ttcttgtttt cttaggaatc atcacaactg tagctgcatt tcaccaagaa tgtagtttac |
| | 27961 | agtcatgtac tcaacatcaa ccatatgtag ttgatgaccc gtgtcctatt cacttctatt |
| | 28021 | ctaaatggta tattagagta ggagctagaa aatcagcacc tttaattgaa ttgtgcgtgg |
| | 28081 | atgaggctgg ttctaaatca cccattcagt acatcgatat cggtaattat acagtttcct |
| | 28141 | gtttaccttt tacaattaat tgccaggaac ctaaattggg tagtcttgta gtgcgttgtt |
| | 28201 | cgttctatga agacttttta gagtatcatg acgttcgtgt tgttttagat ttcatctaaa |
| | 28261 | cgaacaaact aaaatgtctg ataatggacc ccaaaatcag cgaaatgcac cccgcattac |
| | 28321 | gtttggtgga ccctcagatt caactggcag taaccagaat ggagaacgca gtggggcgcg |
| | 28381 | atcaaaacaa cgtcggcccc aaggtttacc caataatact gcgtcttggt tcaccgctct |
| | 28441 | cactcaacat ggcaaggaag accttaaatt ccctcgagga caaggcgttc caattaacac |
| | 28501 | caatagcagt ccagatgacc aaattggcta ctaccgaaga gctaccagac gaattcgtgg |
| | 28561 | tggtgacggt aaaatgaaag atctcagtcc aagatggtat ttctactacc taggaactgg |
| | 28621 | gccagaagct ggacttccct atggtgctaa caaagacggc atcatatggg ttgcaactga |
| | 28681 | gggagccttg aatacaccaa aagatcacat tggcacccgc aatcctgcta acaatgctgc |
| | 28741 | aatcgtgcta caacttcctc aaggaacaac attgccaaaa ggcttctacg cagaagggag |
| | 28801 | cagaggcggc agtcaagcct cttctcgttc ctcatcacgt agtcgcaaca gttcaagaaa |
| | 28861 | ttcaactcca ggcagcagta ggggaacttc tcctgctaga atggctggca atggcggtga |
| | 28921 | tgctgctctt gctttgctgc tgcttgacag attgaaccag cttgagagca aaatgtctgg |
| | 28981 | taaaggccaa caacaacaag gccaaactgt cactaagaaa tctgctgctg aggcttctaa |
| | 29041 | gaagcctcgg caaaaacgta ctgccactaa agcatacaat gtaacacaag ctttcggcag |
| | 29101 | acgtggtcca gaacaaaccc aaggaaattt tggggaccag gaactaatca gacaaggaac |
| | 29161 | tgattacaaa cattggccgc aaattgcaca atttgccccc agcgcttcag cgttcttcgg |
| | 29221 | aatgtcgcgc attggcatgg aagtcacacc ttcgggaacg tggttgacct acacaggtgc |
| | 29281 | catcaaattg gatgacaaag atccaaattt caaagatcaa gtcattttgc tgaataagca |
| | 29341 | tattgacgca tacaaaacat tcccaccaac agagcctaaa aaggacaaaa agaagaaggc |
| | 29401 | tgatgaaact caagccttac cgcagagaca gaagaaacag caaactgtga ctcttcttcc |
| | 29461 | tgctgcagat ttggatgatt tctccaaaca attgcaacaa tccatgagca gtgctgactc |
| | 29521 | aactcaggcc taaactcatg cagaccacac aaggcagatg ggctatataa acgttttcgc |
| | 29581 | ttttccgttt acgatatata gtctactctt gtgcagaatg aattctcgta actacatagc |
| | 29641 | acaagtagat gtagttaact ttaatctcac atagcaatct ttaatcagtg tgtaacatta |
| | 29701 | gggaggactt gaaagagcca ccacattttc accgaggcca cgcggagtac gatcgagtgt |
| | 29761 | acagtgaaca atgctaggga gagctgccta tatggaagag ccctaatgtg taaaattaat |
| | 29821 | tttagtagtg ctatccccat gtgattttaa tagcttctta ggagaatgac aaaaaaaaaa |
| | 29881 | aaaaaaaaaa aaaaaaaaaa aaa |
| Nucleotide sequence | 1 | ttggctagtc aagatgatga atcttcatta tctgatatat tgcaaatcac tcaatatcta |
| of SARS-CoV-2 Spike | 61 | gactttctgt tattattatt gatccaatca aaaaataaat tagaagccgt gggtcattgt |
| protein in the TK locus. | 121 | tatgaatctc tttcagagga atacagacaa ttgacaaaat tcacagactt tcaagatttt |
| SEQ ID NO: 54 | 181 | aaaaaactgt ttaacaaggt ccctattgtt acagatggaa gggtcaaact taataaagga |
| | 241 | tatttgttcg actttgtgat tagtttgatg cgattcaaaa aagaatcctc tctagctacc |
| | 301 | accgcaatag atcctattag atacatagat cctcgtcgtg atatcgcatt ttctaacgtg |
| | 361 | atggatatat taaagttgaa taaagtgaac aataattaat tctttattgt catcggatcc |
| | 421 | cacgatgtgc tagactctct cgtctacgcg gccgcaaaaa ttgaaatttt attttttttt |
| | 481 | tttggaatat aaataatgtt cgtgttccta gtcctactac cgctagtctc ttcccagtgt |
| | 541 | gtaaacctaa caacgagaac acaactacca ccggcgtaca ccaattcttt cacaagagga |
| | 601 | gtatattacc cggacaaggt gttcagatcc tccgtactac attctaccca ggacctattc |
| | 661 | ctaccgttct tctctaacgt aacatggttc cacgcgatcc atgtctctgg aacaaacgga |
| | 721 | acgaagagat tcgataaccc ggtcttgccg ttcaacgatg gtgtatactt tgcgtccacc |
| | 781 | gagaagtcca acatcatcag aggatggatc ttcggaacca ccttggattc taagacccag |
| | 841 | tccttgctaa tcgtcaacaa cgcgaccaac gtcgtcatca aagtctgcga attccagttc |
| | 901 | tgtaacgacc cgtttttggg agtctactac cacaagaaca acaagtcctg gatggaatcc |
| | 961 | gagttcagag tctactcttc cgcgaacaac tgcaccttcg aatatgtatc tcagccgttc |
| | 1021 | ctaatggacc tagagggaaa gcagggaaac ttcaagaacc taagagagtt cgtattcaag |
| | 1081 | aacatcgacg gatacttcaa gatctactcc aagcacaccc cgatcaacct agttagagat |
| | 1141 | ctaccgcaag gattctctgc gctagaaccg ttagtagatt tgccgatcgg aatcaacatc |
| | 1201 | accagattcc agacactact agcgctacac agatcttacc taacgccggg agattcttct |
| | 1261 | tctggatgga ctgctggtgc tgcggcttat tatgtaggat acctacagcc gagaaccttc |
| | 1321 | ctattgaagt acaacgaaaa cggaaccatc accgatgccg tagattgtgc tctagatccg |
| | 1381 | ctatccgaaa cgaagtgcac cctaaagtct ttcaccgtcg agaagggaat ctaccagacc |
| | 1441 | tccaacttta gagtacagcc gaccgaatcc atcgtcagat ttccgaacat cacgaaccta |
| | 1501 | tgtccgttcg gagaagtgtt caacgcgaca agatttgcgt ctgtctatgc gtggaacaga |
| | 1561 | aaaagaatca gtaactgcgt cgcggactac tccgtcctat acaactctgc ctctttctcc |
| | 1621 | acgttcaaat gctacggtgt atccccgaca aagctaaacg atctatgctt caccaacgtc |
| | 1681 | tacgcggact ccttcgtaat cagaggagat gaagttagac agattgcgcc gggacaaact |
| | 1741 | ggaaagatcg cggattataa ctacaagcta ccggacgact tcaccggatg tgtaattgcg |
| | 1801 | tggaattcga acaacctaga ctccaaagtc ggaggaaact acaactactt gtacagacta |
| | 1861 | ttcagaaagt ccaacctaaa gccgttcgag agagacatct ccaccgaaat ctatcaggct |
| | 1921 | ggatctacac cgtgtaatgg tgtcgaagga ttcaactgct acttcccgct acagtcttac |
| | 1981 | ggatttcaac cgacaaacgg tgtaggatat cagccgtaca gagtcgtcgt actatccttc |
| | 2041 | gaactactac atgctccggc gacagtatgt ggaccgaaaa agtctaccaa cctagtcaag |
| | 2101 | aacaaatgcg tcaactttaa cttcaacgga ctaaccggaa ccggtgtcct aaccgaatct |
| | 2161 | aacaagaagt ttctaccgtt ccagcagttc ggaagagata tcgcggatac aacagacgct |
| | 2221 | gtcagagatc cgcaaacctt ggagatccta gatatcaccc cgtgttcttt cggtggtgtc |
| | 2281 | tctgtaatta ctccgggaac gaacacctcc aatcaagtag cggtactata ccaggacgtg |
| | 2341 | aactgtacag aagtaccggt agctattcac gcggatcaac taacaccaac ttggagagtg |
| | 2401 | tactccaccg gatctaacgt attccaaaca agagcgggat gtctaatcgg agcggaacac |
| | 2461 | gtaaacaact cctacgaatg tgatatcccg attggagcgg gaatctgtgc gtcttaccaa |
| | 2521 | acacaaacaa actccccgag aagagcgaga tctgtagcct ctcaatctat tatcgcctac |
| | 2581 | accatgtcct tgggagccga aaattctgtc gcgtactcca acaattctat cgcgatcccg |
| | 2641 | acaaacttca ccatctctgt aacaaccgag atcctaccgg tgtctatgac caagacatct |
| | 2701 | gtcgattgca ccatgtacat ctgcggagat tccaccgagt gctccaacct actactacag |
| | 2761 | tacggatctt tctgtaccca gctaaacaga gcgttgactg gaatcgctgt agagcaggat |
| | 2821 | aagaacaccc aagaggtatt cgcgcaagtc aagcagatct ataagactcc gccgatcaag |
| | 2881 | gacttcggag gttttaactt ctctcagatc ttgccggatc cgtccaaacc gtctaagaga |
| | 2941 | tctttcatcg aggacctact attcaacaaa gtcaccctag ctgacgcggg attcatcaaa |
| | 3001 | caatacggag attgcttggg agacattgcg gcgagagatc taatttgcgc gcagaagttt |
| | 3061 | aacggattga cagtactacc gccgctacta accgatgaga tgattgcgca gtacacgtct |
| | 3121 | gctctattgg cgggaacaat tacaagtgga tggacatttg gagccggtgc cgctctacaa |
| | 3181 | attccgtttg ctatgcaaat ggcgtacaga ttcaacggaa tcggagtaac ccagaacgtc |
| | 3241 | ttgtacgaga accagaagct aatcgcgaac cagttcaatt ccgcgatcgg aaagatccag |
| | 3301 | gacagtctat cttctactgc ttcggcgttg ggaaagctac aggatgtagt aaatcaaaac |
| | 3361 | gcgcaggcgc taaacacctt ggtcaagcaa ctatcctcta acttcggagc gatctcgtcc |
| | 3421 | gtcctaaacg acatcttatc cagactagat aaggtcgaag cggaggtcca gatcgataga |
| | 3481 | ctaatcactg gaagattgca gtccctacag acctacgtaa cacagcaact aattagagcg |
| | 3541 | gcggagatta gagcctctgc taatctagct gcgaccaaga tgtccgaatg tgtcttggga |
| | 3601 | caatccaaga gagtcgactt ttgcggaaag ggataccacc taatgtcttt tccacaatct |
| | 3661 | gcgccgcatg gtgtcgtatt cctacatgta acatatgtgc cggcgcaaga aaagaacttt |
| | 3721 | acaacagctc cagcgatctg ccatgatgga aaagctcatt ttccgagaga gggagtcttt |
| | 3781 | gtctctaacg gaactcattg gttcgtcacc cagagaaact tttacgagcc gcagatcatc |
| | 3841 | accaccgaca acacatttgt ttcgggaaac tgcgacgtgg tcatcggaat cgtaaacaat |
| | 3901 | accgtctacg atccgttgca gccggaacta gactccttca aagaagagtt ggacaagtac |
| | 3961 | tttaagaacc acacctctcc ggatgtcgac ttgggagata tttctggaat caacgcgtcc |
| | 4021 | gtcgtcaaca tccagaaaga aatcgataga ttgaacgagg tcgcgaagaa cttgaacgag |
| | 4081 | tccctaatcg acctacaaga gctaggaaaa tacgagcagt acatcaagtg gccgtggtac |
| | 4141 | atttggctag gattcattgc tggactaatt gcgatcgtca tggtcaccat catgctatgc |
| | 4201 | tgtatgacct cctgttgctc ctgtctaaag ggatgttgtt cctgcggatc ctgttgcaag |
| | 4261 | ttcgatgaag atgatagtga accggtccta aagggtgtca agctacacta cacataaaag |
| | 4321 | cttgtcgact attatatttt ttatctaaaa aactaaaaat aaacattgat taaattttaa |
| | 4381 | tataatactt aaaaatggat gttgtgtcgt tagataaacc gtttatgtat tttgaggaaa |
| | 4441 | ttgataatga gttagattac gaaccagaaa gtgcaaatga ggtcgcaaaa aaactaccgt |
| | 4501 | atcaaggaca gttaaaacta ttactaggag aattattttt tcttagtaag ttacagcgac |
| | 4561 | acggtatatt agatggtgcc accgtagtgt atataggatc ggctcctggt acacatatac |
| | 4621 | gttatttgag agatcatttc tataatttag gaatgattat caaatggatg ctaattgacg |
| | 4681 | gacgccatca tgatcctatt ctaaatggat tgcgtgatgt gactctagta tggtcatag |
| Nucleotide sequence | 1 | gagtattcta ggtgtttcta tagaatgtaa gaagtcatcg acattactta cttttttgac |
| of SARS-CoV-2 Spike | 61 | cgtgcgtaaa atgacccgag tatttaatag atttccagat atggcttatt atcgaggaga |
| protein in the HPXV200 | 121 | ctgtttaaaa gccgtttatg taacaatgac ttataaaaat actaaaactg gagagactga |
| (B22R) locus. | 181 | ttacacgtac ctctctaatg ggggttgcct gcatactatc gtaatggggt cgatggttga |
| SEQ ID NO: 55 | 241 | ttattgatta gtatattcct tattcttttt attcacacaa aaagaacatt tttataaaca |
| | 301 | tgaaaccact gtctaaatgt aattatgatc ttgatttata gatgaagatc agcctttaga |
| | 361 | ggattttaac cagtatgttt aatatgaaaa aaataaacat aacatatttt gagattaagc |
| | 421 | gctattgtgc ttaattattt tgctctataa actgaatata tagccacaat tattgacggg |
| | 481 | cttgtttatg accggcaatc ggatcccacg atgtgctaga ctctctcgtc tacgcggccg |
| | 541 | caaaaattga aattttattt tttttttttg gaatataaat aatgttcgtg ttcctagtcc |
| | 601 | tactaccgct agtctcttcc cagtgtgtaa acctaacaac gagaacacaa ctaccaccgg |
| | 661 | cgtacaccaa ttctttcaca agaggagtat attacccgga caaggtgttc agatcctccg |
| | 721 | tactacattc tacccaggac ctattcctac cgttcttctc taacgtaaca tggttccacg |
| | 781 | cgatccatgt ctctggaaca aacggaacga agagattcga taacccggtc ttgccgttca |
| | 841 | acgatggtgt atactttgcg tccaccgaga agtccaacat catcagagga tggatcttcg |
| | 901 | gaaccacctt ggattctaag acccagtcct tgctaatcgt caacaacgcg accaacgtcg |
| | 961 | tcatcaaagt ctgcgaattc cagttctgta acgacccgtt tttgggagtc tactaccaca |
| | 1021 | agaacaacaa gtcctggatg gaatccgagt tcagagtcta ctcttccgcg aacaactgca |
| | 1081 | ccttcgaata tgtatctcag ccgttcctaa tggacctaga gggaaagcag ggaaacttca |
| | 1141 | agaacctaag agagttcgta ttcaagaaca tcgacggata cttcaagatc tactccaagc |
| | 1201 | acaccccgat caacctagtt agagatctac cgcaaggatt ctctgcgcta gaaccgttag |
| | 1261 | tagatttgcc gatcggaatc aacatcacca gattccagac actactagcg ctacacagat |
| | 1321 | cttacctaac gccgggagat tcttcttctg gatggactgc tggtgctgcg gcttattatg |
| | 1381 | taggatacct acagccgaga accttcctat tgaagtacaa cgaaaacgga accatcaccg |
| | 1441 | atgccgtaga ttgtgctcta gatccgctat ccgaaacgaa gtgcacccta aagtctttca |
| | 1501 | ccgtcgagaa gggaatctac cagacctcca actttagagt acagccgacc gaatccatcg |
| | 1561 | tcagatttcc gaacatcacg aacctatgtc cgttcggaga agtgttcaac gcgacaagat |
| | 1621 | ttgcgtctgt ctatgcgtgg aacagaaaaa gaatcagtaa ctgcgtcgcg gactactccg |
| | 1681 | tcctatacaa ctctgcctct ttctccacgt tcaaatgcta cggtgtatcc ccgacaaagc |
| | 1741 | taaacgatct atgcttcacc aacgtctacg cggactcctt cgtaatcaga ggagatgaag |
| | 1801 | ttagacagat tgcgccggga caaactggaa agatcgcgga ttataactac aagctaccgg |
| | 1861 | acgacttcac cggatgtgta attgcgtgga attcgaacaa cctagactcc aaagtcggag |
| | 1921 | gaaactacaa ctacttgtac agactattca gaaagtccaa cctaaagccg ttcgagagag |
| | 1981 | acatctccac cgaaatctat caggctggat ctacaccgtg taatggtgtc gaaggattca |
| | 2041 | actgctactt cccgctacag tcttacggat ttcaaccgac aaacggtgta ggatatcagc |
| | 2101 | cgtacagagt cgtcgtacta tccttcgaac tactacatgc tccggcgaca gtatgtggac |
| | 2161 | cgaaaaagtc taccaaccta gtcaagaaca aatgcgtcaa ctttaacttc aacggactaa |
| | 2221 | ccggaaccgg tgtcctaacc gaatctaaca agaagtttct accgttccag cagttcggaa |
| | 2281 | gagatatcgc ggatacaaca gacgctgtca gagatccgca aaccttggag atcctagata |
| | 2341 | tcaccccgtg ttctttcggt ggtgtctctg taattactcc gggaacgaac acctccaatc |
| | 2401 | aagtagcggt actataccag gacgtgaact gtacagaagt accggtagct attcacgcgg |
| | 2461 | atcaactaac accaacttgg agagtgtact ccaccggatc taacgtattc caaacaagag |
| | 2521 | cgggatgtct aatcggagcg gaacacgtaa acaactccta cgaatgtgat atcccgattg |
| | 2581 | gagcgggaat ctgtgcgtct taccaaacac aaacaaactc cccgagaaga gcgagatctg |
| | 2641 | tagcctctca atctattatc gcctacacca tgtccttggg agccgaaaat tctgtcgcgt |
| | 2701 | actccaacaa ttctatcgcg atcccgacaa acttcaccat ctctgtaaca accgagatcc |
| | 2761 | taccggtgtc tatgaccaag acatctgtcg attgcaccat gtacatctgc ggagattcca |
| | 2821 | ccgagtgctc caacctacta ctacagtacg gatctttctg tacccagcta aacagagcgt |
| | 2881 | tgactggaat cgctgtagag caggataaga acacccaaga ggtattcgcg caagtcaagc |
| | 2941 | agatctataa gactccgccg atcaaggact tcggaggttt taacttctct cagatcttgc |
| | 3001 | cggatccgtc caaaccgtct aagagatctt tcatcgagga cctactattc aacaaagtca |
| | 3061 | ccctagctga cgcgggattc atcaaacaat acggagattg cttgggagac attgcggcga |
| | 3121 | gagatctaat ttgcgcgcag aagtttaacg gattgacagt actaccgccg ctactaaccg |
| | 3181 | atgagatgat tgcgcagtac acgtctgctc tattggcggg aacaattaca agtggatgga |
| | 3241 | catttggagc cggtgccgct ctacaaattc cgtttgctat gcaaatggcg tacagattca |
| | 3301 | acggaatcgg agtaacccag aacgtcttgt acgagaacca gaagctaatc gcgaaccagt |
| | 3361 | tcaattccgc gatcggaaag atccaggaca gtctatcttc tactgcttcg gcgttgggaa |
| | 3421 | agctacagga tgtagtaaat caaaacgcgc aggcgctaaa caccttggtc aagcaactat |
| | 3481 | cctctaactt cggagcgatc tcgtccgtcc taaacgacat cttatccaga ctagataagg |
| | 3541 | tcgaagcgga ggtccagatc gatagactaa tcactggaag attgcagtcc ctacagacct |
| | 3601 | acgtaacaca gcaactaatt agagcggcgg agattagagc ctctgctaat ctagctgcga |
| | 3661 | ccaagatgtc cgaatgtgtc ttgggacaat ccaagagagt cgacttttgc ggaaagggat |
| | 3721 | accacctaat gtcttttcca caatctgcgc cgcatggtgt cgtattccta catgtaacat |
| | 3781 | atgtgccggc gcaagaaaag aactttacaa cagctccagc gatctgccat gatggaaaag |
| | 3841 | ctcattttcc gagagaggga gtctttgtct ctaacggaac tcattggttc gtcacccaga |
| | 3901 | gaaactttta cgagccgcag atcatcacca ccgacaacac atttgtttcg ggaaactgcg |
| | 3961 | acgtggtcat cggaatcgta aacaataccg tctacgatcc gttgcagccg gaactagact |
| | 4021 | ccttcaaaga agagttggac aagtacttta agaaccacac ctctccggat gtcgacttgg |
| | 4081 | gagatatttc tggaatcaac gcgtccgtcg tcaacatcca gaaagaaatc gatagattga |
| | 4141 | acgaggtcgc gaagaacttg aacgagtccc taatcgacct acaagagcta ggaaaatacg |
| | 4201 | agcagtacat caagtggccg tggtacattt ggctaggatt cattgctgga ctaattgcga |
| | 4261 | tcgtcatggt caccatcatg ctatgctgta tgacctcctg ttgctcctgt ctaaagggat |
| | 4321 | gttgttcctg cggatcctgt tgcaagttcg atgaagatga tagtgaaccg gtcctaaagg |
| | 4381 | gtgtcaagct acactacaca taaaagcttg tcgactaaaa tagtttaact cttttaaaac |
| | 4441 | cagtttggta ctggaatttc agttcattac tcgttgagaa attgatgatt tttttaaaat |
| | 4501 | gatattactt ttatatgctt gcatcgcaga atgatattca caagtattat taaaaatgag |
| | 4561 | tatcggtagt tacattacca tatcatccat gctcatatgg atctccatcc attatataat |
| | 4621 | caatgataca tgtattaaaa tactttccga ataagtcttt taaatattgt attaattatg |
| | 4681 | aaaaactatg ctatgcgagt atgatgcaaa gatgtttaat gatacgatac tagattttat |
| | 4741 | ctctagcgag agatgtcgtt agaatcattt atcataacta cgtttaataa taattcatca |
| | 4801 | acgaatatcg ataacatgtg tcatttatac tttaaatacg ttaaagtctg tccgtcttct |
| | 4861 | ctattgttta gactgtttgt agaatgctgt gatataaaca aactagtaga aggta |
| Nucleotide sequence | 1 | atttacggat tcaccaataa aaataaacta gagaaactta gtactaataa ggaactagaa |
| of HPXV Delta TK | 61 | tcgtatagtt ctagccctct tcaagaaccc attaggttaa atgattttct gggactattg |
| Left Arm and Right Arm | 121 | gaatgtatta aaaagaatat tcctctaaca gatattccga caaaggattg attactataa |
| (SEQ ID NO: 62) | 181 | atggagaatg ttcctaatgt atactttaat cctgtgttta tagagcccac gtttaaacat |
| | 241 | tctttattaa gtgtttataa acacagatta atagttttat ttgaagtatt cattgtattc |
| | 301 | attctaatat atgtattttt tagatctgaa ttaaatatgt tcttcatgcc taaacgaaaa |
| | 361 | atacccgatc ctattgatag attacgacgt gctaatctag cgtgtgaaga cgataagtta |
| | 421 | atgatctatg gattaccatg gatgacaact caaacatctg cgttatcaat aaatagtaaa |
| | 481 | ccgatagtgt ataaagattg tgcaaagctt ttgcgatcaa taaatggatc acaaccagta |
| | 541 | tctcttaacg atgttcttcg cagatgatga ttcatttttt aagtatttgg ctagtcaaga |
| | 601 | tgatgaatct tcattatctg atatattgca aatcactcaa tatctagact ttctgttatt |
| | 661 | attattgatc caatcaaaaa ataaattaga agccgtgggt cattgttatg aatctctttc |
| | 721 | agaggaatac agacaattga caaaattcac agactttcaa gattttaaaa aactgtttaa |
| | 781 | caaggtccct attgttacag atggaagggt caaacttaat aaaggatatt tgttcgactt |
| | 841 | tgtgattagt ttgatgcgat tcaaaaaaga atcctctcta gctaccaccg caatagatcc |
| | 901 | tattagatac atagatcctc gtcgtgatat cgcattttct aacgtgatgg atatattaaa |
| | 961 | gttgaataaa gtgaacaata attaattctt tattgtcatc tattatattt tttatctaaa |
| | 1021 | aaactaaaaa taaacattga ttaaatttta atataatact taaaaatgga tgttgtgtcg |
| | 1081 | ttagataaac cgtttatgta ttttgaggaa attgataatg agttagatta cgaaccagaa |
| | 1141 | agtgcaaatg aggtcgcaaa aaaactaccg tatcaaggac agttaaaact attactagga |
| | 1201 | gaattatttt ttcttagtaa gttacagcga cacggtatat tagatggtgc caccgtagtg |
| | 1261 | tatataggat cggctcctgg tacacatata cgttatttga gagatcattt ctataattta |
| | 1321 | ggaatgatta tcaaatggat gctaattgac ggacgccatc atgatcctat tctaaatgga |
| | 1381 | ttgcgtgatg tgactctagt gactcggttc gttgatgagg aatatctacg atccatcaaa |
| | 1441 | aaacaactgc atccttctaa gattatttta atttctgatg taagatccaa acgaggagga |
| | 1501 | aatgaaccta gtacggcgga tttactaagt aattacgctc tacaaaatgt catgattagt |
| | 1561 | attttaaacc ccgtggcatc tagtcttaaa tggagatgcc cgtttccaga tcaatggatc |
| | 1621 | aaggactttt atatcccaca cggtaataaa atgttacaac cttttgctcc ttcatattca |
| | 1681 | gctgaaatga gattattaag tatttatacc ggtgagaaca tgagactgac tcgagttacc |
| | 1741 | aaattagacg ctgtaaatta tgaaaaaaag atgtactacc ttaataagat cgtccgtaac |
| | 1801 | aaagtagttg ttaactttga ttatcctaat caggaatatg actattttca catgtacttt |
| | 1861 | atgctgagga ccgtatactg caataaaaca tttcctacta ctaaagcaaa ggtactattt |
| | 1921 | ctacaacaat ctatatttcg tttcttaaat attccaacaa catcaactga aaaagttagt |
| | 1981 | catgaaccaa tacaacgtaa |
| Nucleotide sequence of | 1 | atttacggat tcaccaataa aaataaacta gagaaactta gtactaataa ggaactagaa |
| HPXV_COVID- | 61 | tcgtatagtt ctagccctct tcaagaaccc attaggttaa atgattttct gggactattg |
| 19_Spike_Delta_T5NT | 121 | gaatgtatta aaaagaatat tcctctaaca gatattccga caaaggattg attactataa |
| (SEQ ID NO: 63) | 181 | atggagaatg ttcctaatgt atactttaat cctgtgttta tagagcccac gtttaaacat |
| | 241 | tctttattaa gtgtttataa acacagatta atagttttat ttgaagtatt cattgtattc |
| | 301 | attctaatat atgtattttt tagatctgaa ttaaatatgt tcttcatgcc taaacgaaaa |
| | 361 | atacccgatc ctattgatag attacgacgt gctaatctag cgtgtgaaga cgataagtta |
| | 421 | atgatctatg gattaccatg gatgacaact caaacatctg cgttatcaat aaatagtaaa |
| | 481 | ccgatagtgt ataaagattg tgcaaagctt ttgcgatcaa taaatggatc acaaccagta |
| | 541 | tctcttaacg atgttcttcg cagatgatga ttcatttttt aagtatttgg ctagtcaaga |
| | 601 | tgatgaatct tcattatctg atatattgca aatcactcaa tatctagact ttctgttatt |
| | 661 | attattgatc caatcaaaaa ataaattaga agccgtgggt cattgttatg aatctctttc |
| | 721 | agaggaatac agacaattga caaaattcac agactttcaa gattttaaaa aactgtttaa |
| | 781 | caaggtccct attgttacag atggaagggt caaacttaat aaaggatatt tgttcgactt |
| | 841 | tgtgattagt ttgatgcgat tcaaaaaaga atcctctcta gctaccaccg caatagatcc |
| | 901 | tattagatac atagatcctc gtcgtgatat cgcattttct aacgtgatgg atatattaaa |
| | 961 | gttgaataaa gtgaacaata attaattctt tattgtcatc ttttattttt tttttttgga |
| | 1021 | atataaatat ccggtaaaat tgaaaaaata tacactaatt agcgtctcgt ttcagacgct |
| | 1081 | agctcgaggt tgggagctct ccggatccaa gcttatcgat ttcgaacccg gggtaccgaa |
| | 1141 | ttcctcgagg ttgggagctc tccggatcca agcttatcga tttcgaaccc ggggtaccga |
| | 1201 | attcctcgag atgtttgttt tccttgtttt attgccacta gtctctagtc agtgtgttaa |
| | 1261 | tcttacaacc agaactcaat taccccctgc atacactaat tctttcacac gtggtgttta |
| | 1321 | ttaccctgac aaagttttca gatcctcagt tttacattca actcaggact tgttcttacc |
| | 1381 | tttcttttcc aatgttactt ggttccatgc tatacatgtc tctgggacca atggtactaa |
| | 1441 | gaggtttgat aaccctgtcc taccatttaa tgatggtgtt tattttgctt ccactgagaa |
| | 1501 | gtctaacata ataagaggct ggatttttgg tactacttta gattcgaaga cccagtccct |
| | 1561 | acttattgtt aataacgcta ctaatgttgt tattaaagtc tgtgaatttc aattttgtaa |
| | 1621 | tgatccattt ttgggtgttt attaccacaa aaacaacaaa agttggatgg aaagtgagtt |
| | 1681 | cagagtttat tctagtgcga ataattgcac ttttgaatat gtctctcagc cttttcttat |
| | 1741 | ggaccttgaa ggaaaacagg gtaatttcaa aaatcttagg gaatttgtgt ttaagaatat |
| | 1801 | tgatggttat tttaaaatat attctaagca cacgcctatt aatttagtgc gtgatctccc |
| | 1861 | tcagggtttt tcggctttag aaccattggt agatttgcca ataggtatta acatcactag |
| | 1921 | gtttcaaact ttacttgctt tacatagaag ttatttgact cctggtgatt cttcttcagg |
| | 1981 | ttggacagct ggtgctgcag cttattatgt gggttatctt caacctagga cttttctatt |
| | 2041 | aaaatataat gaaaatggaa ccattacaga tgctgtagac tgtgcacttg accctctctc |
| | 2101 | agaaacaaag tgtacgttga aatccttcac tgtagaaaaa ggaatctatc aaacttctaa |
| | 2161 | ctttagagtc caaccaacag aatctattgt tagatttcct aatattacaa acttgtgccc |
| | 2221 | ttttggtgaa gtttttaacg ccaccagatt tgcatctgtt tatgcttgga acaggaagag |
| | 2281 | aatcagcaac tgtgttgctg attattctgt cctatataat tccgcatcat tttccacttt |
| | 2341 | taagtgttat ggagtgtctc ctactaaatt aaatgatctc tgctttacta atgtctatgc |
| | 2401 | agattcattt gtaattagag gtgatgaagt cagacaaatc gctccagggc aaactggaaa |
| | 2461 | gattgctgat tataattata aattaccaga tgattttaca ggctgcgtta tagcttggaa |
| | 2521 | ttctaacaat cttgattcta aggttggtgg taattataat tacctgtata gattgtttag |
| | 2581 | gaagtctaat ctcaaacctt ttgagagaga tatttcaact gaaatctatc aggccggtag |
| | 2641 | cacaccttgt aatggtgttg aaggttttaa ttgttacttt cctttacaat catatggttt |
| | 2701 | ccaacccact aatggtgttg gttaccaacc atacagagta gtagtacttt cttttgaact |
| | 2761 | tctacatgca ccagcaactg tttgtggacc taaaaagtct actaatttgg ttaaaaacaa |
| | 2821 | atgtgtcaat ttcaacttca atggtttaac aggcacaggt gttcttactg agtctaacaa |
| | 2881 | aaagtttctg cctttccaac aatttggcag agacattgct gacactactg atgctgtccg |
| | 2941 | tgatccacag acacttgaga ttcttgacat tacaccatgt tcttttggtg gtgtcagtgt |
| | 3001 | tataacacca ggaacaaata cttctaacca ggttgctgtt ctttatcagg atgttaactg |
| | 3061 | cacagaagtc cctgttgcta ttcatgcaga tcaacttact cctacttggc gtgtttattc |
| | 3121 | tacaggttct aatgtttttc aaacacgtgc aggctgttta ataggggctg aacatgtcaa |
| | 3181 | caactcatat gagtgtgaca tacccattgg tgcaggtata tgcgctagtt atcagactca |
| | 3241 | gactaattct cctcggcggg cacgtagtgt agctagtcaa tccatcattg cctacactat |
| | 3301 | gtcacttggt gcagaaaatt cagttgctta ctctaataac tctattgcca tacccacaaa |
| | 3361 | ttttactatt agtgttacca cagaaattct accagtgtct atgaccaaga catcagtaga |
| | 3421 | ttgtacaatg tacatttgtg gtgattcaac tgaatgcagc aatcttttgt tgcaatatgg |
| | 3481 | cagtttctgt acacaattaa accgtgcttt aactggaata gctgttgaac aagacaaaaa |
| | 3541 | cacccaagaa gtttttgcac aagtcaaaca aatttacaaa acaccaccaa ttaaagattt |
| | 3601 | tggtggtttt aatttttcac aaatattacc agatccatca aaaccaagca agaggtcatt |
| | 3661 | tattgaagat ctacttttca acaaagtgac acttgcagat gctggcttca tcaaacaata |
| | 3721 | tggtgattgc cttggtgata ttgctgctag agacctcatt tgtgcacaaa agtttaacgg |
| | 3781 | ccttactgtt ttgccacctt tgctcacaga tgaaatgatt gctcaataca cttctgcact |
| | 3841 | gttagcgggt acaatcactt ctggttggac ctttggtgca ggtgctgcat tacaaatacc |
| | 3901 | atttgctatg caaatggctt ataggtttaa tggtattgga gttacacaga atgttctcta |
| | 3961 | tgagaaccaa aaattgattg ccaaccaatt taatagtgct attggcaaaa ttcaagactc |
| | 4021 | actttcttcc acagcaagtg cacttggaaa acttcaagat gtggtcaacc aaaatgcaca |
| | 4081 | agctttaaac acgcttgtta aacaacttag ctccaatttt ggtgcaattt caagtgtttt |
| | 4141 | aaatgatatc ctttcacgtc ttgacaaagt tgaggctgaa gtgcaaattg ataggttgat |
| | 4201 | cacaggcaga cttcaaagtt tgcagacata tgtgactcaa caattaatta gagctgcaga |
| | 4261 | aatcagagct tctgctaatc ttgctgctac taaaatgtca gagtgtgtac ttggacaatc |
| | 4321 | aaaaagagtt gatttctgtg gaaagggcta tcatcttatg tccttccctc agtcagcacc |
| | 4381 | tcatggtgta gtcttcttgc atgtgactta tgtccctgca caagaaaaga acttcacaac |
| | 4441 | tgctcctgcc atttgtcatg atggaaaagc acactttcct cgtgaaggtg tctttgtttc |
| | 4501 | aaatggcaca cactggtttg taacacaaag gaacttttat gaaccacaaa tcattactac |
| | 4561 | agacaacaca tttgtgtctg gtaactgtga tgttgtaata ggaattgtca acaacacagt |
| | 4621 | ttatgatcct ttgcaacctg aattagactc attcaaggag gagttagata aatattttaa |
| | 4681 | gaatcataca tcaccagatg ttgatttagg tgacatctct ggcattaatg cttcagttgt |
| | 4741 | aaacattcaa aaagaaattg accgcctcaa tgaggttgcc aagaatttaa atgaatctct |
| | 4801 | catcgatctc caagaacttg gaaagtatga gcagtatata aaatggccat ggtacatttg |
| | 4861 | gctaggtttt atagctggct tgattgccat agtaatggtg acaattatgc tttgctgtat |
| | 4921 | gaccagttgc tgtagttgtc tcaagggctg ttgttcttgt ggatcctgct gcaaatttga |
| | 4981 | tgaagacgac tctgagccag tgctcaaagg agtcaaatta cattacacat aatattatat |
| | 5041 | tttttatcta aaaaactaaa aataaacatt gattaaattt taatataata cttaaaaatg |
| | 5101 | gatgttgtgt cgttagataa accgtttatg tattttgagg aaattgataa tgagttagat |
| | 5161 | tacgaaccag aaagtgcaaa tgaggtcgca aaaaaactac cgtatcaagg acagttaaaa |
| | 5221 | ctattactag gagaattatt ttttcttagt aagttacagc gacacggtat attagatggt |
| | 5281 | gccaccgtag tgtatatagg atcggctcct ggtacacata tacgttattt gagagatcat |
| | 5341 | ttctataatt taggaatgat tatcaaatgg atgctaattg acggacgcca tcatgatcct |
| | 5401 | attctaaatg gattgcgtga tgtgactcta gtgactcggt tcgttgatga ggaatatcta |
| | 5461 | cgatccatca aaaaacaact gcatccttct aagattattt taatttctga tgtaagatcc |
| | 5521 | aaacgaggag gaaatgaacc tagtacggcg gatttactaa gtaattacgc tctacaaaat |
| | 5581 | gtcatgatta gtattttaaa ccccgtggca tctagtctta aatggagatg cccgtttcca |
| | 5641 | gatcaatgga tcaaggactt ttatatccca cacggtaata aaatgttaca accttttgct |
| | 5701 | ccttcatatt cagctgaaat gagattatta agtatttata ccggtgagaa catgagactg |
| | 5761 | actcgagtta ccaaattaga cgctgtaaat tatgaaaaaa agatgtacta ccttaataag |
| | 5821 | atcgtccgta acaaagtagt tgttaacttt gattatccta atcaggaata tgactatttt |
| | 5881 | cacatgtact ttatgctgag gaccgtatac tgcaataaaa catttcctac tactaaagca |
| | 5941 | aaggtactat ttctacaaca atctatattt cgtttcttaa atattccaac aacatcaact |
| | 6001 | gaaaaagtta gtcatgaacc aatacaacgt aa |
| Nucleotide sequence of | 1 | atttacggat tcaccaataa aaataaacta gagaaactta gtactaataa ggaactagaa |
| HPXV_SARS_Ad_Spike_Delta_T5NT | 61 | tcgtatagtt ctagccctct tcaagaaccc attaggttaa atgattttct gggactattg |
| (SEQ ID NO: 64) | 121 | gaatgtatta aaaagaatat tcctctaaca gatattccga caaaggattg attactataa |
| | 181 | atggagaatg ttcctaatgt atactttaat cctgtgttta tagagcccac gtttaaacat |
| | 241 | tctttattaa gtgtttataa acacagatta atagttttat ttgaagtatt cattgtattc |
| | 301 | attctaatat atgtattttt tagatctgaa ttaaatatgt tcttcatgcc taaacgaaaa |
| | 361 | atacccgatc ctattgatag attacgacgt gctaatctag cgtgtgaaga cgataagtta |
| | 421 | atgatctatg gattaccatg gatgacaact caaacatctg cgttatcaat aaatagtaaa |
| | 481 | ccgatagtgt ataaagattg tgcaaagctt ttgcgatcaa taaatggatc acaaccagta |
| | 541 | tctcttaacg atgttcttcg cagatgatga ttcatttttt aagtatttgg ctagtcaaga |
| | 601 | tgatgaatct tcattatctg atatattgca aatcactcaa tatctagact ttctgttatt |
| | 661 | attattgatc caatcaaaaa ataaattaga agccgtgggt cattgttatg aatctctttc |
| | 721 | agaggaatac agacaattga caaaattcac agactttcaa gattttaaaa aactgtttaa |
| | 781 | caaggtccct attgttacag atggaagggt caaacttaat aaaggatatt tgttcgactt |
| | 841 | tgtgattagt ttgatgcgat tcaaaaaaga atcctctcta gctaccaccg caatagatcc |
| | 901 | tattagatac atagatcctc gtcgtgatat cgcattttct aacgtgatgg atatattaaa |
| | 961 | gttgaataaa gtgaacaata attaattctt tattgtcatc ttttattttt tttttttgga |
| | 1021 | atataaatat ccggtaaaat tgaaaaaata tacactaatt agcgtctcgt ttcagacgct |
| | 1081 | agctcgaggt tgggagctct ccggatccaa gcttatcgat ttcgaacccg gggtaccgaa |
| | 1141 | ttcctcgagg ttgggagctc tccggatcca agcttatcga tttcgaaccc ggggtaccga |
| | 1201 | attcctcgag atgtttattt tcttattatt tcttactctc actagtggta gtgaccttga |
| | 1261 | ccggtgcacc acttttgatg atgttcaagc tcctaattac actcaacata cttcatctat |
| | 1321 | gaggggggtt tactatcctg atgaaatttt tagatcagac actctttatt taactcagga |
| | 1381 | tttatttctt ccattttatt ctaatgttac agggtttcat actattaatc atacgtttgg |
| | 1441 | caaccctgtc atacctttta aggatggtat ttattttgct gccacagaga aatcaaatgt |
| | 1501 | tgtccgtggt tgggtttttg gttctaccat gaacaacaag tcacagtcgg tgattattat |
| | 1561 | taacaattct actaatgttg ttatacgagc atgtaacttt gaattgtgtg acaacccttt |
| | 1621 | ctttgctgtt tctaaaccca tgggtacaca gacacatact atgatattcg ataatgcatt |
| | 1681 | taattgcact ttcgagtaca tatctgatgc cttttcgctt gatgtttcag aaaagtcagg |
| | 1741 | taattttaaa cacttacgag agtttgtgtt taaaaataaa gatgggtttc tctatgttta |
| | 1801 | taagggctat caacctatag atgtagttcg tgatctacct tctggtttta acactttgaa |
| | 1861 | acctattttt aagttgcctc ttggtattaa cattacaaat tttagagcca ttcttacagc |
| | 1921 | cttttcacct gctcaagaca tttggggcac gtcagctgca gcctattttg ttggctattt |
| | 1981 | aaagccaact acatttatgc tcaagtatga tgaaaatggt acaatcacag atgctgttga |
| | 2041 | ttgttctcaa aatccacttg ctgaactcaa atgctctgtt aagagctttg agattgacaa |
| | 2101 | aggaatttac cagacctcta atttcagggt tgttccctca ggagatgttg tgagattccc |
| | 2161 | taatattaca aacttgtgtc cttttggaga ggtttttaat gctactaaat tcccttctgt |
| | 2221 | ctatgcatgg gagagaaaaa aaatttctaa ttgtgttgct gattactctg tgctctacaa |
| | 2281 | ctcaacattc ttttcaacct ttaagtgcta tggcgtttct gccactaagt tgaatgatct |
| | 2341 | ttgcttctcc aatgtctatg cagattcttt tgtagtcaag ggagatgatg taagacaaat |
| | 2401 | agcgccagga caaactggtg ttattgctga ttataattat aaattgccag atgatttcat |
| | 2461 | gggttgtgtc cttgcttgga atactaggaa cattgatgct acttcaactg gtaatcataa |
| | 2521 | ttataaatat aggtatctta gacatggcaa gcttaggccc tttgagagag acatatctaa |
| | 2581 | tgtgcctttc tcccctgatg gcaaaccttg caccccacct gctcttaatt gttattggcc |
| | 2641 | attaaatgat tatggttttt acaccactac tggcattggc taccaacctt acagagttgt |
| | 2701 | agtactttct tttgaacttt taaatgcacc ggccacggtt tgtggaccaa aattatccac |
| | 2761 | tgaccttatt aagaaccagt gtgtcaattt taattttaat ggactcactg gtactggtgt |
| | 2821 | gttaactcct tcttcaaaga gatttcaacc atttcaacaa tttggccgtg atgtttctga |
| | 2881 | tttcactgat tccgttcgag atcctaaaac atctgaaata ttagacattt caccttgctc |
| | 2941 | ttttgggggt gtaagtgtaa ttacacctgg aacaaatgct tcatctgaag ttgctgttct |
| | 3001 | atatcaagat gttaactgca ctgatgtttc tacagcaatt catgcagatc aactcacacc |
| | 3061 | agcttggcgc atatattcta ctggaaacaa tgtattccag actcaagcag gctgtcttat |
| | 3121 | aggagctgag catgtcgaca cttcttatga gtgcgacatt cctattggag ctggcatttg |
| | 3181 | tgctagttac catacagttt ctttattacg tagtactagc caaaaatcta ttgtggctta |
| | 3241 | tactatgtct ttaggtgctg atagttcaat tgcttactct aataacacca ttgctatacc |
| | 3301 | tactaacttt tcaattagca ttactacaga agtaatgcct gtttctatgg ctaaaacctc |
| | 3361 | cgtagattgt aatatgtaca tctgcggaga ttctactgaa tgtgctaatt tgcttctcca |
| | 3421 | atatggtagc ttttgcacac aactaaatcg tgcactctca ggtattgctg ctgaacagga |
| | 3481 | tcgcaacaca cgtgaagtgt tcgctcaagt caaacaaatg tacaaaaccc caactttgaa |
| | 3541 | atattttggt ggttttaatt tttcacaaat attacctgac cctctaaagc caactaagag |
| | 3601 | gtcttttatt gaggacttgc tctttaataa ggtgacactc gctgatgctg gcttcatgaa |
| | 3661 | gcaatatggc gaatgcctag gtgatattaa tgctagagat ctcatttgtg cgcagaagtt |
| | 3721 | caatggactt acagtgttgc cacctctgct cactgatgat atgattgctg cctacactgc |
| | 3781 | tgctctagtt agtggtactg ccactgctgg atggacattt ggtgctggcg ctgctcttca |
| | 3841 | aatacctttt gctatgcaaa tggcatatag gttcaatggc attggagtta cccaaaatgt |
| | 3901 | tctctatgag aaccaaaaac aaatcgccaa ccaatttaac aaggcgatta gtcaaattca |
| | 3961 | agaatcactt acaacaacat caactgcatt gggcaagctg caagacgttg ttaaccagaa |
| | 4021 | tgctcaagca ttaaacacac ttgttaaaca acttagctct aattttggtg caatttcaag |
| | 4081 | tgtgctaaat gatatccttt cgcgacttga taaagtcgag gcggaggtac aaattgacag |
| | 4141 | gttaattaca ggcagacttc aaagccttca aacctatgta acacaacaac taatcagggc |
| | 4201 | tgctgaaatc agggcttctg ctaatcttgc tgctactaaa atgtctgagt gtgttcttgg |
| | 4261 | acaatcaaaa agagttgact tttgtggaaa gggctaccac cttatgtcct tcccacaagc |
| | 4321 | agccccgcat ggtgttgtct tcctacatgt cacgtatgtg ccatcccagg agaggaactt |
| | 4381 | caccacagcg ccagcaattt gtcatgaagg caaagcatac ttccctcgtg aaggtgtttt |
| | 4441 | cgtgtttaat ggcacttctt ggtttattac acagaggaac ttcttttctc cacaaataat |
| | 4501 | tactacagac aatacatttg tctcaggaaa ttgtgatgtc gttattggca tcattaacaa |
| | 4561 | cacagtttat gatcctctgc aacctgagct cgactcattc aaagaagagc tggacaagta |
| | 4621 | cttcaaaaat catacatcac cagatgttga tcttggcgac atttcaggca ttaacgcttc |
| | 4681 | tgtcgtcaac attcaaaaag aaattgaccg cctcaatgag gtcgctaaaa atttaaatga |
| | 4741 | atcactcatt gaccttcaag aattgggaaa atatgagcaa tatattaaat ggccttggta |
| | 4801 | tgtttggctc ggcttcattg ctggactaat tgccatcgtc atggttacaa tcttgctttg |
| | 4861 | ttgcatgact agttgttgca gttgcctcaa gggtgcatgc tcttgtggtt cttgctgcaa |
| | 4921 | gtttgatgag gatgactctg agccagttct caagggtgtc aaattacatt acacataata |
| | 4981 | ttatattttt tatctaaaaa actaaaaata aacattgatt aaattttaat ataatactta |
| | 5041 | aaaatggatg ttgtgtcgtt agataaaccg tttatgtatt ttgaggaaat tgataatgag |
| | 5101 | ttagattacg aaccagaaag tgcaaatgag gtcgcaaaaa aactaccgta tcaaggacag |
| | 5161 | ttaaaactat tactaggaga attatttttt cttagtaagt tacagcgaca cggtatatta |
| | 5221 | gatggtgcca ccgtagtgta tataggatcg gctcctggta cacatatacg ttatttgaga |
| | 5281 | gatcatttct ataatttagg aatgattatc aaatggatgc taattgacgg acgccatcat |
| | 5341 | gatcctattc taaatggatt gcgtgatgtg actctagtga ctcggttcgt tgatgaggaa |
| | 5401 | tatctacgat ccatcaaaaa acaactgcat ccttctaaga ttattttaat ttctgatgta |
| | 5461 | agatccaaac gaggaggaaa tgaacctagt acggcggatt tactaagtaa ttacgctcta |
| | 5521 | caaaatgtca tgattagtat tttaaacccc gtggcatcta gtcttaaatg gagatgcccg |
| | 5581 | tttccagatc aatggatcaa ggacttttat atcccacacg gtaataaaat gttacaacct |
| | 5641 | tttgctcctt catattcagc tgaaatgaga ttattaagta tttataccgg tgagaacatg |
| | 5701 | agactgactc gagttaccaa attagacgct gtaaattatg aaaaaaagat gtactacctt |
| | 5761 | aataagatcg tccgtaacaa agtagttgtt aactttgatt atcctaatca ggaatatgac |
| | 5821 | tattttcaca tgtactttat gctgaggacc gtatactgca ataaaacatt tcctactact |
| | 5881 | aaagcaaagg tactatttct acaacaatct atatttcgtt tcttaaatat tccaacaaca |
| | 5941 | tcaactgaaa aagttagtca tgaaccaata caacgtaa |
| Nucleotide sequence of | 1 | atttacggat tcaccaataa aaataaacta gagaaactta gtactaataa ggaactagaa |
| synVACV_SARS_Ad_Spike_deltaT5NT | 61 | tcgtatagtt ctagccctct tcaagaaccc attaggttaa atgattttct gggactattg |
| (SEQ ID NO: 65) | 121 | gaatgtgtta aaaagaatat tcctctaaca gatattccga caaaggattg attactataa |
| | 181 | atggagaatg ttcctaatgt atactttaat cctgtgttta tagagcccac gtttaaacat |
| | 241 | tctttattaa gtgtttataa acacagatta atagttttat ttgaagtatt cgttgtattc |
| | 301 | attctaatat atgtattttt tagatctgaa ttaaatatgt tcttcatgcc taaacgaaaa |
| | 361 | atacccgatc ctattgatag attacgacgt gctaatctag cgtgtgaaga cgataaatta |
| | 421 | atgatctatg gattaccatg gatgacaact caaacatctg cgttatcaat aaatagtaaa |
| | 481 | ccgatagtgt ataaagattg tgcaaagctt ttgcgatcaa taaatggatc acaaccagta |
| | 541 | tctcttaacg atgttcttcg cagatgatga ttcatttttt aagtatttgg ctagtcaaga |
| | 601 | tgatgaatct tcattatctg atatattgca aatcactcaa tatctagact ttctgttatt |
| | 661 | attattgatc caatcaaaaa ataaattaga agccgtgggt cattgttatg aatctctttc |
| | 721 | agaggaatac agacaattga caaaattcac agactctcaa gattttaaaa aactgtttaa |
| | 781 | caaggtccct attgttacag atggaagggt caaacttaat aaaggatatt tgttcgactt |
| | 841 | tgtgattagt ttgatgcgat tcaaaaaaga atcagctcta gctaccaccg caatagatcc |
| | 901 | tgttagatac atagatcctc gtcgcgatat cgcattttct aacgtgatgg atatattaaa |
| | 961 | gtcgaataaa gtgaacaata attaattctt tattgtcatc ttttattttt tttttttgga |
| | 1021 | atataaatat ccggtaaaat tgaaaaaata tacactaatt agcgtctcgt ttcagacgct |
| | 1081 | agctcgaggt tgggagctct ccggatccaa gcttatcgat ttcgaacccg gggtaccgaa |
| | 1141 | ttcctcgagg ttgggagctc tccggatcca agcttatcga tttcgaaccc ggggtaccga |
| | 1201 | attcctcgag atgtttattt tcttattatt tcttactctc actagtggta gtgaccttga |
| | 1261 | ccggtgcacc acttttgatg atgttcaagc tcctaattac actcaacata cttcatctat |
| | 1321 | gaggggggtt tactatcctg atgaaatttt tagatcagac actctttatt taactcagga |
| | 1381 | tttatttctt ccattttatt ctaatgttac agggtttcat actattaatc atacgtttgg |
| | 1441 | caaccctgtc atacctttta aggatggtat ttattttgct gccacagaga aatcaaatgt |
| | 1501 | tgtccgtggt tgggtttttg gttctaccat gaacaacaag tcacagtcgg tgattattat |
| | 1561 | taacaattct actaatgttg ttatacgagc atgtaacttt gaattgtgtg acaacccttt |
| | 1621 | ctttgctgtt tctaaaccca tgggtacaca gacacatact atgatattcg ataatgcatt |
| | 1681 | taattgcact ttcgagtaca tatctgatgc cttttcgctt gatgtttcag aaaagtcagg |
| | 1741 | taattttaaa cacttacgag agtttgtgtt taaaaataaa gatgggtttc tctatgttta |
| | 1801 | taagggctat caacctatag atgtagttcg tgatctacct tctggtttta acactttgaa |
| | 1861 | acctattttt aagttgcctc ttggtattaa cattacaaat tttagagcca ttcttacagc |
| | 1921 | cttttcacct gctcaagaca tttggggcac gtcagctgca gcctattttg ttggctattt |
| | 1981 | aaagccaact acatttatgc tcaagtatga tgaaaatggt acaatcacag atgctgttga |
| | 2041 | ttgttctcaa aatccacttg ctgaactcaa atgctctgtt aagagctttg agattgacaa |
| | 2101 | aggaatttac cagacctcta atttcagggt tgttccctca ggagatgttg tgagattccc |
| | 2161 | taatattaca aacttgtgtc cttttggaga ggtttttaat gctactaaat tcccttctgt |
| | 2221 | ctatgcatgg gagagaaaaa aaatttctaa ttgtgttgct gattactctg tgctctacaa |
| | 2281 | ctcaacattc ttttcaacct ttaagtgcta tggcgtttct gccactaagt tgaatgatct |
| | 2341 | ttgcttctcc aatgtctatg cagattcttt tgtagtcaag ggagatgatg taagacaaat |
| | 2401 | agcgccagga caaactggtg ttattgctga ttataattat aaattgccag atgatttcat |
| | 2461 | gggttgtgtc cttgcttgga atactaggaa cattgatgct acttcaactg gtaatcataa |
| | 2521 | ttataaatat aggtatctta gacatggcaa gcttaggccc tttgagagag acatatctaa |
| | 2581 | tgtgcctttc tcccctgatg gcaaaccttg caccccacct gctcttaatt gttattggcc |
| | 2641 | attaaatgat tatggttttt acaccactac tggcattggc taccaacctt acagagttgt |
| | 2701 | agtactttct tttgaacttt taaatgcacc ggccacggtt tgtggaccaa aattatccac |
| | 2761 | tgaccttatt aagaaccagt gtgtcaattt taattttaat ggactcactg gtactggtgt |
| | 2821 | gttaactcct tcttcaaaga gatttcaacc atttcaacaa tttggccgtg atgtttctga |
| | 2881 | tttcactgat tccgttcgag atcctaaaac atctgaaata ttagacattt caccttgctc |
| | 2941 | ttttgggggt gtaagtgtaa ttacacctgg aacaaatgct tcatctgaag ttgctgttct |
| | 3001 | atatcaagat gttaactgca ctgatgtttc tacagcaatt catgcagatc aactcacacc |
| | 3061 | agcttggcgc atatattcta ctggaaacaa tgtattccag actcaagcag gctgtcttat |
| | 3121 | aggagctgag catgtcgaca cttcttatga gtgcgacatt cctattggag ctggcatttg |
| | 3181 | tgctagttac catacagttt ctttattacg tagtactagc caaaaatcta ttgtggctta |
| | 3241 | tactatgtct ttaggtgctg atagttcaat tgcttactct aataacacca ttgctatacc |
| | 3301 | tactaacttt tcaattagca ttactacaga agtaatgcct gtttctatgg ctaaaacctc |
| | 3361 | cgtagattgt aatatgtaca tctgcggaga ttctactgaa tgtgctaatt tgcttctcca |
| | 3421 | atatggtagc ttttgcacac aactaaatcg tgcactctca ggtattgctg ctgaacagga |
| | 3481 | tcgcaacaca cgtgaagtgt tcgctcaagt caaacaaatg tacaaaaccc caactttgaa |
| | 3541 | atattttggt ggttttaatt tttcacaaat attacctgac cctctaaagc caactaagag |
| | 3601 | gtcttttatt gaggacttgc tctttaataa ggtgacactc gctgatgctg gcttcatgaa |
| | 3661 | gcaatatggc gaatgcctag gtgatattaa tgctagagat ctcatttgtg cgcagaagtt |
| | 3721 | caatggactt acagtgttgc cacctctgct cactgatgat atgattgctg cctacactgc |
| | 3781 | tgctctagtt agtggtactg ccactgctgg atggacattt ggtgctggcg ctgctcttca |
| | 3841 | aatacctttt gctatgcaaa tggcatatag gttcaatggc attggagtta cccaaaatgt |
| | 3901 | tctctatgag aaccaaaaac aaatcgccaa ccaatttaac aaggcgatta gtcaaattca |
| | 3961 | agaatcactt acaacaacat caactgcatt gggcaagctg caagacgttg ttaaccagaa |
| | 4021 | tgctcaagca ttaaacacac ttgttaaaca acttagctct aattttggtg caatttcaag |
| | 4081 | tgtgctaaat gatatccttt cgcgacttga taaagtcgag gcggaggtac aaattgacag |
| | 4141 | gttaattaca ggcagacttc aaagccttca aacctatgta acacaacaac taatcagggc |
| | 4201 | tgctgaaatc agggcttctg ctaatcttgc tgctactaaa atgtctgagt gtgttcttgg |
| | 4261 | acaatcaaaa agagttgact tttgtggaaa gggctaccac cttatgtcct tcccacaagc |
| | 4321 | agccccgcat ggtgttgtct tcctacatgt cacgtatgtg ccatcccagg agaggaactt |
| | 4381 | caccacagcg ccagcaattt gtcatgaagg caaagcatac ttccctcgtg aaggtgtttt |
| | 4441 | cgtgtttaat ggcacttctt ggtttattac acagaggaac ttcttttctc cacaaataat |
| | 4501 | tactacagac aatacatttg tctcaggaaa ttgtgatgtc gttattggca tcattaacaa |
| | 4561 | cacagtttat gatcctctgc aacctgagct cgactcattc aaagaagagc tggacaagta |
| | 4621 | cttcaaaaat catacatcac cagatgttga tcttggcgac atttcaggca ttaacgcttc |
| | 4681 | tgtcgtcaac attcaaaaag aaattgaccg cctcaatgag gtcgctaaaa atttaaatga |
| | 4741 | atcactcatt gaccttcaag aattgggaaa atatgagcaa tatattaaat ggccttggta |
| | 4801 | tgtttggctc ggcttcattg ctggactaat tgccatcgtc atggttacaa tcttgctttg |
| | 4861 | ttgcatgact agttgttgca gttgcctcaa gggtgcatgc tcttgtggtt cttgctgcaa |
| | 4921 | gtttgatgag gatgactctg agccagttct caagggtgtc aaattacatt acacataata |
| | 4981 | ttatattttt tatctaaaaa actaaaaata aacattgatt aaattttaat ataatactta |
| | 5041 | aaaatggatg ttgtgtcgtt agataaaccg tttatgtatt ttgaggaaat tgataatgag |
| | 5101 | ttagattacg aaccagaaag tgcaaatgag gtcgcaaaaa aactgccgta tcaaggacag |
| | 5161 | ttaaaactat tactaggaga attatttttt cttagtaagt tacagcgaca cggtatatta |
| | 5221 | gatggtgcca ccgtagtgta tataggatct gctcccggta cacatatacg ttatttgaga |
| | 5281 | gatcatttct ataatttagg agtgatcatc aaatggatgc taattgacgg ccgccatcat |
| | 5341 | gatcctattt taaatggatt gcgtgatgtg actctagtga ctcggttcgt tgatgaggaa |
| | 5401 | tatctacgat ccatcaaaaa acaactgcat ccttctaaga ttattttaat ttctgatgtg |
| | 5461 | agatccaaac gaggaggaaa tgaacctagt acggcggatt tactaagtaa ttacgctcta |
| | 5521 | caaaatgtca tgattagtat tttaaacccc gtggcatcta gtcttaaatg gagatgcccg |
| | 5581 | tttccagatc aatggatcaa ggacttttat atcccacacg gtaataaaat gttacaacct |
| | 5641 | tttgctcctt catattcagc tgaaatgaga ttattaagta tttataccgg tgagaacatg |
| | 5701 | agactgactc gagttaccaa attagacgct gtaaattatg aaaaaaagat gtactacctt |
| | 5761 | aataagatcg tccgtaacaa agtagttgtt aactttgatt atcctaatca ggaatatgac |
| | 5821 | tattttcaca tgtactttat gctgaggacc gtgtactgca ataaaacatt tcctactact |
| | 5881 | aaagcaaagg tactatttct acaacaatct atatttcgtt tcttaaatat tccaacaaca |
| | 5941 | tcaactgaaa aagttagtca tgaaccaata caacgtaa |

### Examples

### Example 1. Generation of the Synthetic Horsepox Virus

The synthetic horsepox virus (scHPXV) is generated following the methods disclosed in US 2018/0251736, incorporated herein by reference in its entirety.

The design of the synthetic HPXV genome is based on the previously described genome sequence for HPXV (strain MNR-76; GenBank accession DQ792504) (Tulman E R, Delhon G, Afonso C L, Lu Z, Zsak L, Sandybaev N T, et al. Genome of horsepox virus. Journal of virology. 2006; 80(18):9244-58). The 212,633 bp genome is divided into 10 overlapping fragments. These fragments are designed so that they shared at least 1.0 kbp of overlapping sequence (i.e. homology) with each adjacent fragment, to provide sites where homologous recombination will drive the assembly of full-length genomes. The fragments generated are shown in Table 2. These overlapping sequences will provide sufficient homology to accurately carry out recombination between the co-transfected fragments

TABLE 2
HPXV genome fragments for use to generate the
synthetic HPXV. The size of each fragment and
location within the HPXV genome are indicated.

| | | Location within |
|---|---|---|
| | | HPXV [DQ792504] |
| Fragment Name | Size (bp) | (bp) |
| GA_Left ITR (SEQ ID NO: 15) | 10,095 | 41-10,135 |
| GA_Fragment 1A (SEQ ID NO: 16) | 16,257 | 8505-24,761 |
| GA_Fragment 1B (SEQ ID NO: 17) | 16,287 | 23764-40,050 |
| GA_Fragment 2 (SEQ ID NO: 18) | 31,946 | 38,705-70,650 |
| GA_Fragment 3 (SEQ ID NO: 19) | 25,566 | 68,608-94,173 |
| GA_Fragment 4 (SEQ ID NO: 20) | 28,662 | 92,587-121,248 |
| GA_Fragment 5 (SEQ ID NO: 21) | 30,252 | 119,577-149,828 |
| GA_Fragment 6 (SEQ ID NO: 22) | 30,000 | 147,651-177,650 |
| GA_Fragment 7 (SEQ ID NO: 23) | 28,754 | 176,412-205,165 |
| GA_Right ITR (SEQ ID NO: 24) | 8,484 | 204,110-212,593 |

The resulting synthetic HPXV has been deposited in GenBank as accession number KY349117.

A yfp/gpt cassette under the control of a poxvirus early late promoter is introduced into the HPXV095/J2R locus within GA_Fragment_3, so that reactivation of HPXV (scHPXV YFP-gpt::095) will be easy to visualize under a fluorescence microscope. SFV-catalyzed recombination and reactivation of poxvirus DNA to assemble recombinant poxviruses has previously been described (Yao X D et al. Journal of virology. 2003; 77(13):7281-90; and Yao X D et al. Methods Mol Biol. 2004; 269:51-64; the entire disclosures of each are incorporated by reference herein). Several biological features make this an attractive model system. First, SFV has a narrow host range, productively infecting rabbit cells and certain monkey cell lines, like BGMK. It can infect, but grows very poorly on cells like BSC-40. Second, it grows more slowly compared to Orthopoxviruses, taking approximately 4-5 days to form transformed “foci” in monolayers of cells, a characteristic that is very different from Orthopoxviruses, which produce plaques within 1-2 days in culture. This difference in growth between Leporipoxviruses and Orthopoxviruses allows differentiation of these viruses by performing the reactivation assays in BGMK cells and plating the progeny on BSC-40 cells. In some embodiments, other helper viruses (such as, but not limited to, fowlpox virus) may be used. In some embodiments, different cell combinations may be used.

BGMK cells are infected with SFV at a MOI of 0.5 and then transfected with 5 μg of digested GA_HPXV fragments 2 h later. Five days post transfection, all of the infectious particles are recovered by cell lysis and re-plated on BSC-40 cells, which only efficiently support growth of HPXV. The resulting reactivated scHPXV YFP-gpt::095 plaques are visualized under a fluorescence microscope. The visualization is enabled by the yfp/gpt selectable marker in the HPXV095/J2R locus within Frag_3. Virus plaques are detected in BSC-40 monolayers within 48 h of transfection. The efficiency of recovering scHPXV YFP-gpt::095 is dependent on a number of factors, including DNA transfection efficiency, but ranges up to a few PFU/μg of DNA transfected.

A yfp/gpt cassette under the control of a poxvirus early late promoter is also introduced into the HPXV200 locus within GA_Fragment_7, so that reactivation of HPXV (scHPXV YFP-gpt::200) will be easy to visualize under a fluorescence microscope. SFV-catalyzed recombination and reactivation of poxvirus DNA to assemble recombinant poxviruses has previously been described (Yao X D et al. Journal of virology. 2003; 77(13):7281-90; and Yao X D et al. Methods Mol Biol. 2004; 269:51-64; the entire disclosures of each are incorporated by reference herein). Several biological features make this an attractive model system. First, SFV has a narrow host range, productively infecting rabbit cells and certain monkey cell lines, like BGMK. It can infect, but grows very poorly on cells like BSC-40. Second, it grows more slowly compared to Orthopoxviruses, taking approximately 4-5 days to form transformed “foci” in monolayers of cells, a characteristic that is very different from Orthopoxviruses, which produce plaques within 1-2 days in culture. This difference in growth between Leporipoxviruses and Orthopoxviruses allows differentiation of these viruses by performing the reactivation assays in BGMK cells and plating the progeny on BSC-40 cells. In some embodiments, other helper viruses (such as, but not limited to, fowlpox virus) may be used. In some embodiments, different cell combinations may be used.

BGMK cells are infected with SFV at a MOI of 0.5 and then transfected with 5 μg of digested GA_HPXV fragments 2 hours later. Five days post transfection, all of the infectious particles are recovered by cell lysis and re-plated on BSC-40 cells, which only efficiently support growth of HPXV. The resulting reactivated scHPXV YFP-gpt::200 plaques are visualized under a fluorescence microscope. The visualization is enabled by the yfp/gpt selectable marker in the HPXV200 locus within Frag_7. Virus plaques are detected in BSC-40 monolayers within 48 hours of transfection. The efficiency of recovering scHPXV YFP-gpt::200 is dependent on a number of factors, including DNA transfection efficiency, but ranges up to a few PFU/μg of DNA transfected.

### Example 2. Generation of the Synthetic Vaccinia Virus, Strain ACAM2000

The synthetic vaccinia virus ACAM2000 was generated using the methods disclosed in WO 2019/213452, incorporated herein by reference in its entirety.

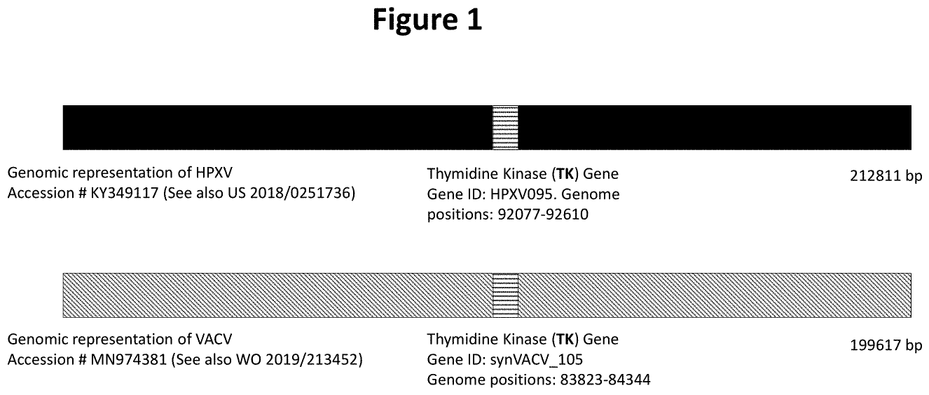

The design of the synthetic VACV (synVACV) genome was based on the previously described genome sequence for VACV ACAM2000 (GenBank accession AY313847) (Osborne J D et al. Vaccine. 2007; 25(52):8807-32). The genome was divided into 9 overlapping fragments (FIG. 1). These fragments were designed so that they shared at least 1.0 kbp of overlapping sequence (i.e. homology) with each adjacent fragment, to provide sites where homologous recombination will drive the assembly of full-length genomes (Table 3). These overlapping sequences provided sufficient homology to accurately carry out recombination between the co-transfected fragments (Yao X D, Evans D H. Journal of Virology. 2003; 77(13):7281-90).

TABLE 3
The VACV ACAM2000 genome fragments used in this study.
The size and the sequence within the VACV ACAM2000
genome [GenBank Accession AY313847] are described.

| Fragment Name | Size (bp) | Sequence |
|---|---|---|
| GA_LITR | 18,525 | SEQ ID NO: 25 |
| ACAM2000 | | |
| GA_FRAG_1 | 24,931 | SEQ ID NO: 26 |
| ACAM2000 | | |
| GA_FRAG_2 | 23,333 | SEQ ID NO: 27 |
| ACAM2000 | | |
| GA_FRAG_3 | 26,445 | SEQ ID NO: 28 |
| ACAM2000 | | |
| GA_FRAG_4 | 26,077 | SEQ ID NO: 29 |
| ACAM2000 | | |
| GA_FRAG_5 | 24,671 | SEQ ID NO: 30 |
| ACAM2000 | | |
| GA_FRAG_6 | 25,970 | SEQ ID NO: 31 |
| ACAM2000 | | |
| GA_FRAG_7 | 28,837 | SEQ ID NO: 32 |
| ACAM2000 | | |
| GA_RITR | 17,641 | SEQ ID NO: 33 |
| ACAM2000 | | |

The resulting synthetic VACV, ACAM 2000 has been deposited in GenBank as accession number MN974381.

### Example 3. Generation of the Engineered SARS-CoV-2 S Protein

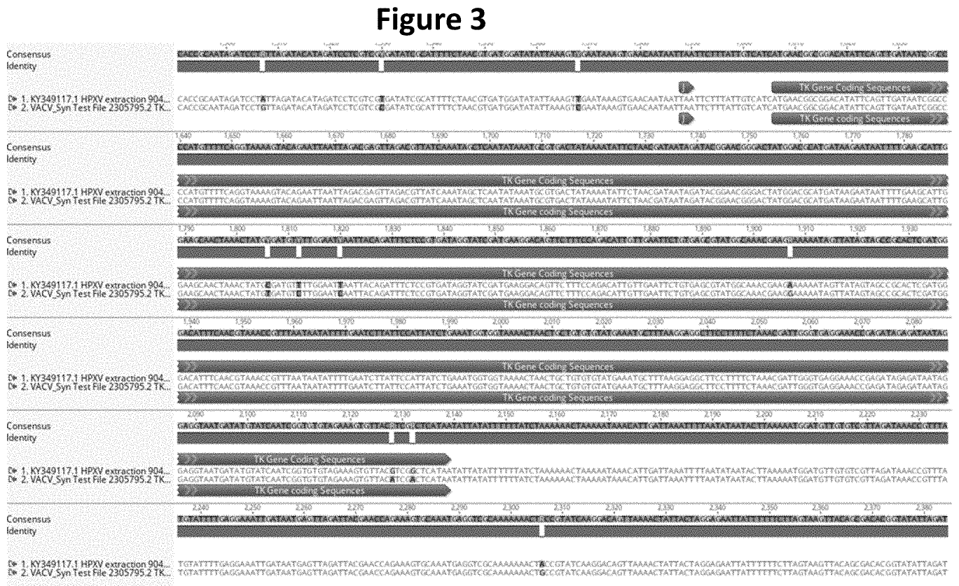

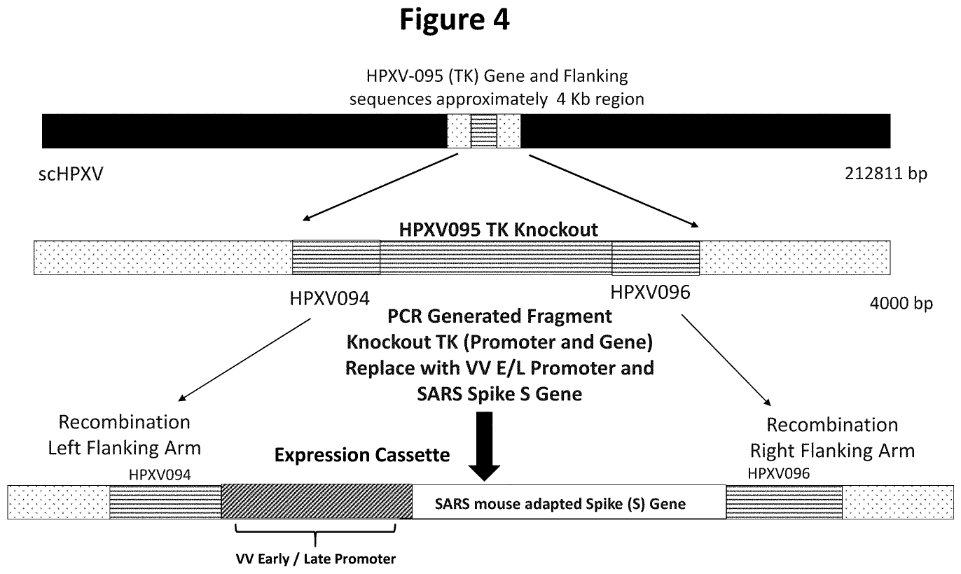

The nucleotide sequence alignment of the synthetic HPXV (Accession number KY349117) and the synthetic VACV (Accession number MN974381) indicates a nucleotide sequence identity of 99% throughout the 4 Kb TK gene locus and a co-linearity (Start and Stop) of the TK gene sequences, which were used for the construction of the ΔTK insertion locus or knockout TK locus. See FIG. 3.

The TK gene is non-essential for viral replication in tissue culture. It also provides a stable insertion site for foreign gene(s) of interest and a selection marker (TK−) in the presence of the nucleotide analog 5-Bromodeoxyuridine (5-BrdU).

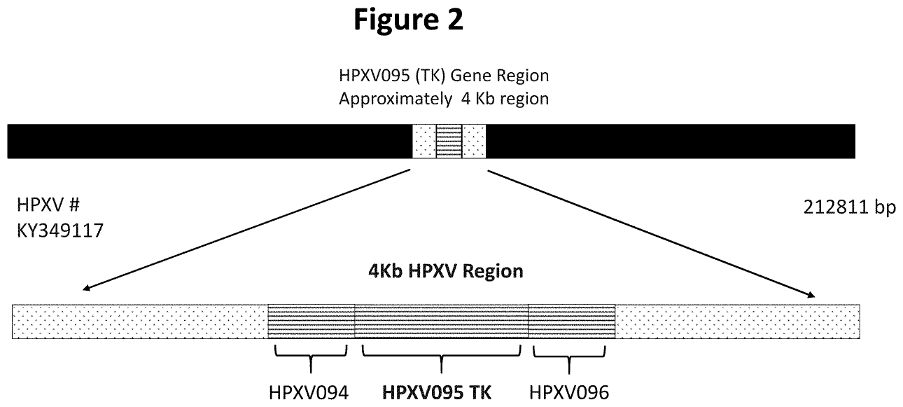

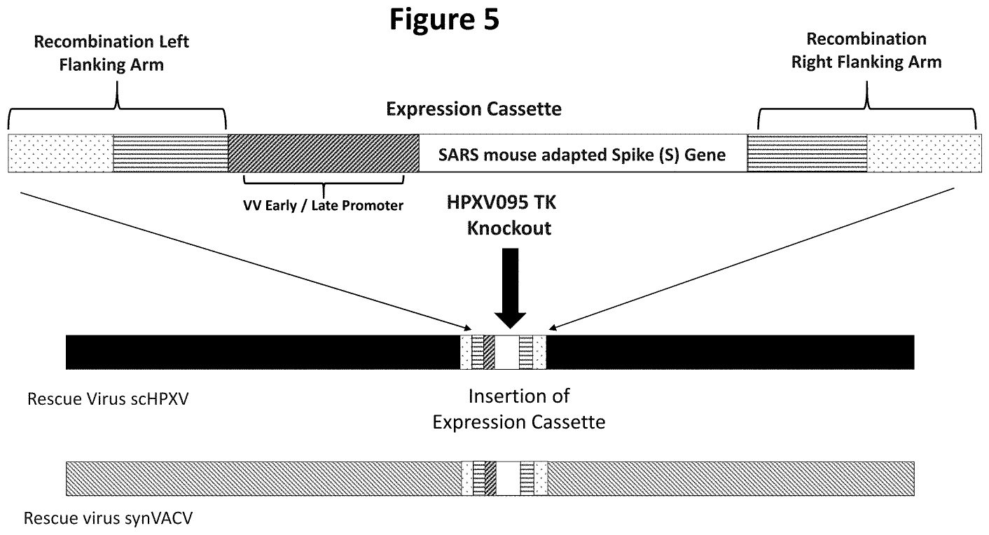

Because of the high level of sequence identity between the synthetic HPXV and the synthetic VACV, the PCR sequence manipulations used for the generation of the expression cassette containing the promoter/gene sequences allow for the use of the same expression cassette with the two different rescue viruses. For the rescue of the transfected PCR fragment comprising the engineered SARS-CoV-2 S protein, virus specific sequences (recombination left and right flanking arms, corresponding to HPXV094 and HPXV096, respectively) allows the recombination of the expression cassette into the viral TK locus. See FIG. 2 and FIG. 5.

A nucleotide sequence alignment of the Spike (S) gene of different SARS-CoV-2 isolates is performed. The viral isolates aligned are the ones published under the following accession numbers NC045512.2, LC521925.1, MN988668.1, MN985325.1, MN975262.1, MN938384.1, LR757998.1, LR757996.1, LR757995.1 and MN908947.3. The alignment of the S genes indicates 100% homology at the nucleotide level between the S gene of the different viral isolates. All viral isolates sequences are isolates with complete genome sequence entries from China, Japan and the US. Early indications from isolate sequence analysis seems to indicate little viral drift. However, if drift is ultimately observed, the same techniques can be used with the modified virus and its proteins and nucleic acid sequences.

The nucleotide sequence encoding the S protein of the SARS-CoV-2 comprises the nucleotide sequence set forth in SEQ ID NO: 9 or SEQ ID NO: 47. The SARS-CoV-2 is not well adapted for infection in mice. Therefore, genomic adaptative mutations are introduced to adapt the virus for infection in mice. In particular, a mutation in the nucleotide sequence is introduced, the mutation resulting in a S protein comprising a Y459H substitution. Table 4 shows genomic adaptative mutations in SARS-CoV virus, that can be adapted and introduced into other regions of the SARS-CoV-2 virus. See Roberts A et al. PLoS Pathog. 2007 January; 3(1): e5. doi: 10.1371.

The six mutations found in a SARS-CoV virus resulting from fifteen passages (and the resulting virus called MA15) and that are lethal for mice following intranasal inoculation are listed in Table 4. The labels in Table 4 are as follows: ORF^{a}: open reading frame; CDS^{b} coding sequence, sequence of nucleotides that corresponds with the sequence of amino acids in a protein (location includes start and stop codon); nsp^{c}; non-structural protein, cleavage product of ORF lab; Main^{pro}: main 3C-like protease; Hel: helicase; RBM^{d}: receptor binding motif (amino acids 424-494).

TABLE 4
Genomic adaptive mutations in SARS-CoV virus

| | | Mutations found in MA15 compared to SARS-CoV (Urbani) | |
|---|---|---|---|
| ORF^{a} | CDS^{b} | Nucleotide change | Amino acid change in SARS-CoV protein |
| 1a | 265-13413 | 10384 C−>T | H133Y nsp5 (Main^{pro})^{c} |
| | | 10793 A−>C | E269A nsp5 (Main^{pro})^{c} |
| | | 12814 A−>G | T67A nsp9c |
| 1b | 13398-21485 | 16177 C−>T | A4V nsp13 (Hel)^{c} |
| S | 21492-25259 | 22797 T−>C | Y436H Spike protein-RBM^{d} |
| M | 26398-27063 | 26428 G−>A | E11K M protein |

For efficient expression of transgenes from poxvirus vectors, heterologous gene coding sequences containing the vaccinia Early Transcription Terminator Signal (ETTS) should be removed, in one embodiment of this disclosure, through coding silent mutagenesis to generate full length transcripts during the early phase of the infection. These sequences have the following sequence: TTTTTNT (T5NT); SEQ ID NO: 14. Removing the ETTS in the S protein coding sequence can positively impact the generation of robust immune responses. See Earl P L et al. J Virol. 1990 May; 64(5):2448-51.

Examples of other mutations introduced in the S protein (SEQ ID NO: 47) in other embodiments of this disclosure are the following: D614G, S943P, K986P and V987P. One or more of these mutations can be introduced in the S protein in those embodiments.

Poxvirus replication occurs in the cytoplasm of the infected cell. The viruses do not enter the nucleus of the infected cell during the replication cycle, and therefore do not utilize the host cell transcriptional apparatus. Because of the cytoplasmic location of replication, poxviruses encode their own transcriptional machinery including the viral RNA polymerase and their own regulatory promoter recognition signals. Therefore, for efficient high-level expression from eukaryotic transgene expression has to be driven from poxvirus promoters. Poxvirus gene expression is controlled by early, intermediate and late promoters and can be defined as early (8 Hours before infection) and late (8 hours post-infection). DNA synthesis occurs 8 hours post infection and is referred to as the temporal boundary for the initiation of late gene expression. Highest levels of transgene antigenic load have usually been achieved through the use of a combination of Early and Late Promoter signals. The promoter used to control transcription of the S protein is an overlapping synthetic early/late promoter comprising the sequence (TTTTATTTTTTTTTTTTGGAATATAAATATCCGGTAAAATTGAAAAAATA SEQ ID NO: 8) including a 160 nucleotides long spacer 3′ of the early promoter and between the RNA start site and the ATG (SEQ ID NO: 42). See FIG. 9. See Di Pilato et al. Journal of General Virology (2015), 96, 2360-2371; incorporated herein by reference in its entirety. It seems that spacers with more than 50 nt would offer greater space to the transcription machinery, possibly accelerating gene expression, and spacers with more than 99 nt offer advantages to early gene expression.

The expression cassette generated comprises the engineered SARS-CoV-2 S protein adapted for mouse infection and where the ETTS sequences have been removed and controlled under the transcription of the overlapping tandem early/late promoter.

### Example 4. Generation of the Recombinant Poxvirus Comprising the Engineered SARS-CoV-2 S Protein

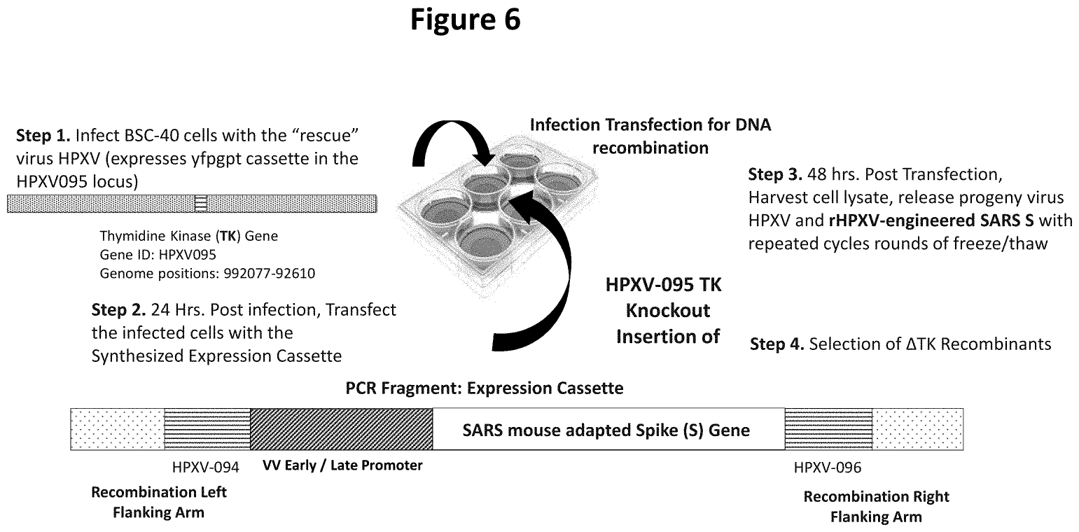

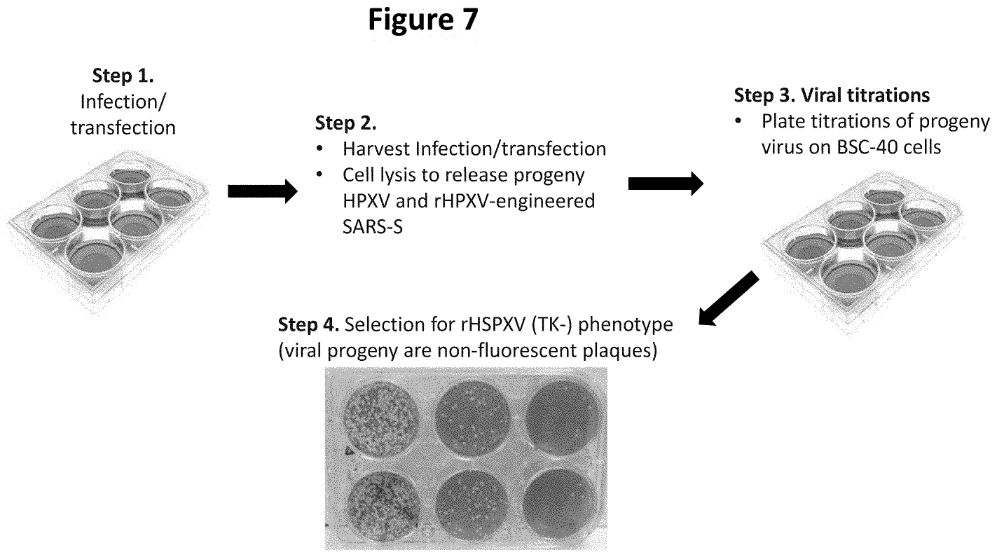

An exemplary method to generate a recombinant horsepox comprising the S protein of SARS-CoV-2 virus is shown in FIGS. 6 and 7 and comprises:
(a) Infection of cells (e.g., Vero cells or BSC-40 cells) with the rescue synthetic horsepox virus and the rescue synthetic VACV, as described above.
(b) The transfection of the infected cells (e.g., Vero cells or BSC-40 cells) with a PCR generated nucleotide fragment comprising the “engineered SARS-CoV-2 S gene expression cassette” is performed 24 hours post-infection. Recombination of the expression cassette occurs through the left and right flanking arms and the expression cassette is inserted into the TK gene locus. Accordingly, HPXV-095 TK locus is knocked-out and the expression cassette is inserted in the TK gene locus. After 30 min at 25° C., 7.2 ml of Eagle medium containing 8% fetal bovine serum was added and the monolayer was incubated for 3.5 hr at 37° C. The culture medium was then removed and replaced by 8 ml fresh Eagle medium containing 8% fetal bovine serum and the incubation was continued at 37° C. for two days. Cells were scraped from the bottles, pelleted by centrifugation (2,000×g, 5 min) and resuspended in 0.5 ml of Eagle medium containing 2.5% fetal bovine serum.
(c) The transfected cells are harvested 48 hours post-infection and the progeny virus of recombinant synthetic horsepoxvirus comprising the engineered SARS-CoV-2 S gene and the synthetic VACV is released of with repeated cycles of freeze/thaw.
(d) Selection of recombinant viruses. Thymidine kinase negative poxvirus recombinants are selected by plaque assay in TK⁻ cells (e.g., TK⁻ Vero cells or TK⁻ BSC-40 cells) with a 1% low melting agarose overlay containing 25 μg/ml BrdU. After three days at 37° C., cell monolayers are stained with 0.005% neutral red, plaques are picked using a sterile Pasteur pipette and placed in 0.5 ml of Eagle medium containing 2.5% fetal bovine serum. The recombinant viral progeny is identified by growth in TK⁻ cells. If the SARS-CoV-2 S gene has been inserted into the virus thymidine kinase (TK) gene, viruses containing inserted DNA will be TK⁻ and can be selected on this basis (Mackett et al., (1982)). Confirmation of the S gene is performed by PCR sequence analysis.

Once a recombinant poxvirus has been identified, a variety of methods can be used to assay the expression of the polypeptide encoded by the inserted gene. These methods include, but are not limited to, black plaque assay (an in situ enzyme immunoassay performed on viral plaques), Western blot analysis, radioimmunoprecipitation (RIPA), and enzyme immunoassay (EIA). Antibodies that recognize the SARS-CoV-2 S may be used.

The sequence of one embodiment of a synthetic horsepox virus comprising a nucleic acid encoding a SARS-CoV-2 virus S protein is SEQ ID NO: 43. The sequence of one embodiment of a synthetic vaccinia virus comprising a nucleic acid encoding a SARS-CoV-2 virus S protein is SEQ ID NO: 44.

### Example 5. Immunization of Mice with a Recombinant Poxvirus Comprising the Engineered SARS-CoV-2 S Protein

Primary chicken embryo fibroblasts (CEF) cells prepared from 10-day-old embryos are grown in minimum essential medium supplemented with 10% FBS and used to propagate and titer the recombinant poxvirus.

BALB/c mice are immunized by single-shot and prime-boost vaccination with 10⁵, 10⁶, 10⁷ or 10⁸ PFU of recombinant synthetic horsepox virus expressing SARS-CoV-2 protein via either scarification, intranasally, intramuscular or subcutaneous inoculations. Animals inoculated with non-recombinant virus (WT) or phosphate-buffered saline (Mock) are used as controls.

Four weeks after the immunization, animals are challenged intranasally with 10⁴ tissue culture 50% infective dose (TCID₅₀) of SARS-CoV-2 as described. (Subbarao, K et al. (2004) J. Virol. 78, 3572-3577). Two days later, the lungs and nasal turbinates of four animals in each group are removed and the SARS-CoV-2 titers are determined.

### Example 6. Immunization of Humans with a Recombinant Poxvirus Engineered SARS-CoV-2 S Protein

Subjects at risk for infection by SARS-CoV-2 S are vaccinated using a recombinant poxvirus engineered SARS-CoV-2 S protein of this disclosure through scarification with a bifurcated needle (standard dose, 2.5×10⁵ to 12.5×10⁵ plaque-forming units) typically into the upper arm. The recombinant poxvirus engineered SARS-CoV-2 S protein can also be administered as a single dose one-shot vaccine (e.g., 1×10⁶ PFU TNX-1800), in which vials containing 100 doses per vial are manufactured. The vaccination protects them from infection. However, subsequent vaccinations may be useful to boost immunity.

Methods regarding clinical trial testing of a vaccine have been previously described (Sadoff, J. et al. (2020) Safety and immunogenicity of the Ad26.COV2.S COVID-19 vaccine candidate: interim results of a phase 1/2a, double-blinded, randomized, placebo-controlled trial, MedRxiv, Pages 1-28; incorporated herein by reference in its entirety). A multi-center phase 1/2a randomized, double-blind, placebo-controlled clinical study designed to assess the safety, reactogenicity and immunogenicity of recombinant poxvirus engineered SARS-CoV-2 S protein is conducted. The engineered SARS-CoV-2 S protein is administered at a dose level, for example, between about 5×10¹⁰ to 1×10¹¹ viral particles (vp) per vaccination, either as a single dose or as a two-dose schedule spaced by, for example, 56 days in healthy adults (18-55 years old) and healthy elderly (≥65 years old). Vaccine elicited S specific antibody levels are measured, for example, by ELISA and neutralizing titers are measured, for example, in a microneutralization assay (see, e.g., methods in Example 11). CD4+T-helper (Th)1 and Th2, and CD8+ immune responses are assessed, for example, by intracellular cytokine staining (ICS).

### Example 7. Generation of Codon-Optimized SARS-CoV-2 Spike Protein (SARS-CoV-2-Spike-Co)

The SARS-CoV-2 Spike protein (SEQ ID NO: 45) was codon-optimized (SARS-CoV-2-Spike-co; SEQ ID NO: 50) for expression during poxvirus infection and was synthesized by GenScript. The synthesized DNA also contains a poxvirus synthetic early/late promoter at nucleotide position 10-48. The synthesized DNA was subcloned into a plasmid containing homology to either the HPXV095 gene locus (SEQ ID NO: 51) or the HPXV200 gene locus (SEQ ID NO: 52). Homologous recombination was used to insert the synthesized DNA by replacing the selectable markers that were previously inserted into the synthetic VACV (synVACV) or synthetic HPXV (scHPXV). The selectable markers were inserted as a fusion between yellow fluorescent protein (YFP) and guanine phosphoriosyltransferase (GPT) into either of the HPXV095 or A2K105 genes, respectively (see methods as disclosed in US 2018/0251736, incorporated herein by reference in its entirety).

### Example 8: Generation of Synthetic Vaccinia Virus TNX-2200

The YFP-GPT selectable marker in the synVACV (see Example 2) thymidine kinase (TK) locus (also referred to as the A2K105 gene locus) is replaced using, for example, homologous recombination with a codon-optimized SARS-CoV-2 Spike (SARS-CoV-2-co) nucleotide sequence to generate the synthetic vaccinia virus TNX-2200. One exemplary procedure is as follows.

Approximately 20 μgrams of plasmid containing the SARS-CoV-2-Spike-co nucleotide sequence flanked by approximately 400 nucleotides homologous to the A2K105 gene was linearized using the restriction enzyme SacI. Following restriction enzyme digestion, the linearized plasmid was further purified to remove residual enzyme. BSC-40 cells were infected with synVACV expressing YFP-GPT in the A2K105 gene locus (synVACVΔ A2K105^{yfp-gpt}) at a MOI of 0.1 for 1 hour. Following infection, the virus inoculum was replaced with OptiMEM media and was incubated for an additional 30 minutes at 37° C. Approximately 5 μgrams of purified linearized plasmid was mixed with Lipofectamine 2000 (ThermoFisher Scientific) at a ratio of 1 μgram of DNA to 3 μL of Lipofectamine 2000 in a total volume of 2 mL of OptiMEM. A DNA-lipid complex formed during approximately 10 minutes of incubation. It was then added to the virus-infected BSC-40 cells.

BSC-40 cells were incubated for 48 hours to allow for homologous recombination to occur. After 48 hours, the plates were scraped to lift virus-infected cells and the mixture was transferred to a conical tube. The cells were lysed following three rounds of freezing at −80° C. and thawing. An appropriate dilution, which can range from 1×10⁻² to 1×10⁻⁵, of the infection/transfection mixture was plated onto BSC-40 cells followed by an agar overlay. Infected cell plates were incubated until non-fluorescent “recombinant” plaques were visualized. These non-fluorescent plaques were marked, and agar plugs were picked and added into a 10 mM Tris pH 8.0 solution. The plaques were subsequently used to infect BSC-40 cells in a second round of infection. This plaque picking process and infection of BSC-40 cells was repeated until YFP was undetectable in the infected cells (ranges between 4-6 rounds of purification). PCR analysis using primers (sA2K J2R Flank Forward Primer 5′ to 3′: ATGCGATTCAAAAAAGAATCAGC (SEQ ID NO: 56) and sA2K J2R Flank Reverse Primer 5′ to 3′: CAATTTCCTCAAAATACATAAACGG (SEQ ID NO: 57)) that amplify the A2K105 gene locus was performed to confirm that the SARS-CoV-2 Spike gene was inserted into the A2K105 locus.

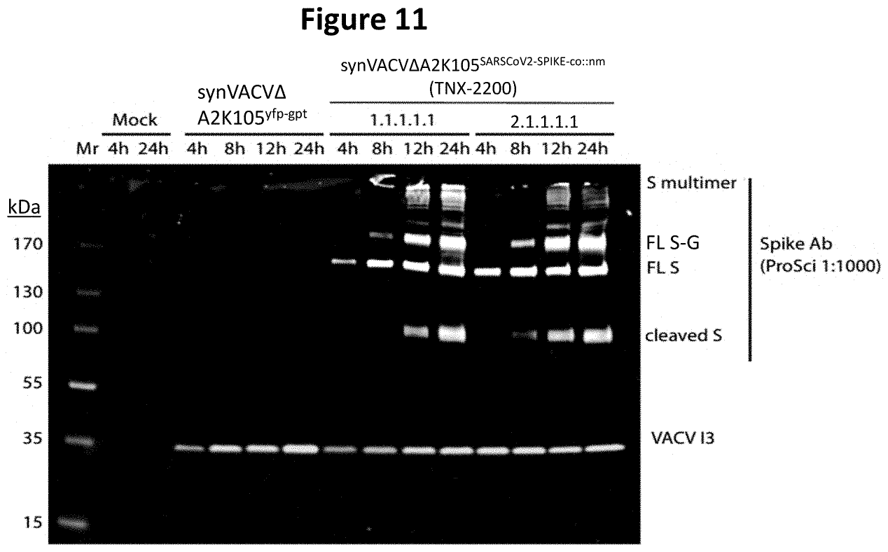

Western blot analysis was performed to test for SARS-Spike-co protein expression in the BSC-40 cells infected with synVACVΔA2K105^{yfp-gpt} or synVACVΔA2K105^{SARSCoV2-SPIKE-co:nm} (TNX-2200) clones 1.1.1.1.1 or 2.1.1.1.1 (FIG. 11). BSC-40 cells were infected with MOI 1.0 with the indicated viruses or with an inoculum without virus (mock), and protein lysates were harvested using RIPA lysis buffer at the indicated time points. SDS-PAGE was used to separate protein lysates and then the protein was transferred onto a nitrocellulose membrane. The membrane was subsequently blotted using anti-SARS-CoV2 Spike (ProSci) or anti-VACV 13 antibodies. Primary antibody binding was detected by blotting the membrane with IRDye secondary antibodies detectable at 800 nm or 680 nm channels (LI-COR). The SARS CoV2 Spike antibody detected different forms of the SARS-CoV-2 Spike protein including the full-length, glycosylated full-length, cleaved, and multimeric forms.

Viral genomic DNA from synVACVΔA2K105^{SARSCoV2-SPIKE-co::nm} (TNX-2200) clones 1.1.1.1.1 and 2.1.1.1.1 was isolated and the DNA was sequenced using Next Generation Sequencing (NGS) with the Illumina MiSeq platform. The sequencing data were analyzed by de novo assembly and mapped to reference software using the CLC Genomics Workbench software (Qiagen).

### Example 9. Generation of Synthetic Horsepox Virus TNX-1800a

The YFP-GPT selectable marker in the scHPXV (see Example 7) thymidine kinase (TK) locus (also referred to as the HPXV095 gene locus) was replaced using, for example, homologous recombination with a codon-optimized SARS-CoV-2 Spike (SARS-CoV-2-co) nucleotide sequence to generate the synthetic vaccinia virus TNX-1800a. One exemplary procedure is as follows.

Approximately 20 μgrams of plasmid containing the SARS-CoV-2-Spike-co nucleotide sequence flanked by approximately 400 nucleotides homologous to the HPXV095 gene was linearized using the restriction enzyme, SacI. Following restriction enzyme digestion, the linearized plasmid was further purified to remove residual enzyme. BSC-40 cells were infected with scHPXV expressing YFP-GPT in the HPXV095 gene locus at a MOI of 0.1 for 1 hour. Following infection, the virus inoculum was replaced with OptiMEM media and was incubated for an additional 30 minutes at 37° C. Approximately 5 μgrams of purified linearized plasmid was mixed with Lipofectamine 2000 (ThermoFisher Scientific) at a ratio of 1 μgram of DNA to 3 of Lipofectamine 2000 in a total volume of 2 mL of OptiMEM. A DNA-lipid complex formed during approximately 10 minutes of incubation. It was then added to the virus-infected BSC-40 cells.

BSC-40 cells were incubated for 48 hours to 72 hours to allow for homologous recombination to occur. Subsequently, the plates were scraped to lift virus-infected cells and the mixture was transferred to a conical tube. The cells were lysed following 3 rounds of freezing at −80° C. and thawing. An appropriate dilution, which can range from 1×10⁻² to 1×10⁻⁵, of the infection/transfection mixture was plated onto BSC-40 cells followed by an agar overlay. Infected cell plates were incubated until non-fluorescent “recombinant” plaques were visualized. These non-fluorescent plaques were marked, and agar plugs were picked and added into a 10 mM Tris pH 8.0 solution. The plaques were subsequently used to infect BSC-40 cells in a second round of infection. This plaque picking process and infection of BSC-40 cells was repeated until YFP was undetectable in the infected cells (ranges between 4-6 rounds of plaque purification). One non-fluorescent plaque was isolated from the low efficiency of homologous recombination in the HPXV-infected cells.

PCR analysis using primers (sA2K/HPXV J2R Flank Forward Primer 5′-3′: TATCGCATTTTCTAACGTGATGG (SEQ ID NO: 58) and sA2K/HPXV J2R Flank Reverse Primer 5′-3′: CCTCATTTGCACTTTCTGGTTC (SEQ ID NO: 59)) that amplify the HPXV095 gene locus was performed to confirm that the SARS-Spike-co gene was inserted into the HPXV095 locus. The viral genomic DNA was subsequently isolated from a preparation of sucrose-purified virus particles and used in Next Generation Sequencing with the Illumina MiSeq platform. The sequence data was analyzed by de novo assembly and mapped to reference software using the CLC Genomics Workbench software (Qiagen).

### Example 10. Generation of Synthetic Horsepox Virus TNX-1800b

The YFP-GPT selectable marker in the scHPXV (see Example 7) HPXV200 gene locus (also referred to as the Variola virus B22R homolog locus) was replaced using, for example, homologous recombination with a codon-optimized SARS-CoV-2 Spike (SARS-CoV-2-co) nucleotide sequence to generate the synthetic vaccinia virus TNX-1800b. One exemplary procedure is as follows.

Approximately 20 μgrams of plasmid containing SARS-CoV-2-Spike-co flanked by approximately 400 nucleotides homologous to the HPXV200 gene was linearized using the restriction enzyme, SacI. Following restriction enzyme digestion, the linearized plasmid was further purified to remove residual enzyme. BSC-40 cells were infected with scHPXV expressing YFP-GPT in the HPXV200 gene locus at a MOI of 0.1 for 1 hour. Following infection, the virus inoculum was replaced with OptiMEM media and incubated for an additional 30 minutes at 37° C. Approximately 5 μgrams of purified linearized plasmid was mixed with Lipofectamine 2000 (ThermoFisher Scientific) at a ratio of 1 μgram of DNA to 3 μL of Lipofectamine 2000 in a total volume of 2 mL of OptiMEM. A DNA-lipid complex formed during approximately 10 minutes of incubation. It was then added to the virus-infected BSC-40 cells.

BSC-40 cells were incubated for 48 hours to 72 hours to allow for homologous recombination to occur. Subsequently, the plates were scraped to lift virus-infected cells and the mixture was transferred to a conical tube. The cells were lysed following three rounds of freezing at −80° C. and thawing. An appropriate dilution, which can range from 1×10⁻² to 1×10⁻⁵, of the infection/transfection mixture was plated onto BSC-40 cells followed by an agar overlay. Infected cell plates were incubated until non-fluorescent “recombinant” plaques were visualized. These non-fluorescent plaques were marked, and agar plugs were picked and added into a 10 mM Tris pH 8.0 solution. These plaques were subsequently used to infect BSC-40 cells in a second round of infection. One non-fluorescent plaque was isolated due to low efficiency of homologous recombination in HPXV-infected cells compared to VACV-infected cells. The plaque picking process was repeated by infecting BSC-40 cells until YFP was undetectable (about 4-6 rounds of plaque purification).

PCR analysis using primers (sHPXV 200 Flank Forward Primer 5′-3′: ATAGCCACAATTATTGACGGGC (SEQ ID NO: 60) and sHPXV 200 Flank Reverse Primer 5′-3′: ggatgatatggtaatgtaactaccgatac (SEQ ID NO: 61)) that amplify the HPXV200 gene locus was performed to confirm that the SARS-Spike-co gene was inserted into the HPXV200 locus. The viral genomic DNA was subsequently isolated from a preparation of sucrose-purified virus particles and used for Next Generation Sequencing with the Illumina MiSeq platform. The sequence was analyzed by de novo assembly and mapped to reference software using the CLC Genomics Workbench software (Qiagen).

### Example 11. SARS-CoV-2 Spike Protein Analysis in TNX-1800a and TNX-1800b

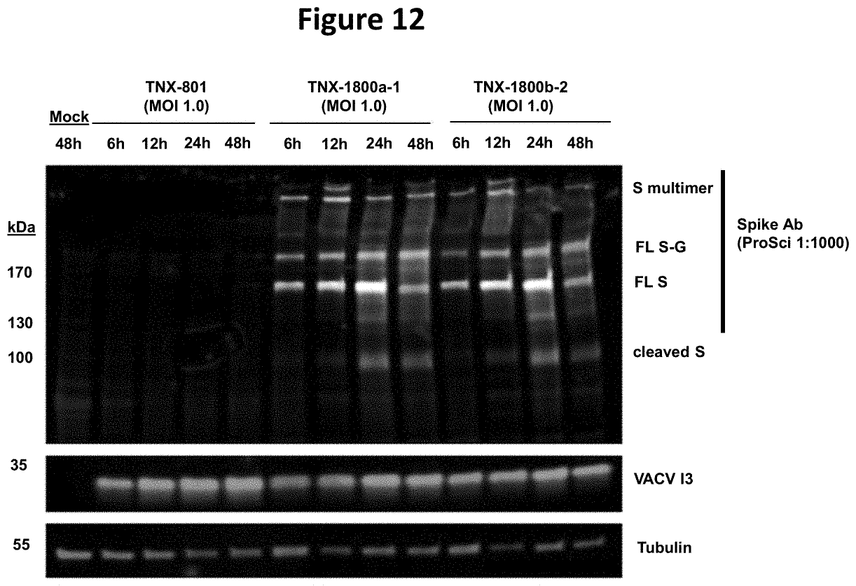

Western blot analysis was performed to assess SARS-Spike-co protein expression in the BSC-40 cells infected with TNX-801, TNX-1800a (clone TNX-1800a-1) and TNX-1800b (clone TNX-1800b-2) (FIG. 12). BSC-40 cells were infected with MOI 1.0 with the indicated viruses and protein lysates were harvested using RIPA buffer at the indicated time points. SDS-PAGE was used to separate protein lysates and then the protein was transferred onto a nitrocellulose membrane. The membrane was subsequently blotted using anti-SARS-CoV2 Spike (ProSci), anti-VACV 13 or anti-Tubulin antibodies. Fluorescently tagged secondary antibodies were used to detect the binding of primary antibodies. The SARS CoV2 Spike antibody detected different forms of the SARS-CoV-2 Spike protein including the full-length, glycosylated full-length, cleaved, and multimeric forms.

### Example 12. Immunization of African Green Monkeys with a Recombinant Poxvirus Engineered SARS-CoV-2 S Protein

Methods of immunization and testing candidate vaccines in African Green Monkeys has been previously described (Hartman, A. et al. (2020) SARS-CoV-2 infection of African green monkeys result in mild respiratory disease discernible by PET/CT imaging and shedding of infectious virus from both respiratory and gastrointestinal tracts. PLOS Pathogens 16(9): e1008903; incorporated herein by reference in its entirety). African Green Monkeys (AGMs) were randomly separated into 6 groups (n=4) and vaccinated with different strains of a synthetic horsepox virus (HPXV). See Table 5 for strain and dose. At day 0, AGMs were vaccinated percutaneously via scarification using a bifurcated needle.

TABLE 5
Doses of HPXV strains Used to Vaccinate African Green Monkeys

| | Number of | Animal | | |
|---|---|---|---|---|
| Group | Animals | Number | HPXV strain | Dose (PFU) |
| 1 | 4 | 1F 16986 | TNX-801 | 2.9 × 10⁶ |
| | | 1F 16994 | | |
| | | 1M 16975 | | |
| | | 1M 16977 | | |
| 2 | 4 | 2F 16985 | TNX-801 | 1.06 × 10⁶ |
| | | 2F 16991 | | |
| | | 2M 16980 | | |
| | | 2M 16983 | | |
| 3 | 4 | 3F 16988 | TNX- | 2.9 × 10⁶ |
| | | 3F 16995 | 1800b-2 | |
| | | 3M 16976 | | |
| | | 3M 16982 | | |
| 4 | 4 | 4F 16989 | TNX- | 1.06 × 10⁶ |
| | | 4F 16990 | 1800b-2 | |
| | | 4M 16972 | | |
| | | 4M 16973 | | |
| 5 | 4 | 5F 16992 | TNX- | 0.6 × 10⁶ |
| | | 5F 16993 | 1800a-1 | |
| | | 5M 16979 | | |
| | | 5M 16981 | | |
| 6 | 4 | 6M 16978 | Vehicle | Not |
| | | 6M 16974 | Control | applicable |
| | | 6F16987 | | |
| | | 6F16984 | | |

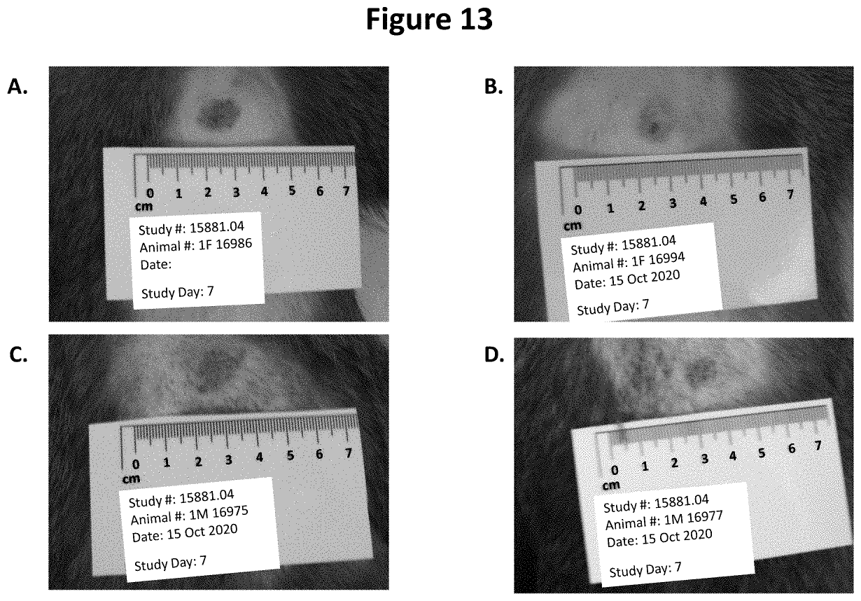

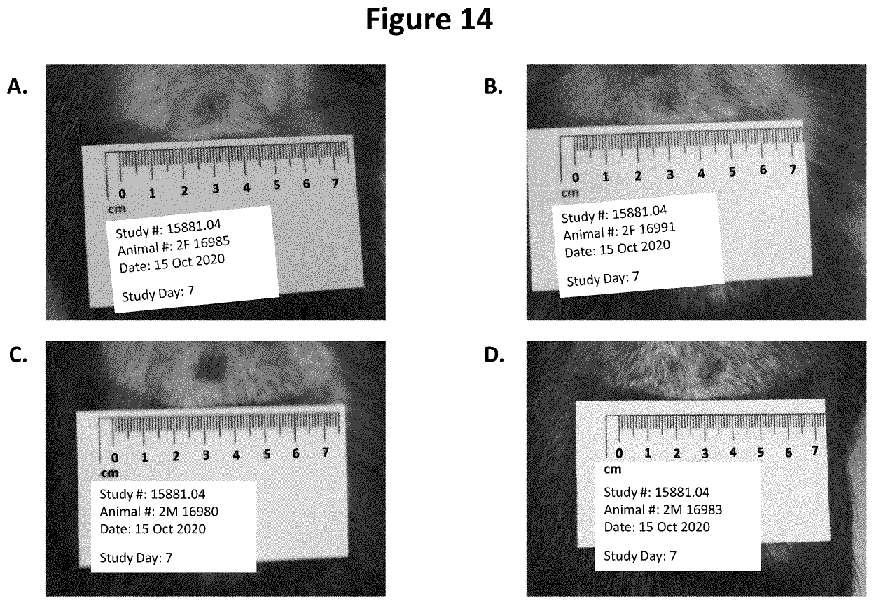

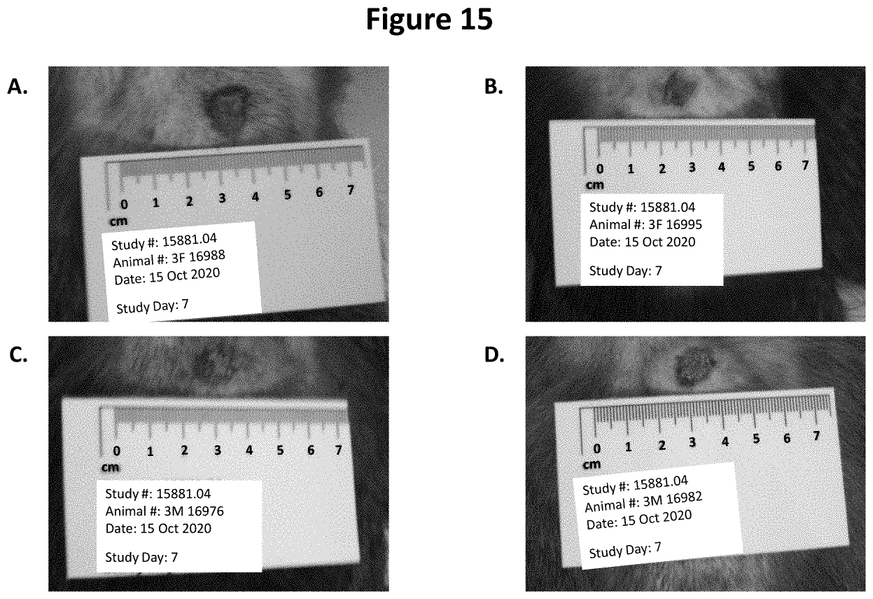

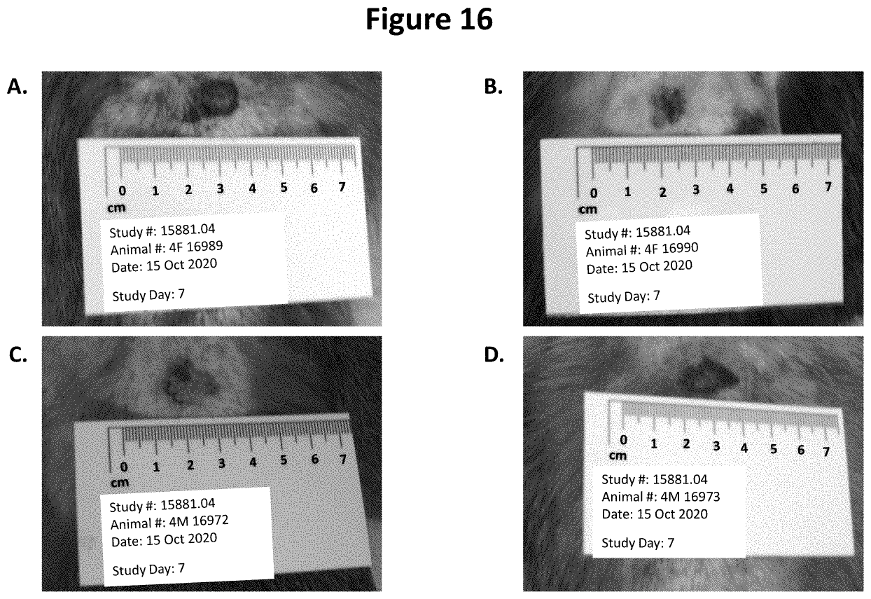

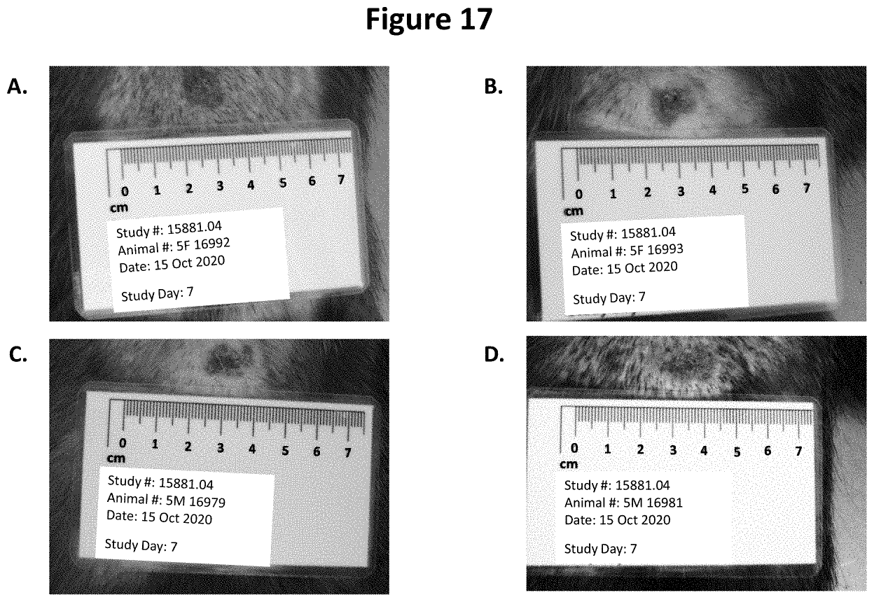

The inoculation site of the AGMs was monitored and after 7 days presented with a cutaneous reaction, also known as a “take”, when vaccinated with TNX-801, TNX-1800b-2 or TNX-1800a-1 regardless of the dose eliciting an immune response, including a T cell immune response (FIGS. 13-17). A “take” has been previously described as a biomarker of a positive vaccine response indicating protective immunity (e.g., T cell immunity) against a vaccinia virus, such as smallpox (Jenner, E., 1800, 2^{nd} Ed. “An Inquiry into the Causes and Effects of the Variolae Vaccinae, a Disease Discovered in Some of the Western Counties of England, Particularly Gloucestershire, and Known by the Name of The Cow Pox”). The “take” is a measure of functional T cell immunity validated by the eradication of smallpox, a respiratory-transmitted disease caused by variola, in the 1960's. The presence of a “take” sited on AGMs after vaccination with TNX-1800b-2 or TNX-1800a-1 is predictive that a T cell immune response will be activated due to the introduction of the SARS-CoV-S protein, a COVID-19 antigen. The T cell immune response is activated when naïve T cells are presented with antigens (e.g., SARS-CoV-2 S protein), leading to naïve T cell differentiation and proliferation. This response also leads to immunological memory by generating memory T cells which provide protection and an accelerated immune response from subsequent challenge by the same antigen. On day 60, the vaccinated AGMs are challenged with SARS-CoV-2 via the intratracheal route and the challenges show that the vaccination provides a protective immunity against the virus. The surviving animals are euthanized on Day 88.

A Microneutralization Assay was performed 14 days after the AGMs were vaccinated with the indicated HPXV strains to assess the anti-SARS-CoV-2 neutralizing titers in the serum. The assay was initially performed in duplicate and a third replicate was performed if the first two replicates were not within a 2-fold dilution of each other. Serum samples were initially heat inactivated at 56° C. for 30-60 minutes after being aliquoted onto a master plate. The master plates can be stored at 4-8° C. for seven days or at −20° C. for three months.

Vero E6 cells (ATCC) at a concentration 2×10⁴ cells per well were seeded into 96-well plates 18-24 hours before addition of the serum test samples. On the day of the assay, master plates were thawed and nine serum test samples were 2-fold serial diluted from 1:5 to 1:640 on a separate 96-well plate/dilution block (columns 1-9). Additionally, each 96-well plate/dilution block contained a positive control serum (column 10), virus controls (column 11) and cell controls (column 12). After dilution, an equal volume of virus stock (1,000 TCID50/mL) is added to columns 1-11. In addition, assay quality control (QC) plates were set up at the same time consisting of positive control serum (columns 1-2), a negative control (columns 3-4), viral input back titer (columns 5-6), virus control (VC; columns 7-9) and cell controls (CC; columns 10-12). At least two QC plate were used per assay. Test and QC plates were incubated at 37° C. for 2-2.5 hours in a 5% CO₂ incubator. After incubation, aliquots of mixtures (sera and virus) for both test and QC plates (including controls) were transferred onto the 96-well plates pre-seeded with Vero E6 cell and incubated for 72±4 hours. Following incubation, plates were removed from the incubator and allowed to rest at room temperature for 20-40 minutes. 100 uL of Cell Titer-Glo (Promega) was added to all wells in the plates, gently mixed and incubated at room temperature for 10-30 minutes. Luminescence was read using an appropriate photometer. Plate cut-off values were calculated using the following formula:

(Average of VC wells+Average of CC wells)/2

Samples with luminescence above or below the plate cut-off are positive and negative for neutralizing antibody, respectively. The individual replicate is assigned a titer that is the reciprocal of the dilution of the last positive dilution (i.e., 1:80=is reported as a titer of 80). Titers are reported as median and geometric mean titers of the accepted replicate titers.

Table 6 shows the level of anti-SARS-CoV-2 neutralizing titers measured in vaccinated AGMs after 14 days of a single vaccination. The AGMs vaccinated with TNX-1800b-2 and TNX1800a-1 generated neutralizing titers (≥1:40 titer) of antibodies against SARS-CoV-2. The TNX-801 (an scHPXV not carrying the S protein expression cassette) vaccinated control animals and the placebo group did not generate anti-SARS-CoV-2 neutralizing titers (≤1:10 titer). Both the 2.9×10⁶ PFU and 1.06×10⁶ PFU doses of TNX-801 and TNX-1800 were well-tolerated.

TABLE 6,
Anti-SARS-CoV-2 neutralizing titers in vaccinated African Green Monkeys

| | | | | | | Geometric |
|---|---|---|---|---|---|---|
| Animal | HPXV | | | | | Mean Titer |
| Number | strain | Dose | Titer 1 | Titer 2 | Median | (GMT) |
| 3M 16982 | TNX- | 2.9 × 10⁶ | 640 | 20 | NQ | NQ |
| D15 | 1800b-2 | | | | | |
| 3M 16976 | TNX- | 2.9 × 10⁶ | 640 | 320 | 480 | 452.55 |
| D15 | 1800b-2 | | | | | |
| 3F 16988 | TNX- | 2.9 × 10⁶ | 320 | 160 | 240 | 226.27 |
| D15 | 1800b-2 | | | | | |
| 3F 16995 | TNX- | 2.9 × 10⁶ | 640 | 640 | 640 | 640.00 |
| D15 | 1800b-2 | | | | | |
| 4M 16973 | TNX- | 1.06 × 10⁶ | 160 | 160 | 160 | 160.00 |
| D14 | 1800b-2 | | | | | |
| 4M 16972 | TNX- | 1.06 × 10⁶ | 640 | 640 | 640 | 640.00 |
| D14 | 1800b-2 | | | | | |
| 4F 16989 | TNX- | 1.06 × 10⁶ | 80 | 80 | 80 | 80.00 |
| D14 | 1800b-2 | | | | | |
| 4F 16990 | TNX- | 1.06 × 10⁶ | 320 | 320 | 320 | 320.00 |
| D14 | 1800b-2 | | | | | |
| 5M 16979 | TNX- | 0.6 × 10⁶ | 320 | 320 | 320 | 320.00 |
| D14 | 1800a-1 | | | | | |
| 5M 16981 | TNX- | 0.6 × 10⁶ | 640 | 320 | 480 | 452.55 |
| D14 | 1800a-1 | | | | | |
| 5F 16993 | TNX- | 0.6 × 10⁶ | 320 | 320 | 320 | 320.00 |
| D14 | 1800a-1 | | | | | |
| 5F 16992 | TNX- | 0.6 × 10⁶ | 320 | 640 | 480 | 452.55 |
| D14 | 1800a-1 | | | | | |

### Example 13. Viral Growth Curves Measured in Cells Infected with Recombinant Poxvirus Engineered SARS-CoV-2 S Protein

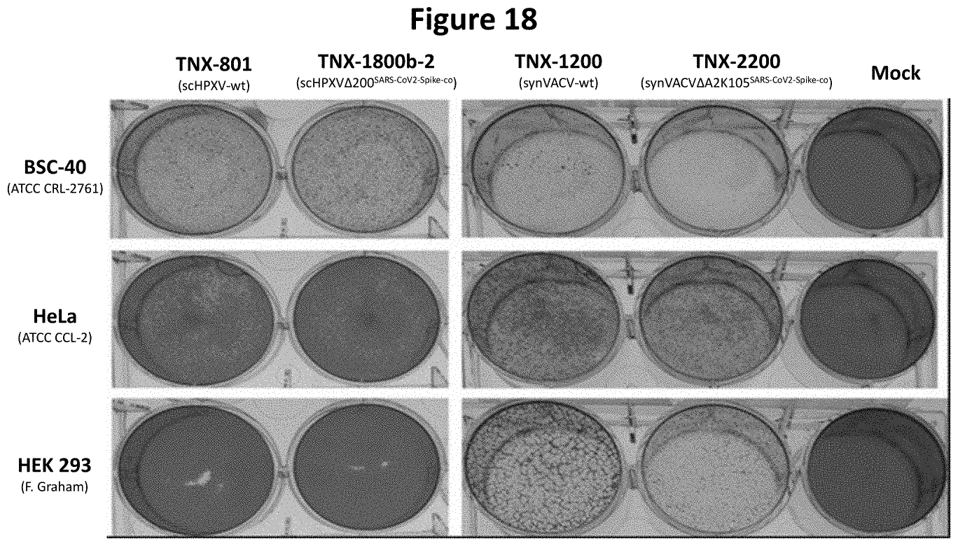

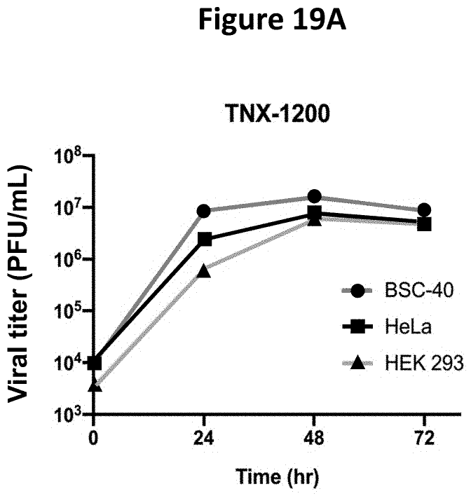

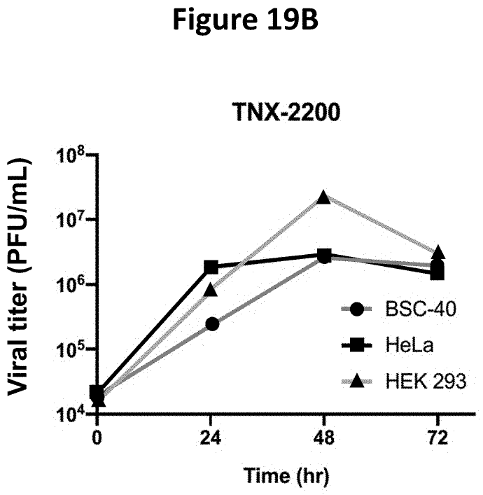

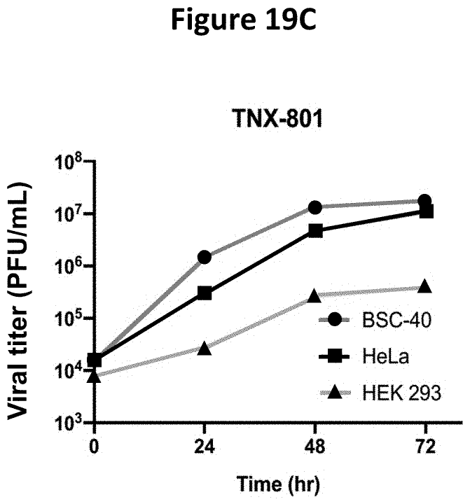

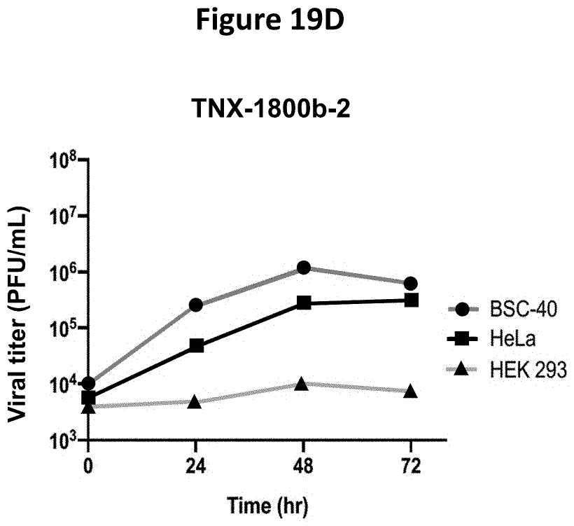

BSC-40, HeLa and HEK 293 cells were seeded into a 6-well plate and subsequently infected with TNX-801, TNX-1800, TNX-1200, or TNX-2200 at a MOI of 0.01. After 48 hours of infection, cells were fixed and stained with 5% formaldehyde containing crystal violet. BSC-40 cells infected with TNX-801 and TNX-1800 had a significant cytopathic effect, while HeLa and HEK 293 cells showed minor and no cytopathic effect, respectively (FIG. 18). BSC-40 HeLa and HEK293 cells infected with TNX-1200 and TNX-2200 had a significant cytopathic effect in all infected cell lines (FIG. 18). Viral titer (PFU/mL) in BSC-40, HeLa and HEK 293 cells was measured over time after 24, 48 and 72 hours of infection with TNX-801, TNX-1800, TNX-1200, or TNX-2200 (FIGS. 19A-D), which corresponds to the cytopathic effect of the viruses as represented in FIG. 18.

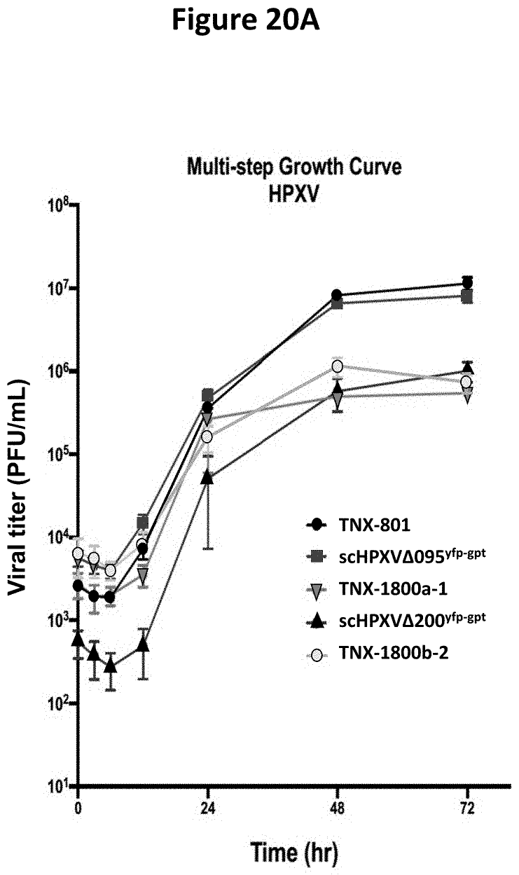

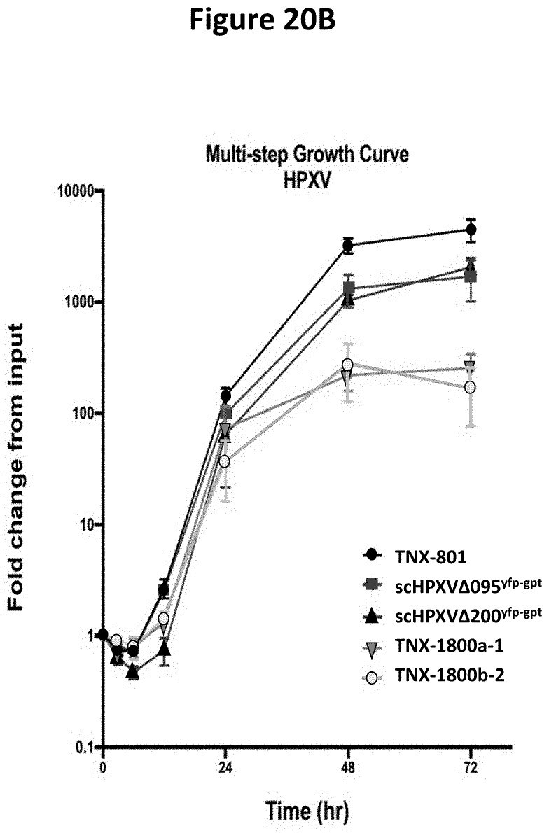

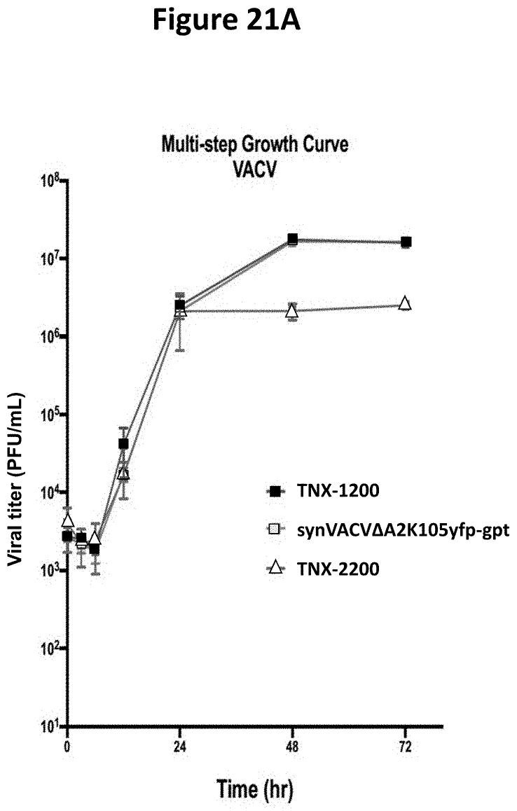

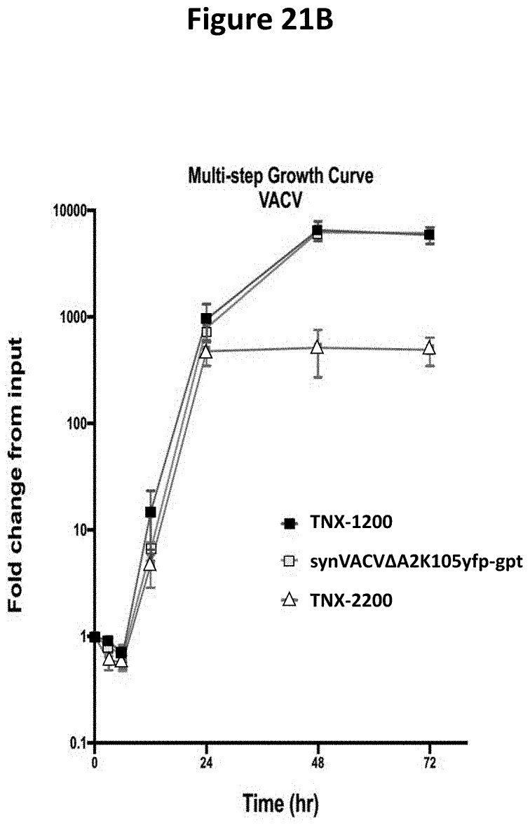

BSC-40 cells were infected with HPXV clones (e.g., _TNX-801, scHPXVΔ095^{yfp-gpt}, TNX-1800a-1, scHPXVΔ200^{yfp-gpt}, or TNX-1800b-2; (FIGS. 20A-B)) or VACV clones (e.g., TNX-1200, TNX-2200 or synVACVΔA2K105^{yfp-gpt}; (FIGS. 21A-B)) at a MOI of 0.01. Viral titer (PFU/mL) was measured at 0, 3, 6, 12, 24, 48 and 72 hours to determine viral growth in infected cells. The presence of SARS-CoV-2 Spike protein slows HPXV clone viral growth by approximately 0.5 log, while it slows VACV clone viral growth by approximately 1 log.

The cytopathic effect seen in Vero cells and BSC-40 cells infected with the various HPXV and VACV clones shows that these cell lines can be used to manufacture the viruses (e.g., TNX-1800 and TNX-801).

### Example 14. Generation of a SARS-CoV-2 Spike Synthetic DNA Expression Cassette and Recombinant scHPXV Transfected with the Cassette

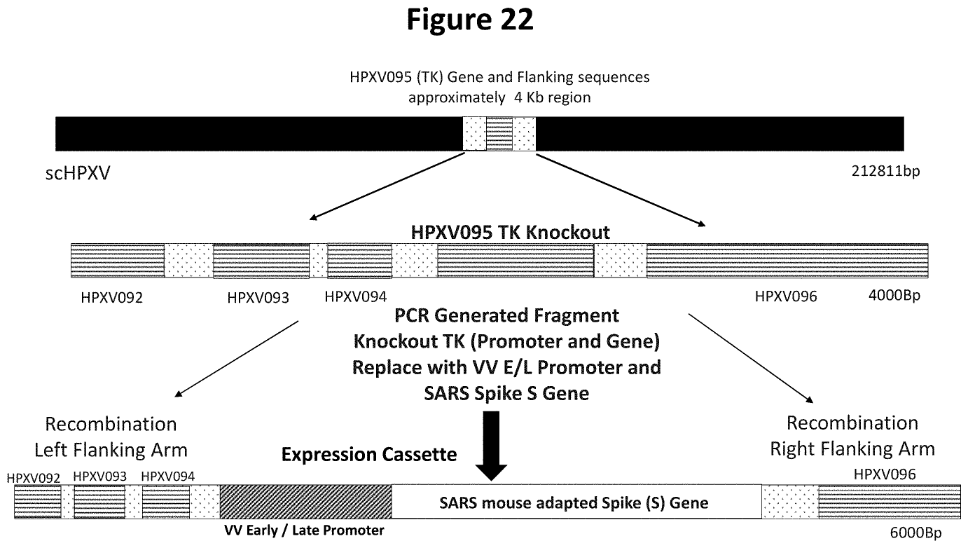

As illustrated in FIG. 22, SARS-CoV-2 Spike (S) nucleotide sequence (SEQ ID NO: 45) is modified by removing the Early Transcription Terminator Signal (T₅NT) (SEQ ID NO: 14) using silent coding mutagenesis thereby retaining the SARS-CoV-2 Spike (S) protein coding sequences.

The location of an insertion site for the heterologous transgene SARS-CoV-2 Spike (S) within the DNA nucleotide sequence of a synthetic chimeric (sc) Horsepox genome is selected (for example the TK gene locus HPXV095; positions 992077-92610; SEQ ID NO:1). The DNA nucleotide sequences proximal to the left and right of the selected HPXV insertion site, which define the Left and Right Flanking arms, are identified (see FIG. 22). Those arms are used to drive homologous nucleotide site specific recombination between the rescue virus and heterologous transgene. A DNA nucleotide sequence encoding a poxvirus-based promoter for driving high levels of SARS-CoV-2 Spike (S) gene expression, such as the vaccinia virus Early/Late Promotor, is also selected.

One exemplary DNA nucleotide sequence of approximately 6 kb for a SARS-CoV-2 Spike (S) synthetic expression cassette, comprising the DNA nucleotide sequences of a Left Flanking Arm, a vaccinia virus Early/Late Promotor operably linked to the modified CoVID-SARS-2 Spike (S) nucleic acid sequence, and a Right Flanking Arm is then synthesized (e.g., by a commercial vendor (e.g., Genewiz)). See FIG. 22.

The SARS-CoV-2 Spike (S) Synthetic expression cassette DNA is then transfected into cells (e.g., BSC-40 cells) infected with an scHPXV. Recombinant horsepox viral progeny containing the SARS-CoV-2 Spike (S) synthetic expression cassette are selected using media containing BrdU so as to prevent viral amplification of the parental virus retaining the original insertion site viral genomic DNA sequences. The recombinant virus is purified using successive rounds of plaque purification. The nucleotide sequence from the purified virus across the entire SARS-CoV-2 Spike (S) heterologous transgene cassette is confirmed by sequence analysis (e.g., PCR sequence analysis). See SEQ ID NO: 63.

Similar constructs and steps can be carried out using a horsepox virus to generate a recombinant scHPXV containing a mouse adapted spike protein expression cassette (see SEQ ID NO: 64) and a vaccinia virus, using, for example, the vaccinia TK gene locus synVACV105; positions 83823-84344 (see SEQ ID NO: 2) to generate a recombinant vaccinia virus containing a mouse adapted spike protein expression cassette (see SEQ ID NO: 65).

### Example 15. Efficacy of Recombinant Poxvirus Carrying an Expression Cassette Encoding a SARS-CoV-2 S Protein in Immunized African Green Monkeys Challenged with SARS-CoV-2

At day 0, African Green Monkeys (AGMs) were vaccinated percutaneously via scarification using a bifurcated needle as described in Example 12. Table 7 shows the level of anti-SARS-CoV-2 neutralizing titers measured in vaccinated AGMs after 0, 7, 15, 21, 29, 41 and 47 days of a single vaccination. The AGMs vaccinated with TNX-1800b-2 and TNX1800a-1 generated neutralizing titers (≥1:40 titer) of antibodies against SARS-CoV-2. The TNX-801 (an scHPXV not carrying the S protein expression cassette) vaccinated control animals and the placebo group did not generate anti-SARS-CoV-2 neutralizing titers (≤1:10 titer). Both the 2.9×10⁶ PFU and 1.06×10⁶ PFU doses of TNX-801 and TNX-1800 were well-tolerated.

TABLE 7
Anti-SARS-CoV-2 neutralizing titers in vaccinated African Green Monkeys

| | | | Titer | Titer | Titer | Titer | | | |
|---|---|---|---|---|---|---|---|---|---|
| HPXV | Dose | Animal | Day | Day | Day | Day | Titer | Titer | Titer |
| strain | (PFU) | Number | 0 | 7 | 15 | 21 | Day 29 | Day 41 | Day 47 |
| TNX-801 | 2.9 × 10⁶ | IM 16977 | NQ | 5.00 | 7.07 | 5.00 | 5.00 | 5.00 | 5.00 |
| | | IM 16975 | 7.07 | 7.07 | 2.50 | 5.00 | 5.00 | 5.00 | 5.00 |
| | | IF 16994 | 5.00 | 5.00 | 2.50 | 5.00 | 5.00 | 5.00 | 5.00 |
| | | IF 16986 | 5.00 | 7.07 | 7.07 | 5.00 | 5.00 | 5.00 | 5.00 |
| TNX-801 | 1.06 × 10⁶ | 2M 16980 | 5.00 | 5.00 | 2.50 | 5.00 | 5.00 | 5.00 | 5.00 |
| | | 2M 16983 | 5.00 | 5.00 | 2.50 | 5.00 | 5.00 | 5.00 | 5.00 |
| | | 2F 16985 | 5.00 | 5.00 | 3.54 | 5.00 | 5.00 | 5.00 | 5.00 |
| | | 2F 16991 | 5.00 | 5.00 | 2.50 | 5.00 | 5.00 | 5.00 | 5.00 |
| TNX- | 2.9 × 10⁶ | 3M 16982 | 5.00 | 5.00 | 113.14 | 113.14 | 40.00 | 56.57 | 1280.00 |
| 1800b-2 | | 3M 16976 | 7.07 | 5.00 | 80.00 | 113.14 | 40.00 | 80.00 | 640.00 |
| | | 3F 16988 | 5.00 | 5.00 | 113.14 | 160.00 | 80.00 | 160.00 | 320.00 |
| | | 3F 16995 | 5.00 | 5.00 | 320.00 | 226.27 | 40.00 | 56.57 | 1280.00 |
| TNX- | 1.06 × 10⁶ | 4M 16973 | 5.00 | 5.00 | 113.14 | 226.27 | 113.14 | 80.00 | 905.10 |
| 1800b-2 | | 4M 16972 | 5.00 | 5.00 | 452.55 | 452.55 | 320.00 | 320.00 | NQ |
| | | 4F 16989 | 5.00 | 5.00 | 56.57 | 28.28 | 14.14 | 40.00 | 1280.00 |
| | | 4F 16990 | 5.00 | 5.00 | 320.00 | 226.27 | 80.00 | 160.00 | 905.10 |
| TNX- | 0.6 × 10⁶ | 5M 16979 | 5.00 | 5.00 | 160.00 | 113.14 | 113.14 | NQ | 226.27 |
| 1800a-1 | | 5M 16981 | 5.00 | 5.00 | 226.27 | 160.00 | 80.00 | 80.00 | 452.55 |
| | | 5F 16993 | 7.07 | 5.00 | 113.14 | NQ | 56.57 | 56.57 | 160.00 |
| | | 5F 16992 | 7.07 | 5.00 | 226.27 | 640.00 | NQ | 226.27 | 452.55 |
| Vehicle | Not | 6M 16978 | 5.00 | 5.00 | 2.50 | 5.00 | 5.00 | 5.00 | 5.00 |
| Control | applicable | 6M 16974 | 5.00 | 5.00 | 3.54 | 5.00 | 5.00 | 5.00 | 5.00 |
| | | 6F16987 | 7.07 | 5.00 | 3.54 | 5.00 | 5.00 | 5.00 | 5.00 |
| | | 6F16984 | 7.07 | 5.00 | 3.54 | 5.00 | 5.00 | 5.00 | 5.00 |

At day 41, the vaccinated AGMs were anesthetized and challenged (also referred to as inoculated) with approximately 2×10⁶ TCID₅₀/animal wild-type SARS-CoV-2 via the 1. intranasal and 2. intratracheal route. The volume of virus was split evenly between each of the two routes (1 mL per route with a 1×106 TCID₅₀/mL virus stock). For the intranasal route, AGMs were anesthetized and inoculated by slowly pipetting 500 μL into each are followed by inhalation. For the intratracheal route, AGMs were anesthetized, and a tube was inserted into the trachea. After the end of the tube was situated approximately at the mid-point of the trachea, a syringe containing the inoculate with the virus was attached to the tube and the inoculate was slowly instilled into the trachea followed by an equal volume of PBS to flush the tube. After the AGMs were inoculated, the animal was returned to its home cage and monitored for recovery from the anesthesia.

An oropharyngeal swab specimen and a tracheal lavage specimen were collected on Day 41 and Day 47 from the inoculated AGMs. The specimens were processed by RT-qPCR methods to measure SARS-CoV-2 copy number. Table 8 shows the SARS-CoV-2 copy number from oropharyngeal swab specimens. Table 9 shows the SARS-CoV-2 copy number from tracheal lavage specimens. At Day 47, AGMs vaccinated with TNX-1800b-2 and TNX-1800a-1 developed protective immunity against SARS-CoV-2.

TABLE 8
RT-qPCR of SARS-CoV-2 Copy Number
per Swab from Oropharyngeal Swab

| | | | Day 41 | Day 47 |
|---|---|---|---|---|
| HPXV | Dose | Animal | (Copy number | (Copy number |
| strain | (PFU) | Number | per swab) | per swab) |
| TNX-801 | 2.9 × 10⁶ | 1M 16977 | 0.00E+00 | 2.59E+06 |
| | | 1M 16975 | 0.00E+00 | 1.75E+05 |
| | | 1F 16994 | 0.00E+00 | 2.61E+03 |
| | | 1F 16986 | 0.00E+00 | 2.22E+04 |
| TNX-801 | 1.06 × 10⁶ | 2M 16980 | 0.00E+00 | 6.69E+03 |
| | | 2M 16983 | 0.00E+00 | 6.33E+04 |
| | | 2F 16985 | 0.00E+00 | 5.56E+04 |
| | | 2F 16991 | 2.47E+02 | 3.75E+03 |
| TNX- | 2.9 × 10⁶ | 3M 16982 | 0.00E+00 | 0.00E+00 |
| 1800b-2 | | 3M 16976 | 1.98E+02 | 0.00E+00 |
| | | 3F 16988 | 4.29E+02 | 0.00E+00 |
| | | 3F 16995 | 0.00E+00 | 0.00E+00 |
| TNX- | 1.06 × 10⁶ | 4M 16973 | 7.59E+03 | 0.00E+00 |
| 1800b-2 | | 4M 16972 | 0.00E+00 | 0.00E+00 |
| | | 4F 16989 | 0.00E+00 | 0.00E+00 |
| | | 4F 16990 | 0.00E+00 | 0.00E+00 |
| TNX- | 0.6 × 10⁶ | 5M 16979 | 0.00E+00 | 0.00E+00 |
| 1800a-1 | | 5M 16981 | 0.00E+00 | 0.00E+00 |
| | | 5F 16993 | 0.00E+00 | 4.68E+02 |
| | | 5F 16992 | 0.00E+00 | 0.00E+00 |
| Vehicle | Not | 6M 16978 | 0.00E+00 | 9.26E+03 |
| Control | applicable | 6M 16974 | 0.00E+00 | 3.66E+04 |
| | | 6F16987 | 0.00E+00 | 0.00E+00 |
| | | 6F16984 | 0.00E+00 | 1.53E+06 |

TABLE 9
RT-qPCR of SARS-CoV-2 Copy Number
per ml. from Tracheal Lavage

| | | | Day 41 | Day 47 |
|---|---|---|---|---|
| HPXV | Dose | Animal | (Copy number | (Copy number |
| strain | (PFU) | Number | per mL) | per mL) |
| TNX-801 | 2.9 × 10⁶ | IM 16977 | 0.00E+00 | 2.11E+06 |
| | | IM 16975 | 0.00E+00 | 0.00E+00 |
| | | IF 16994 | 0.00E+00 | 5.31E+02 |
| | | IF 16986 | 0.00E+00 | 3.61E+02 |
| TNX-801 | 1.06 × 10⁶ | 2M 16980 | 0.00E+00 | 4.50E+04 |
| | | 2M 16983 | 0.00E+00 | 0.00E+00 |
| | | 2F 16985 | 0.00E+00 | 3.95E+05 |
| | | 2F 16991 | 0.00E+00 | 1.72E+04 |
| TNX- | 2.9 × 10⁶ | 3M 16982 | 0.00E+00 | 0.00E+00 |
| 1800b-2 | | 3M 16976 | 0.00E+00 | 0.00E+00 |
| | | 3F 16988 | 0.00E+00 | 0.00E+00 |
| | | 3F 16995 | 0.00E+00 | 0.00E+00 |
| TNX- | 1.06 × 10⁶ | 4M 16973 | 0.00E+00 | 8.42E+02 |
| 1800b-2 | | 4M 16972 | 0.00E+00 | 0.00E+00 |
| | | 4F 16989 | 0.00E+00 | 0.00E+00 |
| | | 4F 16990 | 0.00E+00 | 0.00E+00 |
| TNX- | 0.6 × 10⁶ | 5M 16979 | 0.00E+00 | 0.00E+00 |
| 1800a-1 | | 5M 16981 | 0.00E+00 | 9.34E+03 |
| | | 5F 16993 | 0.00E+00 | 0.00E+00 |
| | | 5F 16992 | 0.00E+00 | 6.82E+02 |
| Vehicle | Not | 6M 16978 | 0.00E+00 | 1.91E+03 |
| Control | applicable | 6M 16974 | 0.00E+00 | 8.13E+03 |
| | | 6F16987 | 0.00E+00 | 1.43E+04 |
| | | 6F16984 | 0.00E+00 | 1.17E+03 |

### Exemplary Embodiments

1. A recombinant poxvirus comprising a nucleic acid encoding a SARS-CoV-2 virus protein, wherein the SARS-CoV-2 protein is selected from the group consisting of the spike protein (S), the membrane protein (M) and the nucleocapsid protein (N), or combinations of two or more of said proteins.
2. The recombinant poxvirus according to embodiment 1, wherein the poxvirus is an orthopoxvirus.
3. The recombinant poxvirus according to embodiment 2, wherein the orthopoxvirus is selected from the group consisting of camelpox (CMLV) virus, cowpox virus (CPXV), ectromelia virus (ECTV), horsepox virus (HPXV), monkeypox virus (MPXV), vaccinia virus (VACV), variola virus (VARV), rabbitpox virus (RPXV), raccoon poxvirus, skunkpox virus, Taterapox virus, Uasin Gishu disease virus and volepox virus.
4. The recombinant poxvirus according to embodiment 2, wherein the orthopoxvirus is a horsepox virus.
5. The recombinant poxvirus according to embodiment 4, wherein the horsepox virus is strain MNR-76.
6. The recombinant poxvirus according to embodiment 2, wherein the orthopoxvirus is a vaccinia virus.
7. The recombinant poxvirus according to embodiment 6, wherein the vaccinia virus is selected from the group of strains consisting of: Western Reserve, Western Reserve Clone 3, Tian Tian, Tian Tian clone TP5, Tian Tian clone TP3, NYCBH, NYCBH clone Acambis 2000 (ACAM 2000), Wyeth, Copenhagen, Lister, Lister 107, Lister-LO, Lister GL-ONC1, Lister GL-ONC2, Lister GL-ONC3, Lister GL-ONC4, Lister CTC1, Lister IMG2 (Turbo FP635), IHD-W, LC16m18, Lederle, Tashkent clone TKT3, Tashkent clone TKT4, USSR, Evans, Praha, L-IVP, V-VET1 or LIVP 6.1.1, Ikeda, EM-63, Malbran, Duke, 3737, CV-1, Connaught Laboratories, Serro 2, CM-01, NYCBH Dryvax clone DPP13, NYCBH Dryvax clone DPP15, NYCBH Dryvax clone DPP20, NYCBH Dryvax clone DPP17, NYCBH Dryvax clone DPP21, VACV-IOC, Mulford 1902, Chorioallantoid Vaccinia virus Ankara (CVA), Modified vaccinia Ankara (MVA), and MVA-BN.
8. The recombinant poxvirus according to any one of embodiments 1-7, wherein the SARS-CoV-2 protein is S protein.
9. The recombinant poxvirus according to any one of embodiments 1-8, wherein the amino acid sequence of the SARS-CoV-2 virus protein is modified with reference to a wild type protein.
10. The recombinant poxvirus according to embodiment 8, wherein the SARS-CoV-2 virus S protein is modified to infect mice.
11. The recombinant poxvirus according to embodiment 8, wherein the amino acid sequence of the SARS-CoV-2 virus S protein comprises one or more substitutions selected from Y459H, D614G, S943P, K986P and V987P, with reference to a wild type S protein (SEQ ID NO: 47).
12. The recombinant poxvirus according to any one of embodiments 1-11, wherein the nucleic acid encoding a SARS-CoV-2 virus protein is located in a region of the poxvirus that is not essential for replication of the poxvirus.
13. The recombinant poxvirus according to embodiment 12, wherein the nucleic acid encoding a SARS-CoV-2 virus protein is located in the thymidine kinase (TK) gene locus of the poxvirus.
14. The recombinant poxvirus according to embodiment 12, wherein the nucleic acid encoding a SARS-CoV-2 virus protein is located in the B22R homolog gene locus of the poxvirus.
15. The recombinant poxvirus according to any one of embodiments 1-14, wherein the nucleic acid encoding a SARS-CoV-2 virus protein is operatively linked to a promoter.
16. The recombinant poxvirus according to embodiment 15, wherein the promoter is a poxvirus-specific promoter.
17. The recombinant poxvirus according to embodiment 16, wherein the poxvirus specific promoter is a vaccinia virus early promoter.
18. The recombinant poxvirus according to embodiment 16, wherein the poxvirus specific promoter is a vaccinia virus late promoter.
19. The recombinant poxvirus according to embodiment 16, wherein the poxvirus specific promoter is a tandem of a vaccinia virus early and late promoter.
20. The recombinant poxvirus according to any one of embodiments 1-19, wherein the poxvirus is a synthetic poxvirus.
21. The recombinant poxvirus according to embodiment 20, wherein the recombinant poxvirus is selected from the group consisting of TNX-2200 (synVACVΔA2K105^{SARS-CoV2-Spike-co}), TNX-2200 clone 1.1.1.1.1, TNX-2200 clone 2.1.1.1.1, TNX-1800 (scHPXVΔ200^{SARS-COV2-Spike-co}), TNX-1800a, TNX-1800a-1, TNX-1800b, and TNX-1800b-2.
22. The recombinant poxvirus according to embodiment 21, wherein the recombinant poxvirus is TNX-1800b-2.
23. The recombinant virus according to embodiment 21, wherein the recombinant poxvirus is TNX-1800a-1.
24. The recombinant poxvirus according to embodiment 20, wherein the recombinant poxvirus comprises any one of SEQ ID NOs: 63, 64 or 65.
25. A pharmaceutical composition comprising a recombinant poxvirus according to any one of embodiments 1-24 and a pharmaceutically acceptable carrier.
26. The pharmaceutical composition according to embodiment 25, wherein the recombinant poxvirus is selected from the group consisting of TNX-2200 (synVACVΔA2K105^{SARS-CoV2-Spike-co}), TNX-2200 clone 1.1.1.1.1, TNX-2200 clone 2.1.1.1.1, TNX-1800 (scHPXVΔ200^{SARS-COV2-Spike-co}), TNX-1800a, TNX-1800a-1, TNX-1800b, and TNX-1800b-2.
27. The pharmaceutical composition according to embodiment 25, wherein the recombinant poxvirus comprises any one of SEQ ID Nos: 63, 64 or 65.
28. The pharmaceutical composition according to embodiment 26, wherein the recombinant poxvirus is TNX-1800b-2.
29. The pharmaceutical composition according to embodiment 26, wherein the recombinant poxvirus is TNX-1800a-1.
30. A cell infected with a recombinant poxvirus according to any one of embodiments 1-29.
31. The cell according to embodiment 30, wherein the cell is a mammalian cell.
32. The cell according to embodiment 31, wherein the mammalian cell is a Vero cell, a Vero E6 cell or a BSC-40 cell.
33. The cell according to embodiment 31, wherein the mammalian cell is a Vero adherent cell, a Vero suspension cell, a BHK-21 cell, an ACE2 Knockout Vero cell, or an MRC-5 cell.
34. The MRC-5 cell according to embodiment 33, grown in the presence of 5% fetal calf serum.
35. The cell according to embodiment 30, wherein the cell is an avian cell.
36. The cell according to embodiment 35, wherein the avian cell is a chicken embryo fibroblast, a duck embryo-derived cell, an EB66® cell, an AGE1.CRpIX® cell, or a DF-1 cell.
37. The cell according to embodiment 30, wherein the cell is an adherent cell.
38. The cell according to embodiment 30, wherein the cell is a suspension cell.
39. A method for selecting a cell that expresses a SARS-CoV-2 virus protein, comprising infecting said cell with a recombinant poxvirus according to any one of embodiments 1-24 and selecting the infected cell expressing said SARS-CoV-2 virus protein.
40. The method for selecting a cell that expresses a SARS-CoV-2 virus protein according to embodiment 39, wherein the recombinant poxvirus selected from the group consisting of TNX-2200 (synVACVΔA2K105^{SARS-CoV2-Spike-co}), TNX-2200 clone 1.1.1.1.1, TNX-2200 clone 2.1.1.1.1, TNX-1800 (scHPXVΔ200^{SARS-COV2-Spike-co}), TNX-1800a, TNX-1800a-1, TNX-1800b, and TNX-1800b-2.
41. The method for selecting a cell that expresses a SARS-CoV-2 virus protein according to embodiment 39, wherein the recombinant poxvirus comprises any one of SEQ ID Nos: 63, 64 or 65.
42. The method for selecting a cell that expresses a SARS-CoV-2 virus protein according to embodiment 40, wherein the recombinant poxvirus is TNX-1800b-2.
43. The method for selecting a cell that expresses a SARS-CoV-2 virus protein according to embodiment 40, wherein the recombinant poxvirus is TNX-1800a-1.
44. A method of inducing an immune response against a SARS-CoV-2 virus in a subject, comprising administering to said subject an immunologically effective amount of the recombinant poxvirus according to any one of embodiments 1-24 or the pharmaceutical composition according to any one of embodiments 25-29.
45. The method of inducing an immune response against a SARS-CoV-2 virus in a subject according to embodiment 44, wherein said immunologically effective amount of the recombinant poxvirus is administered by scarification.
46. The method of inducing an immune response against a SARS-CoV-2 virus in a subject according to embodiment 44, wherein said immune response comprises antibodies that are capable of neutralizing the SARS-CoV-2 virus.
47. The method of inducing an immune response against a SARS-CoV-2 virus in a subject according to embodiment 44, wherein the immunologically effective amount of a recombinant poxvirus is capable of protecting the subject from SARS-CoV-2 virus.
48. The method of inducing an immune response against a SARS-CoV-2 virus in a subject according to embodiment 44, wherein the immunologically effective amount of a recombinant poxvirus reduces or prevents the progression of the virus after SARS-CoV-2 infection in the subject.
49. The method of inducing an immune response against a SARS-CoV-2 virus in a subject according to embodiment 44, wherein the immune response is a T-cell immune response.
50. A method of inducing an immune response against a SARS-CoV-2 virus and a poxvirus comprising administering to said subject an immunologically effective amount of a recombinant poxvirus according to any one of embodiments 1-24 or the pharmaceutical composition according to any one of embodiments 25-29.
51. The method of inducing an immune response against the SARS-CoV-2 virus and the poxvirus according to embodiment 50, wherein said immunologically effective amount of the recombinant poxvirus is administered by scarification.
52. The method of inducing an immune response against the SARS-CoV-2 virus and the poxvirus according to embodiment 50, wherein said immune response comprises antibodies that are capable of neutralizing the SARS-CoV-2 virus and the poxvirus.
53. The method of inducing an immune response against the SARS-CoV-2 virus and the poxvirus according to embodiment 50, wherein the immunologically effective amount of a recombinant poxvirus is capable of protecting the subject from the SARS-CoV-2 virus and the poxvirus.
54. The method of inducing an immune response against the SARS-CoV-2 virus and the poxvirus according to embodiment 50, wherein the immunologically effective amount of a recombinant poxvirus reduces or prevents the progression of the SARS-CoV-2 virus infection and/or the poxvirus infection in the subject.
55. The method of inducing an immune response against the SARS-CoV-2 virus and the poxvirus according to embodiment 50, wherein the immune response is a T-cell immune response.
56. The method of inducing an immune response against the SARS-CoV-2 virus and the poxvirus according to any one of embodiments 50-55, wherein the poxvirus is vaccinia virus, variola, horsepox virus or monkeypox virus.
57. A method of inducing T cell immunity against a SARS-CoV-2 virus comprising administering to said subject an immunologically effective amount of a recombinant poxvirus according to any one of embodiments 1-24 or the pharmaceutical composition according to any one of embodiments 25-29.
58. The method of inducing T cell immunity against a SARS-CoV-2 virus according to embodiment 57, wherein said immunologically effective amount of the recombinant poxvirus is administered by scarification.
59. The method of inducing T cell immunity against a SARS-CoV-2 virus according to embodiment 57, wherein the immunologically effective amount of a recombinant poxvirus is capable of protecting the subject from SARS-CoV-2 virus.
60. The method of inducing T cell immunity against a SARS-CoV-2 virus according to embodiment 57, wherein the immunologically effective amount of a recombinant poxvirus reduces or prevents the progression of the SARS-CoV-2 infection in the subject.
61. A method of inducing T cell immunity against a SARS-CoV-2 virus and a poxvirus comprising administering to said subject an immunologically effective amount of a recombinant poxvirus according to any one of embodiments 1-24 or the pharmaceutical composition according to any one of embodiments 25-29.
62. The method of inducing T cell immunity against the SARS-CoV-2 virus and the poxvirus according to embodiment 61, wherein said immunologically effective amount of the recombinant poxvirus is administered by scarification.
63. The method of inducing T cell immunity against the SARS-CoV-2 virus and the poxvirus according to embodiment 61, wherein the immunologically effective amount of a recombinant poxvirus is capable of protecting the subject from the SARS-CoV-2 virus and the poxvirus.
64. The method of inducing T cell immunity against the SARS-CoV-2 virus and the poxvirus according to embodiment 61, wherein the immunologically effective amount of a recombinant poxvirus reduces or prevents the progression of the SARS-CoV-2 infection and/or poxvirus infection in the subject.
65. The method of inducing T cell immunity against the SARS-CoV-2 virus and the poxvirus according to any one of embodiments 61-64, wherein the poxvirus is vaccinia virus, variola, horsepox virus or monkeypox virus.
66. A method of generating a recombinant poxvirus according to any one of embodiments 1-65, the method comprising:
   (a) Infecting a host cell with a poxvirus;
   (b) Transfecting the infected cell of step (a) with a nucleic acid encoding a SARS-CoV-2 virus protein to generate a recombinant poxvirus; and
   (c) Selecting a recombinant poxvirus, wherein the nucleic acid encoding a SARS-CoV-2 virus protein is located, upon transfection, in a region of the poxvirus that is not essential for the replication of the poxvirus.
67. The method according to any one of embodiments 39-66, wherein the SARS-CoV-2 protein is selected from the group consisting of the S spike protein, the M protein and the N protein, or combinations of two or more of said proteins.
68. The method according to any one of embodiments 39-67, wherein the poxvirus is an orthopoxvirus.
69. The method according to embodiment 68, wherein the orthopoxvirus is selected from the group consisting of camelpox (CMLV) virus, cowpox virus (CPXV), ectromelia virus (ECTV), horsepox virus (HPXV), monkeypox virus (MPXV), vaccinia virus (VACV), variola virus (VARV), rabbitpox virus (RPXV), raccoon poxvirus, skunkpox virus, Taterapox virus, Uasin Gishu disease virus and volepox virus.
70. The method according to embodiment 68, wherein the orthopoxvirus is a horsepox virus.
71. The method according to embodiment 70, wherein the horsepox virus is strain MNR-76.
72. The method according to embodiment 68, wherein the orthopoxvirus is a vaccinia virus.
73. The method according to embodiment 72, wherein the vaccinia virus is selected from the group of strains consisting of: Western Reserve, Western Reserve Clone 3, Tian Tian, Tian Tian clone TP5, Tian Tian clone TP3, NYCBH, NYCBH clone Acambis 2000, Wyeth, Copenhagen, Lister, Lister 107, Lister-LO, Lister GL-ONC1, Lister GL-ONC2, Lister GL-ONC3, Lister GL-ONC4, Lister CTC1, Lister IMG2 (Turbo FP635), IHD-W, LC16m18, Lederle, Tashkent clone TKT3, Tashkent clone TKT4, USSR, Evans, Praha, L-IVP, V-VET1 or LIVP 6.1.1, Ikeda, EM-63, Malbran, Duke, 3737, CV-1, Connaught Laboratories, Serro 2, CM-01, NYCBH Dryvax clone DPP13, NYCBH Dryvax clone DPP15, NYCBH Dryvax clone DPP20, NYCBH Dryvax clone DPP17, NYCBH Dryvax clone DPP21, VACV-IOC, Chorioallantoid Vaccinia virus Ankara (CVA), Modified vaccinia Ankara (MVA), and MVA-BN.
74. The method according to any one of embodiments 39-73, wherein the nucleic acid encoding a SARS-CoV-2 virus protein is located in a region of the poxvirus that is not essential for replication of the poxvirus.
75. The method according to embodiment 74, wherein the nucleic acid encoding a SARS-CoV-2 virus protein is located in the thymidine kinase (TK) gene locus of the poxvirus.
76. The method according to embodiment 74, wherein the nucleic acid encoding a SARS-CoV-2 virus protein is located in the B22R homolog gene locus of the poxvirus.
77. The method according to any one of embodiments 39-76, wherein the nucleic acid encoding a SARS-CoV-2 virus protein is operatively linked to a promoter.
78. The method according to embodiment 77, wherein the promoter is a poxvirus specific promoter.
79. The method according to embodiment 78, wherein the poxvirus specific promoter is a vaccinia virus early promoter.
80. The method according to embodiment 78, wherein the poxvirus specific promoter is a vaccinia virus late promoter.
81. The method according to embodiment 78, wherein the poxvirus specific promoter is a tandem of a vaccinia virus early and late promoter.
82. The method according to any one of embodiments 39-81, wherein the poxvirus is a synthetic poxvirus.
83. A method of reducing or preventing the progression of a SARS-CoV-2 virus infection in a subject in need or at risk thereof comprising administering to said subject an immunologically effective amount of the recombinant poxvirus according to any one of embodiments 1-24 or the pharmaceutical composition according to any one of embodiments 25-29.
84. A method of reducing or preventing the progression of a SARS-CoV-2 virus and a poxvirus infection in a subject in need or at risk thereof comprising administering to said subject an immunologically effective amount of the recombinant poxvirus according to any one of embodiments 1-24 or the pharmaceutical composition of any one of embodiments 25-29.
85. The method of reducing or preventing the progression of a SARS-CoV-2 virus and a poxvirus, wherein the poxvirus is vaccinia virus, variola, horsepox virus or monkeypox virus.
86. A vaccine against a SARS-CoV-2 virus comprising a recombinant virus according to embodiments 1-24 or a pharmaceutical composition according to embodiments 25-29.
87. A bivalent vaccine against a SARS-CoV-2 virus and a poxvirus comprising a recombinant virus according to embodiments 1-24 or a pharmaceutical composition according to embodiments 25-29.
88. A bivalent vaccine against a SARS-CoV-2 virus and a poxvirus, wherein the poxvirus is a vaccinia virus, variola, horsepox virus or monkeypox.

1. A recombinant poxvirus comprising a nucleic acid encoding a SARS-CoV-2 virus protein, wherein the SARS-CoV-2 protein is selected from the group consisting of the spike protein (S), the membrane protein (M) and the nucleocapsid protein (N), or combinations of two or more of said proteins.

2. The recombinant poxvirus according to claim 1, wherein the poxvirus is an orthopoxvirus.

3. The recombinant poxvirus according to claim 2, wherein the orthopoxvirus is selected from the group consisting of camelpox (CMLV) virus, cowpox virus (CPXV), ectromelia virus (ECTV), horsepox virus (HPXV), monkeypox virus (MPXV), vaccinia virus (VACV), variola virus (VARV), rabbitpox virus (RPXV), raccoon poxvirus, skunkpox virus, Taterapox virus, Uasin Gishu disease virus and volepox virus.

4. The recombinant poxvirus according to claim 2, wherein the orthopoxvirus is a horsepox virus or a vaccinia virus.

5. The recombinant poxvirus according to claim 4, wherein the horsepox virus is strain MNR-76 and wherein the vaccinia virus is selected from the group of strains consisting of: Western Reserve, Western Reserve Clone 3, Tian Tian, Tian Tian clone TP5, Tian Tian clone TP3, NYCBH, NYCBH clone Acambis 2000 (ACAM 2000), Wyeth, Copenhagen, Lister, Lister 107, Lister-LO, Lister GL-ONC1, Lister GL-ONC2, Lister GL-ONC3, Lister GL-ONC4, Lister CTC1, Lister IMG2 (Turbo FP635), IHD-W, LC16m18, Lederle, Tashkent clone TKT3, Tashkent clone TKT4, USSR, Evans, Praha, L-IVP, V-VET1 or LIVP 6.1.1, Ikeda, EM-63, Malbran, Duke, 3737, CV-1, Connaught Laboratories, Serro 2, CM-01, NYCBH Dryvax clone DPP13, NYCBH Dryvax clone DPP15, NYCBH Dryvax clone DPP20, NYCBH Dryvax clone DPP17, NYCBH Dryvax clone DPP21, VACV-IOC, Mulford 1902, Chorioallantoid Vaccinia virus Ankara (CVA), Modified vaccinia Ankara (MVA), and MVA-BN.

6-7. (canceled)

8. The recombinant poxvirus according to claim 1, wherein the SARS-CoV-2 protein is the S protein.

9. The recombinant poxvirus according to claim 1, wherein the amino acid sequence of the SARS-CoV-2 virus protein is modified with reference to a wild type protein or modified to infect mice.

10. (canceled)

11. The recombinant poxvirus according to claim 8, wherein the amino acid sequence of the SARS-CoV-2 virus S protein comprises one or more substitutions selected from Y459H, D614G, S943P, K986P and V987P, with reference to a wild type S protein (SEQ ID NO: 47).

12. The recombinant poxvirus according to claim 1, wherein the nucleic acid encoding the SARS-CoV-2 virus protein is located in a region of the poxvirus that is not essential for replication of the poxvirus.

13. The recombinant poxvirus according to claim 12, wherein the nucleic acid encoding a SARS-CoV-2 virus protein is located in the thymidine kinase (TK) gene locus of the poxvirus or in the B22R homolog gene locus of the poxvirus.

14. (canceled)

15. The recombinant poxvirus according to claim 1, wherein the nucleic acid encoding the SARS-CoV-2 virus protein is operatively linked to a promoter.

16. The recombinant poxvirus according to claim 15, wherein the promoter is a poxvirus-specific promoter.

17. The recombinant poxvirus according to claim 16, wherein the poxvirus specific promoter is a vaccinia virus early promoter, a vaccinia virus late promoter, or a tandem of a vaccinia virus early and late promoter.

18-19. (canceled)

20. The recombinant poxvirus according to claim 1, wherein the poxvirus is a synthetic poxvirus.

21. The recombinant poxvirus according to claim 20, wherein the synthetic poxvirus is selected from the group consisting of TNX-2200 (synVACVΔA2K105^{SARS-CoV2-Spike-co}), TNX-2200 clone 1.1.1.1.1, TNX-2200 clone 2.1.1.1.1, TNX-1800 (scHPXVΔ200^{SARS-COV2-Spike-co}), TNX-1800a, TNX-1800a-1, TNX-1800b, and TNX-1800b-2.

22. The recombinant poxvirus according to claim 21, wherein the recombinant poxvirus is TNX-1800b-2 or TNX-1800a-1.

23. (canceled)

24. The recombinant poxvirus according to claim 20, wherein the synthetic poxvirus comprises any one of SEQ ID NOs: 63, 64 or 65.

25. A pharmaceutical composition comprising a recombinant poxvirus according to claim 1 and a pharmaceutically acceptable carrier.

26-29. (canceled)

30. A cell infected with a recombinant poxvirus according to claim 1, wherein the cell is an adherent cell or a suspension cell.

31. The cell according to claim 30, wherein the cell is a mammalian cell or an avian cell.

32. The cell according to claim 31, wherein the mammalian cell is a Vero cell, a Vero E6 cell, a BSC-40 cell, a Vero adherent cell, a Vero suspension cell, a BHK-21 cell, an ACE2 Knockout Vero cell, or an MRC-5 cell, and wherein the avian cell is a chicken embryo fibroblast, a duck embryo-derived cell, an EB66® cell, an AGE1.CRpIX® cell, or a DF-1 cell.

33-38. (canceled)

39. A method for selecting a cell that expresses a SARS-CoV-2 virus protein, comprising infecting a cell with a recombinant poxvirus according to claim 1 and selecting the infected cell expressing said SARS-CoV-2 virus protein.

40-43. (canceled)

44. A method of inducing an immune response against a SARS-CoV-2 virus or a SARS-CoV-2 virus and a poxvirus in a subject, comprising administering to said subject an immunologically effective amount of the recombinant poxvirus according to claim 1.

45. The method of inducing an immune response against a SARS-CoV-2 virus or a SARS-CoV-2 virus and a poxvirus in a subject according to claim 44, wherein said immunologically effective amount of the recombinant poxvirus is administered by scarification.

46. The method of inducing an immune response against a SARS-CoV-2 virus or a SARS-CoV-2 virus and a poxvirus in a subject according to claim 44, wherein said immune response comprises antibodies that are capable of neutralizing the SARS-CoV-2 virus or a SARS-CoV-2 virus and a poxvirus.

47. The method of inducing an immune response against a SARS-CoV-2 virus or a SARS-CoV-2 virus and a poxvirus in a subject according to claim 44, wherein the immunologically effective amount of a recombinant poxvirus is capable of protecting the subject from SARS-CoV-2 virus or a SARS-CoV-2 virus and a poxvirus, or reducing or preventing the progression of a SARS-CoV-2 virus or a SARS-COV-2 and poxvirus infection in the subject.

48. (canceled)

49. The method of inducing an immune response against a SARS-CoV-2 virus or a SARS-CoV-2 virus and a poxvirus in a subject according to claim 44, wherein the immune response is a T-cell immune response.

50-55. (canceled)

56. The method of inducing an immune response against a SARS-CoV-2 virus or a SARS-CoV-2 virus and a poxvirus according to claim 44, wherein the poxvirus is vaccinia virus, variola, horsepox virus or monkeypox virus.

57. A method of inducing T cell immunity against a SARS-CoV-2 virus or a SARS-CoV-2 virus and a poxvirus comprising administering to said subject an immunologically effective amount of a recombinant poxvirus according to claim 1.

58. The method of inducing T cell immunity against a SARS-CoV-2 virus or a SARS-CoV-2 virus and a poxvirus according to claim 57, wherein said immunologically effective amount of the recombinant poxvirus is administered by scarification.

59. The method of inducing T cell immunity against a SARS-CoV-2 virus or a SARS-CoV-2 virus and a poxvirus according to claim 57, wherein the immunologically effective amount of a recombinant poxvirus is capable of protecting the subject from SARS-CoV-2 virus or a SARS-CoV-2 virus and a poxvirus, or reduces or prevents the progression of a SARS-CoV-2 virus or a SARS-CoV-2 and a poxvirus infection in the subject.

60-64. (canceled)

65. The method of inducing T cell immunity against a SARS-CoV-2 virus or SARS-CoV-2 virus and a poxvirus according to claim 57, wherein the poxvirus is vaccinia virus, variola, horsepox virus or monkeypox virus.

66. A method of generating a recombinant poxvirus according to claim 1, the method comprising:
(a) Infecting a host cell with a poxvirus;
(b) Transfecting the infected cell of step (a) with a nucleic acid encoding a SARS-CoV-2 virus protein to generate a recombinant poxvirus; and
(c) Selecting a recombinant poxvirus, wherein the nucleic acid encoding a SARS-CoV-2 virus protein is located, upon transfection, in a region of the poxvirus that is not essential for the replication of the poxvirus.

67-82. (canceled)

83. A method of reducing or preventing the progression of a SARS-CoV-2 virus infection or a SARS-CoV-2 and poxvirus infection in a subject in need or at risk thereof comprising administering to said subject an immunologically effective amount of the recombinant poxvirus according to claim 1.

84-85. (canceled)

86. A vaccine against a SARS-CoV-2 virus comprising a recombinant virus according to claim 1.

87. A bivalent vaccine against a SARS-CoV-2 virus and a poxvirus comprising a recombinant virus according to claim 1.

88. (canceled)
